SEQUENCE LISTING (159 sequences)

SEQ ID NO: 1 - protein, 45 aa, Homo sapiens
GTINVHDVETQFNQYKTEAASRYNLTISDVSVSDVPFPFSAQSGA

SEQ ID NO: 2 - protein, 35 aa, Homo sapiens
QFNQYKTEAASRYNLTISDVSVSDVPFPFSAQSGA

SEQ ID NO: 3 - protein, 35 aa, Homo sapiens
GTINVHDVETQFNQYKTEAASRYNLTISDVSVSDV

SEQ ID NO: 4 - protein, 65 aa, Homo sapiens
SNIKFRPGSVVVQLTLAFREGTINVHDVETQFNQYKTEAASRYNLTISDVSVSDVPFPFSAQSGA

SEQ ID NO: 5 - protein, 454 aa, Mus sp.
EVQLEESGGGLVKPGGSLKLSCAASGFTFSGYAMSWVRQTPEKRLEWVATISSGGTYIYYPDSVKGRFTISRDNAKNTLYLQMSSLRSEDTAMYYCARLGGDNYYEYFDVWGAGTTVTVSSAKTTPPSVYAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 6 - protein, 130 aa, Mus sp.
EVQLEESGGGLVKPGGSLKLSCAASGFTFSGYAMSWVRQTPEKRLEWVATISSGGTYIYYPDSVKGRFTISRDNAKNTLYLQMSSLRSEDTAMYYCARLGGDNYYEYFDVWGAGTTVTVSSAKTTPPSVY

SEQ ID NO: 7 - protein, 226 aa, Mus sp.
DIVITQSTASLGVSLGQRATISCRASKSVSTSGYSYMHWYQQRPGQPPKLLIYLASNLESGVPARFSGSGSGTDFTLNIHPVEEEDAATYYCQHSRELPFTFGGGTKLEIKRADAAPTVSADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 120 aa, Mus sp.
DIVITQSTASLGVSLGQRATISCRASKSVSTSGYSYMHWYQQRPGQPPKLLIYLASNLESGVPARFSGSGSGTDFTLNIHPVEEEDAATYYCQHSRELPFTFGGGTKLEIKRADAAPTVS

SEQ ID NO: 9 - protein, 121 aa, synthetic construct
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 10 - protein, 113 aa, synthetic construct
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 11 - protein, 5 aa, Mus sp.
GYAMS

SEQ ID NO: 12 - protein, 17 aa, Mus sp.
TISSGGTYIYYPDSVKG

SEQ ID NO: 13 - protein, 12 aa, Mus sp.
LGGDNYYEYFDV

SEQ ID NO: 14 - protein, 15 aa, Mus sp.
RASKSVSTSGYSYMH

SEQ ID NO: 15 - protein, 7 aa, Mus sp.
LASNLES

SEQ ID NO: 16 - protein, 9 aa, Mus sp.
QHSRELPFT

SEQ ID NO: 17 - protein, 249 aa, synthetic construct
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSSGGGGSGGGGSGGGGSDIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRT

SEQ ID NO: 18 - protein, 249 aa, synthetic construct
DIVLTQSPASLAVSPGQRATITCRASKSVSTSGYSYMHWYQQKPGQPPKLLIYLASNLESGVPARFSGSGSGTDFTLTINPVEANDTANYYCQHSRELPFTFGGGTKVEIKRTGGGGSGGGGSGGGGSEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYFDVWGKGTTVTVSS

SEQ ID NO: 19 - protein, 456 aa, Mus sp.
EVMLVESGGGLVKPGGSLKLSCAASGFTFSTYAMSWIRQTPEKRLEWVASIGRAGSTYYSDSVKGRFTISRDNVRNILYLQMSSLRSEDTAMYYCARGPIYNDYDEFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 20 - protein, 120 aa, Mus sp.
EVMLVESGGGLVKPGGSLKLSCAASGFTFSTYAMSWIRQTPEKRLEWVASIGRAGSTYYSDSVKGRFTISRDNVRNILYLQMSSLRSEDTAMYYCARGPIYNDYDEFAYWGQGTLVTVSA

SEQ ID NO: 21 - protein, 277 aa, Mus sp.
ADAAPTVSIFPPSSEQLTSGGASVVCFLNNFYPKDINVKWKIDGSERQNGVLNSWTDQDNIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 112 aa, Mus sp.
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELK

SEQ ID NO: 23 - protein, 120 aa, synthetic construct
QVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSS

SEQ ID NO: 24 - protein, 113 aa, synthetic construct
EIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKR

SEQ ID NO: 25 - protein, 5 aa, Mus sp.
TYAMS

SEQ ID NO: 26 - protein, 16 aa, Mus sp.
SIGRAGSTYYSDSVKG

SEQ ID NO: 27 - protein, 12 aa, Mus sp.
GPIYNDYDEFAY

SEQ ID NO: 28 - protein, 17 aa, Mus sp.
KSSQSVLYSSNQKNYLA

SEQ ID NO: 29 - protein, 7 aa, Mus sp.
WASTRES

SEQ ID NO: 30 - protein, 8 aa, Mus sp.
HQYLSSLT

SEQ ID NO: 31 - protein, 248 aa, synthetic construct
QVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSSGGGGSGGGGSGGGGSEIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKR

SEQ ID NO: 32 - protein, 248 aa, synthetic construct
EIVLTQSPATLSLSPGERATLSCKSSQSVLYSSNQKNYLAWYQQKPGQAPRLLIYWASTRESGIPARFSGSGSGTDFTLTISSLEPEDFAVYYCHQYLSSLTFGGGTKVEIKRGGGGSGGGGSGGGGSQVQLVESGGGLVKPGGSLRLSCAASGFTFSTYAMSWIRQAPGKGLEWVASIGRAGSTYYSDSVKGRFTISRDNAKNSLYLQMNSLRAEDTAVYYCARGPIYNDYDEFAYWGQGTLVTVSS

SEQ ID NO: 33 - protein, 122 aa, Mus sp.
EVKVVESGGDLVKPGGSLKLSCVVSGFTFSRYGMSWVRQTPGKRLEWVATISGGGTYIYYPDSVKGRFTISRDNAKNTLYLQMSSLKSEDTAMYHCTRDNYGRNYDYGMDYWGQGTSVTVSS

SEQ ID NO: 34 - protein, 106 aa, Mus sp.
QIVLTQSPAIMSASPGEEVTLTCSATSSVSYIHWFQQRPGTSPKLWIYSTSNLASGVPVRFSGSGYGTSYSLTISRMEAEDAATYYCQQRSSSPFTFGSGTKLEIK

SEQ ID NO: 35 - protein, 122 aa, synthetic construct
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 36 - protein, 106 aa, synthetic construct
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 37 - protein, 5 aa, Mus sp.
RYGMS

SEQ ID NO: 38 - protein, 17 aa, Mus sp.
TISGGGTYIYYPDSVKG

SEQ ID NO: 39 - protein, 13 aa, Mus sp.
DNYGRNYDYGMDY

SEQ ID NO: 40 - protein, 10 aa, Mus sp.
SATSSVSYIH

SEQ ID NO: 41 - protein, 7 aa, Mus sp.
STSNLAS

SEQ ID NO: 42 - protein, 9 aa, Mus sp.
QQRSSSPFT

SEQ ID NO: 43 - protein, 243 aa, synthetic construct
EVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSSGGGGSGGGGSGGGGSEIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIK

SEQ ID NO: 44 - protein, 243 aa, synthetic construct
EIVLTQSPATLSLSPGERATLTCSATSSVSYIHWYQQRPGQSPRLLIYSTSNLASGIPARFSGSGSGSDYTLTISSLEPEDFAVYYCQQRSSSPFTFGSGTKVEIKGGGGSGGGGSGGGGSEVQLVESGGGLVKPGGSLRLSCAASGFTFSRYGMSWVRQAPGKRLEWVSTISGGGTYIYYPDSVKGRFTISRDNAKNTLYLQMNSLRAEDTAVYYCTRDNYGRNYDYGMDYWGQGTLVTVSS

SEQ ID NO: 45 - protein, 121 aa, Mus sp.
EVMLVESGGGLVKPGGSLKLSCAASGITFSTYTMSWVRQTPEKRLEWVATISTGGDKTYYSDSVKGRFTISRDNAKNNLYLQMSSLRSEDTALYYCARGTTAMYYYAMDYWGQGTSVTVSS

SEQ ID NO: 46 - protein, 111 aa, Mus sp.
DIVLTQSPASLAVSLGQRATISCRASKSISTSDYNYIHWYQQKPGQPPKLLIYLASNLESGVPARFSGSGSGTDFTLNIHPVEEEDAATYYCQHSRELPLTFGAGTKLELK

SEQ ID NO: 47 - protein, 121 aa, synthetic construct
EVQLVESGGGLVKPGGSLRLSCAASGITFSTYTMSWVRQAPGKGLEWVATISTGGDKTYYSDSVKGRFTISRDNAKNTLYLQMNSLRAEDTAVYYCARGTTAMYYYAMDYWGQGTTVTVSS

SEQ ID NO: 48 - protein, 111 aa, synthetic construct
DIVLTQSPASLAVSPGQRATITCRASKSISTSDYNYIHWYQQKPGQPPKLLIYLASNLESGVPARFSGSGSGTDFTLTISPVEAEDAATYYCQHSRELPLTFGGGTKVEIK

SEQ ID NO: 49 - protein, 9 aa, Mus sp.
ITFSTYTMS

SEQ ID NO: 50 - protein, 17 aa, Mus sp.
TISTGGDKTYYSDSVKG

SEQ ID NO: 51 - protein, 12 aa, Mus sp.
GTTAMYYYAMDY

SEQ ID NO: 52 - protein, 15 aa, Mus sp.
RASKSISTSDYNYIH

SEQ ID NO: 53 - protein, 7 aa, Mus sp.
LASNLES

SEQ ID NO: 54 - protein, 9 aa, Mus sp.
QHSRELPLT

SEQ ID NO: 55 - protein, 247 aa, synthetic construct
EVQLVESGGGLVKPGGSLRLSCAASGITFSTYTMSWVRQAPGKGLEWVATISTGGDKTYYSDSVKGRFTISRDNAKNTLYLQMNSLRAEDTAVYYCARGTTAMYYYAMDYWGQGTTVTVSSGGGGSGGGGSGGGGSDIVLTQSPASLAVSPGQRATITCRASKSISTSDYNYIHWYQQKPGQPPKLLIYLASNLESGVPARFSGSGSGTDFTLTISPVEAEDAATYYCQHSRELPLTFGGGTKVEIK

SEQ ID NO: 56 - protein, 247 aa, synthetic construct
DIVLTQSPASLAVSPGQRATITCRASKSISTSDYNYIHWYQQKPGQPPKLLIYLASNLESGVPARFSGSGSGTDFTLTISPVEAEDAATYYCQHSRELPLTFGGGTKVEIKGGGGSGGGGSGGGGSEVQLVESGGGLVKPGGSLRLSCAASGITFSTYTMSWVRQAPGKGLEWVATISTGGDKTYYSDSVKGRFTISRDNAKNTLYLQMNSLRAEDTAVYYCARGTTAMYYYAMDYWGQGTTVTVSS

SEQ ID NO: 57 - protein, 118 aa, Mus sp.
EVMLVESGGGLVKPGGSLKLSCAASGFTFSTYAMSWVRQTPEKRLEWVAAISNGGGYTYYPDSLKGRFTISRDNAKNTLYLQMSSLRSEDTATYYCARRYYDHYFDYWGQGTALTVSS

SEQ ID NO: 58 - protein, 112 aa, Mus sp.
DVLMTQTPLSLPVSLGDQASISCRSSQTIVHSNGNTYLEWYLQKPGQSPKLLIYKVSNRFSGVPDRFSGSGSGTDFTLKISRVEAEDLGVYYCFQDSHVPLTFGAGTKLELK

SEQ ID NO: 59 - protein, 118 aa, synthetic construct
EVQLVESGGGLVKPGGSLRLSCAASGFTFSTYAMSWVRQAPGKGLEWVAAISNGGGYTYYPDSLKGRFTISRDNSKNTLYLQMNSLRAEDTAVYYCARRYYDHYFDYWGQGTLVTVSS

SEQ ID NO: 60 - protein, 112 aa, synthetic construct
DIVMTQSPSSLSVSPGERASISCRSSQTIVHSNGNTYLEWYQQKPGQSPKLLIYKVSNRFSGVPDRFSGSGSGTDFTLTISGVEAEDVGVYYCFQDSHVPLTFGGGTKVEIK

SEQ ID NO: 61 - protein, 9 aa, Mus sp.
FTFSTYAMS

SEQ ID NO: 62 - protein, 17 aa, Mus sp.
AISNGGGYTYYPDSLKG

SEQ ID NO: 63 - protein, 9 aa, Mus sp.
RYYDHYFDY

SEQ ID NO: 64 - protein, 16 aa, Mus sp.
RSSQTIVHSNGNTYLE

SEQ ID NO: 65 - protein, 7 aa, Mus sp.
KVSNRFS

SEQ ID NO: 66 - protein, 9 aa, Mus sp.
FQDSHVPLT

SEQ ID NO: 67 - protein, 245 aa, synthetic construct
EVQLVESGGGLVKPGGSLRLSCAASGFTFSTYAMSWVRQAPGKGLEWVAAISNGGGYTYYPDSLKGRFTISRDNSKNTLYLQMNSLRAEDTAVYYCARRYYDHYFDYWGQGTLVTVSSGGGGSGGGGSGGGGSDIVMTQSPSSLSVSPGERASISCRSSQTIVHSNGNTYLEWYQQKPGQSPKLLIYKVSNRFSGVPDRFSGSGSGTDFTLTISGVEAEDVGVYYCFQDSHVPLTFGGGTKVEIK

SEQ ID NO: 68 - protein, 245 aa, synthetic construct
DIVMTQSPSSLSVSPGERASISCRSSQTIVHSNGNTYLEWYQQKPGQSPKLLIYKVSNRFSGVPDRFSGSGSGTDFTLTISGVEAEDVGVYYCFQDSHVPLTFGGGTKVEIKGGGGSGGGGSGGGGSEVQLVESGGGLVKPGGSLRLSCAASGFTFSTYAMSWVRQAPGKGLEWVAAISNGGGYTYYPDSLKGRFTISRDNSKNTLYLQMNSLRAEDTAVYYCARRYYDHYFDYWGQGTLVTVSS

SEQ ID NO: 69 - protein, 119 aa, Mus sp.
EVQLVESGGDLVKPGGSLKLSCAASGFTFSSYGMSWVRQTPDKRLEWVATISNGGRHTFYPDSVKGRFTISRDNAKNTLYLQMSSLKSEDTAMYLCVRQTGTEGWFAYWGQGTLVTVSA

SEQ ID NO: 70 - protein, 112 aa, Mus sp.
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 71 - protein, 119 aa, synthetic construct
EVQLVESGGGLVKPGGSLRLSCAASGFTFSSYGMSWVRQAPGKGLEWVATISNGGRHTFYPDSVKGRFTISRDNAKNTLYLQMNSLRAEDTAVYLCVRQTGTEGWFAYWGQGTLVTVSS

SEQ ID NO: 72 - protein, 112 aa, synthetic construct
DVVMTQTPLSLSVTPGQPASISCKSSQSLLDSDGKTYLNWLLQKPGQSPKRLIYLVSKLDSGVPDRFSGSGSGTDFTLKISRVEAEDVGVYYCWQGTHFPQTFGGGTKVEIK

SEQ ID NO: 73 - protein, 5 aa, Mus sp.
SYGMS

SEQ ID NO: 74 - protein, 17 aa, Mus sp.
TISNGGRHTFYPDSVKG

SEQ ID NO: 75 - protein, 10 aa, Mus sp.
QTGTEGWFAY

SEQ ID NO: 76 - protein, 16 aa, Mus sp.
KSSQSLLDSDGKTYLN

SEQ ID NO: 77 - protein, 7 aa, Mus sp.
LVSKLDS

SEQ ID NO: 78 - protein, 9 aa, Mus sp.
WQGTHFPQT

SEQ ID NO: 79 - protein, 246 aa, synthetic construct
EVQLVESGGGLVKPGGSLRLSCAASGFTFSSYGMSWVRQAPGKGLEWVATISNGGRHTFYPDSVKGRFTISRDNAKNTLYLQMNSLRAEDTAVYLCVRQTGTEGWFAYWGQGTLVTVSSGGGGSGGGGSGGGGSDVVMTQTPLSLSVTPGQPASISCKSSQSLLDSDGKTYLNWLLQKPGQSPKRLIYLVSKLDSGVPDRFSGSGSGTDFTLKISRVEAEDVGVYYCWQGTHFPQTFGGGTKVEIK

SEQ ID NO: 80 - protein, 246 aa, synthetic construct
DVVMTQTPLSLSVTPGQPASISCKSSQSLLDSDGKTYLNWLLQKPGQSPKRLIYLVSKLDSGVPDRFSGSGSGTDFTLKISRVEAEDVGVYYCWQGTHFPQTFGGGTKVEIKGGGGSGGGGSGGGGSEVQLVESGGGLVKPGGSLRLSCAASGFTFSSYGMSWVRQAPGKGLEWVATISNGGRHTFYPDSVKGRFTISRDNAKNTLYLQMNSLRAEDTAVYLCVRQTGTEGWFAYWGQGTLVTVSS

SEQ ID NO: 81 - protein, 119 aa, Mus sp.
QVQLQQSGPELVRPGVSVKISCKGSGYRFTDYAMNWVKQSHAKSLEWIGVISTFSGNTNFNQKFKGKATMTVDKSSSTAYMELARLTSEDSAMYYCARSDYYGPYFDYWGQGTTLTVSS

SEQ ID NO: 82 - protein, 112 aa, Mus sp.
DVLMTQTPLSLPVSLGDQASISCRSSQTIVHSNGNTYLEWYLQKPGQSPKLLIYKVSNRFSGVPDRFSGSGSGTDFTLKINRVEAEDLGVYYCFQGSHVPFTFGSGTKLEIK

SEQ ID NO: 83 - protein, 119 aa, synthetic construct
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSS

SEQ ID NO: 84 - protein, 114 aa, synthetic construct
DIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 85 - protein, 5 aa, Mus sp.
DYAMN

SEQ ID NO: 86 - protein, 17 aa, Mus sp.
VISTFSGNTNFNQKFKG

SEQ ID NO: 87 - protein, 10 aa, Mus sp.
SDYYGPYFDY

SEQ ID NO: 88 - protein, 16 aa, Mus sp.
RSSQTIVHSNGNTYLE

SEQ ID NO: 89 - protein, 7 aa, Mus sp.
KVSNRFS

SEQ ID NO: 90 - protein, 9 aa, Mus sp.
FQGSHVPFT

SEQ ID NO: 91 - protein, 248 aa, synthetic construct
QVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSSGGGGSGGGGSGGGGSDIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRT

SEQ ID NO: 92 - protein, 248 aa, synthetic construct
DIVMTQTPLSLSVTPGQPASISCRSSQTIVHSNGNTYLEWYLQKPGQSPQLLIYKVSNRFSGVPDRFSGSGSGTDFTLKISRVEAEDVGVYYCFQGSHVPFTFGGGTKVEIKRTGGGGSGGGGSGGGGSQVQLVQSGAEVKKPGASVKVSCKASGYTFTDYAMNWVRQAPGQGLEWMGVISTFSGNTNFNQKFKGRVTMTTDTSTSTAYMELRSLRSDDTAVYYCARSDYYGPYFDYWGQGTTLTVSS

SEQ ID NO: 93 - protein, 107 aa, Mus sp.
EVMVVESGGGLVKPGGSLKLSCAASGFTFSGYAMSWVRQTPEKRLEWVATISSGGTYIYYPDSVKGRFTISRDNAKNTLYLQMSSLRSEDTAMYYCARLGGDNYYEY

SEQ ID NO: 94 - protein, 102 aa, Mus sp.
DIVITQTPASLAVSLGQRATISYRASKSVSTSGYSYMHWNQQKPGQPPRLLIYLVSNLESGVPARFSGSGSGTDFTLNIHPVEEEDAATYYCQHIRELTRSE

SEQ ID NO: 95 - protein, 118 aa, synthetic construct
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSS

SEQ ID NO: 96 - protein, 114 aa, synthetic construct
DIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 97 - protein, 5 aa, Mus sp.
GYAMS

SEQ ID NO: 98 - protein, 17 aa, Mus sp.
TISSGGTYIYYPDSVKG

SEQ ID NO: 99 - protein, 9 aa, Mus sp.
LGGDNYYEY

SEQ ID NO: 100 - protein, 15 aa, Mus sp.
RASKSVSTSGYSYMH

SEQ ID NO: 101 - protein, 7 aa, Mus sp.
LVSNLES

SEQ ID NO: 102 - protein, 10 aa, Mus sp.
QHIRELTRSE

SEQ ID NO: 103 - protein, 247 aa, synthetic construct
EVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSSGGGGSGGGGSGGGGSDIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRT

SEQ ID NO: 104 - protein, 247 aa, synthetic construct
DIVMTQSPDSLAVSLGERATINCRASKSVSTSGYSYMHWYQQKPGQPPKLLIYLVSNLESGVPDRFSGSGSGTDFTLTISSLQAEDVAVYYCQHIRELTRSEFGGGTKVEIKRTGGGGSGGGGSGGGGSEVQLVESGGGLVKPGGSLRLSCAASGFTFSGYAMSWVRQAPGKGLEWVSTISSGGTYIYYPDSVKGRFTISRDNAKNSLYLQMNSLRAEDTAVYYCARLGGDNYYEYWGKGTTVTVSS

SEQ ID NO: 105 - protein, 114 aa, Mus sp.
QVQLQQSGAELVKPGASVKLSCKASGYTFTGYFLYWVKQRPGQGLEWIGGINPDNGGIDFNEKFRNKATLTVDKSSSTAYMQLSSLTSEDSAVYYCTLLIGNYWGQGTTLTVSS

SEQ ID NO: 106 - protein, 112 aa, Mus sp.
DVVMTQTPLTLSVTIGQPASISCKSSQSLLHSDGKTYLIWLLQRPGQSPKRLIYLVSKLDSGVPDRFTGSGSGTDFTLKISRVEAEDLGVYFCCQGTHFPWTFGGGTMLEIK

SEQ ID NO: 107 - protein, 114 aa, synthetic construct
QVQLVQSGAEVKKPGASVKVSCKASGYTFTGYFLYWVRQAPGQGLEWIGGINPDNGGIDFNEKFRNRATLTVDKSSSTAYMELSSLRSEDTAVYYCTLLIGNYWGQGTLVTVSS

SEQ ID NO: 108 - protein, 112 aa, synthetic construct
DVVMTQTPSSLSVTPGQPASISCKSSQSLLHSDGKTYLIWLQQKPGQSPQRLIYLVSKLDSGVPDRFSGSGSGTDFTLKISRVEAEDVGVYYCCQGTHFPWTFGQGTKVEIK

SEQ ID NO: 109 - protein, 5 aa, Mus sp.
GYFLY

SEQ ID NO: 110 - protein, 17 aa, Mus sp.
GINPDNGGIDFNEKFRN

SEQ ID NO: 111 - protein, 5 aa, Mus sp.
LIGNY

SEQ ID NO: 112 - protein, 16 aa, Mus sp.
KSSQSLLHSDGKTYLI

SEQ ID NO: 113 - protein, 7 aa, Mus sp.
LVSKLDS

SEQ ID NO: 114 - protein, 9 aa, Mus sp.
CQGTHFPWT

SEQ ID NO: 115 - protein, 241 aa, synthetic construct
QVQLVQSGAEVKKPGASVKVSCKASGYTFTGYFLYWVRQAPGQGLEWIGGINPDNGGIDFNEKFRNRATLTVDKSSSTAYMELSSLRSEDTAVYYCTLLIGNYWGQGTLVTVSSGGGGSGGGGSGGGGSDVVMTQTPSSLSVTPGQPASISCKSSQSLLHSDGKTYLIWLQQKPGQSPQRLIYLVSKLDSGVPDRFSGSGSGTDFTLKISRVEAEDVGVYYCCQGTHFPWTFGQGTKVEIK

SEQ ID NO: 116 - protein, 241 aa, synthetic construct
DVVMTQTPSSLSVTPGQPASISCKSSQSLLHSDGKTYLIWLQQKPGQSPQRLIYLVSKLDSGVPDRFSGSGSGTDFTLKISRVEAEDVGVYYCCQGTHFPWTFGQGTKVEIKGGGGSGGGGSGGGGSQVQLVQSGAEVKKPGASVKVSCKASGYTFTGYFLYWVRQAPGQGLEWIGGINPDNGGIDFNEKFRNRATLTVDKSSSTAYMELSSLRSEDTAVYYCTLLIGNYWGQGTLVTVSS

SEQ ID NO: 117 - protein, 114 aa, Mus sp.
QVQLQQPGAELVQPGASVKLSCKASGYTFTGYFLYWVKQRPGHGLEWIGGIHPSNGDTDFNEKFKNKATLTVDISSSTAYMQLSSLTSEDSAVYYCTLLIGVYWGQGTTLTVSS

SEQ ID NO: 118 - protein, 112 aa, Mus sp.; identical to SEQ ID NO: 106

SEQ ID NO: 119 - protein, 114 aa, synthetic construct
QVQLVQSGAEVKKPGASVKVSCKASGYTFTGYFLYWVRQAPGQGLEWIGGIHPSNGDTDFNEKFKNRATLTVDISSSTAYMELSSLRSDDTAVYYCARLIGVYWGQGTLVTVSS

SEQ ID NO: 120 - protein, 112 aa, synthetic construct
DVVMTQSPLSLSVTLGQPASISCKSSQSLLHSDGKTYLIWLQQRPGQSPRRLIYLVSKLDSGVPDRFSGSGSGTDFTLKISRVEAEDVGVYFCCQGTHFPWTFGQGTKVEIK

SEQ ID NO: 121 - protein, 5 aa, Mus sp.
GYFLY

SEQ ID NO: 122 - protein, 17 aa, Mus sp.
GIHPSNGDTDFNEKFKN

SEQ ID NO: 123 - protein, 5 aa, Mus sp.
LIGVY

SEQ ID NO: 124 - protein, 16 aa, Mus sp.
KSSQSLLHSDGKTYLI

SEQ ID NO: 125 - protein, 7 aa, Mus sp.
LVSKLDS

SEQ ID NO: 126 - protein, 9 aa, Mus sp.
CQGTHFPWT

SEQ ID NO: 127 - protein, 241 aa, synthetic construct
QVQLVQSGAEVKKPGASVKVSCKASGYTFTGYFLYWVRQAPGQGLEWIGGIHPSNGDTDFNEKFKNRATLTVDISSSTAYMELSSLRSDDTAVYYCARLIGVYWGQGTLVTVSSGGGGSGGGGSGGGGSDVVMTQSPLSLSVTLGQPASISCKSSQSLLHSDGKTYLIWLQQRPGQSPRRLIYLVSKLDSGVPDRFSGSGSGTDFTLKISRVEAEDVGVYFCCQGTHFPWTFGQGTKVEIK

SEQ ID NO: 128 - protein, 241 aa, synthetic construct
DVVMTQSPLSLSVTLGQPASISCKSSQSLLHSDGKTYLIWLQQRPGQSPRRLIYLVSKLDSGVPDRFSGSGSGTDFTLKISRVEAEDVGVYFCCQGTHFPWTFGQGTKVEIKGGGGSGGGGSGGGGSQVQLVQSGAEVKKPGASVKVSCKASGYTFTGYFLYWVRQAPGQGLEWIGGIHPSNGDTDFNEKFKNRATLTVDISSSTAYMELSSLRSDDTAVYYCARLIGVYWGQGTLVTVSS

SEQ ID NO: 129 - protein, 117 aa, Mus sp.
QVQLQQSGAELVRPGSSVKISCKASGYAFSTYWMNWVKQRPGQGLEWIGQIYPGDSDTNYNGKFKGKATLTADKSSNTAYMQLSSLTSEDSAVFFCARGNHASMDYWGQGTSVTVSS

SEQ ID NO: 130 - protein, 112 aa, Mus sp.
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQKTHVPWTFGGGTKLEIK

SEQ ID NO: 131 - protein, 117 aa, synthetic construct
EVQLVQSGAEVKKPGATVKISCKVSGYAFSTYWMNWVKQAPGKGLEWIGQIYPGDSDTNYNGKFKGRATLTADKSSNTAYMELSSLRSEDTAVYFCARGNHASMDYWGQGTLVTVSS

SEQ ID NO: 132 - protein, 112 aa, synthetic construct
DVVMTQTPSSLPVSPGERASISCRSSQSLVHSNGNTYLHWYLQKPGQSPKLLIYKVSNRFSGVPDRFSGSGSGTDFTLKISRVEAEDVGVYFCSQKTHVPWTFGQGTKVEIK

SEQ ID NO: 133 - protein, 5 aa, Mus sp.
TYWMN

SEQ ID NO: 134 - protein, 17 aa, Mus sp.
QIYPGDSDTNYNGKFKG

SEQ ID NO: 135 - protein, 8 aa, Mus sp.
GNHASMDY

SEQ ID NO: 136 - protein, 16 aa, Mus sp.
RSSQSLVHSNGNTYLH

SEQ ID NO: 137 - protein, 7 aa, Mus sp.
KVSNRFS

SEQ ID NO: 138 - protein, 9 aa, Mus sp.
SQKTHVPWT

SEQ ID NO: 139 - protein, 244 aa, synthetic construct
EVQLVQSGAEVKKPGATVKISCKVSGYAFSTYWMNWVKQAPGKGLEWIGQIYPGDSDTNYNGKFKGRATLTADKSSNTAYMELSSLRSEDTAVYFCARGNHASMDYWGQGTLVTVSSGGGGSGGGGSGGGGSDVVMTQTPSSLPVSPGERASISCRSSQSLVHSNGNTYLHWYLQKPGQSPKLLIYKVSNRFSGVPDRFSGSGSGTDFTLKISRVEAEDVGVYFCSQKTHVPWTFGQGTKVEIK

SEQ ID NO: 140 - protein, 244 aa, synthetic construct
DVVMTQTPSSLPVSPGERASISCRSSQSLVHSNGNTYLHWYLQKPGQSPKLLIYKVSNRFSGVPDRFSGSGSGTDFTLKISRVEAEDVGVYFCSQKTHVPWTFGQGTKVEIKGGGGSGGGGSGGGGSEVQLVQSGAEVKKPGATVKISCKVSGYAFSTYWMNWVKQAPGKGLEWIGQIYPGDSDTNYNGKFKGRATLTADKSSNTAYMELSSLRSEDTAVYFCARGNHASMDYWGQGTLVTVSS

SEQ ID NO: 141 - protein, 324 aa, Mus sp.
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 142 - protein, 330 aa, Mus sp.
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 143 - protein, 336 aa, Mus sp.
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 144 - protein, 106 aa, Mus sp.
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 145 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 147 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 148 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 15 aa, synthetic construct
GGGGSGGGGSGGGGS

SEQ ID NO: 151 - protein, 21 aa, synthetic construct
DKTHTKPPKPAPELLGGPGTG

SEQ ID NO: 152 - protein, 43 aa, synthetic construct
DKTHTKPPKPAPELLGGPGTGTGGPTIKPPKPPKPAPNLLGGP

SEQ ID NO: 153 - protein, 38 aa, Homo sapiens
SNIKFRPGSVVVQLTLAFREGTINVHDVETQFNQYKTE

SEQ ID NO: 154 - protein, 45 aa, Homo sapiens
VQLTLAFREGTINVHDVETQFNQYKTEAASRYNLTISDVSVSDVP

SEQ ID NO: 155 - protein, 26 aa, Homo sapiens
ASRYNLTISDVSVSDVPFPFSAQSGA

SEQ ID NO: 156 - protein, 19 aa, Homo sapiens
ISDVSVSDVPFPFSAQSGA

SEQ ID NO: 157 - protein, 15 aa, Homo sapiens
SVSDVPFPFSAQSGA

SEQ ID NO: 158 - protein, 30 aa, Homo sapiens
QFNQYKTEAASRYNLTISDVSVSDVPFPFS

SEQ ID NO: 159 - protein, 21 aa, Homo sapiens
ASRYNLTISDVSVSDVPFPFS

(19) United States
(12) Patent Application Publication
(10) Pub. No.: US 2026/0356976 A1
(43) Pub. Date: Nov. 12, 2026

(54) ANTI-MUC1* ANTIBODY DRUG COMPLEXES AND USES THEREOF

(71) Applicant: Minerva Biotechnologies Corporation, Waltham, MA (US)

(72) Inventors: Cynthia Bamdad, Boston, MA (US); Benoit Smagghe, Honolulu, HI (US); Scott Moe, Sudbury, MA (US); Thomas Jeon, Woburn, MA (US)

(21) Appl. No.: 19/117,740

(22) PCT Filed: Oct. 9, 2024
(86) PCT No.: PCT/US2024/050546

§ 371 Date: Apr. 2, 2025

Related U.S. Application Data
(60) Provisional application No. 63/589,590, filed on Oct. 11, 2023.

(51) Int. Cl. C07K 16/30; A61K 47/68; A61P 35/00

(52) U.S. Cl. CPC C07K 16/3092; A61K 47/68031; A61K 47/6849; A61K 47/6889; A61P 35/00; C07K 2317/565; C07K 2317/77

(57) ABSTRACT

Described herein are anti-MUC1* antibodies or antibody fragments and antibody drug complex (ADC) comprising an anti-MUC1* antibody conjugated via a linker to a toxin. The present disclosure also provides compositions comprising the antibodies and antibody drug complexes and methods for treating diseases and disorders such as cancer.

### CROSS-REFERENCE

This application claims the benefit of U.S. Provisional Application No. 63/589,590 filed Oct. 11, 2023, which is incorporated herein by reference in its entirety.

### SEQUENCE LISTING

The instant application contains a Sequence Listing in electronic format. The Sequence Listing is provided as a file entitled 56699-763_601_SL.xml, created on Oct. 1, 2024, which is 151,476 bytes in size. The information in the electronic format of the Sequence Listing is incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

Provided herein are antibody conjugates of the formulas described herein comprising one or more moieties derived from therapeutic agents (e.g., topoisomerase I inhibitors, inhibitors of microtubule assembly), and wherein the conjugates further comprise a polypeptide, such as an antibody. The antibody-drug conjugates may be useful for the treatment of diseases or disorder, for example, a proliferative disease such as a cancer. Also provided herein are uses and methods for treating diseases and disorders using these antibody conjugates.

Disclosed herein is an antibody drug complex (ADC) comprising an antibody conjugated via a linker to a toxin, wherein the antibody binds to a MUC1* extracellular domain. In some embodiments, the antibody binds to a peptide consisting of SEQ ID NO: 157 and to a peptide consisting of SEQ ID NO: 159. In some embodiments, the antibody is a mouse antibody. In some embodiments, the antibody is an IgG1. In some embodiments, the antibody is an IgG2b. In some embodiments, the antibody is a human or humanized antibody. In some embodiments, the antibody is an IgG1. In some embodiments, the antibody is an IgG2, IgG3 or IgG4. In some embodiments, the antibody is an scFv or a camelid antibody. In some embodiments, the antibody comprises: a heavy chain (HC) variable domain having at least 80%, at least 85%, at least 90%, at least 95%, or at least 98% sequence identity to SEQ ID NO: 9, and a light chain (LC) variable domain having at least 80%, at least 85%, at least 90%, at least 95%, or at least 98% sequence identity to SEQ ID NO: 10.

In some embodiments, the antibody comprises a binding region wherein: HC complementarity determining region (CDR) 1 comprises SEQ ID NO: 49; HC CDR2 comprises SEQ ID NO: 50; HC CDR3 comprises SEQ ID NO: 51; LC CDR1 comprises SEQ ID NO: 52; LC CDR2 comprises SEQ ID NO: 53; and LC CDR3 SEQ ID NO: 54. In some embodiments, the antibody comprises: a HC variable domain having at least 80%, at least 85%, at least 90%, at least 95%, or at least 98% sequence identity to SEQ ID NO: 47, and a LC variable domain having at least 80%, at least 85%, at least 90%, at least 95%, or at least 98% sequence identity to SEQ ID NO: 48. In some embodiments, the antibody comprises a binding region wherein: HC complementarity determining region (CDR) 1 comprises SEQ ID NO: 11; HC CDR2 comprises SEQ ID NO: 12; HC CDR3 comprises SEQ ID NO: 13; LC CDR1 comprises SEQ ID NO: 14; LC CDR2 comprises SEQ ID NO: 15; and LC CDR3 SEQ ID NO: 16. In some embodiments, the antibody comprises: a HC variable domain having at least 80%, at least 85%, at least 90%, at least 95%, or at least 98% sequence identity to SEQ ID NO: 23, and a LC variable domain having at least 80%, at least 85%, at least 90%, at least 95%, or at least 98% sequence identity to SEQ ID NO: 24. In some embodiments, the antibody comprises a binding region wherein: HC CDR1 comprises SEQ ID NO: 25; HC CDR2 comprises SEQ ID NO: 26; HC CDR3 comprises SEQ ID NO: 27; LC CDR1 comprises SEQ ID NO: 28; LC CDR2 comprises SEQ ID NO: 29; and LC CDR3 comprises SEQ ID NO: 30.

In some embodiments, the antibody comprises a binding region wherein: (a) HC CDR1 comprises SEQ ID NO: 37, HC CDR2 comprises SEQ ID NO: 38, HC CDR3 comprises SEQ ID NO: 39, LC CDR1 comprises SEQ ID NO: 40, LC CDR2 comprises SEQ ID NO: 41, and LC CDR3 comprises SEQ ID NO: 42; or (b) HC CDR1 comprises SEQ ID NO: 49, HC CDR2 comprises SEQ ID NO: 50, HC CDR3 comprises SEQ ID NO: 51, LC CDR1 comprises SEQ ID NO: 52, LC CDR2 comprises SEQ ID NO: 53, and LC CDR3 comprises SEQ ID NO: 54.

In some embodiments, the antibody comprises: (a) a HC variable domain having at least 80%, at least 85%, at least 90%, at least 95%, or at least 98% sequence identity to SEQ ID NO: 59, and a LC variable domain having at least 80%, at least 85%, at least 90%, at least 95%, or at least 98% sequence identity to SEQ ID NO: 60; (b) a HC variable domain having at least 80%, at least 85%, at least 90%, at least 95%, or at least 98% sequence identity to SEQ ID NO: 71, and a LC variable domain having at least 80%, at least 85%, at least 90%, at least 95%, or at least 98% sequence identity to SEQ ID NO: 72; (c) a HC variable domain having at least 80%, at least 85%, at least 90%, at least 95%, or at least 98% sequence identity to SEQ ID NO: 83, and a LC variable domain having at least 80%, at least 85%, at least 90%, at least 95%, or at least 98% sequence identity to SEQ ID NO: 84; (d) a HC variable domain having at least 80%, at least 85%, at least 90%, at least 95%, or at least 98% sequence identity to SEQ ID NO: 95, and a LC variable domain having at least 80%, at least 85%, at least 90%, at least 95%, or at least 98% sequence identity to SEQ ID NO: 96; (e) a HC variable domain having at least 80%, at least 85%, at least 90%, at least 95%, or at least 98% sequence identity to SEQ ID NO: 107, and a LC variable domain having at least 80%, at least 85%, at least 90%, at least 95%, or at least 98% sequence identity to SEQ ID NO: 108; or (f) a HC variable domain having at least 80%, at least 85%, at least 90%, at least 95%, or at least 98% sequence identity to SEQ ID NO: 119, and a LC variable domain having at least 80%, at least 85%, at least 90%, at least 95%, or at least 98% sequence identity to SEQ ID NO: 120.

In some embodiments, the antibody comprises a binding region wherein: (a) HC CDR1 comprises SEQ ID NO: 61, HC CDR2 comprises SEQ ID NO: 62, HC CDR3 comprises SEQ ID NO: 63, LC CDR1 comprises SEQ ID NO: 64, LC CDR2 comprises SEQ ID NO: 65, and LC CDR3 comprises SEQ ID NO: 66; (b) HC CDR1 comprises SEQ ID NO: 73, HC CDR2 comprises SEQ ID NO: 74, HC CDR3 comprises SEQ ID NO: 75, LC CDR1 comprises SEQ ID NO: 76, LC CDR2 comprises SEQ ID NO: 77, and LC CDR3 comprises SEQ ID NO: 78; (c) HC CDR1 comprises SEQ ID NO: 85, HC CDR2 comprises SEQ ID NO: 86, HC CDR3 comprises SEQ ID NO: 87, LC CDR1 comprises SEQ ID NO: 88, LC CDR2 comprises SEQ ID NO: 89, and LC CDR3 comprises SEQ ID NO: 90; (d) HC CDR1 comprises SEQ ID NO: 97, HC CDR2 comprises SEQ ID NO: 98, HC CDR3 comprises SEQ ID NO: 99, LC CDR1 comprises SEQ ID NO: 100, LC CDR2 comprises SEQ ID NO: 101, and LC CDR3 comprises SEQ ID NO: 102; (e) HC CDR1 comprises SEQ ID NO: 109; HC CDR2 comprises SEQ ID NO: 110, HC CDR3 comprises SEQ ID NO: 111, LC CDR1 comprises SEQ ID NO: 112, LC CDR2 comprises SEQ ID NO: 113, and LC CDR3 comprises SEQ ID NO: 114; or (f) HC CDR1 comprises SEQ ID NO: 121, HC CDR2 comprises SEQ ID NO: 122, HC CDR3 comprises SEQ ID NO: 123, LC CDR1 comprises SEQ ID NO: 124, LC CDR2 comprises SEQ ID NO: 125, and LC CDR3 comprises SEQ ID NO: 126.

In some embodiments, the toxin inhibits tubulin polymerization. In some embodiments, the toxin is an auristatin. In some embodiments, the auristatin is monomethyl auristatin E (MMAE). In some embodiments, the auristatin is monomethyl auristatin F (MMAF). In some embodiments, the toxin inhibits topoisomerase 1. In some embodiments, the toxin is a camptothecin derivative. In some embodiments, the toxin is exatecan. In some embodiments, the toxin is DXd. In some embodiments, the toxin inhibits RNA polymerase II. In some embodiments, the toxin is α-Amanitin.

In some embodiments, the linker is a cleavable linker. In some embodiments, the linker is cleavable by a lysosomal protease. In some embodiments, the linker is cleavable by cathepsin B, cathepsin L or cathepsin D. In some embodiments, the linker is cleavable by a glucuronidase. In some embodiments, the linker can be uncoupled by hydrolysis. In some embodiments, the is succinimidocaproyl valine-citroline para-aminobenzyloxycarbonyl (SC-VC-PAB). In some embodiments, the linker is succinimidocaproyl valine-alanine para-aminobenzyloxycarbonyl (SC-VA-PAB). succinimidocaproyl-glycine-glycine-phenylalanine-glycine-aminomethyl (SC-GGFG-AM). In some embodiments, the linker is succinimidocaproyl-glycine-glycine-glycine-glycine-aminomethyl MC-GGGG-AM). In some embodiments, the linker is succinimidocaproyl-glycine-glycine-valine-alanine-aminomethyl (SC-GGVA-AM). In some embodiments, the linker is succinimidocaproyl-glycine-glycine-valine glycine-aminomethyl (SC-GGVG-AM). In some embodiments, the linker is succinimidocaproyl-valine-alanine-aminomethyl (SC-VA-AM). In some embodiments, the linker is succinimidocaproyl-valine-alanine (SC-VA). In some embodiments, the linker is succinimidocaproyl-glycine-glycine-valine-alanine (SC-GGVA), In some embodiments, the linker is succinimidocaproyl-(SC-MAC-glucuronide).

In some embodiments, the linker and toxin together have the following structure when covalently bound to a thiol sulphur(S) of the antibody:

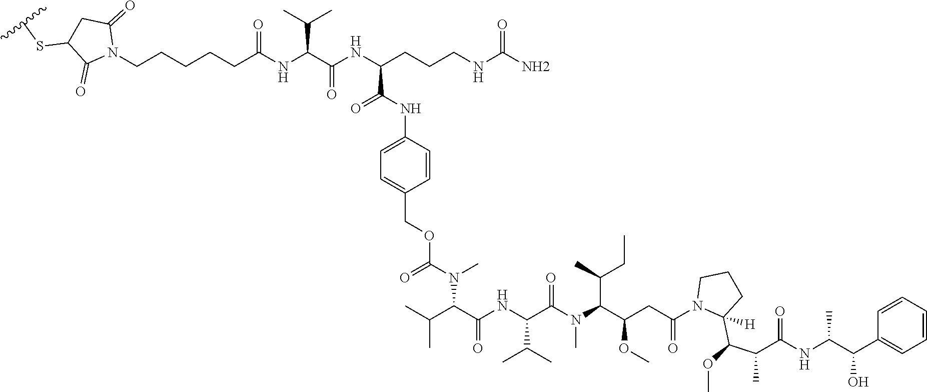 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

In some embodiments, the linker and toxin together have the following structure when covalently bound to a thiol sulphur(S) of the antibody:

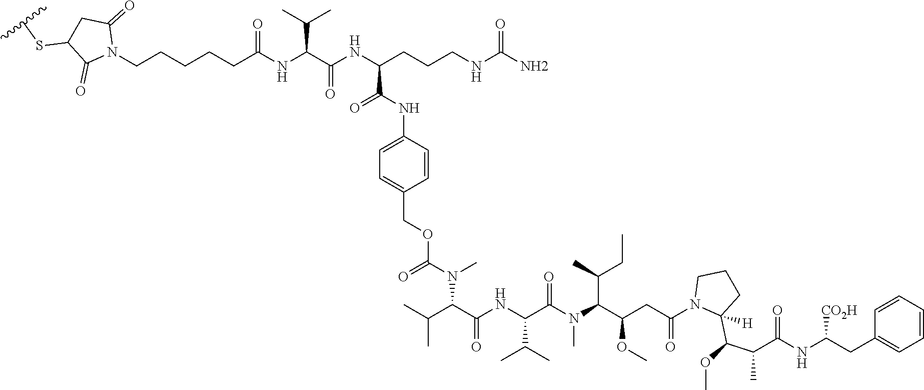 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@@H](Cc3ccccc3)C(=O)O)OC)C(C)C)C(C)C)cc2)C(C)C)C1=O]

In some embodiments, the linker and toxin together have the following structure when covalently bound to a thiol sulphur(S) of the antibody:

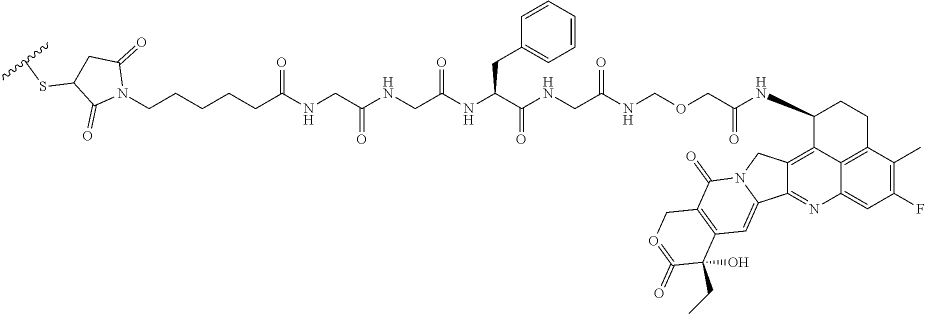

In some embodiments, the linker and toxin together have the following structure when covalently bound to a thiol sulphur(S) of the antibody:

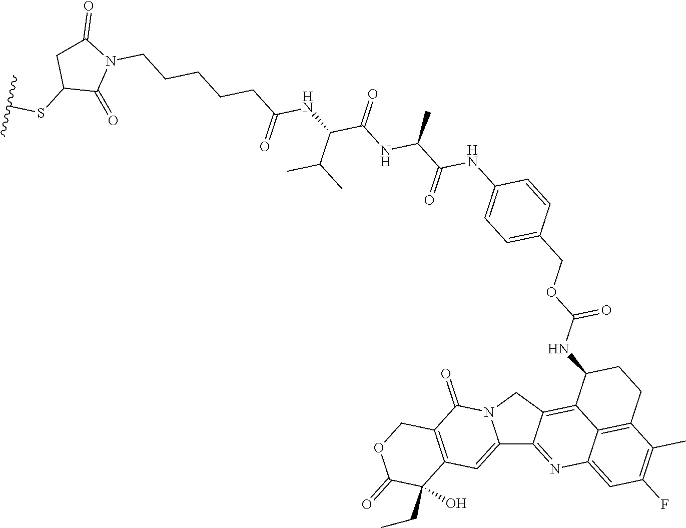 [SMILES: *SC1CC(=O)N(CCCCCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

In some embodiments, the linker and toxin together have the following structure when covalently bound to a thiol sulphur(S) of the antibody:

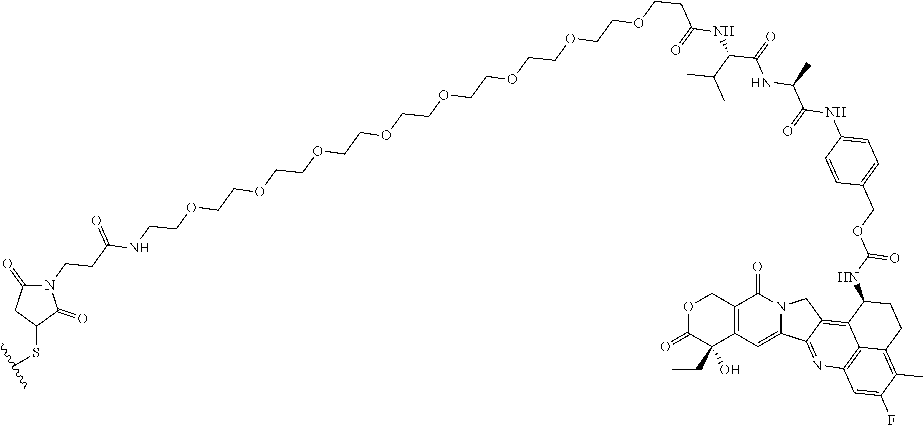 [SMILES: *SC1CC(=O)N(CCC(=O)NCCOCCOCCOCCOCCOCCOCCOCCOCCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

In some embodiments, the linker and toxin together have the following structure when covalently bound to a thiol sulphur(S) of the antibody:

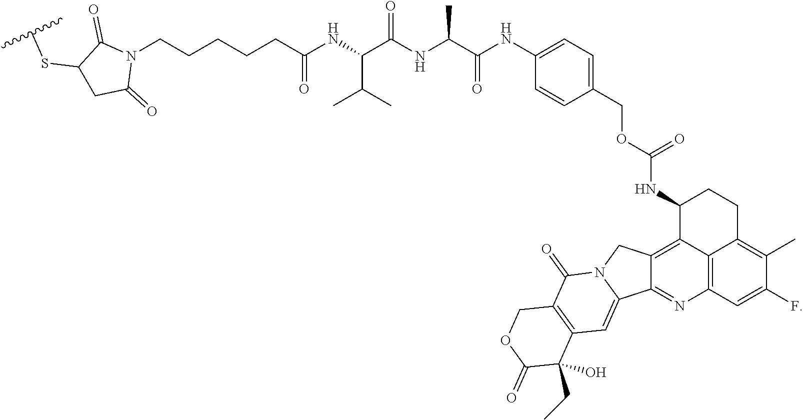 [SMILES: *SC1CC(=O)N(CCCCCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

In some embodiments, the linker and toxin together have the following structure when covalently bound to a thiol sulphur(S) of the antibody:

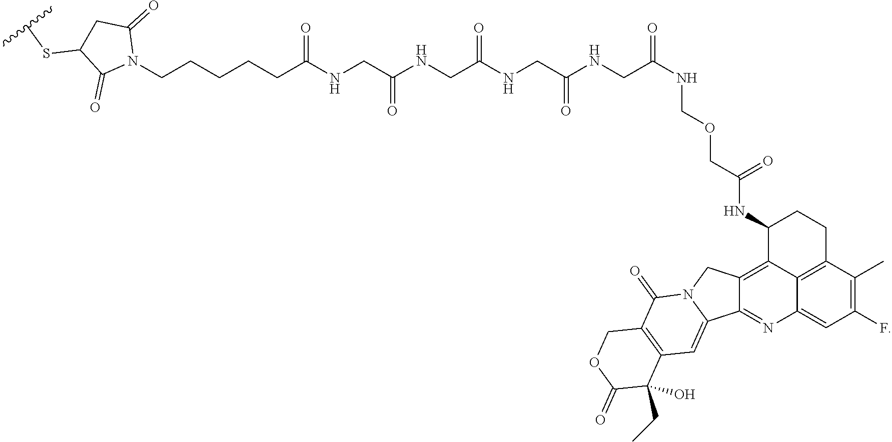 [SMILES: *SC1CC(=O)N(CCCCCC(=O)NCC(=O)NCC(=O)NCC(=O)NCC(=O)NCOCC(=O)N[C@H]2CCc3c(C)c(F)cc4nc5c(c2c34)Cn2c-5cc3c(c2=O)COC(=O)[C@]3(O)CC)C1=O]

In some embodiments, the linker and toxin together have the following structure when covalently bound to a thiol sulphur(S) of the antibody:

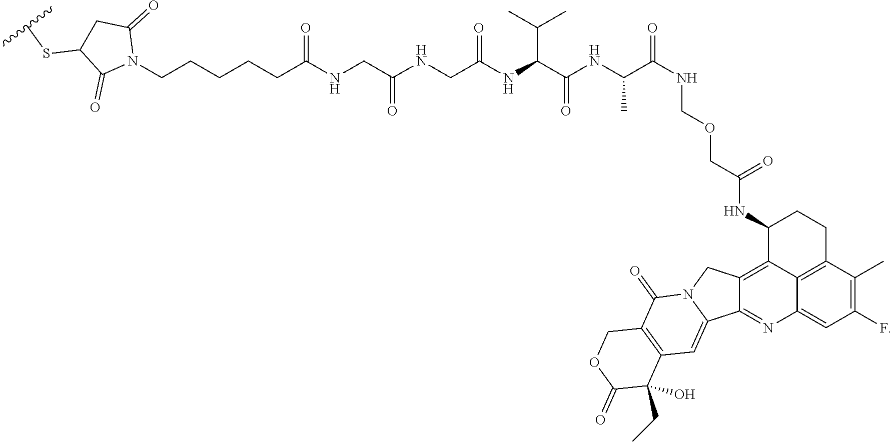 [SMILES: *SC1CC(=O)N(CCCCCC(=O)NCC(=O)NCC(=O)N[C@H](C(=O)N[C@@H](C)C(=O)NCOCC(=O)N[C@H]2CCc3c(C)c(F)cc4nc5c(c2c34)Cn2c-5cc3c(c2=O)COC(=O)[C@]3(O)CC)C(C)C)C1=O]

In some embodiments, the linker and toxin together have the following structure when covalently bound to a thiol sulphur(S) of the antibody:

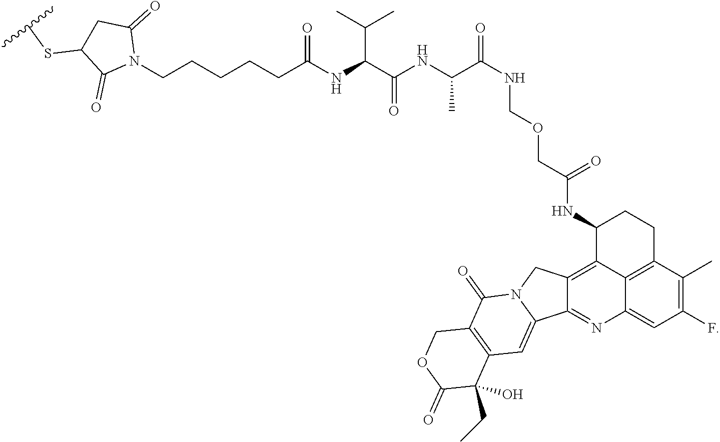 [SMILES: *SC1CC(=O)N(CCCCCC(=O)N[C@H](C(=O)N[C@@H](C)C(=O)NCOCC(=O)N[C@H]2CCc3c(C)c(F)cc4nc5c(c2c34)Cn2c-5cc3c(c2=O)COC(=O)[C@]3(O)CC)C(C)C)C1=O]

In some embodiments, the linker and toxin together have the following structure when covalently bound to a thiol sulphur(S) of the antibody:

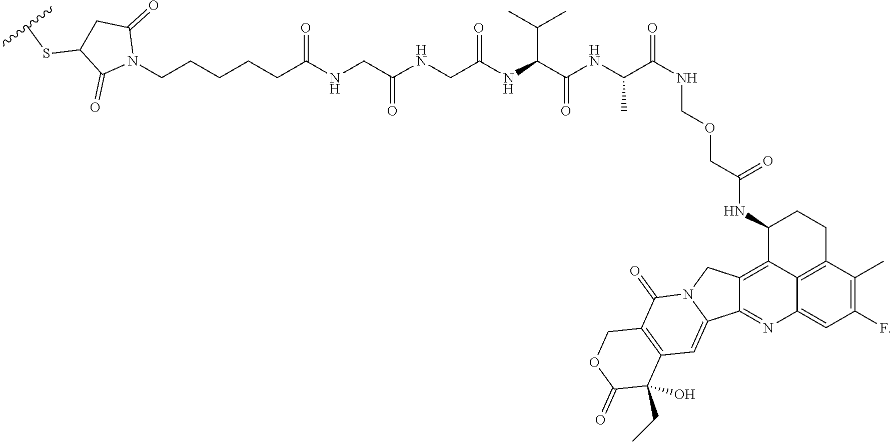 [SMILES: *SC1CC(=O)N(CCCCCC(=O)NCC(=O)NCC(=O)N[C@H](C(=O)N[C@@H](C)C(=O)NCOCC(=O)N[C@H]2CCc3c(C)c(F)cc4nc5c(c2c34)Cn2c-5cc3c(c2=O)COC(=O)[C@]3(O)CC)C(C)C)C1=O]

In some embodiments, the linker and toxin together have the following structure when covalently bound to a thiol sulphur(S) of the antibody:

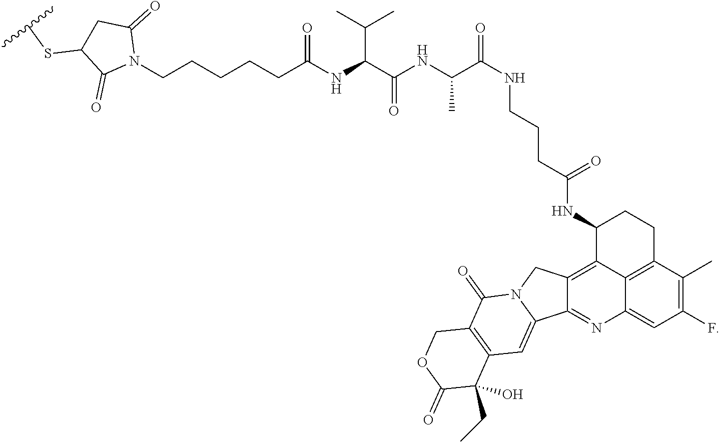 [SMILES: *SC1CC(=O)N(CCCCCC(=O)N[C@H](C(=O)N[C@@H](C)C(=O)NCCCC(=O)N[C@H]2CCc3c(C)c(F)cc4nc5c(c2c34)Cn2c-5cc3c(c2=O)COC(=O)[C@]3(O)CC)C(C)C)C1=O]

In some embodiments, the linker and toxin together have the following structure when covalently bound to a thiol sulphur(S) of the antibody:

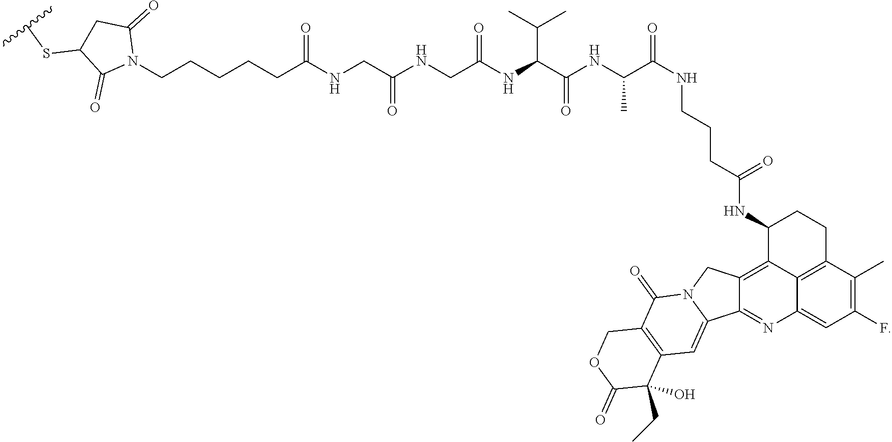 [SMILES: *SC1CC(=O)N(CCCCCC(=O)NCC(=O)NCC(=O)N[C@H](C(=O)N[C@@H](C)C(=O)NCCCC(=O)N[C@H]2CCc3c(C)c(F)cc4nc5c(c2c34)Cn2c-5cc3c(c2=O)COC(=O)[C@]3(O)CC)C(C)C)C1=O]

In some embodiments, the linker and toxin together have the following structure when covalently bound to a thiol sulphur(S) of the antibody:

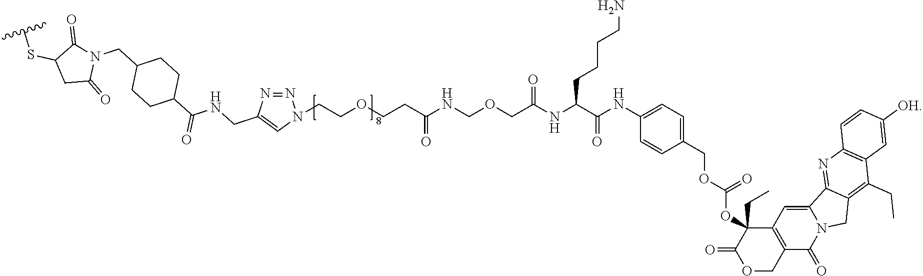

In some embodiments, the linker and toxin together have the following structure when covalently bound to a thiol sulphur(S) of the antibody:

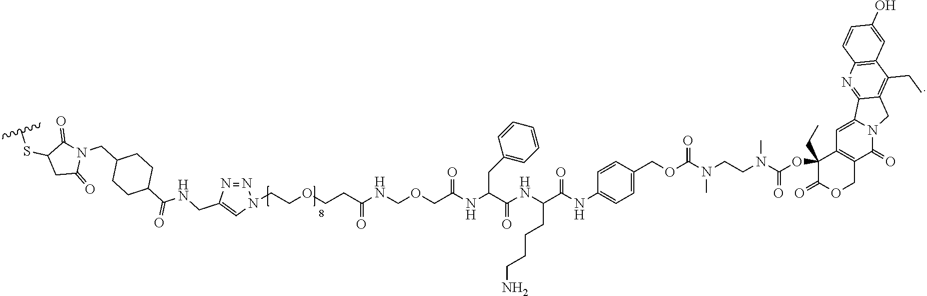

In some embodiments, the linker and toxin together have the following structure when covalently bound to a thiol sulphur(S) of the antibody:

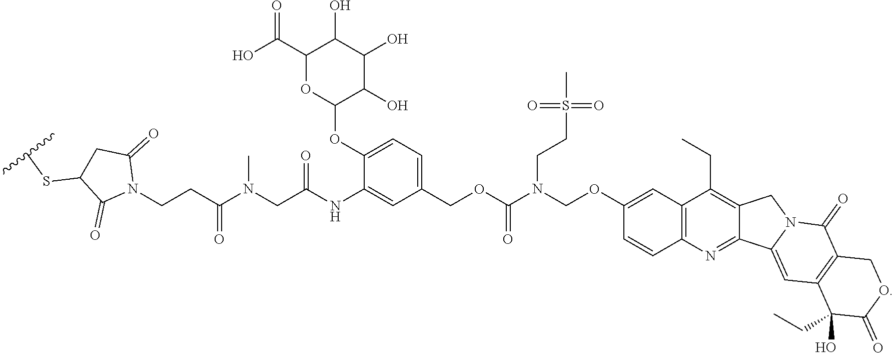 [SMILES: *SC1CC(=O)N(CCC(=O)N(C)CC(=O)Nc2cc(COC(=O)N(CCS(C)(=O)=O)COc3ccc4nc5c(c(CC)c4c3)Cn3c-5cc4c(c3=O)COC(=O)[C@]4(O)CC)ccc2OC2OC(C(=O)O)C(O)C(O)C2O)C1=O]

In some embodiments, the linker and toxin together have the following structure when covalently bound to a thiol sulphur(S) of the antibody:

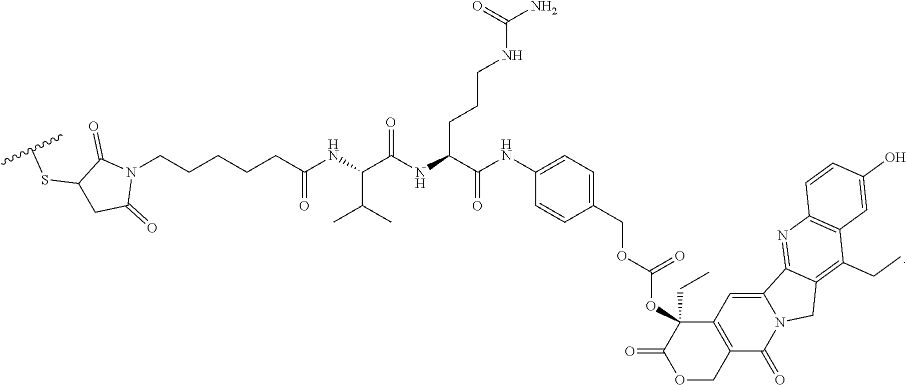 [SMILES: *SC1CC(=O)N(CCCCCC(=O)N[C@H](C(=O)N[C@@H](CCCNC(N)=O)C(=O)Nc2ccc(COC(=O)O[C@]3(CC)C(=O)OCc4c3cc3n(c4=O)Cc4c-3nc3ccc(O)cc3c4CC)cc2)C(C)C)C1=O]

In some embodiments, the linker and toxin together have the following structure when covalently bound to a thiol sulphur(S) of the antibody:

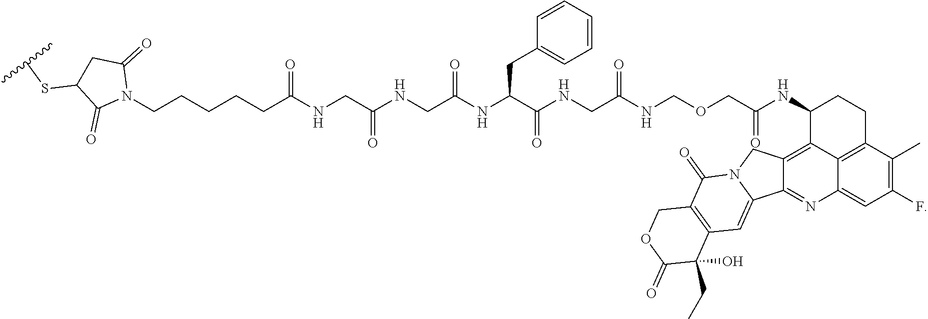 [SMILES: *SC1CC(=O)N(CCCCCC(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)C1=O]

In some embodiments, the drug to antibody ratio (DAR) of the antibody drug complex is at least 2. In some embodiments, the drug to antibody ratio (DAR) of the antibody drug complex is at least 3, at least 4, at least 6, or at least 8. In some embodiments, the drug to antibody ratio (DAR) of the antibody drug complex is about 2, about 4, about 6, about 8 or about 10. In some embodiments, the drug to antibody ratio (DAR) of the antibody drug complex from 1-3, from 2-4, from 3-5, from 4-6, from 5-7, from 6-8, from 7-9, from 8-10, or from 9-11.

Disclosed herein is a method of treating a MUC1* positive cancer in a subject comprising administering an ADC disclosed herein to the subject. Disclosed herein is an ADC for use in treating a MUC1* positive cancer. In some embodiments, the MUC1* positive cancer is a breast cancer. In some embodiments, the MUC1* positive cancer is a pancreatic cancer. In some embodiments, the MUC1* positive cancer is a lung cancer. In some embodiments, the MUC1* positive cancer is a prostate cancer. In some embodiments, the MUC1* positive cancer is an ovarian cancer.

Disclosed herein is a method of determining if a patient's cancer is suitable for treatment with an anti-MUC1* ADC comprising determining the accessibility of a MUC1* epitope on the surface of cancer cells. In some embodiments, the method comprises immunohistochemical staining of a tumor section with an anti-MUC1* antibody. In some embodiments, a tumor suitable for treatment with an anti-MUC1* ADC has high expression of an epitope accessible by the antibody (or MUC1* binding fragment thereof) of the antibody. In some embodiments, a cancer suitable for treatment with a C2 ADC has high expression of the C2 epitope. In some embodiments, a cancer suitable for treatment with a 3C2B1 ADC has high expression of the 3C2B1 epitope. In some embodiments, high expression is determined by an H-score. An H-score of at least 75, at least 100, at least 125, at least 150, at least 200, or at least 225 may be considered high expression, whereas an H-score of less than 150, less than 125, less than 100, less than 75, or less than 50 may be considered low expression. In some embodiments, a cancer may have a high H-score indicating suitability for treatment by a 3C2B1 ADC while having a low H-score for C2, or vice versa.

Disclosed herein is a method of treating an anti-MUC1* cancer comprising administering a MUC1* ADC (e.g. a C2 ADC or a 3C2B1 ADC) to an individual whose cancer was determined to be suitable for treatment with the MUC1* ADC. Likewise, the invention encompasses a MUC1* ADC for use in treating an individual whose cancer was determined to be suitable for treatment with the MUC1* ADC and the manufacture of a medicament comprising a MUC1* ADC for use in treating an individual whose cancer was determined to be suitable for treatment with the MUC1* ADC.

Disclosed herein is a method of treating a MUC1* positive cancer with heterogeneous MUC1* expression comprising administering a MUC1* ADC comprising a linker toxin moiety with a bystander effect. In some embodiments the MUC1* positive cancer has a H-score of less than 150, less than 125, less than 100, less than 75, or less than 50 despite comprising some cells with high MUC1* expression, including cells with surface MUC1* staining of 2+, 3+ or 4+. In some embodiments, the MUC1* positive cancer is any one of a breast cancer, a pancreatic cancer, a lung cancer, an ovarian cancer or an esophageal cancer. In some embodiments, the MUC1* ADC is a C2 ADC, a 20A10 ADC, or a 3C2B1 ADC. In some embodiments, the MUC1* ADC is any MUC1* ADC described in this application.

Disclosed is a method of preparing a MUC1* ADC comprising filtering the anti-MUC1* antibody in a buffer at a pH of about 6. In some embodiments, the pH is 6.0.

Disclosed herein is a reactive linker toxin complex comprising a specific structure. Disclosed herein is a method of synthesizing an ADC comprising contacting an antibody (or fragment thereof) with a reactive linker toxin complex comprising a specific structure. The specific structure may be selected from any of the following:

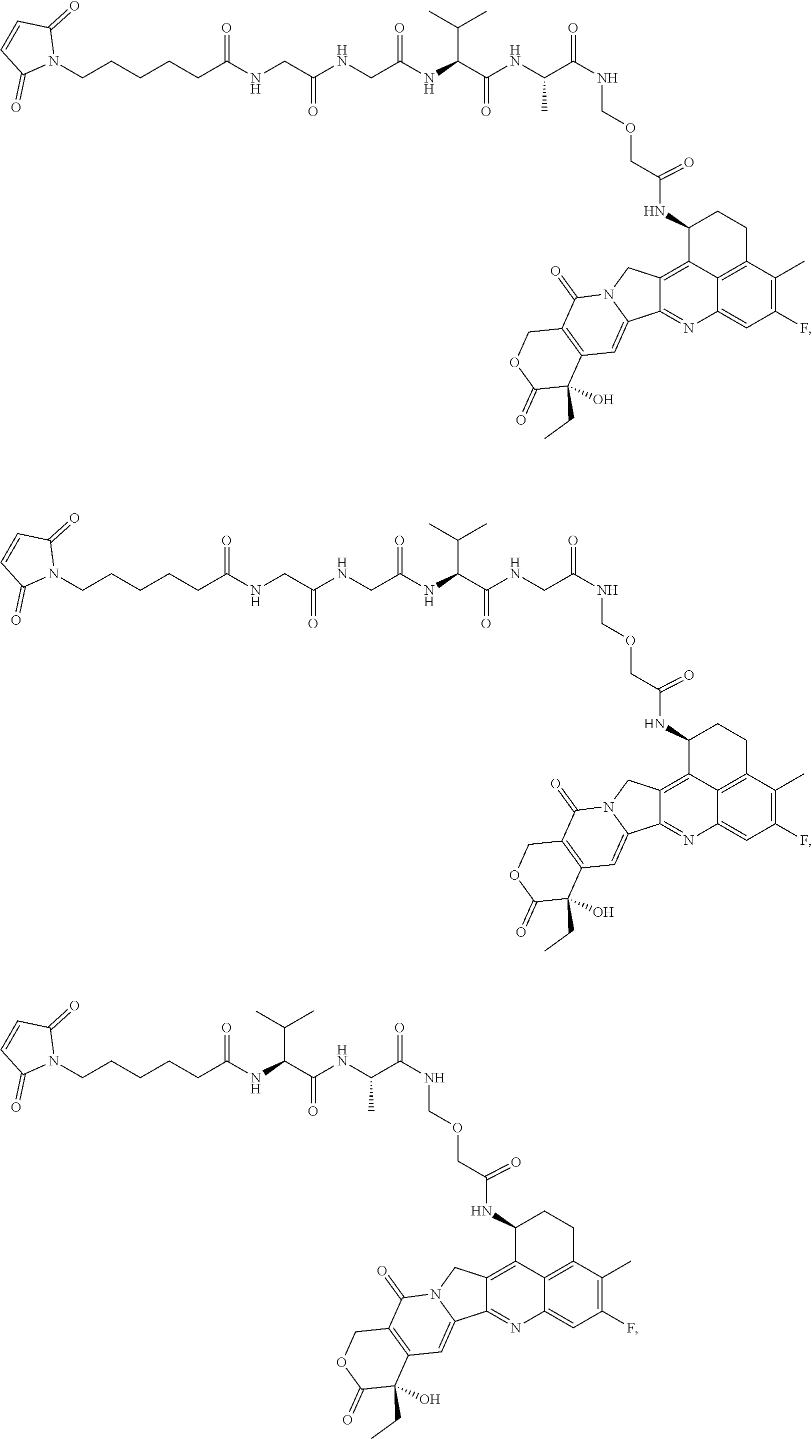 [SMILES: CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)CNC(=O)CNC(=O)CCCCCN1C(=O)C=CC1=O)C(C)C)CC3 ; CC[C@@]1(O)C(=O)OCc2c1cc1n(c2=O)Cc2c-1nc1cc(F)c(C)c3c1c2[C@@H](NC(=O)COCNC(=O)CNC(=O)[C@@H](NC(=O)CNC(=O)CNC(=O)CCCCCN1C(=O)C=CC1=O)C(C)C)CC3 ; CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)CCCCCN1C(=O)C=CC1=O)C(C)C)CC3]

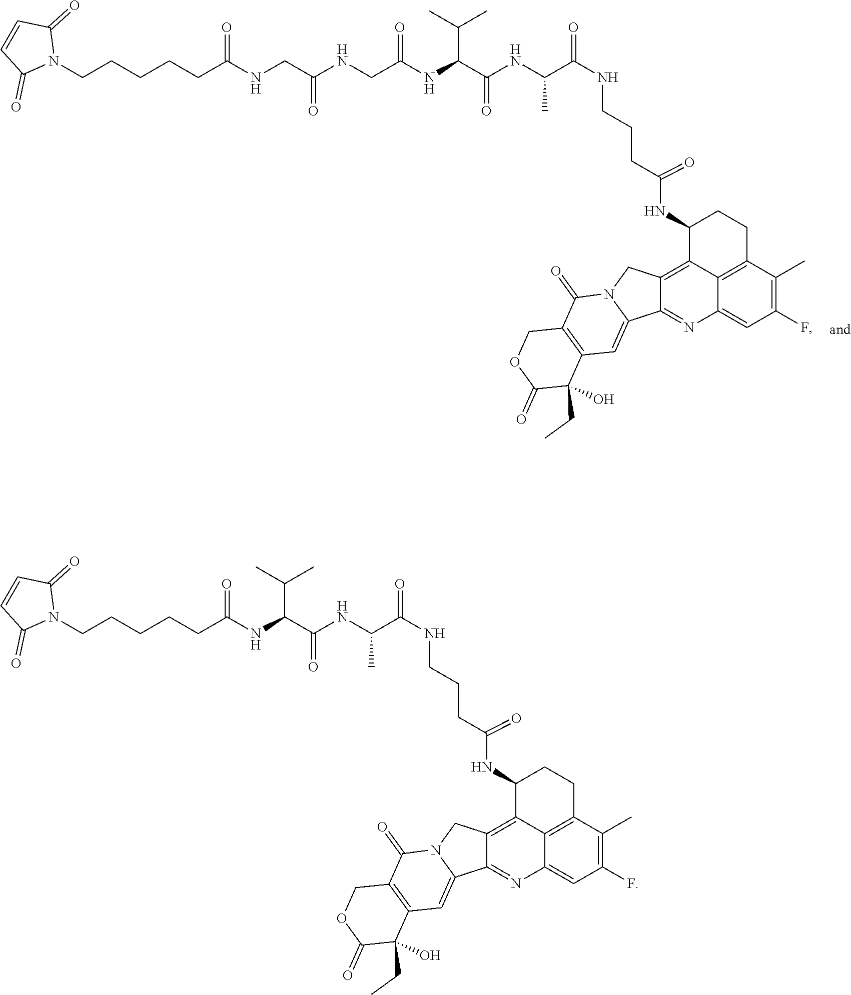 [SMILES: CC[C@@]1(O)C(=O)OCc2c1cc1n(c2=O)Cc2c-1nc1cc(F)c(C)c3c1c2[C@@H](NC(=O)CCCNC(=O)[C@H](C)NC(=O)[C@@H](NC(=O)CNC(=O)CNC(=O)CCCCCN1C(=O)C=CC1=O)C(C)C)CC3 ; CC[C@@]1(O)C(=O)OCc2c1cc1n(c2=O)Cc2c-1nc1cc(F)c(C)c3c1c2[C@@H](NC(=O)CCCNC(=O)[C@H](C)NC(=O)[C@@H](NC(=O)CCCCCN1C(=O)C=CC1=O)C(C)C)CC3]

### BRIEF DESCRIPTION OF THE DRAWINGS

FIG. 1A shows peptides derived from the MUC1* extracellular domain: PSMGFR 45mer (SEQ ID NO: 1), PSMGFR N-10 (SEQ ID NO: 2), PSMGFR C-10 (SEQ ID NO: 3), PSMGFR N+18 (SEQ ID NO: 153), and PSMGFR 9+C-9 (SEQ ID NO: 154). FIG. 1B quantifies binding of the C2 and 3C2B1 anti-MUC1* antibodies to the peptides of FIG. 1A.

FIG. 2A shows additional peptides derived from the MUC1* extracellular domain: PSMGFR N-19 (SEQ ID NO: 155), PSMGFR N-26 (SEQ ID NO: 156), PSMGFR N-30 (SEQ ID NO: 157), PSMGFR C-5 (SEQ ID NO: 158), PSMGFR N-19 C-5, (SEQ ID NO: 159), PSMGFR 45mer (SEQ ID NO: 1), PSMGFR N-10 (SEQ ID NO: 2), and PSMGFR C-10 (SEQ ID NO: 3). FIG. 2B quantifies binding of the 3C2B1 anti-MUC1* antibody to the peptides of FIG. 2A.

FIGS. 3A-3X show the internalization of anti-MUC1* antibodies C2 (FIGS. 3A-3L) and 3C2B1 (FIGS. 3M-3X) into T47D breast cancer cells (T47D-wt) or T47D cells transduced to express a MUC1* construct comprising a 45 amino acid extracellular domain (T47D-MUC1*45). FIG. 3Y quantifies the results in FIGS. 3A-3X.

FIGS. 4A1-4D28 show the internalization of anti-MUC1* antibodies C2 (FIGS. 4A1-4A28 and 4C1-4C28) and 3C2B1 (FIGS. 4B1-4B28 and 4D1-4D28) into H1975 lung cancer cells (H1975-wt) or H1975-MUC1*45 cells. FIG. 4E quantifies the results in FIGS. 4A1-4D28.

FIGS. 5A-5N show the internalization of anti-MUC1* antibody 3C2B1 into HPAFII pancreatic cancer cells and OE19 esophageal cancer cells. FIG. 5O quantifies the results in FIGS. 5A-5N.

FIGS. 6A1-6B14 show the internalization of 3C2B1-deruxtecan into H1975-wt or H1975-MUC1*45 lung cancer cells. FIG. 6C quantifies the results in FIGS. 6A1-6B14.

FIGS. 7A-7R show staining of breast cancer tumor sections with anti-MUC1* antibodies and hematoxylin.

FIGS. 8A-8R show staining of lung cancer tumor sections with anti-MUC1* antibodies and hematoxylin.

FIGS. 9A-9R show staining of pancreatic cancer tumor sections with anti-MUC1* antibodies and hematoxylin.

FIGS. 10A-10B show chemical structures of MC-VC-PAB-MMAE (FIG. 10A) and the MMAE toxin released by lysosomal cleavage of MC-VC-PAB-MMAE (FIG. 10B).

FIGS. 11A-11B show chemical structures of MC-VC-PAB-MMAF (FIG. 11A) and the MMAF toxin released by lysosomal cleavage of MC-VC-PAB-MMAF (FIG. 11B).

FIGS. 12A-12B show chemical structures of deruxtecan (FIG. 12A) and the DXd toxin released by lysosomal cleavage of deruxtecan (FIG. 12B).

FIGS. 13A-13B show chemical structures of MC-VC-PAB-exatecan (FIG. 13A) and the exatecan toxin released by lysosomal cleavage of MC-VC-PAB-exatecan (FIG. 13B).

FIGS. 14A-14B show chemical structures of MC-PEG8-Val-Ala-PABC-exatecan (FIG. 14A) and the exatecan toxin released by lysosomal cleavage of MC-PEG8-Val-Ala-PABC-exatecan (FIG. 14B).

FIGS. 15A-15B show chemical structures of MC-VA-PAB-exatecan (FIG. 15A) and the exatecan toxin released by lysosomal cleavage of MC-VA-PAB-exatecan (FIG. 15B).

FIGS. 16A-16B show chemical structures of MC-GGGG-AM-DXd (FIG. 16A) and the DXd toxin released by lysosomal cleavage of MC-GGGG-AM-DXd (FIG. 16B).

FIGS. 17A-17B show chemical structures of MC-GGVA-AM-DXd (FIG. 17A) and the DXd toxin released by lysosomal cleavage of MC-GGVA-AM-DXd (FIG. 17B).

FIGS. 18A-18B show chemical structures of MC-GGVG-AM-DXd (FIG. 18A) and the DXd toxin released by lysosomal cleavage of MC-GGVG-AM-DXd (FIG. 18B).

FIGS. 19A-19B show chemical structures of MC-VA-AM-DXd (FIG. 19A) and the DXd toxin released by lysosomal cleavage of MC-VA-AM-DXd (FIG. 19B).

FIGS. 20A-20B show chemical structures of MC-VA-GAB-exatecan (FIG. 20A) and the GAB-exatecan toxin released by lysosomal cleavage of MC-VA-GAB-exatecan (FIG. 20B).

FIGS. 21A-21B show chemical structures of MC-GGVA-GAB-exatecan (FIG. 21A) and the GAB-exatecan toxin released by lysosomal cleavage of MC-GGVA-GAB-exatecan (FIG. 21B).

FIGS. 22A-22B show chemical structures of CL2A-SN38 (FIG. 22A) and the SN38 toxin released by lysosomal cleavage of CL2A-SN38 (FIG. 22B).

FIGS. 23A-23B show chemical structures of CL2E-SN38 (FIG. 23A) and the SN38 toxin released by lysosomal cleavage of CL2A-SN38 (FIG. 23B).

FIGS. 24A-24B show chemical structures of MAC-glucuronide-SN38 (FIG. 24A) and the SN38 toxin released by lysosomal cleavage of MAC-glucuronide-SN38 (FIG. 24B).

FIGS. 25A-25B show chemical structures of MC-VC-PAB-SN38 (FIG. 25A) and the SN38 toxin released by lysosomal cleavage of MC-VC-PAB-SN38 (FIG. 25B).

FIGS. 26A-26B show examples of in vitro cytotoxicity result collected with an xCELLigence instrument and the determination of IC50 values. In these examples, CPFAC1 pancreatic cancer cells were treated with C2-deruxtecan (DAR 7.7) (FIG. 26A) or 3C2B1-deruxtecan (DAR 9.2) (FIG. 26B).

FIGS. 27A-27H show charts showing that anti-MUC1* ADCs inhibit growth of breast cancer tumors. FIGS. 27A-27B shows charts of treatment of T47D xenograft tumors with C2-MMAE, 20A10-MMAE or a PBS control. FIGS. 27C-27D show charts of treatment of T47D-MUC1* xenograft tumors with C2-MMAE, 20A10-MMAE or a PBS control. FIGS. 6E-6F show charts of treatment of T47D xenograft tumors with C2-deruxtecan, C2-exatecan or a PBS control. FIGS. 6G-6H show the treatment of T47D-MUC1* xenograft tumors with C2-deruxtecan, C2-exatecan or a PBS control. FIGS. 27A, 27C, 27E, and 27G show tumor size determined by radiance measurements on an IVIS instrument. Injection dates are marked with. FIGS. 27B, 27D, 27F, and 27H shows the weights of the T47D xenograft tumors of FIGS. 27A, 27C, 27E, and 27G, respectively.

FIGS. 28A-28F shows charts showing that anti-MUC1* ADCs inhibit growth of pancreatic cancer tumors. FIGS. 28A-28C show charts of treatment of HPAF II-MUC1* xenograft tumors with C2-MMAE or a PBS control. FIGS. 28D-28F show charts of treatment of HPAF II-MUC1* xenograft tumors with C2-deruxtecan, C2-exatecan, or a PBS control. In FIGS. 28A and 28D, tumor size was determined by radiance measurements on an IVIS instrument. Injection dates are marked with. FIGS. 28B and 28E shows weight of the HPAF II-MUC1* xenograft tumors from the mice of FIGS. 28A and 6D, respectively, after treatment. FIGS. 28C and 7F show survival curves of the mice of FIGS. 28A and 28D, respectively.

FIGS. 29A-29D shows charts showing that anti-MUC1* ADCs inhibit growth of lung cancer tumors. FIG. 29A show charts of the treatment of NCI-H1975 xenograft tumors with C2-MMAE, 20A10-MMAE or a PBS control. Tumor size was determined by radiance measurements on an IVIS instrument. Injection dates are marked with ▾. FIG. 29B show survival curves of the C2-MMAE, 20A10-MMAE, and PBS control treated mice with NCI-H1975 xenograft tumors. FIG. 29C show charts of the treatment of NCI-H1975 xenograft tumors with C2-deruxtecan, C2-exatecan or a PBS control. Tumor size was determined by measurement with a caliper. Injection dates are marked with ▾. FIG. 29D show survival curves of the C2-deruxtecan, C2-exatecan, and PBS control treated mice with NCI-H1975 xenograft tumors.

FIG. 30 is a table showing cytotoxicity of the indicated MUC1* ADCs against indicated cancer cells. IC₅₀ values were determined from xCELLigence data.

FIG. 31 is a table showing cytotoxicity of the indicated MUC1* ADCs against indicated cancer cells. IC₅₀ values were determined from PrestoBlue data.

FIGS. 32A1-32D20 shows immunofluorescent photographs of non-target HEK-293-TN cells or low MUC1* positive H1975-wt lung cancer cells 120 hours after the addition of either C2-MC-GGFG-AM-DXd or C2-MC-VA-PAB-exatecan. The cell population was doped with H1975-MUC1*45-GFP cells as indicated. FIGS. 31A1-31A20 show the effect of C2-MC-GGFG-AM-DXd on the non-target cells. FIGS. 31B1-31B20 show that ADC's effect on MUC1* positive H1975 wildtype cells or H1975-wt doped with 5%, 10% or 30% H1975 cells engineered to overexpress MUC1*45. FIGS. 31C1-31C20 show the effect of C2-MC-VA-PAB-exatecan on the non-target cells. FIGS. 31D1-31D20 show that ADC's effect on MUC1* positive H1975 wildtype cells or H1975-wt doped with 5%, 10% or 30% H1975 cells engineered to overexpress MUC1*45.

FIGS. 33A-33H show graphs of the integrated pixel density of non-target cancer cells as a function of doping cell population with cells expressing high amounts of MUC1*45. FIGS. 33A-33D show the effect when the cells were treated with C2-MC-GGFG-AM-DXd. FIGS. 33E-33H show the effect when the cells were treated with C2-MC-VA-PAB-exatecan.

FIGS. 34A-34H show graphs of the integrated pixel density of target low MUC1* expressing cancer cells as a function of doping cell population with cells expressing high amounts of MUC1*45. FIGS. 34A-34D show the effect on target cells treated with C2-MC-GGFG-AM-DXd. FIGS. 34E-34H show the effect on target cells treated with C2-MC-VA-PAB-exatecan.

FIG. 35 is a table showing cytotoxicity of the indicated MUC1* ADCs against indicated cancer cells. IC₅₀ values were determined from xCELLigence data.

FIG. 36 is a table showing cytotoxicity of the indicated MUC1* ADCs against indicated cancer cells. IC₅₀ values were determined from xCELLigence data.

FIGS. 37A-37C show Kaplan-Meier survival curves for mice xenografted with human H1975 lung cancer cells (FIG. 37A), H2110 lung cancer cells (FIG. 37B) or CFPAC pancreatic cancer cells, wherein the animals were treated with either PBS as a control, C2-deruxtecan or 3C2B1-deruxtecan.

### DETAILED DESCRIPTION OF THE INVENTION

The present application relates to anti-MUC1* antibody conjugates and methods of making and using them. MUC1* is a cleaved form of mucin-1 (MUC1) that functions in cancer cells as a growth factor receptor that binds to ligands such as NME1 and NME7. Ligands with two MUC1* binding sites dimerize MUC1* to drive cancer growth and metastasis. MUC1* is an ideal target for cancer drugs as it is aberrantly expressed on over 75% of all cancers and is likely overexpressed on an even higher percentage of metastatic cancers (Mahanta et al. PLOS ONE 3 (4): e2054 (2008); Fessler et al., Breast Cancer Res Treat. 118 (1): 113-124 (2009)). Cleavage and release of the bulk of the extracellular domain of MUC1 unmasks the ligand binding site. After cleavage, most of the extracellular domain of MUC1 is shed from the cell surface. The shed portion includes numerous tandem repeat sequences near the N-terminus of full-length MUC1. The remaining portion, MUC1*, has a truncated extracellular domain that comprises most or all of the 45 amino acid primary growth factor receptor sequence, PSMGFR (SEQ ID NO: 1). Anti-MUC1* antibodies generated against a PSMGFR peptide, such as C2, bind to the cleaved (MUC1*) form of MUC1 on cancer cells but do not bind to full length MUC1. Recombinant expression of a MUC1* polypeptide with a 45 amino acid extracellular domain (MUC1*45) confers responsiveness to the MUC1* ligand NME7-AB and renders cancer cells more susceptible to killing by CAR T cells targeted by a MUC1* binding single chain antibody derived from C2. WO2010042562; WO2010042891. However, the N-terminus of MUC1* has not been precisely determined and may differ depending upon which extracellular proteases are active in a tumor.

Many agents currently administered to a patient parenterally are not targeted, resulting in systemic delivery of the agent to cells and tissues of the body where it is unnecessary, and often undesirable. This may result in adverse drug side effects, and often limits the dose of a drug (e.g., chemotherapeutic (anti-cancer), cytotoxic, enzyme inhibitor agents and antiviral or antimicrobial drugs) that can be administered.

A major goal has been to develop methods for specifically targeting therapeutic agents to cells and tissues. The benefits of such treatment include avoiding the general physiological effects of inappropriate delivery of such agents to other cells and tissues. These and other limitations and problems of the past are addressed by the disclosure herein.

### CERTAIN TERMINOLOGY

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. Generally, the procedures for cell culture, cell infection, antibody production and molecular biology methods are methods commonly used in the art. Such standard techniques can be found, for example, in reference manuals such as, for example, Sambrook et al. (2000) and Ausubel et al. (1994).

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention.

As used herein, ranges and amounts can be expressed as “about” a particular value or range. About also includes the exact amount. For example “about 1 mg” means “about 1 mg” and also “1 mg.” The terms “about” and “approximately” generally include an amount that would be expected to be within experimental error.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “polypeptide,” “peptide” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds.

Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a β-ester, a thioamide, phosphonamide, carbamate, hydroxylate, and the like (see, e.g., Spatola, (1983) Chem. Biochem. Amino Acids and Proteins 7:267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acids are grouped as hydrophobic amino acids, polar amino acids, non-polar amino acids, and charged amino acids. Hydrophobic amino acids include small hydrophobic amino acids and large hydrophobic amino acids. Small hydrophobic amino acid can be glycine, alanine, proline, and analogs thereof. Large hydrophobic amino acids can be valine, leucine, isoleucine, phenylalanine, methionine, tryptophan, and analogs thereof. Polar amino acids can be serine, threonine, asparagine, glutamine, cysteine, tyrosine, and analogs thereof. Non-polar amino acids can be glycine, alanine, valine, leucine, isoleucine, methionine, phenylalanine, tryptophan, proline, and analogs thereof. Charged amino acids can be lysine, arginine, histidine, aspartate, glutamate, and analogs thereof. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids are either D amino acids or L amino acids.

As used in the specification and appended claims, unless specified to the contrary, the following terms have the meaning indicated below.

The compounds disclosed herein, in some embodiments, contain one or more asymmetric centers and thus give rise to enantiomers, diastereomers, and other stereoisomeric forms that are defined, in terms of absolute stereochemistry, as (R)- or(S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

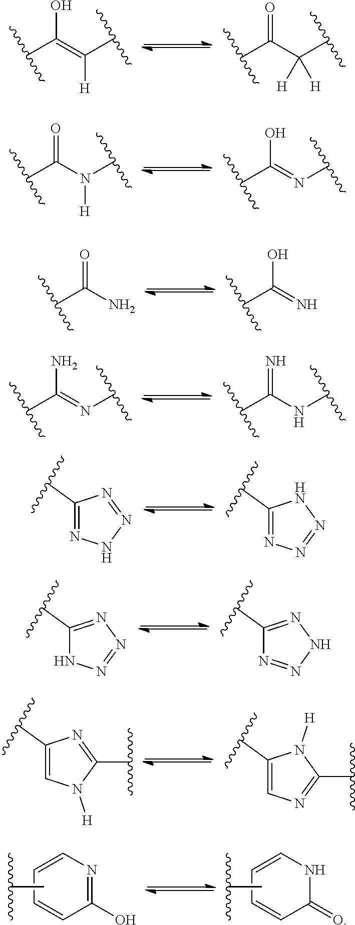

“Pharmaceutically acceptable salt” includes both acid and base addition salts. A pharmaceutically acceptable salt of any one of the compounds or conjugates described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

As used herein, the terms “antibody” and “immunoglobulin” are terms of art and can be used interchangeably herein, and refer to a molecule with an antigen binding site that specifically binds an antigen. In certain embodiments, an isolated antibody (e.g., monoclonal antibody) described herein, or an antigen-binding fragment thereof, which specifically binds to a protein of interest.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain variable fragments (scFv), camelized antibodies, affibodies, Fab fragments, F(ab′) fragments, F(ab′) 2 fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

The CDR sequence(s) for the antibodies disclosed herein, or the anti-MUC1* binding domain sequences disclosed herein, may be defined or determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226); or (iii) the ImMunoGeneTics (IMGT) numbering system, for example, as described in Lefranc, M.-P., 1999, The Immunologist, 7:132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “human antibody” as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Bol. 5 (2001) 368-374). In some instances, human antibodies are also produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al, Proc. Natl. Acad. Sci. USA 90 (1993) 2551-2555; Jakobovits, A., et al, Nature 362 (1993) 255-258; Bruggemann, M., et al, Year Immunol. 7 (1993) 33-40). In additional instances, human antibodies are also produced in phage display libraries (Hoogenboom, H. R., and Winter, G., J. Mol. Biol. 227 (1992) 381-388; Marks, J. D., et al, J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss, p. 77 (1985); and Boerner, P., et al, J. Immunol. 147 (1991) 86-95). A “humanized antibody” is a non-human antibody that has been engineered to more closely resemble a human antibody by, for example, replacing amino acid residues in non-human constant regions and framework regions with amino acid residues from similar human constant and framework regions (Reichmann, L. R. et al, Nature 332 (1988) 323-327).

As used herein, an “antigen” is a moiety or molecule that contains an epitope to which an antibody can specifically bind. As such, an antigen is specifically bound by an antibody. In a specific embodiment, the antigen, to which an antibody described herein binds, is a protein of interest, for example, MUC1*, or a fragment thereof.

As used herein, the term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

As used herein, the term “percent (%) amino acid sequence identity” or “sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS ALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen.

As used herein, an “epitope” is a term known in the art and refers to a localized region of an antigen to which an antibody can specifically bind. An epitope can be a linear epitope of contiguous amino acids or can comprise amino acids from two or more non-contiguous regions of the antigen.

As used herein, the terms “binds,” “binds to,” “specifically binds” or “specifically binds to” in the context of antibody binding refer to antibody binding to an antigen (e.g., epitope) as such binding is understood by one skilled in the art. In a specific embodiment, molecules that specifically bind to an antigen bind to the antigen with an affinity (Ka) that is at least 2 logs, 2.5 logs, 3 logs, 4 logs lower (higher affinity) than the Ka when the molecules bind to another antigen. In another specific embodiment, molecules that specifically bind to an antigen do not cross react with other proteins.

A “linking moiety” or “linker” (e.g., noted as L) is a molecule or structure that conjugates, or allows conjugation of, a polypeptide (e.g., an antibody) to another molecule such as therapeutic agent or toxin. The linker can be generated by reacting a linker precursor that comprises one or more reactive termini for conjugation to a polypeptide (e.g., an antibody) through a conjugation moiety (e.g., a maleimide group), or for conjugation to another molecule such as a therapeutic agent. The polypeptide conjugation reactive terminus of the linker is typically a site that is capable of conjugation to the polypeptide (e.g., an antibody) through a cysteine thiol group on the polypeptide (e.g., an antibody), and so is typically a thiol-reactive group such as a maleimide or a dibromomaleimide, or as defined herein.

As known in the art, the reaction product of a maleimide group and a cysteine thiol group on a polypeptide comprises a succinimidocaproyl (SC) moiety

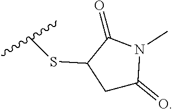 [SMILES: *SC1CC(=O)N(C)C1=O]

As used herein, “maleimidocaproyl” or “MC” in the context of an ADC refers to the succinimidocaproyl (SC) reaction product rather than the maleimidocaproyl (MC) precursor.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. In some embodiments, a therapeutic benefit is achieved by slowing or arresting the progression of a disease. For prophylactic benefit, the compositions are, in some embodiments, administered to a patient at risk of developing a particular disease, or to a patient reporting one or more of the physiological symptoms of a disease, even though a diagnosis of this disease has not been made.

### MUC1* Binding Domains

In some embodiments, disclosed herein is an anti-MUC1* antibody that comprises a MUC1* binding domain. In some embodiments, the MUC1* binding domain comprises an antibody or antigen binding fragment or variant thereof. In some embodiments, the antibody or antigen binding fragment or variant thereof is a monoclonal antibody. In some embodiments, the antibody or antigen binding fragment or variant thereof is a human antibody, a murine antibody, a humanized antibody, or a chimeric antibody. In some embodiments, the MUC1* binding domain comprises a monovalent Fab, a bivalent Fab′2, a single-chain variable fragment (scFv), or functional fragment or variant thereof. In some embodiments, the MUC1* binding domain comprises an IgG1, IgG2, IgG3, or IgG4 domain. In some embodiments, the MUC1* binding domain comprises an IgG1 domain. In some embodiments, the MUC1* binding domain comprises an IgG2 domain. In some embodiments, the MUC1* binding domain comprises an IgG3 domain. In some embodiments, the MUC1* binding domain comprises an IgG4 domain.

In some embodiments, the antibody, or functional fragment or functional variant thereof that binds specifically to MUC1* comprises an anti-MUC1* heavy chain and an anti-MUC1* light chain.

In some embodiments, the anti-MUC1* heavy chain comprises an anti-MUC1* heavy chain variable domain. In some embodiments, the anti-MUC1* heavy chain variable domain comprises a variable domain of an IgG1, IgG2, IgG3, or IgG4 heavy chain. In some embodiments, the anti-MUC1* light chain comprises an anti-MUC1* light chain variable domain. In some embodiments, the anti-MUC1* light chain variable domain comprises a variable domain of a Kappa or Lambda light chain.

In some embodiments, the anti-MUC1* heavy chain variable domain comprises the variable domain of an IgG1 heavy chain and the anti-MUC1* light chain variable domain comprises the variable domain of a Kappa or Lambda light chain. In some embodiments, the anti-MUC1* heavy chain variable domain comprises the variable domain of an IgG2 heavy chain and the anti-MUC1* light chain variable domain comprises the variable domain of a Kappa or Lambda light chain. In some embodiments, the anti-MUC1* heavy chain variable domain comprises the variable domain of an IgG3 heavy chain and the anti-MUC1* light chain variable domain comprises the variable domain of a Kappa or Lambda light chain. In some embodiments, the anti-MUC1* heavy chain variable domain comprises the variable domain of an IgG4 heavy chain and the anti-MUC1* light chain variable domain comprises the variable domain of a Kappa or Lambda light chain.

In some embodiments, the anti-MUC1* heavy chain variable domain comprises the variable domain of an IgG1 heavy chain and the anti-MUC1* light chain variable domain comprises the variable domain of a Kappa light chain. In some embodiments, the anti-MUC1* heavy chain variable domain comprises the variable domain of an IgG2 heavy chain and the anti-MUC1* light chain variable domain comprises the variable domain of a Kappa light chain. In some embodiments, the anti-MUC1* heavy chain variable domain comprises the variable domain of an IgG3 heavy chain and the anti-MUC1* light chain variable domain comprises the variable domain of a Kappa light chain. In some embodiments, the anti-MUC1* heavy chain variable domain comprises the variable domain of an IgG4 heavy chain and the anti-MUC1* light chain variable domain comprises the variable domain of a Kappa light chain.

In some embodiments, the anti-MUC1* heavy chain variable domain comprises the variable domain of an IgG1 heavy chain and the anti-MUC1* light chain variable domain comprises the variable domain of a Lambda light chain. In some embodiments, the anti-MUC1* heavy chain variable domain comprises the variable domain of an IgG2 heavy chain and the anti-MUC1* light chain variable domain comprises the variable domain of a Lambda light chain. In some embodiments, the anti-MUC1* heavy chain variable domain comprises the variable domain of an IgG3 heavy chain and the anti-MUC1* light chain variable domain comprises the variable domain of a Lambda light chain. In some embodiments, the anti-MUC1* heavy chain variable domain comprises the variable domain of an IgG4 heavy chain and the anti-MUC1* light chain variable domain comprises the variable domain of a Lambda light chain.

In some embodiments, the antibody, or functional fragment or functional variant thereof, that binds specifically to MUC1* comprises a single-chain variable fragment (scFv) or an antigen-binding fragment (Fab). In some embodiments, the antibody, or functional fragment or functional variant thereof, that binds specifically to MUC1* comprises a single-chain variable fragment. In some embodiments, the antibody, or functional fragment or functional variant thereof, that binds specifically to MUC1* comprises an antigen-binding fragment (Fab).

In some embodiments, the anti-MUC1* heavy chain variable domain comprises complementarity determining regions (CDRs): HC-CDR1, HC-CDR2, and HC-CDR3, and the anti-MUC1* light chain variable domain comprises LC-CDR1, LC-CDR2, and LC-CDR3. In some embodiments HC-CDR1 comprises SEQ ID NO: 49, HC-CDR2 comprises SEQ ID NO: 50, HC-CDR3 comprises SEQ ID NO: 51, LC-CDR1 comprises SEQ ID NO: 52, LC-CDR2 comprises SEQ ID NO: 53, and LC-CDR3 comprises SEQ ID NO: 54. In some embodiments HC-CDR1 comprises SEQ ID NO: 11, HC-CDR2 comprises SEQ ID NO: 12, HC-CDR3 comprises SEQ ID NO: 13, LC-CDR1 comprises SEQ ID NO: 14, LC-CDR2 comprises SEQ ID NO: 15, and LC-CDR3 comprises SEQ ID NO: 16. In some embodiments HC-CDR1 comprises SEQ ID NO: 25, HC-CDR2 comprises SEQ ID NO: 26, HC-CDR3 comprises SEQ ID NO: 27, LC-CDR1 comprises SEQ ID NO: 28, LC-CDR2 comprises SEQ ID NO: 29, and LC-CDR3 comprises SEQ ID NO: 30. In some embodiments HC-CDR1 comprises SEQ ID NO: 37, HC-CDR2 comprises SEQ ID NO: 38, HC-CDR3 comprises SEQ ID NO: 39, LC-CDR1 comprises SEQ ID NO: 40, LC-CDR2 comprises SEQ ID NO: 41, and LC-CDR3 comprises SEQ ID NO: 42. In some embodiments HC-CDR1 comprises SEQ ID NO: 49, HC-CDR2 comprises SEQ ID NO: 50, HC-CDR3 comprises SEQ ID NO: 51, LC-CDR1 comprises SEQ ID NO: 52, LC-CDR2 comprises SEQ ID NO: 53, and LC-CDR3 comprises SEQ ID NO: 54. In some embodiments HC-CDR1 comprises SEQ ID NO: 61, HC-CDR2 comprises SEQ ID NO: 62, HC-CDR3 comprises SEQ ID NO: 63, LC-CDR1 comprises SEQ ID NO: 64, LC-CDR2 comprises SEQ ID NO: 65, and LC-CDR3 comprises SEQ ID NO: 66. In some embodiments HC-CDR1 comprises SEQ ID NO: 73, HC-CDR2 comprises SEQ ID NO: 74, HC-CDR3 comprises SEQ ID NO: 75, LC-CDR1 comprises SEQ ID NO: 76, LC-CDR2 comprises SEQ ID NO: 77, and LC-CDR3 comprises SEQ ID NO: 78. In some embodiments HC-CDR1 comprises SEQ ID NO: 85, HC-CDR2 comprises SEQ ID NO: 86, HC-CDR3 comprises SEQ ID NO: 87, LC-CDR1 comprises SEQ ID NO: 88, LC-CDR2 comprises SEQ ID NO: 89, and LC-CDR3 comprises SEQ ID NO: 90. In some embodiments HC-CDR1 comprises SEQ ID NO: 97, HC-CDR2 comprises SEQ ID NO: 98, HC-CDR3 comprises SEQ ID NO: 99, LC-CDR1 comprises SEQ ID NO: 100, LC-CDR2 comprises SEQ ID NO: 101, and LC-CDR3 comprises SEQ ID NO: 102. In some embodiments HC-CDR1 comprises SEQ ID NO: 109, HC-CDR2 comprises SEQ ID NO: 110, HC-CDR3 comprises SEQ ID NO: 111, LC-CDR1 comprises SEQ ID NO: 112, LC-CDR2 comprises SEQ ID NO: 113, and LC-CDR3 comprises SEQ ID NO: 114. In some embodiments HC-CDR1 comprises SEQ ID NO: 121, HC-CDR2 comprises SEQ ID NO: 122, HC-CDR3 comprises SEQ ID NO: 123, LC-CDR1 comprises SEQ ID NO: 124, LC-CDR2 comprises SEQ ID NO: 125, and LC-CDR3 comprises SEQ ID NO: 126. In some embodiments HC-CDR1 comprises SEQ ID NO: 133, HC-CDR2 comprises SEQ ID NO: 134, HC-CDR3 comprises SEQ ID NO: 135, LC-CDR1 comprises SEQ ID NO: 136, LC-CDR2 comprises SEQ ID NO: 137, and LC-CDR3 comprises SEQ ID NO: 138.

In some embodiments, the anti-MUC1* heavy chain comprises an amino acid sequence with at least 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 47. In some embodiments, the antibody comprises: a heavy chain (HC) variable domain having at least 80%, at least 85%, at least 90%, at least 95%, or at least 98% sequence identity to SEQ ID NO: 9, 23, 35, 59, 71, 83, 95, 107, 119, or 131. In some embodiments, the anti-MUC1* light chain comprises an amino acid sequence with at least 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NOs: 48. In some embodiments, the anti-MUC1* light chain (LC) variable domain comprise an amino acid sequence at least 80%, at least 85%, at least 90%, at least 95%, or at least 98% sequence identity to SEQ ID NO: 10, 24, 36, 60, 72, 84, 96, 108, 120, or 132.

In some embodiments, the antibody comprises: a heavy chain (HC) variable domain having at least 80%, at least 85%, at least 90%, at least 95%, or at least 98% sequence identity to SEQ ID NO: 47, and a light chain (LC) variable domain having at least 80%, at least 85%, at least 90%, at least 95%, or at least 98% sequence identity to SEQ ID NO: 48.

In some embodiments, the antibody comprises: a heavy chain (HC) variable domain having at least 80%, at least 85%, at least 90%, at least 95%, or at least 98% sequence identity to SEQ ID NO: 9, and a light chain (LC) variable domain having at least 80%, at least 85%, at least 90%, at least 95%, or at least 98% sequence identity to SEQ ID NO: 10.

In some embodiments, the antibody comprises: a HC variable domain having at least 80%, at least 85%, at least 90%, at least 95%, or at least 98% sequence identity to SEQ ID NO: 23, and a LC variable domain having at least 80%, at least 85%, at least 90%, at least 95%, or at least 98% sequence identity to SEQ ID NO: 24.

Sequences of exemplary antibodies and antibody fragments that bind to the extracellular domain of MUC1* and specifically bind to peptides consisting of the sequences of SEQ ID NOs: 1, 2, and 155-159 are presented in Tables 1-3.

TABLE 1
MUC1* Binding Domain CDR sequences

| | SEQ ID | | SEQ ID | | SEQ ID | |
|---|---|---|---|---|---|---|
| Construct | NO | MUC1* HC-CDR1 | NO | MUC1* HC-CDR2 | NO | MUC1* HC-CDR3 |
| Anti-MUC1* | 49 | ITFSTYTMS | 50 | TISTGGDKTYYSDSVKG | 51 | GTTAMYYYAMDY |
| 3C2B1 | | | | | | |
| Anti-MUC1* | 25 | TYAMS | 26 | SIGRAGSTYYSDSVKG | 27 | GPIYNDYDEFAY |
| 20A10 | | | | | | |
| Anti-MUC1* | 11 | GYAMS | 12 | TISSGGTYIYYPDSVKG | 13 | LGGDNYYEYFDV |
| C2 | | | | | | |
| | SEQ ID | | SEQ ID | | SEQ | |
| Construct | NO | MUC1* LC-CDR1 | NO | MUC1* LC-CDR2 | ID NO | MUC1* LC-CDR3 |
| Anti-MUC1* | 52 | RASKSISTSDYNYIH | 53 | LASNLES | 54 | QHSRELPLT |
| 3C2B1 | | | | | | |
| Anti-MUC1* | 28 | KSSQSVLYSSNQKNYLA | 29 | WASTRES | 30 | HQYLSSLT |
| 20A10 | | | | | | |
| Anti-MUC1* | 14 | RASKSVSTSGYSYMH | 15 | LASNLES | 16 | QHSRELPFT |
| C2 | | | | | | |

TABLE 2
Humanized heavy chain variable domain and light chain variable domain
sequences of exemplary anti-MUC1* binding domains

| Construct | SEQ ID NO | Sequence |
|---|---|---|
| Anti-MUC1* 3C2B1 HC | 47 | EVQLVESGGGLVKPGGSLRLSCAASGITFSTYTMSWVRQ |
| Variable domain | | APGKGLEWVATISTGGDKTYYSDSVKGRFTISRDNAKNT |
| | | LYLQMNSLRAEDTAVYYCARGTTAMYYYAMDYWGQG |
| | | TTVTVSS |
| Anti-MUC1* 3C2B1 LC | 48 | DIVLTQSPASLAVSPGQRATITCRASKSISTSDYNYIH |
| Variable domain | | WYQQKPGQPPKLLIYLASNLESGVPARFSGSGSGTD |
| | | FTLTISPVEAEDAATYYCQHSRELPLTFGGGTKVEIK |
| Anti-MUC1* C2 HC | 9 | EVQLVESGGGLVKPGGSLRLSCAASGFTFSGYAMSWVR |
| Variable domain | | QAPGKGLEWVSTISSGGTYIYYPDSVKGRFTISRDNAKNS |
| | | LYLQMNSLRAEDTAVYYCARLGGDNYYEYFDVWGKGT |
| | | TVTVSS |
| Anti-MUC1* C2 LC | 10 | DIVLTQSPASLAVSPGQRATITCRASKSVSTSGYSYMHW |
| Variable domain | | YQQKPGQPPKLLIYLASNLESGVPARFSGSGSGTDFTLTI |
| | | NPVEANDTANYYCQHSRELPFTFGGGTKVEIKRT |
| Anti-MUC1* 20A10 HC | 23 | QVQLVESGGGLVKPGGSLRLSCAASGFTFSTYAMSWIRQ |
| Variable domain | | APGKGLEWVASIGRAGSTYYSDSVKGRFTISRDNAKNSL |
| | | YLQMNSLRAEDTAVYYCARGPIYNDYDEFAYWGQGTL |
| | | VTVSS |
| Anti-MUC1* 20A10 LC | 24 | EIVLTQSPATLSLSPGERATLSCKSSQSVLYSSNQKNYLA |
| Variable domain | | WYQQKPGQAPRLLIYWASTRESGIPARFSGSGSGTDFTLT |
| | | ISSLEPEDFAVYYCHQYLSSLTFGGGTKVEIKR |

TABLE 3
anti-MUC1* single chain variable fragment (scFv) sequences

| Construct | SEQ ID NO | Sequence |
|---|---|---|
| Anti-MUC1* 3C2B1 scFv | 55 | EVQLVESGGGLVKPGGSLRLSCAASGITFSTYTMSWVRQ |
| Humanized, VH-VL, GS3 | | APGKGLEWVATISTGGDKTYYSDSVKGRFTISRDNAKNT |
| | | LYLQMNSLRAEDTAVYYCARGTTAMYYYAMDYWGQG |
| | | TTVTVSSGGGGSGGGGSGGGGSDIVLTQSPASLAVSPGQ |
| | | RATITCRASKSISTSDYNYIHWYQQKPGQPPKLLIYLASN |
| | | LESGVPARFSGSGSGTDFTLTISPVEAEDAATYYCQHSRE |
| | | LPLTFGGGTKVEIK |
| Anti-MUC1* 3C2B1 scFv | 56 | DIVLTQSPASLAVSPGQRATITCRASKSISTSDYNYIHWY |
| Humanized, VL-VH, GS3 | | QQKPGQPPKLLIYLASNLESGVPARFSGSGSGTDFTLTISP |
| | | VEAEDAATYYCQHSRELPLTFGGGTKVEIKGGGGSGGG |
| | | GSGGGGSEVQLVESGGGLVKPGGSLRLSCAASGITESTY |
| | | TMSWVRQAPGKGLEWVATISTGGDKTYYSDSVKGRFTI |
| | | SRDNAKNTLYLQMNSLRAEDTAVYYCARGTTAMYYYA |
| | | MDYWGQGTTVTVSS |
| Anti-MUC1* 20A10 scFv | 31 | QVQLVESGGGLVKPGGSLRLSCAASGFTFSTYAMSWIRQ |
| | | APGKGLEWVASIGRAGSTYYSDSVKGRFTISRDNAKNSL |
| | | YLQMNSLRAEDTAVYYCARGPIYNDYDEFAYWGQGTL |
| | | VTVSSGGGGSGGGGSGGGGSEIVLTQSPATLSLSPGERAT |
| | | LSCKSSQSVLYSSNQKNYLAWYQQKPGQAPRLLIYWAS |
| | | TRESGIPARFSGSGSGTDFTLTISSLEPEDFAVYYCHQYLS |
| | | SLTFGGGTKVEIKR |
| Anti-MUC1* C2 scFv | 17 | EVQLVESGGGLVKPGGSLRLSCAASGFTFSGYAMSWVR |
| | | QAPGKGLEWVSTISSGGTYIYYPDSVKGRFTISRDNAKNS |
| | | LYLQMNSLRAEDTAVYYCARLGGDNYYEYFDVWGKGT |
| | | TVTVSSGGGGSGGGGSGGGGSDIVLTQSPASLAVSPGQR |
| | | ATITCRASKSVSTSGYSYMHWYQQKPGQPPKLLIYLASN |
| | | LESGVPARFSGSGSGTDFTLTINPVEANDTANYYCQHSRE |
| | | LPFTFGGGTKVEIKRT |

In some embodiments, the antibody further comprises a fragment crystallizable (Fc) region. In some embodiments, the Fc region comprises an IgG CH2 domain and an IgG CH3 domain. In some embodiments, the Fc region comprises a heterodimeric Fc region. In some embodiments, the heterodimeric Fc region comprises a (n) (e.g., human) IgG1, IgG2, IgG3, or IgG4 domain. In some embodiments, the heterodimeric Fc region comprises a (n) (e.g., human) IgG1 domain. In some embodiments, the heterodimeric Fc region comprises a (n) (e.g., human) IgG2 domain. In some embodiments, the heterodimeric Fc region comprises a (n) (e.g., human) IgG3 domain. In some embodiments, the heterodimeric Fc region comprises a (n) (e.g., human) IgG4 domain.

In some embodiments, the heterodimeric Fc region, wherein the heterodimeric Fc region comprises a knob chain and a hole chain, forming a knob-into-hole (KIH) structure (Spiess et al. Molecular Immunology 67, 95-106 (2015)), format. In some embodiments, the knob chain comprises a (n) (e.g., human) IgG1, IgG2, IgG3, or IgG4 domain. In some embodiments, the knob chain comprises a (n) (e.g., human) IgG1 domain. In some embodiments, the knob chain comprises a (n) (e.g., human) IgG2 domain. In some embodiments, the knob chain comprises a (n) (e.g., human) IgG3 domain. In some embodiments, the knob chain comprises a (n) (e.g., human) IgG4 domain. In some embodiments, the hole chain comprises a (n) (e.g., human) IgG1, IgG2, IgG3, or IgG4 domain. In some embodiments, the hole chain comprises a (n) (e.g., human) IgG1 domain. In some embodiments, the hole chain comprises a (n) (e.g., human) IgG2 domain. In some embodiments, the hole chain comprises a (n) (e.g., human) IgG3 domain. In some embodiments, the hole chain comprises a (n) (e.g., human) IgG4 domain.

In some embodiments, the target cell is a cancer cell. In some embodiments the cancer is breast cancer, colon cancer, prostate cancer, pancreatic cancer, or lung cancer.

In some embodiments, the antibody binds to a cancer cell that expresses MUC1* on its surface.

### Antibody-Drug Conjugates

An antibody-drug conjugate (ADC) allows the targeted delivery of therapeutic agent(s) to specific cells and/or tissues. In certain embodiments, the ADC comprises an agent (e.g., a therapeutic agent) capable of inhibiting topoisomerase (e.g., topoisomerase I (TopoI)) or inhibiting tubulin assembly into microtubules. In certain embodiments, the ADC comprises a toxin (e.g, an agent that inhibits an enzyme or metabolic process of a cell).

In another aspect, provided herein are conjugates, e.g., antibody-drug conjugates. The ADC comprises a therapeutic agent (indicated as X); a linking moiety (indicated as L); a coupling moiety (indicated as R); and a conjugation moiety (indicated as Z) to an antibody (indicated as [symbol] ) by nucleophilic attack by a thiol group of a cysteine. In some embodiments, intracellular cleavage of the L linker allows the separation of the therapeutic) agent X from the [symbol] , thereby promoting the uptake or retention therapeutic agent X into cells or tissue retention or permitting the therapeutic X to transport to or be transported to its site of action.

In another aspect, provided herein are methods of making antibody drug conjugates by reacting an antibody with a linker and therapeutic agent or toxin. The antibody and the linker may react via a nucleophilic attack by a thiol group of a cysteine on a electrophilic group of the linker (e.g. a maleimide group). The linker and therapeutic agent may be combined as a linker-toxin molecule.

In certain embodiments, the ADC comprises Formula (I): [Ab]-[Z-L-R-X] y wherein: [Ab] is an antibody or antibody fragment that binds specifically to the extracellular domain of MUC1*; Z is a conjugation moiety that forms a covalent bond with a sulfur atom of a cysteine residue of [Ab]; L is a di- or tri- or tetra-peptide linking moiety having Z bonded to its N-terminus and R bonded to its C-terminus; R is a coupling moiety; X is a toxin moiety; and y is an integer from 1 to 10. In some embodiments, [Ab] binds specifically to SEQ ID NO: 1. In some embodiments, [Ab] binds specifically to SEQ ID NO: 2, SEQ ID NO: 155, SEQ ID NO: 156, SEQ ID NO: 157, SEQ ID NO: 158, and/or SEQ ID NO: 159. In some embodiments, [Ab] comprises three heavy chain (HC) complementarity determining region (CDRs): MUC1* HC-CDR1, MUC1* HC-CDR2, and MUC1* HC-CDR3 and three light chain (LC) complementarity determining region (CDRs): MUC1* LC-CDR1, MUC1* LC-CDR2, and MUC1* LC-CDR3. In some embodiments, the MUC1* HC-CDR1 comprises SEQ ID NO: 49, the MUC1* HC-CDR2 comprises SEQ ID NO: 50, the MUC1* HC-CDR3 comprises SEQ ID NO: 51, the MUC1* LC-CDR1 comprises SEQ ID NO: 52, the MUC1* LC-CDR2 comprises SEQ ID NO: 53, and the MUC1* LC-CDR3 comprises SEQ ID NO: 54. In some embodiments, the MUC1* HC-CDR1, the MUC1* HC-CDR2, the MUC1* HC-CDR3, the MUC1* LC-CDR1, the MUC1* LC-CDR2, and the MUC1* LC-CDR3 comprises amino acid sequences selected from SEQ ID NOs: 11-16, 25-29, 37-42, 61-66, 73-78, 85-90, 97-102, 109-114, 121-126, or 133-138. L may be susceptible to cleavage by a protease. The protease may be a lysosomal protease. The protease may be cathepsin B or cathepsin D. R may be a self-cleaving moiety. In some embodiments, R comprises para-aminobenzyloxycarbonyl (PAB) or aminomethyl (AM). X may be derived from a compound capable of inhibiting topoisomerase I or a compound capable of inhibiting tubulin formation. The topoisomerase I inhibitor may be exatecan, DXd, or SN38. The tubulin inhibitor may be MMAE or MMAF.

The ADC can comprise a structure, such as those provided below, wherein n is 1 to 10, indicating that more than one linker/toxin moiety can be conjugated to each antibody.

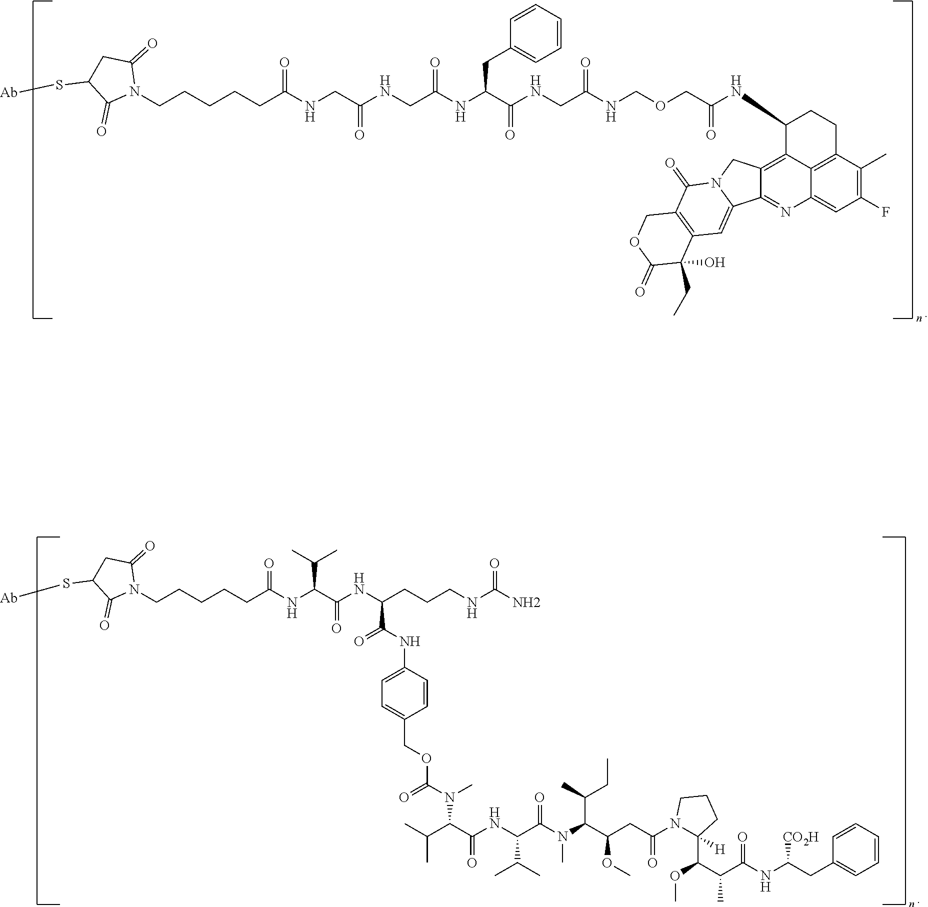

The efficacy of an ADC is governed, in part, by the drug to antibody ratio or the DAR. Basically, the more toxins you attach to your antibody, the more cell killing there is. However, attaching too many toxins to an antibody can destabilize the antibody or sterically hinder the interaction between the antibody and the target antigen. Hydrophobic interaction chromatography (HIC) is a bioanalytical technique that is used to determine the drug-antibody ratio (DAR) of antibody-drug-conjugates (ADC's). An HIC column on a high-pressure liquid chromatography (HPLC) system is used for the analysis and characterization of ADCs using a salt gradient buffer. HIC separates proteins according to differences in their surface hydrophobicity; for example, the more drug that is bound to the antibody, the longer the retention time.

In certain embodiments, the DAR for a conjugate or population of conjugates provided herein ranges from 1 to 20. In certain embodiments, the DAR ranges from 1 to 15. In certain embodiments, the DAR ranges from 1 to 10. In certain embodiments, the DAR ranges from 1 to 8. In certain embodiments, the DAR ranges from 1 to 7. In certain embodiments, the DAR ranges from 1 to 6. In certain embodiments, the DAR ranges from 1 to 5. In certain embodiments, the DAR ranges from 1 to 4. In certain embodiments, the DAR ranges from 3 to 9. In certain embodiments, the DAR ranges from 3 to 7. In certain embodiments, the DAR ranges from 3 to 5. In certain embodiments, the DAR ranges from 3 to 9. In certain embodiments, the DAR ranges from 5 to 12. In certain embodiments, the DAR ranges from 5 to 10. In certain embodiments, the DAR ranges from 5 to 8. In certain embodiments, the DAR ranges from 7 to 12. In certain embodiments, the DAR ranges from 7 to 10. In certain embodiments, the DAR ranges from 7 to 8. In certain embodiments, the DAR ranges from 8 to 10.

In certain embodiments, the DAR for a conjugate or population of conjugates provided herein is about 1, about 2, about 3, about 4, about 5, about 6, about 7, about 8, about 9, about 10, about 11, or about 12. In some embodiments, the DAR is about 3.1, about 3.2, about 3.3, about 3.4, about 3.5, about 3.6, about 3.7, about 3.8, or about 3.9. In some embodiments, the DAR is about 7.1, about 7.2, about 7.3, about 7.4, about 7.5, about 7.6, about 7.7, about 7.8, about 7.9, or about 8.0.

In certain embodiments, fewer than the theoretical maximum of units are conjugated to the polypeptide, e.g., antibody, during a conjugation reaction.

In certain embodiments, an amino acid that attaches to a unit is in the heavy chain of an antibody. In certain embodiments, an amino acid that attaches to a unit is in the light chain of an antibody. In certain embodiments, an amino acid that attaches to a unit is in the hinge region of an antibody. In certain embodiments, an amino acid that attaches to a unit is in the Fc region of an antibody. In certain embodiments, an amino acid that attaches to a unit is in the constant region (e.g., CH1, CH2, or CH3 of a heavy chain, or CH1 of a light chain) of an antibody. In yet other embodiments, an amino acid that attaches to a unit or a drug unit is in the VH framework regions of an antibody. In yet other embodiments, an amino acid that attaches to unit is in the VL framework regions of an antibody.

It is to be understood that the preparation of the conjugates described herein may result in a mixture of conjugates with a distribution of one or more units attached to a polypeptide (i.e., heterogenous), for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

The present disclosure provides an antibody-drug conjugate (ADC) comprising a monoclonal antibody, or an antigen-binding fragment thereof, directed against MUC1* conjugated to a cytotoxin. The term “antibody-drug conjugate,” as used herein, refers to a compound comprising a monoclonal antibody (mAb) attached to a cytotoxic agent (generally a small molecule drug with a high systemic toxicity) via chemical linkers. In some embodiments, an ADC may comprise a small molecule cytotoxin that has been chemically modified to contain a linker. The linker is then used to conjugate the cytotoxin to the antibody, or antigen-binding fragment thereof. Upon binding to the target antigen on the surface of a cell, the ADC is internalized and trafficked to the lysosome where the cytotoxin is released by either proteolysis of a cleavable linker (e.g., by cathepsin B found in the lysosome) or by proteolytic degradation of the antibody, if attached to the cytotoxin via a non-cleavable linker. The cytotoxin then translocates out of the lysosome and into the cytosol or nucleus, where it can then bind to its target, depending on its mechanism of action.

The antibody-drug conjugate described herein may comprise a whole antibody or an antibody fragment. The parent antibody may be murine, rabbit, human, humanized, camelid or other species.

The ADC may comprise an antigen-binding fragment of an antibody. The terms “antibody fragment,” “antigen-binding fragment,” “functional fragment of an antibody,” and “antigen-binding portion” are used interchangeably herein and refer to one or more fragments or portions of an antibody that retain the ability to specifically bind to an antigen (see, generally, Holliger et al., Nat. Biotech., 23 (9): 1 126-1129 (2005)). The antibody fragment may comprise, for example, one or more CDRs (e.g. 6 CDRs), the variable region (or portions thereof), the constant region (or portions thereof), or combinations thereof. Examples of antibody fragments include, but are not limited to, (i) a Fab fragment, which is a monovalent fragment consisting of the VL, VH, CL, and CH1 domains; (ii) a F(ab′) 2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242:423-426 (1988); Huston et al., Proc. Natl. Acad. Sci. USA, 85:5879-5883 (1988); and Osbourn et al., Nat. Biotechnol., 16:778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites.

The terms “cytotoxin” and “cytotoxic agent” refer to any molecule that inhibits or prevents the function of cells and/or causes destruction of cells (cell death), and/or exerts anti-proliferative effects. It will be appreciated that a cytotoxin or cytotoxic agent of an ADC also is referred to in the art as the “payload” of the ADC. A number of classes of cytotoxic agents are known in the art to have potential utility in ADC molecules and can be used in the ADC described herein. Such classes of cytotoxic agents include, for example, anti-microtubule agents (e.g., auristatins and maytansinoids), pyrrolobenzodiazepines (PBDs), RNA polymerase II inhibitors (e.g., amatoxins), and DNA alkylating agents (e.g., indolinobenzodiazepine pseudodimers). Examples of specific cytotoxic agents that may be used in the ADC described herein include, but are not limited to, amanitins, auristatins, calicheamicin, daunomycins, doxorubicins, duocarmycins, dolastatins, enediynes, lexitropsins, taxanes, puromycins, maytansinoids, vinca alkaloids, tubulysins, and pyrrolobenzodiazepines (PBDs). More specifically, the cytotoxic agent may be, for example AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM1, DM4, vinblastine, methotrexate, netropsin, or derivatives or analogs thereof.

Auristatins represent a class of highly potent antimitotic agents that have shown substantial preclinical activity at well-tolerated doses. Examples of auristatins that may be used in connection with the ADC described herein include, but are not limited to, monomethyl auristatin E (MMAE) and the related molecule monomethyl auristatin F (MMAF).

In one embodiment, the cytotoxic agent may be a pyrrolobenzodiazepine (PBD) or a PBD derivative. PBD translocates to the nucleus where it crosslinks DNA, preventing replication during mitosis, damaging DNA by inducing single strand breaks, and subsequently leading to apoptosis. Some PBDs also have the ability to recognize and bind to specific sequences of DNA.

The anti-MUC1* monoclonal antibody described herein comprises at least one cytotoxin molecule conjugated thereto; however, the anti-MUC1* monoclonal antibody may comprise any suitable number of cytotoxin molecules conjugated thereto (e.g., 1, 2, 3, 4, or more cytotoxin molecules) to achieve a desired therapeutic effect.

The disclosure also provides a composition comprising the above-described antibody or antibody-drug conjugate and a pharmaceutically acceptable (e.g., physiologically acceptable) carrier. Any suitable carrier known in the art can be used within the context of the invention. The choice of carrier will be determined, in part, by the particular site to which the composition may be administered and the particular method used to administer the composition. The composition optionally may be sterile. The compositions can be generated in accordance with conventional techniques described in, e.g., Remington: The Science and Practice of Pharmacy, 21st Edition, Lippincott Williams & Wilkins, Philadelphia, Pa. (2001).

The composition desirably comprises the antibody or antibody-drug conjugate in an amount that is effective to treat or prevent a MUC1* expressing cancer. Thus, the disclosure provides a method of killing MUC1* positive cells, which comprises contacting the cells that express MUC1* with the antibody or antibody-drug conjugate described herein, or a composition comprising the antibody or ADC described herein, whereby the antibody or antibody-drug conjugate binds to MUC1* on the cells and kills the cells.

The disclosure also provides use of the antibody or ADC described herein, or the composition comprising the antibody or ADC, in the manufacture of a medicament for treating MUC1* positive cancer.

As demonstrated herein, MUC1* is expressed on a variety of cancer types. As such, the disclosure provides a method of killing such cancer cells, which comprises contacting the cancer cells that express MUC1* with the antibody-drug conjugate described herein, or a composition comprising the ADC described herein, whereby the antibody-drug conjugate binds to MUC1* on the cells and kills the cells.

The antibody-drug conjugate described herein, or a composition comprising the antibody-drug conjugate, may be contacted with a population of cells that expresses MUC1* ex vivo, in vivo, or in vitro, preferably in vivo.

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the antibody or ADC or the composition comprising the antibody or ADC and a pharmaceutically acceptable carrier. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the antibody or ADC to elicit a desired response in the individual. For example, a therapeutically effective amount of the ADC of the invention is an amount which binds to MUC1* on the MUC1* positive cells and destroys them.

Alternatively, the pharmacologic and/or physiologic effect may be prophylactic, i.e., the effect completely or partially prevents a disease or symptom thereof. In this respect, the inventive method comprises administering a “prophylactically effective amount” of the ADC or a composition comprising the ADC to a mammal that is predisposed to a cancer that expresses MUC1*. A “prophylactically effective amount” refers to an amount effective, at dosages and for periods of time necessary, to achieve a desired prophylactic result (e.g., prevention of disease onset).

Therapeutic or prophylactic efficacy can be monitored by periodic assessment of treated patients. In one embodiment, the ADC described herein inhibits or suppresses proliferation of MUC1*-expressing cells by at least about 10% (e.g., at least about 20%, at least about 30%, at least about 40%, at least about 50%, at least about 60%, at least about 70%, at least about 80%, at least about 90%, or at least about 100%). Cell proliferation can be measured using any suitable method known in the art, such as measuring incorporation of labeled nucleosides (e.g., 3H-thymidine or bromodeoxyuridine Brd(U)) into genomic DNA (see, e.g., Madhavan, H. N., J. Stem Cells Regen. Med., 3 (1): 12-14 (2007)).

The antibody or ADC described herein, or a composition comprising the antibody ADC, can be administered to a mammal (e.g., a human) using standard administration techniques, including, for example, intravenous, intraperitoneal, subcutaneous. More preferably, the antibody or ADC or composition containing the same is administered to a mammal by intravenous injection.

The antibody or ADC described herein, or the composition comprising the antibody or ADC, can be administered with one or more additional therapeutic agents, which can be co-administered to the mammal. The term “co-administering,” as used herein, refers to administering one or more additional therapeutic agents and the antibody or ADC described herein, or the antibody or ADC-containing composition, sufficiently close in time such that the antibody or ADC can enhance the effect of one or more additional therapeutic agents, or vice versa. In this regard, the antibody or ADC or the composition containing the same may be administered first, and the one or more additional therapeutic agents may be administered second, or vice versa. For example, the antibody or ADC or composition containing the same may be administered in combination with other agents (e.g., as an adjuvant) for the treatment or prevention of MUC1* positive cancer. In this respect, the antibody or ADC or antibody or ADC-containing composition can be used in combination with at least one other anticancer agent including, for example, any suitable chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer), and/or surgery.

As one example of how antibodies of the invention can be incorporated into ADCs, antibody drug conjugates, for the treatment of MUC1* positive cancers, a toxin was attached to anti-MUC1* antibody C2. In this particular example, the toxin is MMAE, monomethyl auristatin, although there are a variety of toxins and functional linkers known to those skilled in the art that facilitate ADC-directed killing of target cells, especially cancer cells. One of the in vitro methods for characterizing each ADC is to measure the ability of the antibody to bind the target antigen before as well as after the coupling of the toxin to the antibody. The specificity of the antibody can be compromised by the process of chemically conjugating a number toxins to the antibody. This phenomenon is due more to the intrinsic stability, or instability, of each antibody than being due to elements of the coupling process. In many cases, the payload is coupled to the antibody via binding to free thiols that are generated by breaking, or reducing, disulfide bonds. Disulfide bonding holds together the two heavy chains as well as supports the structure of the variable regions, which are the antibody recognition units. A significant challenge is reducing enough disulfide bonds to allow for the attachment of multiple toxins, without breaking the disulfide bonds that maintain critical antibody structure. Unexpectedly, C2 is a very stable, well-behaved antibody that allowed the attachment of many toxins without corrupting the structure of the antibody or altering its ability to recognize its target antigen. A challenge encountered when attaching payloads to antibody 20A10 involved the timing of the disulfide reduction and the conjugation of the toxin. Most protocols for ADC coupling call for iterative disulfide reduction, then testing for the number of free thiols. If this is a prolonged process, the free thiols can re-oxidize, which may recreate the original structure or could create a new structure that results in loss of antibody target specificity. In preliminary studies, this situation occurred when attempting to conjugate toxins to antibody 20A10, which is an IgG2b isotype.

### Antibodies

An antibody (Ab) that binds to a polypeptide of interest binds as “binding” in this context is understood by one skilled in the art. For example, an antibody, or a conjugate as described herein comprising such Ab, may bind to other polypeptides or proteins, generally with lower affinity as determined by, e.g., immunoassays or other assays known in the art. In a specific embodiment, an Ab, or a conjugate as described herein comprising such Ab that specifically bind to a polypeptide of interest binds to the polypeptide of interest with an affinity that is at least 2 logs, 2.5 logs, 3 logs, 4 logs or greater than the affinity when Ab or the conjugate bind to another polypeptide. In another specific embodiment, an Ab, or a conjugate as described herein comprising such Ab, does not specifically bind a polypeptide other than the polypeptide of interest. In a specific embodiment, an Ab, or a conjugate as described herein comprising Ab, specifically binds to a polypeptide of interest with an affinity (Kd) less than or equal to 20 mM. In particular embodiments, such binding is with an affinity (Kd) less than or equal to about 20 mM, about 10 mM, about 1 mM, about 100 μM, about 10 μM, about 1 μM, about 100 nM, about 10 nM, or about 1 nM. Unless otherwise noted, “binds,” “binds to,” “specifically binds” or “specifically binds to” in this context are used interchangeably.

In some embodiments, the target cell is a cancer cell. In some embodiments the cancer is breast cancer, colon cancer, prostate cancer, pancreatic cancer, or lung cancer.

In some embodiments, the antibody binds to a cancer cell that expresses MUC1* on the surface.

In certain embodiments, the antibody comprises about 10, about 20, about 30, about 40, about 50, about 100, about 150, about 200, about 250, about 300, about 350, about 400, about 450, about 500, about 550, about 600, about 650, about 700, about 750, about 800, about 850, about 900, or about 950 amino acids.

In certain embodiments, the antibody comprises about 10-50, about 50-100, about 100-150, about 150-200, about 200-250, about 250-300, about 300-350, about 350-400, about 400-450, about 450-500, about 500-600, about 600-700, about 700-800, about 800-900, or about 900-1000 amino acids.

In certain embodiments, the conjugate comprises an antibody, Ab. In certain embodiments, the Ab is a monoclonal antibody. In certain embodiments, the Ab is a human antibody. In certain embodiments, the Ab is a humanized antibody. Humanized antibodies of the invention include, but are not limited to, a humanized C2 antibody comprising SEQ ID NOs: 9-10, a humanized 20A10 antibody comprising SEQ ID NOs: 23-24, a humanized E6 antibody comprising SEQ ID NOs: 35-36, a humanized 3C2B1 antibody comprising SEQ ID NOs: 47-48, a humanized 5C6F3 antibody comprising SEQ ID NOs: 59-60, a humanized 25E6 antibody comprising SEQ ID NOs: 71-72, a humanized C3 antibody comprising SEQ ID NOs: 83-84, a humanized C8 antibody comprising SEQ ID NOs: 95-96, a humanized 18G12 antibody comprising SEQ ID NOs: 107-108, a humanized 28F9 antibody comprising SEQ ID NOs: 119-120, or a humanized 1E4 antibody comprising SEQ ID NOs: 131-132. All of the forgoing antibodies bound to the PSMGFR peptide, as expected.

In certain embodiments, the Ab is a chimeric antibody. In certain embodiments, the Ab is a full-length antibody that comprises two heavy chains and two light chains. In particular embodiments, the Ab is an IgG antibody, e.g., is an IgG1, IgG2, IgG3 or IgG4 antibody. In certain embodiments, the Ab is a single chain antibody. In yet other embodiments, the Ab is an antigen-binding fragment of an antibody, e.g., a Fab fragment.

In particular embodiments, the Ab is an IgG1 antibody. In particular embodiments, the Ab is an IgG2b antibody.

In some embodiments, the antibody, or functional fragment or functional variant thereof binds specifically to MUC1*. In some embodiments, the antibody comprises an anti-MUC1* heavy chain and an anti-MUC1* light chain.

In some embodiments, the anti-MUC1* heavy chain comprises an anti-MUC1* heavy chain variable domain. In some embodiments, the anti-MUC1* heavy chain variable domain comprises a variable domain of an IgG1, IgG2, IgG3, or IgG4 heavy chain. In some embodiments, the anti-MUC1* light chain comprises an anti-MUC1* light chain variable domain. In some embodiments, the anti-MUC1* light chain variable domain comprises a variable domain of a Kappa or Lambda light chain.

In some embodiments, the anti-MUC1* heavy chain variable domain comprises the variable domain of an IgG1 heavy chain and the anti-MUC1* light chain variable domain comprises the variable domain of a Kappa or Lambda light chain. In some embodiments, the anti-MUC1* heavy chain variable domain comprises the variable domain of an IgG2 heavy chain and the anti-MUC1* light chain variable domain comprises the variable domain of a Kappa or Lambda light chain. In some embodiments, the anti-MUC1* heavy chain variable domain comprises the variable domain of an IgG3 heavy chain and the anti-MUC1* light chain variable domain comprises the variable domain of a Kappa or Lambda light chain. In some embodiments, the anti-MUC1* heavy chain variable domain comprises the variable domain of an IgG4 heavy chain and the anti-MUC1* light chain variable domain comprises the variable domain of a Kappa or Lambda light chain.

In some embodiments, the anti-MUC1* heavy chain variable domain comprises the variable domain of an IgG1 heavy chain and the anti-MUC1* light chain variable domain comprises the variable domain of a Kappa light chain. In some embodiments, the anti-MUC1* heavy chain variable domain comprises the variable domain of an IgG2 heavy chain and the anti-MUC1* light chain variable domain comprises the variable domain of a Kappa light chain. In some embodiments, the anti-MUC1* heavy chain variable domain comprises the variable domain of an IgG3 heavy chain and the anti-MUC1* light chain variable domain comprises the variable domain of a Kappa light chain. In some embodiments, the anti-MUC1* heavy chain variable domain comprises the variable domain of an IgG4 heavy chain and the anti-MUC1* light chain variable domain comprises the variable domain of a Kappa light chain.

In some embodiments, the anti-MUC1* heavy chain variable domain comprises the variable domain of an IgG1 heavy chain and the anti-MUC1* light chain variable domain comprises the variable domain of a Lambda light chain. In some embodiments, the anti-MUC1* heavy chain variable domain comprises the variable domain of an IgG2 heavy chain and the anti-MUC1* light chain variable domain comprises the variable domain of a Lambda light chain. In some embodiments, the anti-MUC1* heavy chain variable domain comprises the variable domain of an IgG3 heavy chain and the anti-MUC1* light chain variable domain comprises the variable domain of a Lambda light chain. In some embodiments, the anti-MUC1* heavy chain variable domain comprises the variable domain of an IgG4 heavy chain and the anti-MUC1* light chain variable domain comprises the variable domain of a Lambda light chain.

In some embodiments, the antibody, or functional fragment or functional variant thereof, that binds specifically to MUC1* comprises a single-chain variable fragment (scFv) or an antigen-binding fragment (Fab). In some embodiments, the antibody, or functional fragment or functional variant thereof, that binds specifically to MUC1* comprises a single-chain variable fragment. In some embodiments, the antibody, or functional fragment or functional variant thereof, that binds specifically to MUC1* comprises an antigen-binding fragment (Fab).

In some embodiments, the anti-MUC1* heavy chain variable domain comprises complementarity determining regions (CDRs): HC-CDR1, HC-CDR2, and HC-CDR3, and wherein the HC-CDR1, the HC-CDR2, and the HC-CDR3 of the anti-MUC1* heavy chain variable domain comprise amino acid sequences according to HC-CDR1: SEQ ID NO:25 or 11; HC-CDR2: SEQ ID NO: 26 or 12; HC-CDR3: SEQ ID NO: 27 or 13; and wherein the CDRs comprise from 0-2 amino acid modification(s) (e.g., 0 or 1 amino acid modification(s)) in at least one of the HC-CDR1, HC-CDR2, or HC-CDR3. In some embodiments, the antibody comprises a binding region wherein: HC complementarity determining region (CDR) 1 comprises SEQ ID NO: 11; HC CDR2 comprises SEQ ID NO: 12; and HC CDR3 comprises SEQ ID NO: 13. In some embodiments, the antibody comprises a binding region wherein: HC CDR1 comprises SEQ ID NO: 25; HC CDR2 comprises SEQ ID NO: 26; and HC CDR3 comprises SEQ ID NO: 27. In some embodiments, the antibody comprises a binding region wherein: HC CDR1 comprises SEQ ID NO: 61, HC CDR2 comprises SEQ ID NO: 62, HC CDR3 comprises SEQ ID NO: 63. In some embodiments, the antibody comprises a binding region wherein: (b) HC CDR1 comprises SEQ ID NO: 73, HC CDR2 comprises SEQ ID NO: 74, HC CDR3 comprises SEQ ID NO: 75, In some embodiments, the antibody comprises a binding region wherein: HC CDR1 comprises SEQ ID NO: 85, HC CDR2 comprises SEQ ID NO: 86, HC CDR3 comprises SEQ ID NO: 87, In some embodiments, the antibody comprises a binding region wherein: HC CDR1 comprises SEQ ID NO: 97, HC CDR2 comprises SEQ ID NO: 98, HC CDR3 comprises SEQ ID NO: 99, In some embodiments, the antibody comprises a binding region wherein: HC CDR1 comprises SEQ ID NO: 109; HC CDR2 comprises SEQ ID NO: 110, HC CDR3 comprises SEQ ID NO: 111. In some embodiments, the antibody comprises a binding region wherein: HC CDR1 comprises SEQ ID NO: 121, HC CDR2 comprises SEQ ID NO: 122, HC CDR3 comprises SEQ ID NO: 123

In some embodiments, the anti-MUC1* light chain variable domain comprises complementarity determining regions (CDRs): LC-CDR1, LC-CDR2, and LC-CDR3, and wherein the LC-CDR1, the LC-CDR2, and the LC-CDR3 of the anti-MUC1* light chain variable domain comprises amino acid sequences according to LC-CDR1: SEQ ID NO: 28 or 14; LC-CDR2: SEQ ID NO: 29 or 15; LC-CDR3: SEQ ID NO: 30 or 16; and wherein the CDRs comprise from 0-2 amino acid modification(s) (e.g., 0 or 1 amino acid modification(s)) in at least one of the LC-CDR1, LC-CDR2, or LC-CDR3. In some embodiments, the antibody comprises a binding region wherein: LC CDR1 comprises SEQ ID NO: 14; LC CDR2 comprises SEQ ID NO: 15; and LC CDR3 SEQ ID NO: 16. In some embodiments, the antibody comprises a binding region wherein: LC CDR1 comprises SEQ ID NO: 28; LC CDR2 comprises SEQ ID NO: 29; and LC CDR3 comprises SEQ ID NO: 30. In some embodiments, the antibody comprises a binding region wherein: LC CDR1 comprises SEQ ID NO: 40, LC CDR2 comprises SEQ ID NO: 41, and LC CDR3 comprises SEQ ID NO: 42; In some embodiments, the antibody comprises a binding region wherein: LC CDR1 comprises SEQ ID NO: 52, LC CDR2 comprises SEQ ID NO: 53, and LC CDR3 comprises SEQ ID NO: 54. In some embodiments, the antibody comprises a binding region wherein: LC CDR1 comprises SEQ ID NO: 64, LC CDR2 comprises SEQ ID NO: 65, and LC CDR3 comprises SEQ ID NO: 66. In some embodiments, the antibody comprises a binding region wherein: LC CDR1 comprises SEQ ID NO: 76, LC CDR2 comprises SEQ ID NO: 77, and LC CDR3 comprises SEQ ID NO: 78; In some embodiments, the antibody comprises a binding region wherein: LC CDR1 comprises SEQ ID NO: 88, LC CDR2 comprises SEQ ID NO: 89, and LC CDR3 comprises SEQ ID NO: 90. In some embodiments, the antibody comprises a binding region wherein: LC CDR1 comprises SEQ ID NO: 100, LC CDR2 comprises SEQ ID NO: 101, and LC CDR3 comprises SEQ ID NO: 102. In some embodiments, the antibody comprises a binding region wherein: LC CDR1 comprises SEQ ID NO: 112, LC CDR2 comprises SEQ ID NO: 113, and LC CDR3 comprises SEQ ID NO: 114. In some embodiments, the antibody comprises a binding region wherein: LC CDR1 comprises SEQ ID NO: 124, LC CDR2 comprises SEQ ID NO: 125, and LC CDR3 comprises SEQ ID NO: 126.

In some embodiments, the anti-MUC1* heavy chain comprises an amino acid sequence with at least 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NOS: 154 or 158 . . . . In some embodiments, the antibody comprises: a heavy chain (HC) variable domain having at least 80%, at least 85%, at least 90%, at least 95%, or at least 98% sequence identity to SEQ ID NO: 9, 23, 35, 47, 59, 71, 83, 95, 107, 119, or 131. In some embodiments, the anti-MUC1* light chain comprises an amino acid sequence with at least 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NOs: 156 or 160. In some embodiments, the anti-MUC1* light chain (LC) variable domain comprises an amino acid sequence at least 80%, at least 85%, at least 90%, at least 95%, or at least 98% sequence identity to SEQ ID NO: 10, 24, 36, 48, 60, 72, 84, 96, 108, 120, or 132.

In some embodiments, the antibody comprises a binding region wherein: HC complementarity determining region (CDR) 1 comprises SEQ ID NO: 11; HC CDR2 comprises SEQ ID NO: 12; HC CDR3 comprises SEQ ID NO: 13; LC CDR1 comprises SEQ ID NO: 14; LC CDR2 comprises SEQ ID NO: 15; and LC CDR3 SEQ ID NO: 16.

In some embodiments, the antibody comprises: a HC variable domain having at least 80%, at least 85%, at least 90%, at least 95%, or at least 98% sequence identity to SEQ ID NO: 23, and a LC variable domain having at least 80%, at least 85%, at least 90%, at least 95%, or at least 98% sequence identity to SEQ ID NO: 24.

In some embodiments, the antibody comprises a binding region wherein: HC CDR1 comprises SEQ ID NO: 25; HC CDR2 comprises SEQ ID NO: 26; HC CDR3 comprises SEQ ID NO: 27; LC CDR1 comprises SEQ ID NO: 28; LC CDR2 comprises SEQ ID NO: 29; and LC CDR3 comprises SEQ ID NO: 30.

In some embodiments, the antibody comprises: (a) a HC variable domain having at least 80%, at least 85%, at least 90%, at least 95%, or at least 98% sequence identity to SEQ ID NO: 35, and a LC variable domain having at least 80%, at least 85%, at least 90%, at least 95%, or at least 98% sequence identity to SEQ ID NO: 36; (b) a HC variable domain having at least 80%, at least 85%, at least 90%, at least 95%, or at least 98% sequence identity to SEQ ID NO: 47, and a LC variable domain having at least 80%, at least 85%, at least 90%, at least 95%, or at least 98% sequence identity to SEQ ID NO: 48;

In some embodiments, the antibody comprises a binding region wherein: (a) HC CDR1 comprises SEQ ID NO: 37, HC CDR2 comprises SEQ ID NO: 38, HC CDR3 comprises SEQ ID NO: 39, LC CDR1 comprises SEQ ID NO: 40, LC CDR2 comprises SEQ ID NO: 41, and LC CDR3 comprises SEQ ID NO: 42;

In some embodiments, the antibody comprises a binding region wherein: HC CDR1 comprises SEQ ID NO: 49, HC CDR2 comprises SEQ ID NO: 50, HC CDR3 comprises SEQ ID NO: 51, LC CDR1 comprises SEQ ID NO: 52, LC CDR2 comprises SEQ ID NO: 53, and LC CDR3 comprises SEQ ID NO: 54.

In some embodiments, the antibody comprises a binding region wherein: (a) HC CDR1 comprises SEQ ID NO: 37, HC CDR2 comprises SEQ ID NO: 38, HC CDR3 comprises SEQ ID NO: 39, LC CDR1 comprises SEQ ID NO: 40, LC CDR2 comprises SEQ ID NO: 41, and LC CDR3 comprises SEQ ID NO: 42; or (b) HC CDR1 comprises SEQ ID NO: 49, HC CDR2 comprises SEQ ID NO: 50, HC CDR3 comprises SEQ ID NO: 51, LC CDR1 comprises SEQ ID NO: 52, LC CDR2 comprises SEQ ID NO: 53, and LC CDR3 comprises SEQ ID NO: 54.

In some embodiments, the antibody comprises: (a) a HC variable domain having at least 80%, at least 85%, at least 90%, at least 95%, or at least 98% sequence identity to SEQ ID NO: 59, and a LC variable domain having at least 80%, at least 85%, at least 90%, at least 95%, or at least 98% sequence identity to SEQ ID NO: 60; (b) a HC variable domain having at least 80%, at least 85%, at least 90%, at least 95%, or at least 98% sequence identity to SEQ ID NO: 71, and a LC variable domain having at least 80%, at least 85%, at least 90%, at least 95%, or at least 98% sequence identity to SEQ ID NO: 72; (c) a HC variable domain having at least 80%, at least 85%, at least 90%, at least 95%, or at least 98% sequence identity to SEQ ID NO: 83, and a LC variable domain having at least 80%, at least 85%, at least 90%, at least 95%, or at least 98% sequence identity to SEQ ID NO: 84; (d) a HC variable domain having at least 80%, at least 85%, at least 90%, at least 95%, or at least 98% sequence identity to SEQ ID NO: 95, and a LC variable domain having at least 80%, at least 85%, at least 90%, at least 95%, or at least 98% sequence identity to SEQ ID NO: 96; (e) a HC variable domain having at least 80%, at least 85%, at least 90%, at least 95%, or at least 98% sequence identity to SEQ ID NO: 107, and a LC variable domain having at least 80%, at least 85%, at least 90%, at least 95%, or at least 98% sequence identity to SEQ ID NO: 108; or (f) a HC variable domain having at least 80%, at least 85%, at least 90%, at least 95%, or at least 98% sequence identity to SEQ ID NO: 119, and a LC variable domain having at least 80%, at least 85%, at least 90%, at least 95%, or at least 98% sequence identity to SEQ ID NO: 120. In some embodiments, the antibody comprises a binding region wherein: (a) HC CDR1 comprises SEQ ID NO: 61, HC CDR2 comprises SEQ ID NO: 62, HC CDR3 comprises SEQ ID NO: 63, LC CDR1 comprises SEQ ID NO: 64, LC CDR2 comprises SEQ ID NO: 65, and LC CDR3 comprises SEQ ID NO: 66; (b) HC CDR1 comprises SEQ ID NO: 73, HC CDR2 comprises SEQ ID NO: 74, HC CDR3 comprises SEQ ID NO: 75, LC CDR1 comprises SEQ ID NO: 76, LC CDR2 comprises SEQ ID NO: 77, and (c) LC CDR3 comprises SEQ ID NO: 78; HC CDR1 comprises SEQ ID NO: 85, HC CDR2 comprises SEQ ID NO: 86, HC CDR3 comprises SEQ ID NO: 87, LC CDR1 comprises SEQ ID NO: 88, LC CDR2 comprises SEQ ID NO: 89, and LC CDR3 comprises SEQ ID NO: 90; (d) HC CDR1 comprises SEQ ID NO: 97, HC CDR2 comprises SEQ ID NO: 98, HC CDR3 comprises SEQ ID NO: 99, LC CDR1 comprises SEQ ID NO: 100, LC CDR2 comprises SEQ ID NO: 101, and LC CDR3 comprises SEQ ID NO: 102; (e) HC CDR1 comprises SEQ ID NO: 109; HC CDR2 comprises SEQ ID NO: 110, HC CDR3 comprises SEQ ID NO: 111, LC CDR1 comprises SEQ ID NO: 112, LC CDR2 comprises SEQ ID NO: 113, and LC CDR3 comprises SEQ ID NO: 114; or (f) HC CDR1 comprises SEQ ID NO: 121, HC CDR2 comprises SEQ ID NO: 122, HC CDR3 comprises SEQ ID NO: 123, LC CDR1 comprises SEQ ID NO: 124, LC CDR2 comprises SEQ ID NO: 125, and LC CDR3 comprises SEQ ID NO: 126.

Antibody drug conjugates (ADCs) work when the variable regions of the antibody portion recognize a molecule expressed on the outside of the cancer cell. After the antibody binds to the target antigen, the entire ADC is internalized. Antibody internalization is a requirement for the function of ADC cell killing. Often, cell internalization of the ADC biochemically alters the toxin making it more potent in some cases, while in other cases the alteration traps the toxin inside the cell to reduce off-target toxicities. Not all receptors are internalized after ligand binding or specially after antibody binding which can dimerize the receptor, making internalization more difficult if not impossible.

Single chain antibodies can be constructed by linking the heavy chain and light chain variable domains of the antibodies above with a linker such as one of the following:

GS3 Linker
(SEQ ID NO: 150)
GGGGSGGGGSGGGGS
IgG no Cys Linker
(SEQ ID NO: 151)
DKTHTKPPKPAPELLGGPGTG
X4 Linker
(SEQ ID NO: 152)
DKTHTKPPKPAPELLGGPGTGTGGPTIKPPKPPKPAPNLLGGP

### Payload

Antibody drug conjugates (ADCs) combine the target specificity of an antibody (e.g., a monoclonal antibody) with the potency of a small molecule drug (known as payload or cytotoxic group) by connecting them into a single ADC molecule that retain the properties of both. The improved selectivity and potency of ADCs leads to superior safety and efficacy resulting in broader therapeutic windows compared to conventional chemotherapeutic drugs. The term “payload” and “cytotoxic group” are used interchangeably herein.

In some embodiments, the payload is a topoisomerase inhibitor. In some embodiments, the topoisomerase inhibitor is a topoisomerase I inhibitor. In some embodiments, the topoisomerase inhibitor is exatecan or a derivative thereof. In some embodiments, the topoisomerase inhibitor is deruxtecan or a derivative thereof. In some embodiments, the topoisomerase inhibitor is SN38 or a derivative thereof.

In some embodiments, the payload is an inhibitor of microtubule assembly. In some embodiments, the inhibitor of microtubule assembly is monomethylauristatin E (MMAE) or monomethylauristatin F (MMAF).

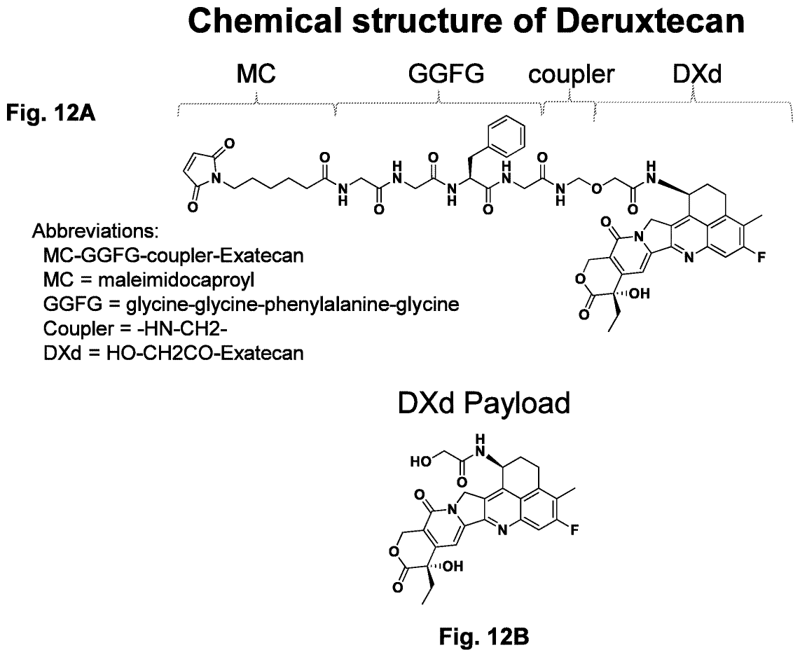

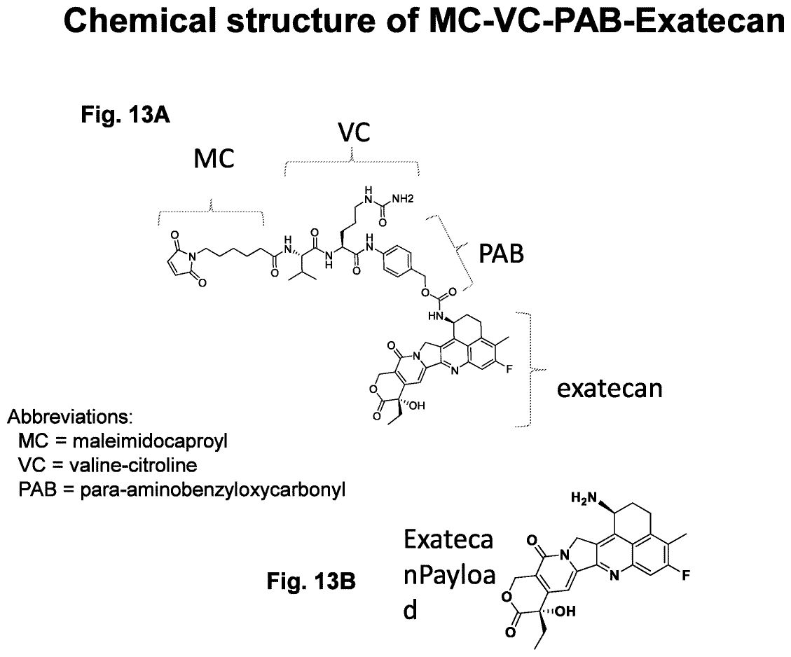

Some of the more recent toxic payloads used in ADC format, such as deruxtecan and SN38, belong to the exatecan family of topoisomerase I inhibitors. One such recent payload coupler configuration used in ADC format is deruxtecan, shown in FIGS. 12A-12B. Here, a maleimidocaproyl (MC) portion facilitates coupling to a Cysteine on the antibody. The maleimidocaproyl is connected to the toxic payload, DXd, via a glycine phenylalanine linker, GGFG, a coupler, AM, HN-CH2-which connects to the DXd. DXd is a topoisomerase I inhibitor, which is the mechanism by which it inhibits cell division. In another example shown in FIGS. 13A-13B, an exatecan payload was attached to antibodies via a para-aminobenzyloxycarbonyl (PAB) portion, connected to a valine-citrulline (VC) portion, that is in turn connected to a maleimidocaproyl (MC) portion that facilitates coupling to a Cysteine on the antibody.

Bystander effects occur when an ADC payload released after ADC binding to a target cell exerts cytotoxic activity on neighboring cells. Bystander killing can be detrimental if the neighboring cells are heathy tissue or beneficial if the neighboring cells are cancer cells. When tumors have heterogeneous expression of the ADC target antigen, bystander effects can have the beneficial effect of killing cancer cells with low target antigen expression. MMAF was designed to have less bystander killing and greater tumor cell selectivity compared to MMAE by reducing its membrane permeability.

Exatecans and deruxtecans can also have bystander effects. Clinical trials for Enhertu, an ADC targeting HER2+ breast cancers, were plagued by severe and life-threatening side effects, such as pneumonitis. Up to 16% of the Enhertu patients suffered from treatment induced pneumonitis. Enhertu did however receive FDA approval as it reportedly increased survival for metastatic breast cancer patients by nearly two years. It is not completely clear whether these side effects are due to the payload or to the HER2 target, which is also expressed on normal lung and normal heart. The first two patients treated with a HER2 targeting CAR T cell product died shortly after the infusion. Follow-on research indicated that the fatalities were due to the use of an antibody that had an extremely high affinity for HER2. More recently, yet another patient died after being treated with a different HER2-targeting ADC. These results underscore the importance of selecting an antibody with superior cancer selectivity to avoid these life-threatening toxicities. 32CB1, C2 and 20A10 unexpectedly have an extremely high degree of cancer specificity and elicited no toxicities in animal studies, where each antibody was incorporated into several ADC formats.

### Linkers

In various embodiments, a linker is used to conjugate an antibody to a toxin or other molecule (e.g., a compound capable of inhibiting topoisomerase I or a compound capable of inhibiting tubulin formation). In some embodiments the linker is a linker -L-. In certain embodiments, the linker -L- comprises one or more of carbon atoms, nitrogen atoms, sulfur atoms, oxygen atoms, and combinations thereof. In certain embodiments, the linker -L- comprises one or more amino acids. In certain embodiments, the linker -L- comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker -L- comprises a linear structure. In certain embodiments, the linker -L- comprises a di-, tri-, or tetra-peptide linking moiety.

The linker can comprise at least one glycine. The linker can comprise at least one glycine and a phenylalanine. The linker can comprise a structure of:

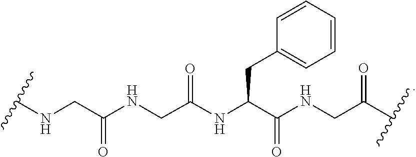 [SMILES: *NCC(=O)NCC(=O)N[C@@H](Cc1ccccc1)C(=O)NCC(*)=O]

The linker can comprise a valine. The linker can comprise a citrulline. The linker can comprise a valine and a citrulline. The linker can comprise a dipeptide linking moiety having the structure of:

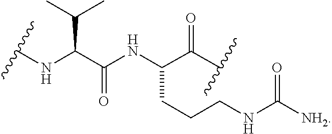 [SMILES: *N[C@H](C(=O)N[C@@H](CCCNC(N)=O)C(*)=O)C(C)C]

The linker can comprise a dipeptide comprising valine and alanine or valine and citrulline. The linker can comprise a tetrapeptide comprising four glycine residues, three glycine residues, or two glycine residues. The linker can comprise GGFG, GGVA, GGVG, or GGVA.

In certain embodiments, the linker is a cleavable linker. In some embodiments, the linker is cleavable by a lysosomal protease. In some embodiments, the linker is cleavable by cathepsin B, cathepsin L or cathepsin D. In some embodiments, the linker is maleimidocaproyl valine-citroline para-aminobenzyloxycarbonyl (MC-VC-PAB). In some embodiments, the linker is maleimidocaproyl valine-alanine para-aminobenzyloxycarbonyl (MC-VA-PAB).

In some embodiments, the linker comprises a conjugation moiety (e.g., Z) that is able to form a covalent bond, or forms a covalent bond, with an amino acid of a polypeptide. For example, the linker can comprise a maleimide. In certain embodiments, the linker comprises

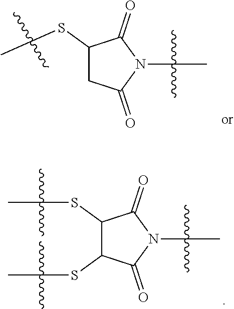 [SMILES: *SC1CC(=O)N(*)C1=O ; *SC1C(=O)N(*)C(=O)C1S*]

In certain embodiments, the linker comprises

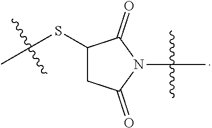 [SMILES: *SC1CC(=O)N(*)C1=O]

In certain embodiments, the linker comprises

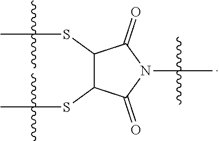 [SMILES: *SC1C(=O)N(*)C(=O)C1S*]

In certain embodiments, the linker is covalently bound to a sulfur that is part of cysteine residue of an antibody.

In certain embodiments, Z is a conjugation moiety capable of forming a covalent bond with an amino acid of a polypeptide. Z can bind to the N-terminus of the linker. Z can comprise a maleimide. In specific embodiments, the amino acid is a cysteine. In certain embodiments, Z is

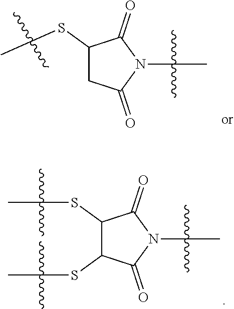 [SMILES: *SC1CC(=O)N(*)C1=O ; *SC1C(=O)N(*)C(=O)C1S*]

In certain embodiments, Z is

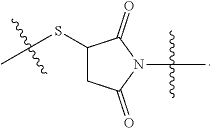 [SMILES: *SC1CC(=O)N(*)C1=O]

In certain embodiments, Z is

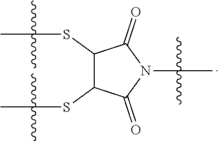 [SMILES: *SC1C(=O)N(*)C(=O)C1S*]

The linker can comprise a coupling moiety, R. In certain embodiments, R is a coupling moiety capable of binding a payload to a C-terminus of the linker. R can comprise a moiety having the structure of:

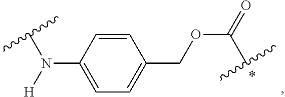 [SMILES: *Nc1ccc(COC(*)=O)cc1]

wherein the * indicates the point of attachment for the payload. R can comprise a group of structure

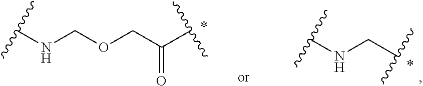

wherein the * indicates the point of attachment for the payload. R can comprise a group having the structure of:

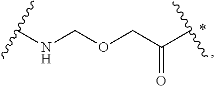 [SMILES: *NCOCC(*)=O]

wherein the * indicates the point of attachment for the payload (e.g., cytotoxic group). R can comprise a group having the structure of:

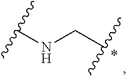 [SMILES: *CN*]

wherein the * indicates the point of attachment for the payload.

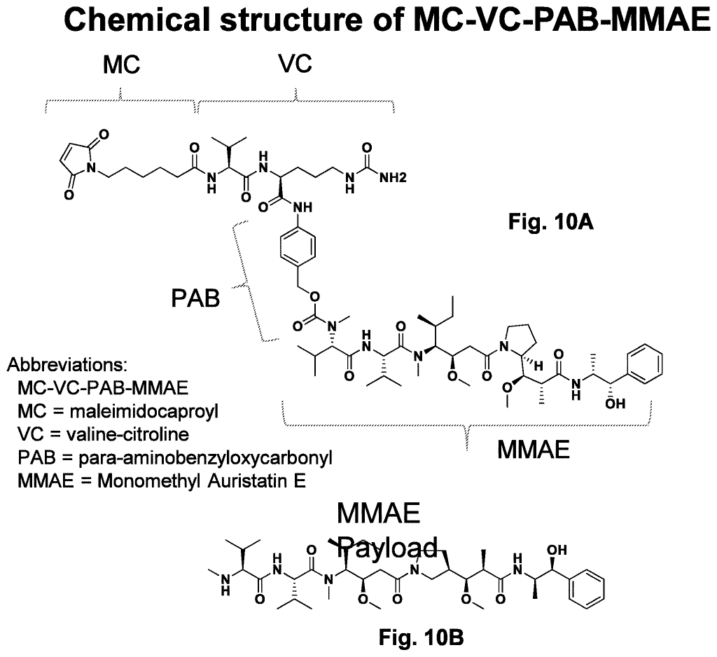

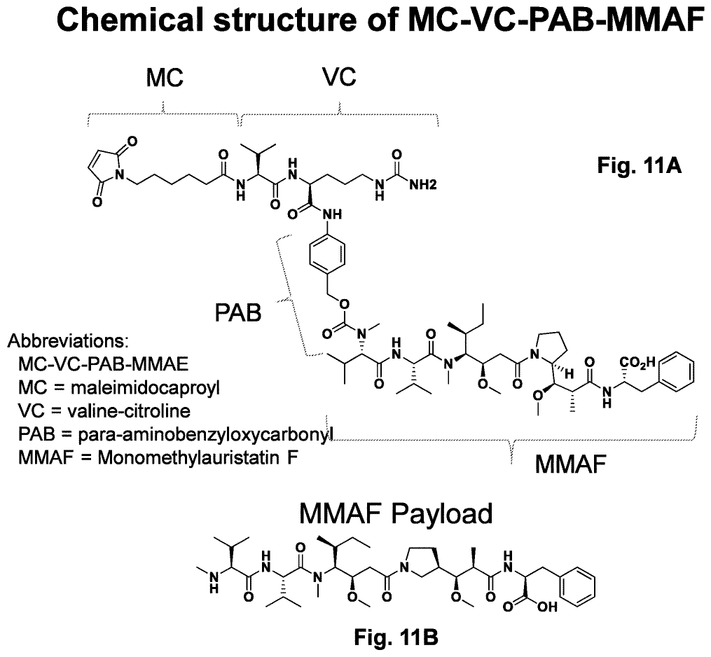

FIGS. 10A and 10B shows the chemical structure of payload monomethyl Auristatin E, MMAE, as well as convenient linker molecules and reactive molecules to facilitate chemical coupling of the payload to an antibody. In this instance, MMAE is connected to the antibody via a para-aminobenzyloxycarbonyl (PAB) portion, connected to a valine-citrulline (VC) portion, that is in turn connected to a maleimidocaproyl (MC) portion that facilitates coupling to a Cysteine on the antibody. Once the ADC has been internalized, cathepsin B enzymatically cleaves the payload from the antibody, which is facilitated by both PAB and VC. The payload, Monomethyl Auristatin E (MMAE), inhibits cell division by blocking the polymerization of tubulin.

Monomethyl Auristatin F, called MMAF, is another example of a toxic payload that inhibits cell division by blocking the polymerization of tubulin. In the example shown in FIGS. 11A and 11B, the payload, MMAF, is connected to the antibody via a para-aminobenzyloxycarbonyl (PAB) portion, connected to a valine-citrulline (VC) portion, that is in turn connected to a maleimidocaproyl (MC) portion that facilitates coupling to a Cysteine on the antibody. Once the ADC has been internalized, cathepsin B enzymatically cleaves the payload from the antibody. Unlike MMAE, MMAF contains a carboxylic acid, which makes it difficult for the payload to exit the cell after the payload has been cleaved from the antibody.

Some of the more recent toxic payloads used in ADC format, such as deruxtecan and SN38, belong to the exatecan family of topoisomerase I inhibitors. One such recent payload coupler configuration used in ADC format is deruxtecan, shown in FIG. 12A. Here, a maleimidocaproyl (MC) portion facilitates coupling to a Cysteine on the antibody. The maleimidocaproyl is connected to the toxic payload, DXd, via a glycine phenylalanine linker, GGFG, a coupler, AM, HN-CH2-which connects to the DXd. DXd (shown in FIG. 12B) is a topoisomerase I inhibitor, which is the mechanism by which it inhibits cell division. In another example shown in FIGS. 13A and 13B, we attached an exatecan payload to our antibodies via a para-aminobenzyloxycarbonyl (PAB) portion, connected to a valine-citroline (VC) portion, that is in turn connected to a maleimidocaproyl (MC) portion that facilitates coupling to a Cysteine on the antibody.

In some embodiments, the linker comprises a carbonate or carbamate group susceptible to hydrolysis in aqueous environments. Carbonate is more unstable than carbamate, which can lead to faster toxin release. In some embodiments, the linker comprises a glucuronide susceptible to cleavage by β-glucuronidase enzymes, which are enriched in some tumors.

### Linker-Toxin Molecules and Moieties

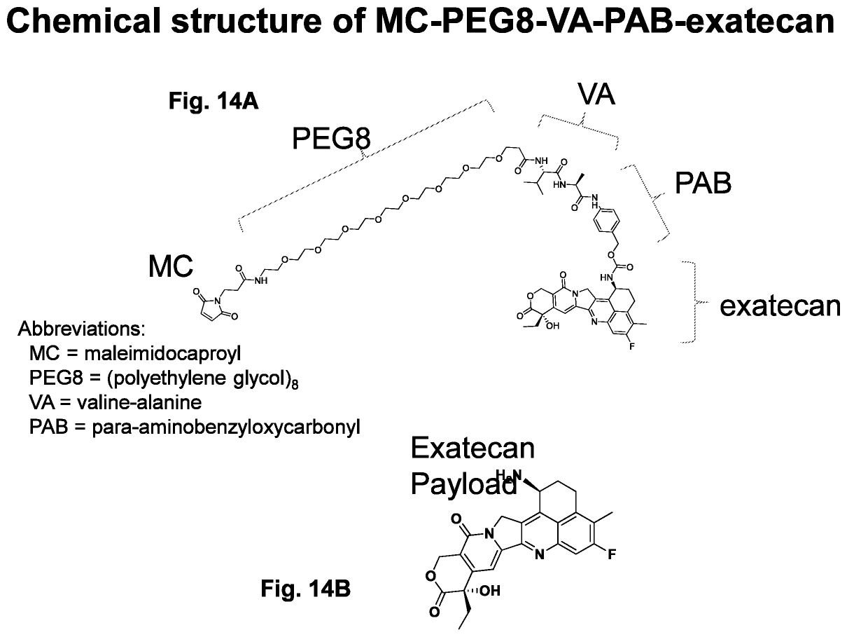

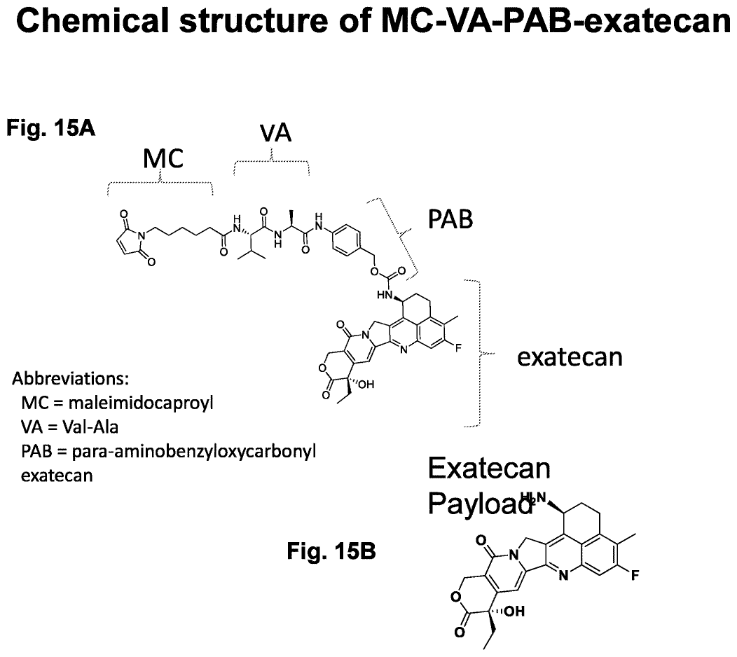

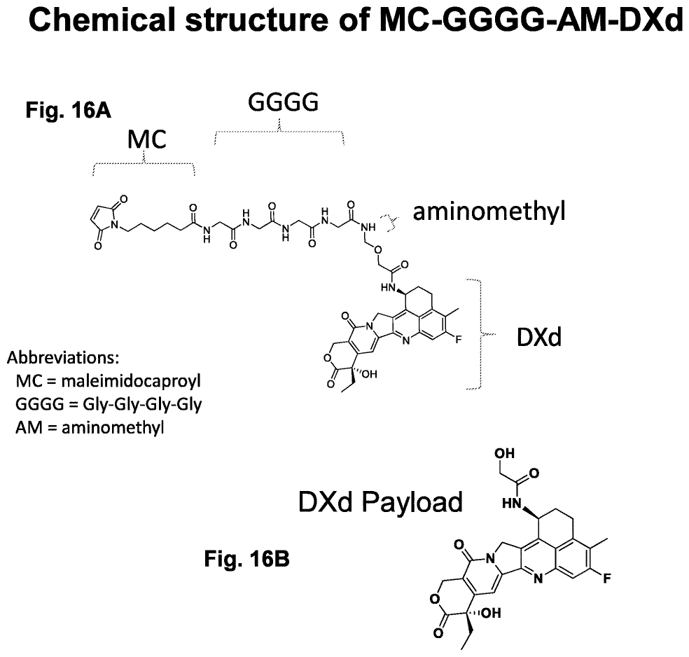

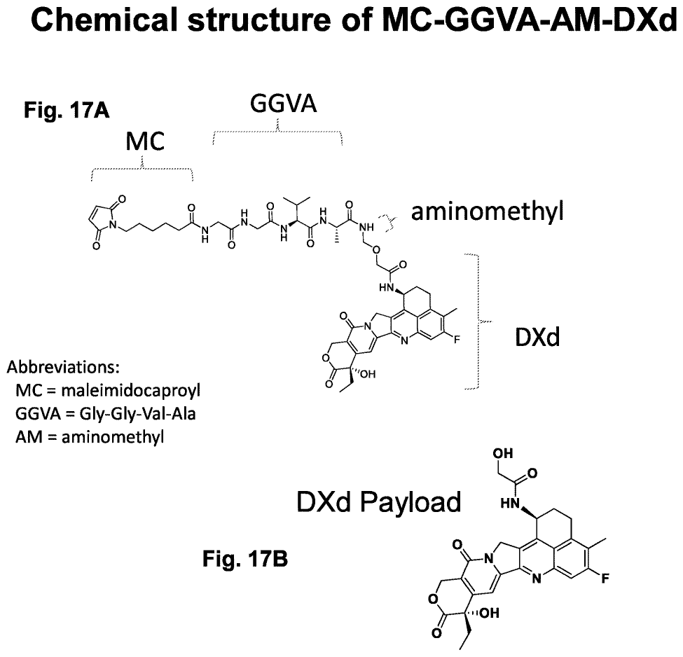

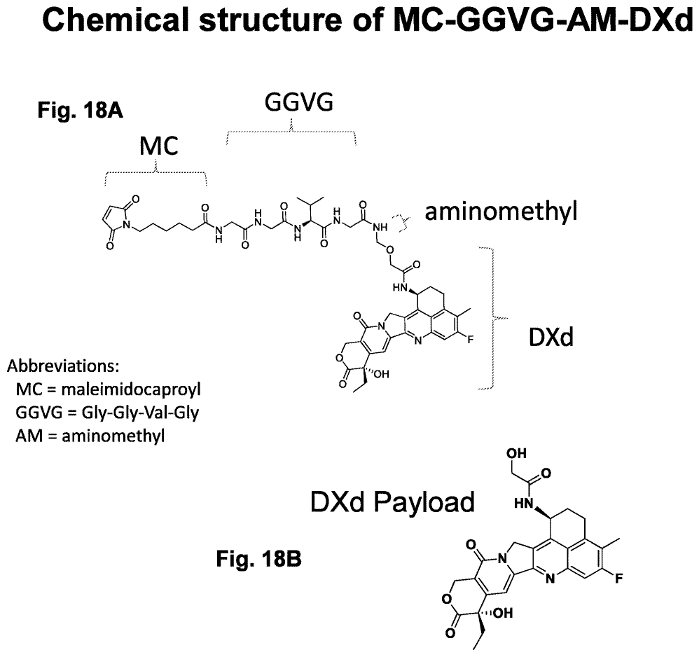

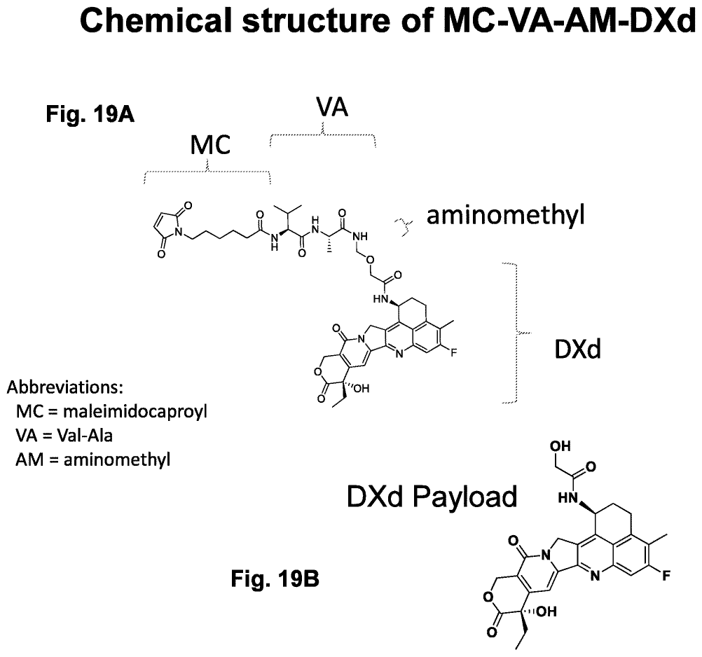

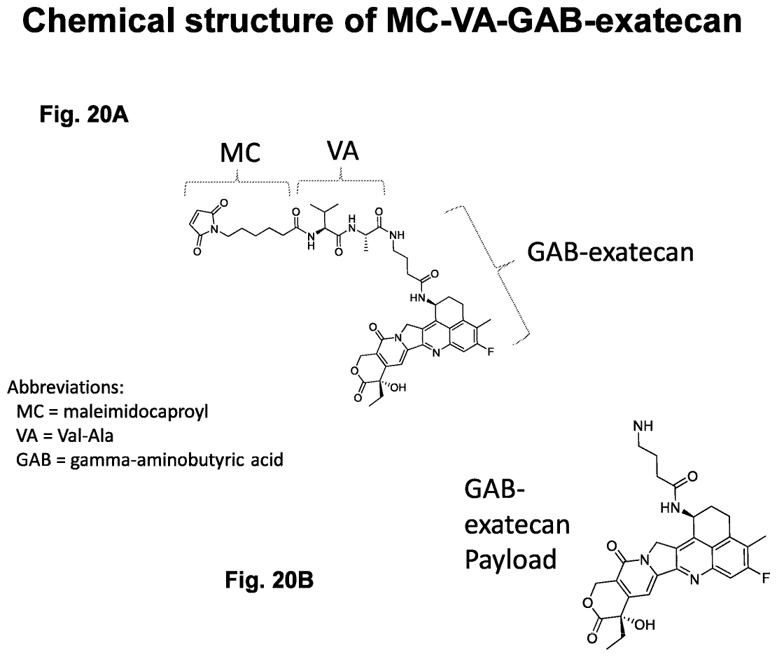

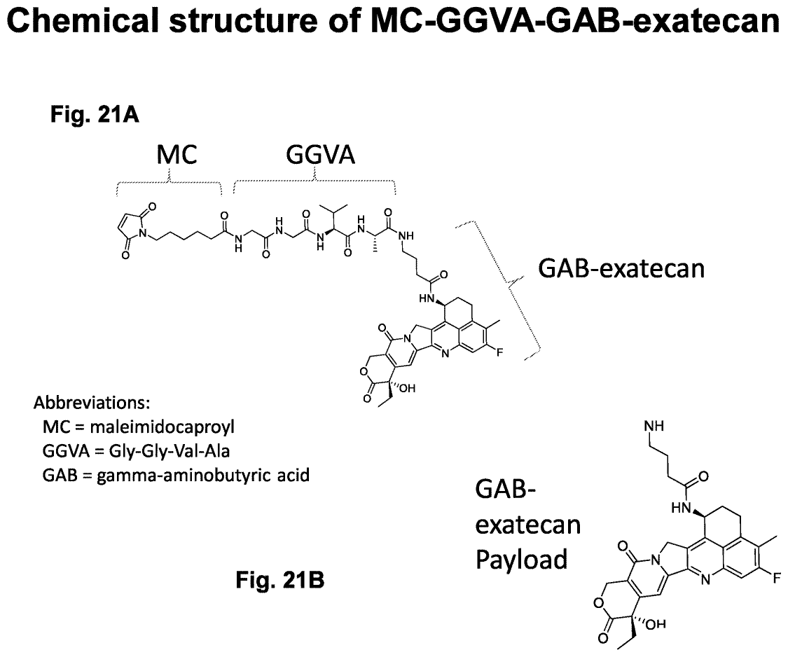

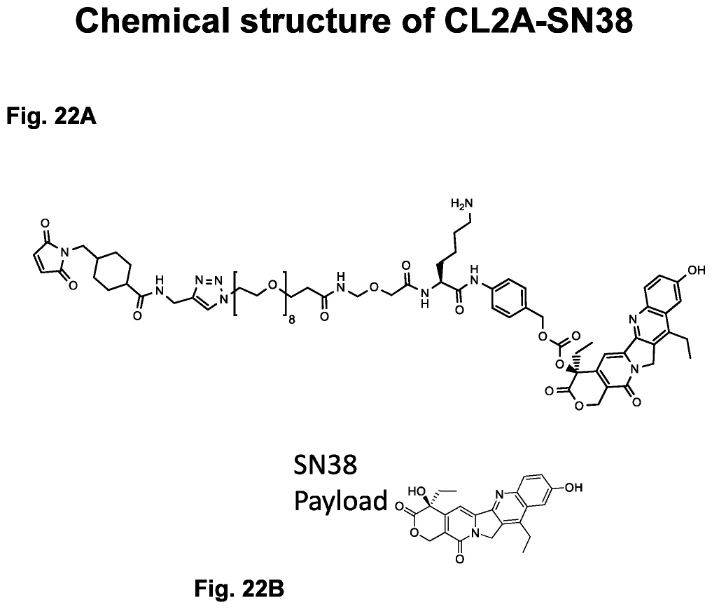

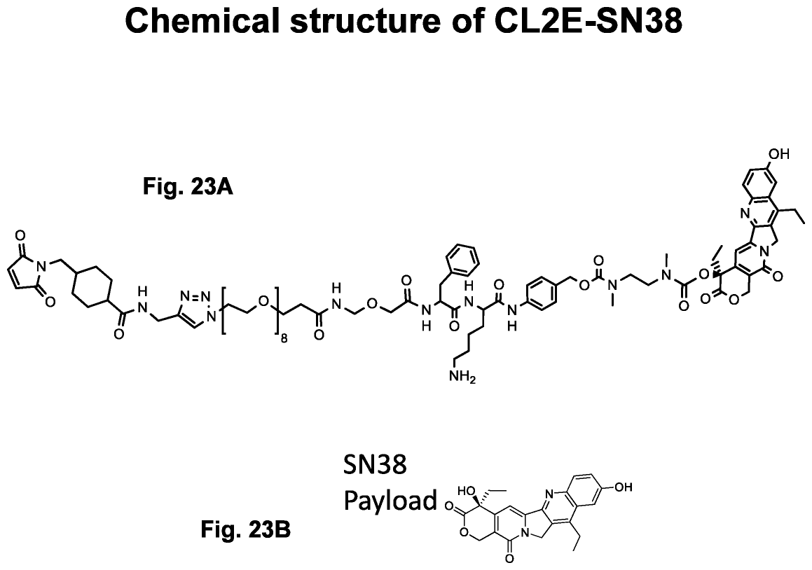

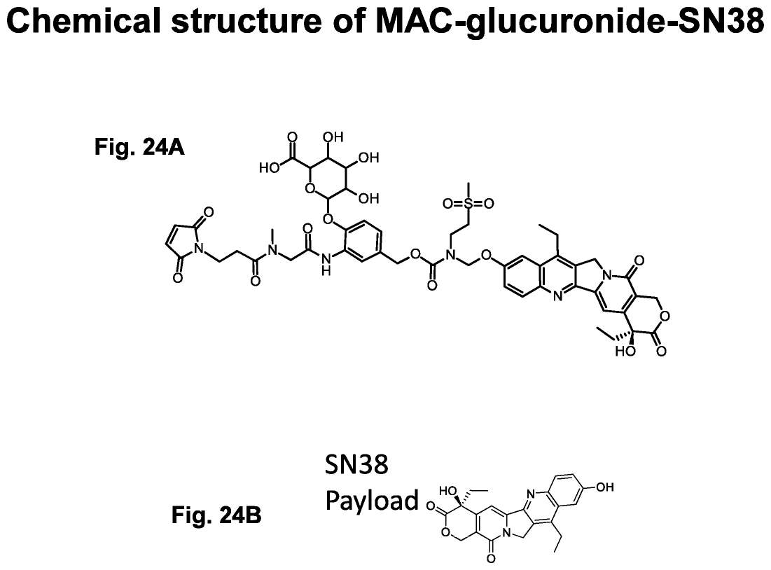

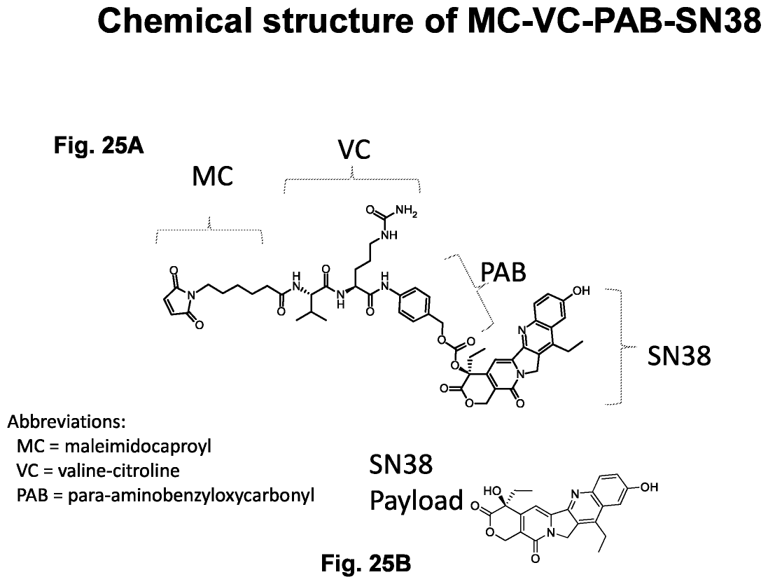

In various aspects, molecules comprising a linker and a therapeutic agent (e.g. a toxin) may be used to generate ADCs. The ADCs may comprise a linker and a therapeutic agent (e.g. a toxin) that are bound to the antibody. A linker molecule comprising a maleimide group may react with a sulfur from a cysteine residue of an antibody to conjugate the antibody to the linker and therapeutic agent. The linker may comprise a sulfur from a cysteine residue of the antibody. In some embodiments, the linker and toxin together have a structure shown in one of FIGS. 10-25.

### Pharmaceutical Compositions

In another aspect, provided herein are pharmaceutical compositions comprising the conjugates (e.g., ADCs) as disclosed herein. In some embodiments, the pharmaceutical composition comprises the conjugate of Formula (I) and a pharmaceutically acceptable carrier. In some embodiments, the pharmaceutical composition comprises an antibody drug conjugate as disclosed herein and a pharmaceutically acceptable carrier.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen.

In certain embodiments, a pharmaceutical composition disclosed herein further comprises a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers.

In certain embodiments, a pharmaceutical composition disclosed herein is administered to a subject by any suitable administration route, including but not limited to, parenteral (intravenous, subcutaneous, intraperitoneal, intramuscular, intravascular, intrathecal, intravitreal, infusion, or local) administration.

Formulations suitable for intramuscular, subcutaneous, peritumoral, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

### Additional Embodiments

Disclosed herein is a conjugate for Formula (I): [Ab]-[Z-L-R-X] y Formula (I), wherein: X is a moiety derived from a compound capable of inhibiting topoisomerase I or a compound capable of inhibiting microtubule assembly; R is a coupling moiety; L is a di- or tri- or tetra-peptide linking moiety having Z bonded to N-terminus and R bonded to the C-terminus; [Ab] is an antibody comprising an anti-MUC1* binding domain comprising three heavy chain (HC) complementarity determining region (CDRs): MUC1* HC-CDR1, MUC1* HC-CDR2, and MUC1* HC-CDR3; wherein the MUC1* HC-CDR1, the MUC1* HC-CDR2, and the MUC1* HC-CDR3 of the MUC1* binding domain comprises amino acid sequences selected from those set forth in Table 1 or a group of CDR sequences of the Sequences of anti-MUC1* antibodies section; wherein the MUC1* binding domain comprises three light chain (LC) complementarity determining region (CDRs): MUC1* LC-CDR1, MUC1* LC-CDR2, and MUC1* LC-CDR3; wherein the MUC1* LC-CDR1, the MUC1* LC-CDR2, and the MUC1* LC-CDR3 of the MUC1* binding domain comprises amino acid sequences selected from those set forth in Table 1 or a group of CDR sequences of the Sequences of anti-MUC1* antibodies section; Z is a conjugation moiety forms a covalent bond with a sulfur atom of a cysteine residue of the antibody; and wherein y is an integer from 1 to 10.

L can comprise a valine and a citrulline. L can comprise a glycine and a phenylalanine. R can comprise a para-aminobenzyloxycarbonyl. R can comprise a moiety comprising the structure of:

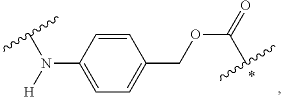 [SMILES: *Nc1ccc(COC(*)=O)cc1]

wherein the * indicates the point of attachment for the X group. R can comprise a moiety comprising the structure of:

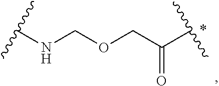 [SMILES: *NCOCC(*)=O]

wherein the * indicates the point of attachment for the X group. R can comprise a moiety comprising the structure of:

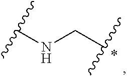 [SMILES: *CN*]

wherein the * indicates the point of attachment for the X group

L can be a dipeptide linking moiety comprising the structure of:

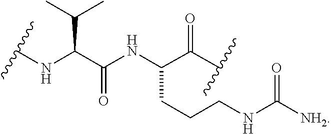 [SMILES: *N[C@H](C(=O)N[C@@H](CCCNC(N)=O)C(*)=O)C(C)C]

L can be a tetra-peptide linking moiety comprising the structure of:

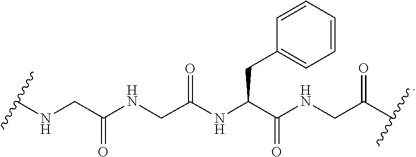 [SMILES: *NCC(=O)NCC(=O)N[C@@H](Cc1ccccc1)C(=O)NCC(*)=O]

The compound capable of inhibiting microtubule assembly can be MMAE or MMAF. The compound capable of inhibiting topoisomerase I can be exatecan or DXd. R can comprise a moiety comprising the structure of:

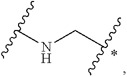 [SMILES: *CN*]

wherein the * indicates the point of attachment for the X group, and wherein compound capable of inhibiting topoisomerase I is DXd. R can comprise a moiety comprising the structure of:

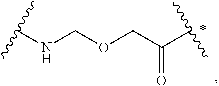 [SMILES: *NCOCC(*)=O]

wherein the * indicates the point of attachment for the compound capable of inhibiting topoisomerase I group, and wherein X is exatecan.

The antibody can be isotype IgG1 or IgG2. The antibody can be isotype IgG2b.

The anti-MUC1* binding domain can comprise a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence selected from SEQ ID NOs: 47, 9 or 23.

The anti-MUC1* binding domain can comprise a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence selected from SEQ ID NOs: 48, 10 or 24.

The anti-MUC1* binding domain can comprise a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence selected from SEQ ID NOs: 55, 56, 31 or 17.

Disclosed herein is an antibody conjugate comprising an antibody comprising an anti-MUC1* binding domain, wherein the antibody is conjugated to a payload via a maleimide-cysteine bond, wherein the payload comprises a linker and a cytotoxic compound, wherein the cytotoxic compound comprises a tubulin inhibitor or a topoisomerase I inhibitor. The linker can comprise a valine. The linker can comprise a citrulline. The linker can comprise a valine and a citrulline. The linker can be a dipeptide linking moiety comprising the structure of:

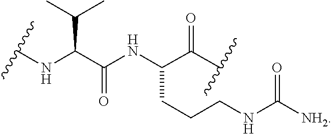 [SMILES: *N[C@H](C(=O)N[C@@H](CCCNC(N)=O)C(*)=O)C(C)C]

The linker can comprise at least one glycine. The linker can comprise at least one glycine and a phenylalanine. The linker can comprise a structure of:

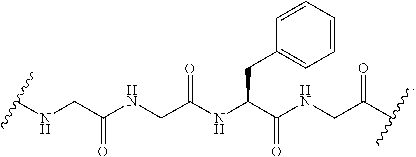 [SMILES: *NCC(=O)NCC(=O)N[C@@H](Cc1ccccc1)C(=O)NCC(*)=O]

The linker can comprise a para-aminobenzyloxycarbonyl. The linker can comprise a group of structure

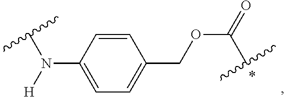 [SMILES: *Nc1ccc(COC(*)=O)cc1]

wherein the * indicates the point of attachment for the cytotoxic compound. The linker can comprise a group of structure

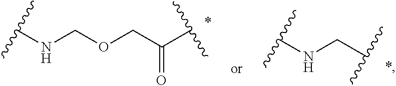

wherein the * indicates the point of attachment for the cytotoxic group. The tubulin inhibitor can be MMAE or MMAF. The topoisomerase I inhibitor can be exatecan or DXd, or a derivative thereof. The linker can comprise a group comprising the structure of:

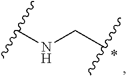 [SMILES: *CN*]

wherein the * indicates the point of attachment for the cytotoxic compound, and wherein the topoisomerase I inhibitor is DXd. or a derivative thereof. The linker can comprise a group comprising the structure of:

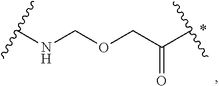 [SMILES: *NCOCC(*)=O]

wherein the * indicates the point of attachment for the cytotoxic compound, and wherein the topoisomerase I inhibitor is exatecan, or a derivative thereof.

The antibody isotype can be IgG1 or IgG2. The antibody isotype can beIgG2b. The antibody can be conjugated to at least two payloads. The antibody can be conjugated to at least three payloads. The antibody can be conjugated to at least four payloads. The antibody can be conjugated to at least five payloads. The antibody can be conjugated to at least six payloads. The antibody can be conjugated to at least seven payloads. The antibody can be conjugated to at least eight payloads. The anti-MUC1* binding domain can comprise three light chain (LC) complementarity determining region (CDRs): LC-CDR1, LC-CDR2, and LC-CDR3; wherein the LC-CDR1, the LC-CDR2, and the LC-CDR3 of the MUC1* binding domain can comprise amino acid sequences selected from those set forth in Table 1 or a group of CDR sequences of the Sequences of anti-MUC1* antibodies section; and wherein at least one of the LC-CDR1, LC-CDR2 and LC-CDR3 can comprise from 0-2 amino acid modification(s). The anti-MUC1* binding domain can comprise three heavy chain (HC) complementarity determining region (CDRs): HC-CDR1, HC-CDR2, and HC-CDR3; wherein the HC-CDR1, the HC-CDR2, and the HC-CDR3 of the MUC1* binding domain can comprise amino acid sequences selected from those set forth in Table 1 or a group of CDR sequence of the Sequences of anti-MUC1* antibodies section; and wherein at least one of the HC-CDR1, HC-CDR2 and HC-CDR3 can comprise from 0-2 amino acid modification(s). The anti-MUC1* binding domain can comprise a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 2 or a heavy chain variable domain sequence of the Sequences of anti-MUC1* antibodies section. The anti-MUC1* binding domain can comprise a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 2 or a light chain variable domain sequence of the Sequences of anti-MUC1* antibodies section. The anti-MUC1* binding domain can comprise a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3 or an scFv sequence of the Sequences of anti-MUC1* antibodies section.

The antibody can comprise an Fc domain. The Fc domain can be a heterodimeric Fc domain. The MUC1* binding domain can comprise a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence selected from Table 2 or a heavy chain variable domain sequence of the Sequences of anti-MUC1* antibodies section. The MUC1* binding domain a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence selected from Table 2 or a light chain variable domain sequence of the Sequences of anti-MUC1* antibodies section. The MUC1* binding domain can comprise a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence selected from Table 3 or an scFv sequence of the Sequences of anti-MUC1* antibodies section.

This disclosure encompasses conjugates of Formula (I): [Ab]-[Z-L-R-X] y comprising combinations of Ab, L and X (Ab/L/X) such as (3C2B1/VC/MMAE), (3C2B1/VC/MMAF), (3C2B1/VC/exatecan), (3C2B1/VC/DXd), (3C2B1/VA/MMAE), (3C2B1/VA/MMAF), (3C2B1/VA/exatecan), (3C2B1/VA/DXd), (3C2B1/GGFG/MMAE), (3C2B1/GGFG/MMAF), (3C2B1/GGFG/exatecan), (3C2B1/GGFG/DXd), (3C2B1/GGGG/MMAE), (3C2B1/GGGG/MMAF), (3C2B1/GGGG/exatecan), (3C2B1/GGGG/DXd), (3C2B1/GGVA/MMAE), (3C2B1/GGVA/MMAF), (3C2B1/GGVA/exatecan), (3C2B1/GGVA/DXd), (3C2B1/GGVG/MMAE), (3C2B1/GGVG/MMAF), (3C2B1/GGVG/exatecan), (3C2B1/GGVG/DXd), (3C2B1/VC/SN38), (3C2B1/VA/SN38), (3C2B1/GGFG/SN38), (3C2B1/GGGG/SN38), (3C2B1/GGVA/SN38), (3C2B1/GGVG/SN38), (C2/VC/SN38), (C2/VA/SN38), (C2/GGFG/SN38), (C2/GGGG/SN38), (C2/GGVA/SN38), (C2/GGVG/SN38), (C2/VC/MMAE), (C2/VC/MMAF), (C2/VC/exatecan), (C2/VC/DXd), (C2/VA/MMAE), (C2/VA/MMAF), (C2/VA/exatecan), (C2/VA/DXd), (C2/GGFG/MMAE), (C2/GGFG/MMAF), (C2/GGFG/exatecan), (C2/GGFG/DXd), (C2/GGGG/MMAE), (C2/GGGG/MMAF), (C2/GGGG/exatecan), (C2/GGGG/DXd), (C2/GGVA/MMAE), (C2/GGVA/MMAF), (C2/GGVA/exatecan), (C2/GGVA/DXd), (C2/GGVG/MMAE), (C2/GGVG/MMAF), (C2/GGVG/exatecan), (C2/GGVG/DXd), (20A10/VC/MMAE), (20A10/VC/MMAF), (20A10/VC/exatecan), (20A10/VC/DXd), (20A10/VA/MMAE), (20A10/VA/MMAF), (20A10/VA/exatecan), (20A10/VA/DXd), (20A10/GGFG/MMAE), (20A10/GGFG/MMAF), (20A10/GGFG/exatecan), (20A10/GGFG/DXd), (20A10/GGGG/MMAE), (20A10/GGGG/MMAF), (20A10/GGGG/exatecan), (20A10/GGGG/DXd), (20A10/GGVA/MMAE), (20A10/GGVA/MMAF), (20A10/GGVA/exatecan), (20A10/GGVA/DXd), (20A10/GGVG/MMAE), (20A10/GGVG/MMAF), (20A10/GGVG/exatecan), and (20A10/GGVG/DXd).

Disclosed herein is a method of treating cancer comprising administering an antibody of the disclosure to a subject in need thereof. In some embodiments, the cancer expresses MUC1*. The cancer can be breast cancer, colon cancer, prostate cancer, pancreatic cancer, or lung cancer.

### Differences Between Anti-MUC1* Antibodies and Antibody-Drug Conjugates

The C2 and 3C2B1 antibodies both bind to conformational epitopes near the C-terminus of the MUC1 extracellular domain. Surprisingly, assays on cancer cells lines and human tumors revealed that C2 and 3C2B1 bind distinct epitopes that impact their ability to treat different types of cancer and specific cancers from different patients. The C2 epitope is more common in breast cancers and in cell lines transduced to express MUC1* with a 45 amino acid extracellular domain (MUC1*45). The 3C2B1 epitope is more accessible in some lung, pancreatic, and esophageal cancers, and even some breast cancers. Curiously, MUC1*45 expression inhibits internalization of the 3C2B1 antibody and ADCs targeted by the 3C2B1 antibody into breast and lung cancer cells and also reduces the efficacy of 3C2B1-deruxtecan against human lung cancer tumors in mice, whereas it enhances C2 internalization and the efficacy of C2-deruxtecan. These results suggest that ADCs targeted by the 3C2B1 antibody will have increased clinical efficacy against certain MUC1* positive cancers. In another embodiment, a MUC1* positive cancer is treated with a mixture of MUC1* ADCs targeted by the C2 and 3C2B1 antibodies.

ADCs targeted by the 3C2B1 antibody are also expected to have clinical expression against MUC1* positive cancers (of any cancer type) if the 3C2B1 epitope is highly accessible on the surface of cells within cancerous tissue. Accessibility of the 3C2B1 epitope can be determined by measuring an H-score, as described by Meyerholz and Ram. See Meyerholz D. K. and Beck A. P., Principles and approaches for reproducible scoring of tissue stains in research. Lab Invest, 2018. 98 (7): p. 844-855; Ram S, Vizcarra P, Whalen P, Deng S, Painter CL, Jackson-Fisher A, et al. (2021) Pixelwise H-score: A novel digital image analysis-based metric to quantify membrane biomarker expression from immunohistochemistry images. PLOS ONE 16 (9): e0245638. MUC1* expression is quantified on a scale of 0, 1, 2, or 3 in different regions of a tumor section stained with 3C2B1, and the percentage of the tumor having each expression level is determined. The H-score is then calculated by summing (percentage x expression level) at each expression level. The maximum possible H-score is 300. Early tumors typically have a lower H-score. High H scores are a characteristic of early cancers, whereas later stage cancers are characterized by low H scores.

Accordingly, in some embodiments, a MUC1* positive tumor is suitable for treatment with an ADC targeted by the 3C2B1 antibody (a 3C2B1 ADC) if it has a H-score of at least 75, at least 100, at least 125, at least 150, at least 175, at least 200, at least 225, or at least 250, as determined after immunohistochemical staining a tumor section with an antibody comprising the CDRs of 3C2B1 (a high 3C2B1 H-score). In other embodiments, a MUC1* positive tumor is suitable for treatment with a 3C2B1 ADC even though it has an H-score of less than 100, less than 75, or less than 50, as determined after immunohistochemical staining a tumor section with an antibody comprising the CDRs of C2 (a low C2 H-score).

In other embodiments, a cancer patient is determined to have a MUC1* positive tumor suitable for treatment with a 3C2B1 ADC as described above and is then treated with a 3C2B1 ADC. Likewise, a 3C2B1 ADC may be formulated into an injectable medicament for treating a patient with a cancer suitable for treatment with a 3C2B1 ADC.

### EXAMPLES

### Example 1. Anti-MUC1* Antibodies

MUC1* is a cancer-specific antigen produced by proteolytic cleavage of the extracellular domain of MUC1. The extracellular domain of MUC1* comprises about 45 amino acids. Monoclonal antibodies generated against the PSMGFR peptide (SEQ ID NO: 1) bind to MUC1*, as described in WO 2020/146902. Table 4 presents the binding properties of eleven antibodies that bound to the PSMGFR peptide.

Cancer selective anti-MUC1* antibodies bind to a membrane proximal conformational epitope within the MUC1* extracellular domain, as described in WO 2020/146902. Such antibodies can be identified by screening for antibodies that bind (by ELISA) to the PSMGFR N-10 peptide (SEQ ID NO: 2), which has a 10 amino acid deletion from the N-terminus of PSMGFR, but do not bind (by ELISA) to the PSMGFR C-10 peptide (SEQ ID NO: 3), which has a 10 amino acid deletion from the C-terminus of PSMGFR.

The binding strength of anti-MUC1* antibodies is evaluated by determining their ability to displace NME7-AB (a MUC1* ligand) from MUC1* immobilized on the plastic surface of a 96-well plate. Because the cancer-selective MUC1* epitope is a conformational epitope, cancer-selective anti-MUC1* antibodies should not recognize MUC1* on a western blot, where the epitope is linearized. MMP9 is a protease that cleaves MUC1 to generate MUC1*. MMP9 is a predictor of poor prognosis for breast cancers. Some cancer-selective anti-MUC1* antibodies, particularly breast cancer selective antibodies, should bind to cells engineered to express MUC1 after they are contacted with MMP9 to convert the MUC1 to MUC1*.

Cancer-selectivity of the anti-MUC1* antibodies was further evaluated by flow cytometry and immunohistochemistry. The flow cytometry assay compared antibody binding to selected MUC1* positive and MUC1* negative cancer cell lines described in Table 5. The immunohistochemistry assay compared antibody binding to a panel of breast, pancreatic, and esophageal tumors and to normal tissues from the heart, lung, brain, liver, kidney, breast, bone marrow, eye, adrenal, skeletal muscle, ureter, and fallopian tube. The results demonstrate that certain antibodies in Table 4 are cancer-selective.

TABLE 4
Binding properties of anti-MUC1* antibodies

| | | | | | | | Recognizes | | |
|---|---|---|---|---|---|---|---|---|---|
| | | | | Does | Displaces | Does not | MUC1 | | |
| | | | | not | NME7AB | recognize | after | Cancer | Cancer |
| | Epitope | Binds | Binds | bind | from | a linear | cleavage by | selective | selective |
| mAb | (partial) | PSMGFR | N-10 | C-10 | MUC1* | epitope | MMP9 | by FACS | by IHC |
| C2 | FPFS | ++++ | ++++ | ++++ | ++++ | ++++ | ++++ | ++++ | ++++ |
| 20A10 | FPFS | ++++ | ++++ | ++++ | ++++ | ++++ | ++++ | ++++ | ++++ |
| E6 | FPFS | ++++ | ++++ | ++++ | ++++ | ++++ | ++++ | ++++ | ++++ |
| 3C2B1 | FPFS | ++++ | ++++ | ++++ | ++++ | ++++ | ++ | ++++ | ++++ |
| 5C6F3 | SVSDV | ++++ | ++++ | + | ++++ | ++++ | ++++ | ++++ | ++ |
| 25E6 | ASRYNLT | ++++ | ++++ | 0 | ++++ | 0 | ++++ | ++++ | ++ |
| C3 | ASRYNLT | ++++ | ++ | 0 | 0 | ++++ | 0 | ND | + |
| C8 | ASRYNLT | ++++ | ++ | 0 | 0 | ++++ | 0 | ND | + |
| 18G12 | ASRYNLT | ++++ | ++++ | 0 | ++++ | ++++ | 0 | + | + |
| 28F9 | ASRYNLT | ++++ | ++++ | 0 | ++++ | ++++ | 0 | + | + |
| 1E4 | QFNQYK | ++++ | ++++ | 0 | ++++ | 0 | ++++ | + | + |
| | TEA | | | | | | | | |

TABLE 5
Cancer cell lines used to characterize antibodies by flow cytometry

| | | | MUC1 |
|---|---|---|---|
| | | MUC1* | (full-length) |
| Cell line | Tissue | [C2 binding] | [VU4H5 binding] |
| HCT-116 | Colon | − | − |
| MDA-MB-231 | Breast | − | − |
| HCT-MUC1* | Colon | ++++ | − |
| HCT-MUC1-18 | Colon | − | ++++ |
| T47D | Breast | +++ | ++ |
| 1500 | Breast | +++ | ++ |
| NCI-H292 | Lung | ++ | ++ |
| NCI-H1975 | Lung | +++ | ++ |
| SKOV-3 | Ovarian | + | + |
| HPAF-II | Pancreatic | +++ | ++ |
| Capan-1 | Pancreatic | ++ | + |
| DU145 | Prostate | + | + |

### Epitope Mapping of Anti-MUC1* Antibody 3C2B1

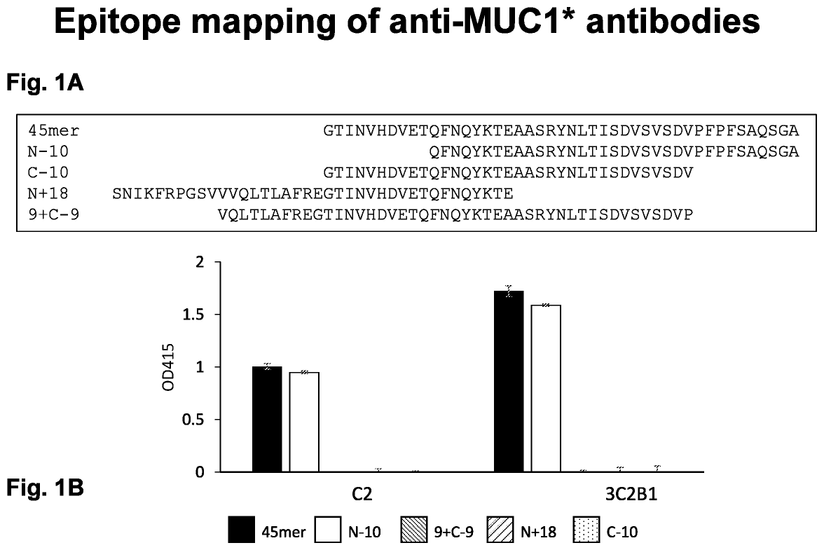

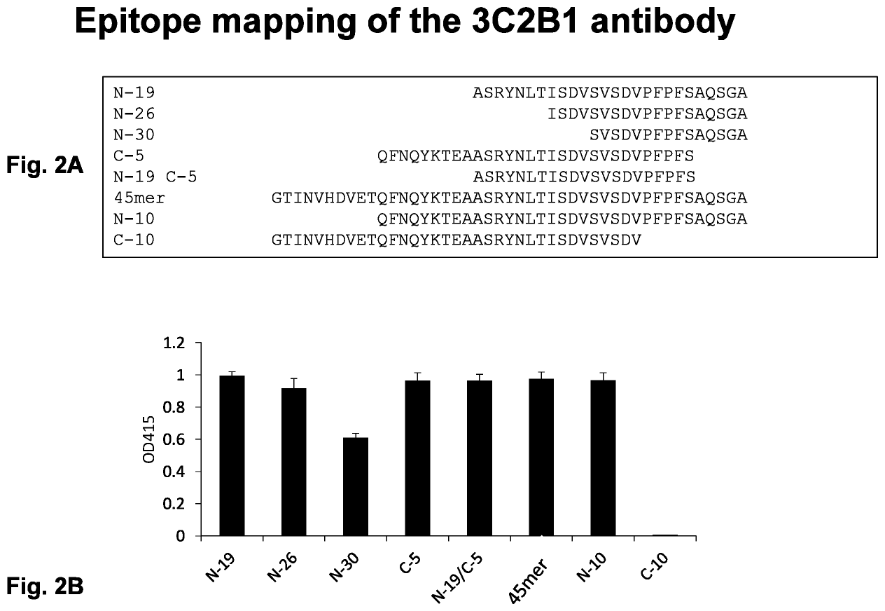

The C2 and 3C2B1 antibodies both bind to the PSMGFR 45mer and a variant PSMGFR peptide with a 10 amino acid N-terminal deletion (PSMGFR N-10), but do not bind to a variant PSMGFR peptide with a 10 amino acid C-terminal deletion (PSMGFR C-10) (FIGS. 1A, 1B). These results indicate that both antibodies bind to epitopes comprising amino acids located no more than 10 amino acids from the C-terminus of PSMGFR. The 3C2B1 epitope was mapped more precisely with additional PSMGFR peptide variants (FIGS. 2A, 2B). 3C2B1 bound to the N-30 and N-19 C-5 variant PSMGFR peptides, indicating that a 10 amino acid sequence located 6-15 amino acids from the transmembrane domain is sufficient for 3C2B1 binding.

### Example 2. Internalization of Anti-MUC1* Antibodies into Cancer Cell Lines

Some of the latest antibody drug conjugates (ADCs) are most effective when they are internalized by the cell after antibody binding to an antigen expressed on the cell surface. Once internalized their linkers can be cleaved by low pH, high levels of GSH, high levels of ferrous iron or by proteases, such as cathepsins to release toxin inside the target cell. We therefore investigated the internalization of C2 and 3C2B1 in various cancer cell lines by coupling the antibodies to pHrodo Deep Red, a pH-sensitive fluorophore with enhanced fluorescence in lysosomes. The results demonstrate that C2 and 3C2B1 bind to distinct MUC1* epitopes and that the MUC1* polypeptides they bind to are internalized at different rates in different cell lines. The results also suggest that ADCs comprising the 3C2B1 antibody may deliver toxin faster to lung, pancreatic, and esophageal cancers.

### Antibody Internalization by T47D Breast Cancer Cells

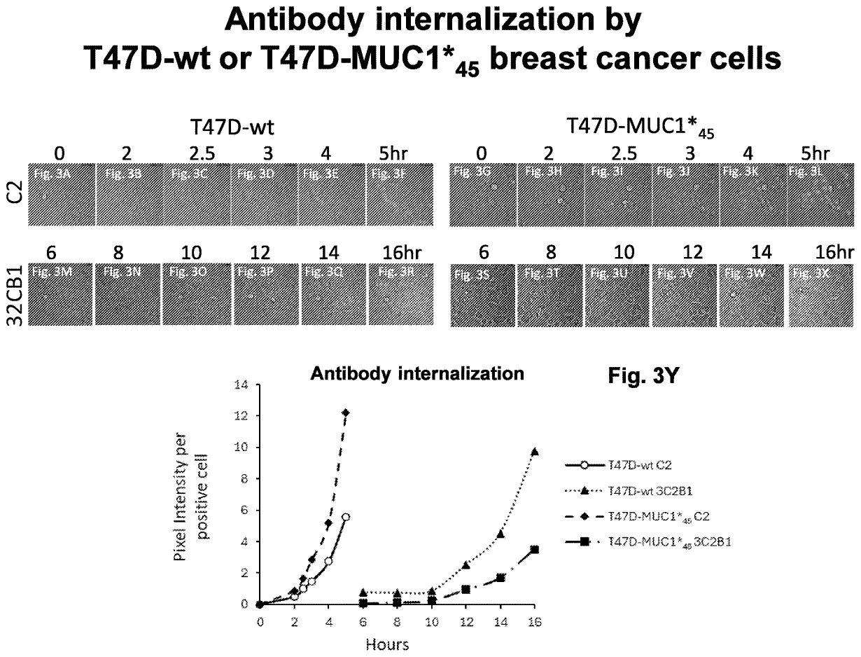

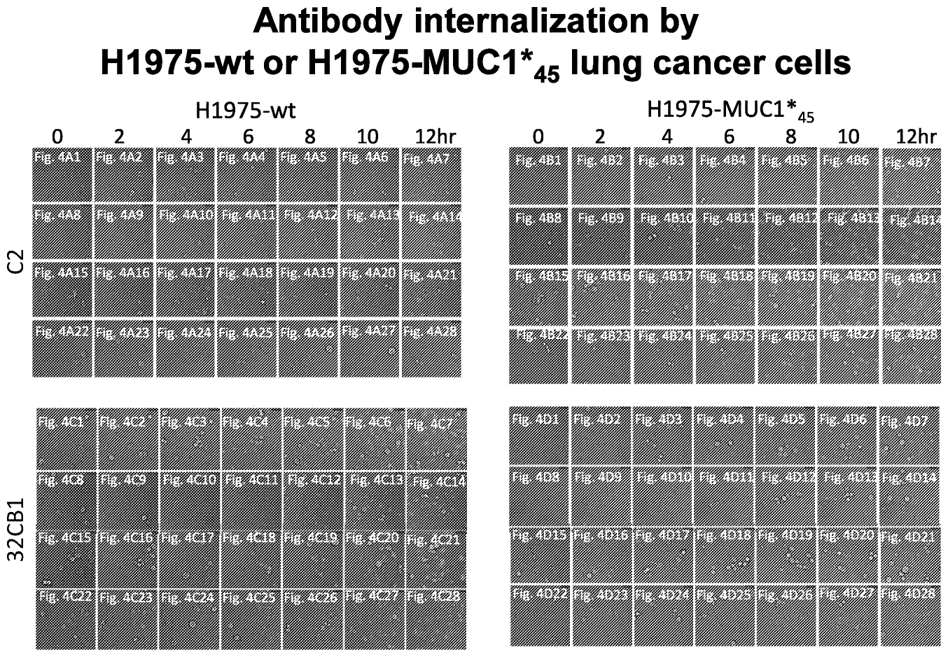

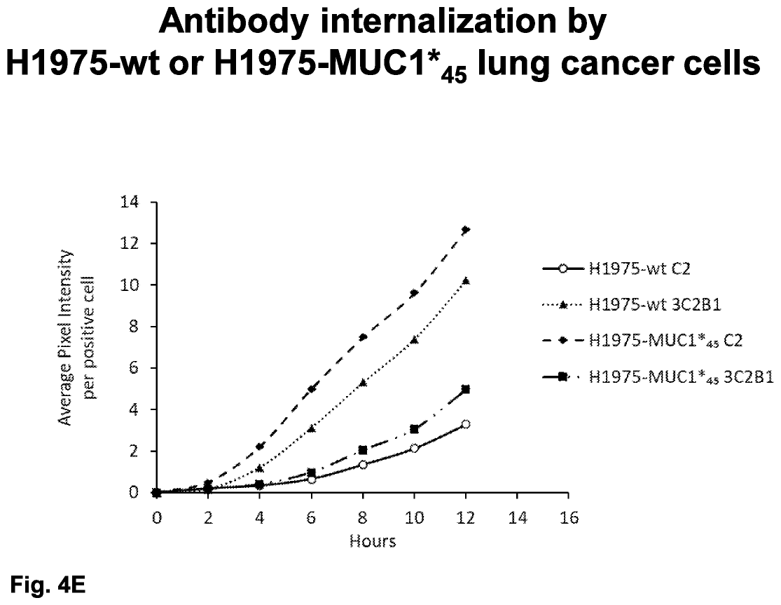

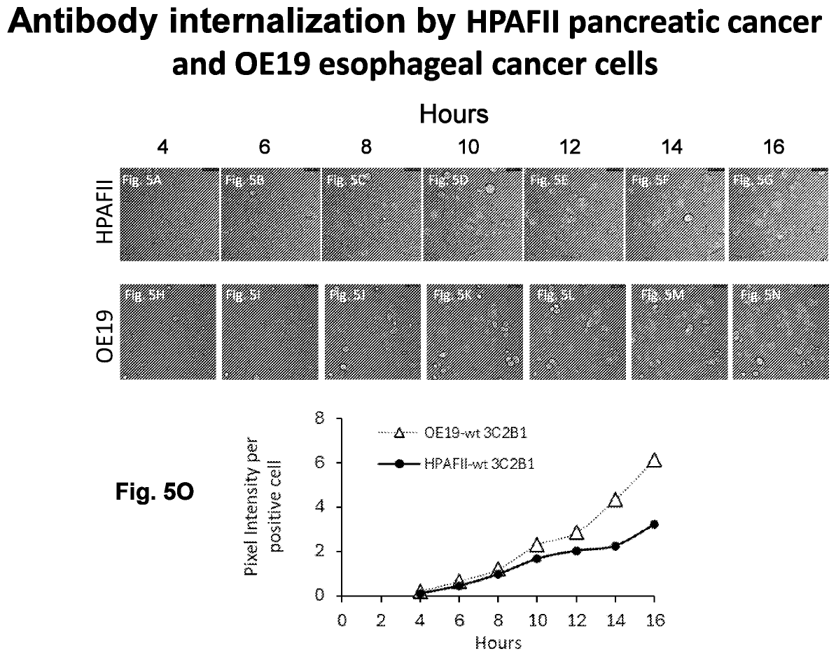

Antibodies C2 and 3C2B1 were tested for their ability to be internalized by T47D breast cancer cells (T47D-wt) or T47D cells that were engineered to express high levels of a recombinant MUC1* polypeptide with a 45 amino acid extracellular domain (T47D-MUC1*45), as described in WO2010042562 and WO2010042891. MUC1*45 was previously shown to bind the activating ligand NME7-AB and enhance the cytotoxic activity of T cells expressing a chimeric antigen receptor (CAR) comprising an scFv derived from the C2 antibody. C2 internalization was detected within 2.5 hours in T47D-wt cells (FIGS. 3A-3F) and was increased by MUC1*45 expression (FIGS. 3G-3L). In contrast, 3C2B1 was internalized more slowly by T47D-wt cells (FIG. 3M-3R). Unexpectedly, MUC1*45 expression inhibited 3C2B1 internalization (FIG. 3S-3X). These results are quantified in FIG. 3Y. Less than half of the T47D-MUC1* cells internalized detectable 3C2B1 after 14 hours compared to more intense C2 internalization into essentially all T47D-MUC1* cells after 4 hours (compare FIGS. 3W and 3K).

### Antibody Internalization by H1975 Lung Cancer Cells

Antibodies C2 and 3C2B1 were tested for their ability to be internalized by H1975 lung cancer cells (H1975-wt) and H1975 cells that were engineered to express high levels of a recombinant MUC1* polypeptide with a 45 amino acid extracellular domain (H1975-MUC1*45). In contrast to the T47D breast cancer cells, H1975-wt cells internalized 3C2B1 faster than they internalized C2 (FIGS. 4A1-4A28, 4C1-4C28 and 4E). Two (2) hours post incubation with H1975-wt cells, about half the cells had internalized C2. By 6 hours, about ⅔ of the cells had internalized C2 and essentially all the cells had internalized C2 by 10 hours. Conversely, about one third of H1975-wt cells had internalized 3C2B1 by 2 hours, more than a half by 4 hours and essentially all the cells had internalized the antibody by 6 hours. Similar to the T47D cells, MUC1*₄₅ expression increased the internalization rate of C2 and decreased the internalization rate of 3C2B1 (FIGS. 4B1-4B28, 4D1-4D28 and 4E). Essentially all H1975-MUC1*₄₅ cells had internalized antibody C2 by 4 hours, while 3C2B1 wasn't internalized by all the cells until 8 hours post incubation.

### Antibody Internalization by Pancreatic and Esophageal Cancer Cells

Internalization of antibody 3C2B1 into HPAFII pancreatic cancer cells (FIGS. 5A-5G) and OE19 esophageal cancer cells (FIGS. 5H-5N) was detectable after 8 hours and nearly complete at 16 hours. The results are quantified in FIG. 5O.

### Internalization of a 3C2B1-ADC

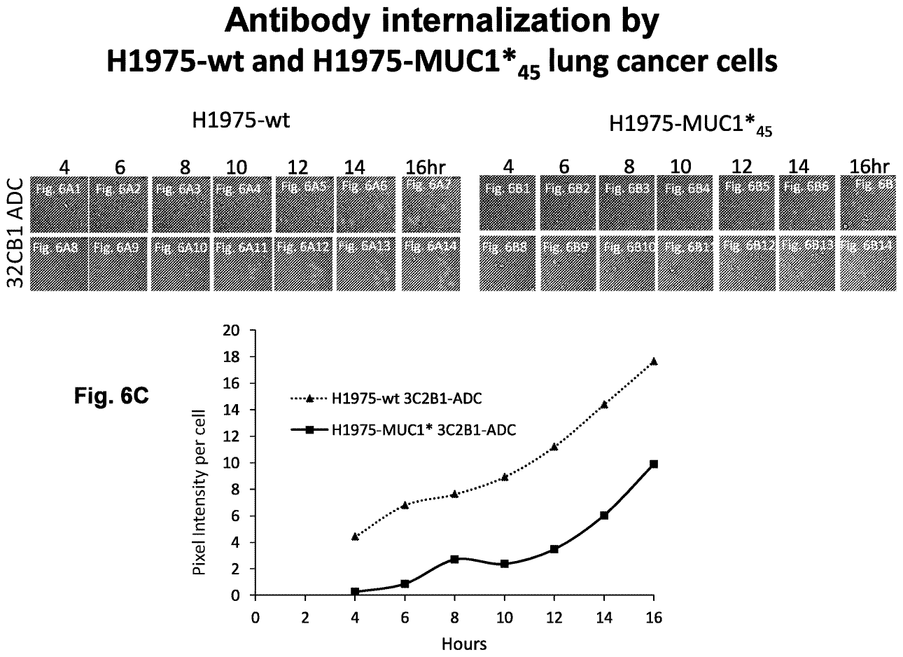

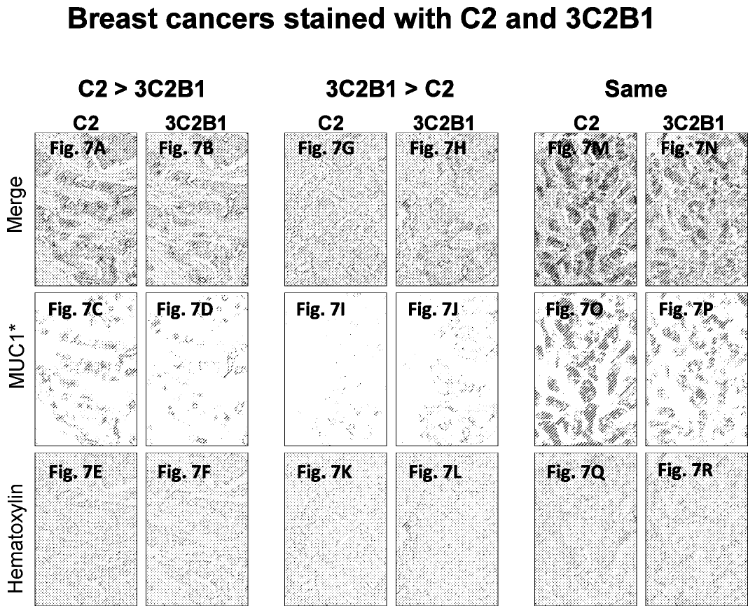

An antibody drug conjugate (ADC) comprising the 3C2B1 conjugated to a toxin (3C2B1-Deruxtecan) is internalized by H1975 cells at about the same rate as the 3C2B1 antibody alone, indicating that conjugation to the toxin did not block internalization (FIG. 6A1-6A14). MUC1*₄₅ expression inhibited 3C2B1-Deruxtecan internalization, like it did to the antibody alone (FIG. 6B1-6B14). These results are quantified in FIG. 6C.

### Antibody Internalization Protocol

Antibodies C2 and 3C2B1 were coupled to a pH sensitive fluorophore using the pHrodo Deep Red TFP Ester kit (Thermo cat #P35356), according to manufacturer instructions. Cancer cells were seeded at ~50,000 cells/well onto u-Slide 8-well tissue culture-treated, chambered coverslip wells (Ibidi 80806) in their recommended media with 10% FBS. Cells were cultured for 48 hours. Slides were transferred into microscope chamber (37 °C—5% CO2) and equilibrated for ~30 minutes. The pHrodo Deep Red coupled antibody was then diluted to a concentration of 200 to 300 μg/mL into pre-warmed media supplemented with 10% FBS. Media was removed from the chamber slides and the diluted antibodies were separately added. Fluorescent (655 nm) and phase contrast images at were collected every 10 min for 12 h or 16 h with a Leica Thunder Live Cell 3D microscope.

Internalization of the fluorescently labeled antibodies was quantified as follows. Images were imported into ImageJ. The selection tool was used to draw a region of interest (ROI) on the image. The integrated pixel density (mean gray-scale value x area) was then measured by the ImageJ software. Positively-stained cells were manually counted and the integrated pixel density was divided by the number of positive cells to yield “Pixel Intensity per positive cell”.

### Example 4. Immunohistochemistry (IHC) of Tissue Microarrays

Because our internalization results indicated that the C2 and 3C2B1 antibodies bind to distinct MUC1* epitopes and are internalized at different rates in different cancer cell lines, we next tested for differences in C2 and 3C2B1 binding to serial sections of human tumors from different individuals using tissue microarrays representing various forms of breast, lung and pancreatic cancers. Tissue microarrays are typically made up of hundreds of tissue specimens, where each specimen in the array comes from a different patient. The breast cancer array contained patient specimens of all types of invasive breast cancers, including less common forms such as carcinomas with medullary features and papillary carcinomas. The lung cancer array contained specimens from patients diagnosed with squamous cell carcinoma, adenocarcinoma, mucinous adenocarcinoma, adenosquamous carcinoma and large cell carcinoma. The pancreatic cancer tissue microarray contained specimens from patients with ductal adenocarcinomas. Table 6 summarizes the IHC results.

Of the 109 breast cancer specimens, 56 stained positive with C2, 3C2B1, or both. Twenty-two (22) of the specimens stained robustly with C2 and stained less well or not at all with 3C2B1. The converse, 3C2B1 staining better than C2, was true in only 5 of the 109 specimens. Twenty-nine (29) specimens were stained essentially the same with C2 and 3C2B1 antibody. Examples of different types of staining of breast cancer specimens are shown in FIGS. 7A-7R.

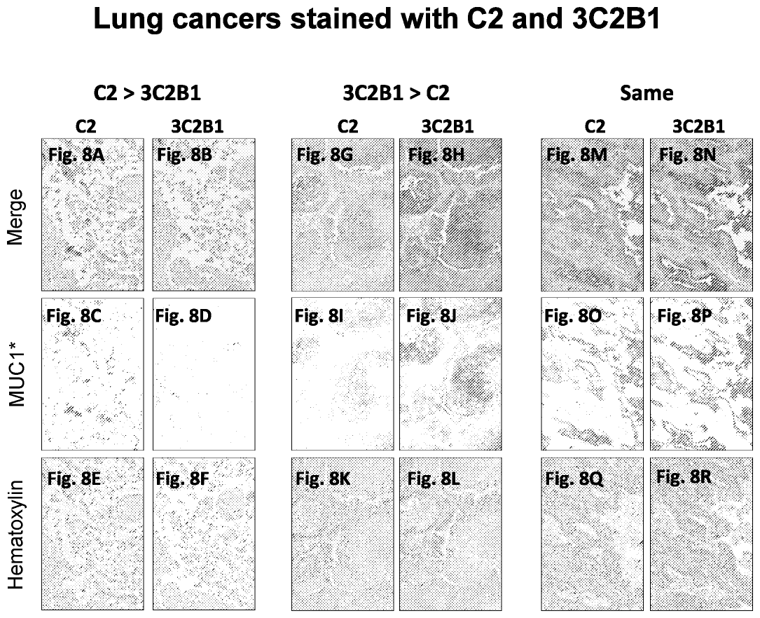

Of the 108 lung cancer specimens, a total of 61 stained positive with C2, 3C2B1, or both. Surprisingly, and in contrast to the result for breast cancer, 19 lung cancer specimens stained robustly with 3C2B1 and stained less well or not at all with C2. The converse, C2 staining better than 3C2B1, was true in only 5 of the 108 specimens, and these specimens stained weakly with both antibodies. A total of 37 specimens were stained essentially the same with 3C2B1 and C2 antibody. A large number of the specimens that stained the same with both antibodies had a very high MUC1* expression staining a 3-4+ on the staining intensity scale ranging from 0-4. Additionally, patient-associated data showed that those specimens were overall at a later stage of cancer. Overall, the results show that antibody 3C2B1 recognizes lung cancers better than C2 and therefore would be a preferred treatment for lung cancers. Examples of different types of staining of lung cancer specimens are shown in FIGS. 8A-8R.

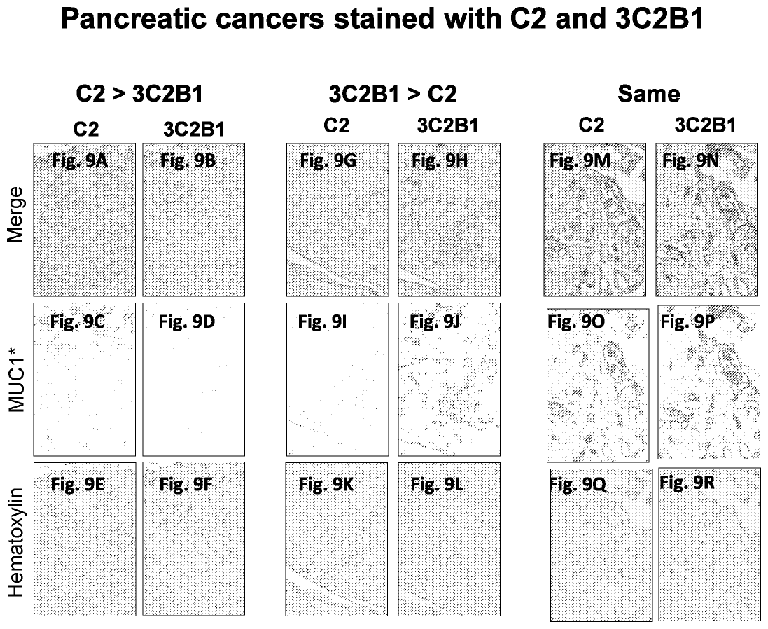

Similar to the lung cancer results, antibody 3C2B1 stained pancreatic cancers better than C2. Of the 40 specimens from 40 different pancreatic cancer patients, 27 were positively stained by C2, 3C2B1, or both. Eighteen (18) stained more robustly with 3C2B1 than C2 or were not stained by C2 at all. One specimen stained slightly better with C2 and 8 specimens stained essentially the same with either antibody. Examples of different types of staining of pancreatic cancer specimens are shown in FIGS. 9A-9R.

TABLE 6
Anti-MUC1* antibody selectivity for cancer subtypes

| Cancer type | Specimens | Positive | C2 > 3C2B1 | 3C2B1 > C2 | Same |
|---|---|---|---|---|---|
| Breast | 109 | 56 | 22 | 5 | 29 |
| Lung | 108 | 61 | 6 | 19 | 36 |
| Pancreatic | 40 | 27 | 1 | 18 | 8 |

### Immunohistochemistry Protocol

Cancerous tissue arrays were purchased from Tissue Array (Derwood, MD). After deparaffinization, antigen retrieval (pH 6.0) was performed for 10 min. The sections incubated in blocking buffer (10% normal goat serum and 5% BSA in TBS) at room temperature for 1 hr before incubating in either C2 or 3C2B1 in 1% BSA-TBS overnight at 4 °C. In this particular assay, both antibodies were used at a concentration of 15 μg/mL. After washing with TBS-T, sections were incubated with goat anti-mouse conjugated-HRP (Jackson ImmunoResearch Laboratories) in TBS. Sections were developed using Betazoid DAB chromogen (Biocare Medical), counterstained with Modified Mayer's Hematoxylin (Abcam) and NH4OH (Thermo Fisher Scientific), and dehydrated in ethanol (Fisher Scientific) and xylene (Millipore Sigma). Sections were cover-slipped and imaged using the Aperio ScanScope CS (Aperio Technologies, Inc/Leica Biosystems).

### Example 5. Structures and Synthesis of Anti-MUC1* ADCs

The Antibody Drug Complexes (ADCs) evaluated in these examples are presented in Table 7.

TABLE 7
ADC structures

| | | SEQ ID NO | | | |
|---|---|---|---|---|---|
| | | of Antibody | | Figure depicting | |
| | | Variable Domains | | the structure | |
| | | Heavy | Light | Linker- | Released |
| Compound | Common name | Chain | Chain | Toxin | Toxin |
| I | C2 MC-VC-PAB MMAE | 5 | 7 | 10A | 10B |
| II | 20A10 MC-VC-PAB MMAE | 19 | 21 | 10A | 10B |
| III | C2 MC-VC-PAB MMAF | 5 | 7 | 11A | 11B |
| IV | 20A10 MC-VC-PAB MMAF | 19 | 21 | 11A | 11B |
| V | C2 deruxtecan* | 5 | 7 | 12A | 12B |
| VI | 20A10 deruxtecan* | 19 | 21 | 12A | 12B |
| VII | C2 MC-VC-PAB exatecan | 5 | 7 | 13A | 13B |
| VIII | C2 MC-PEG8-VA-PAB exatecan | 5 | 7 | 14A | 14B |
| IX | 20A10 MC-VC-PAB exatecan | 19 | 21 | 13A | 13B |
| X | C2 MC-VA-PAB exatecan | 5 | 7 | 15A | 15B |
| XI | C2 MC-GGGG-AM DXd | 5 | 7 | 16A | 16B |
| XII | C2 MC-GGVA-AM DXd | 5 | 7 | 17A | 17B |
| XIII | C2 MC-GGVG-AM DXd | 5 | 7 | 18A | 18B |
| XIV | C2 MC-VA-AM DXd | 5 | 7 | 19A | 19B |
| XV | C2 MC-VA GAB-exatecan | 5 | 7 | 20A | 20B |
| XVI | C2 MC-GGVA GAB-exatecan | 5 | 7 | 21A | 21B |
| XVII | C2 CL2A SN38 | 5 | 7 | 22A | 22B |
| XVIII | C2 CL2E SN38 | 5 | 7 | 23A | 23B |
| XIX | C2 MAC-glucuronide SN38 | 5 | 7 | 24A | 24B |
| XX | C2 MC-VC-PAB SN38 | 5 | 7 | 25A | 25B |
| XXI | 3C2B1 deruxtecan* | 45 | 46 | 12A | 12B |
| XXII | 3C2B1 MC-VA-PAB exatecan | 45 | 46 | 15A | 15B |

*Deruxtecan is also known as MC-GGFG-AM DXd

### A. Methods

### Instrumentation and Materials for ADC Synthesis and Analysis

HPLC analyses were obtained on HP1090 Series II chromatographs and a HP1100 chromatograph using a Waters Corp (Milford, MA) HIC column Cat. No., 186007582, 4.6×35 mm, 2.5 μm particle size and a Tosoh Bioscience, Inc., (Grove City, OH) SEC column TSK Gel, Cat. No. 008541, 7.8 mm×30 cm, 5 μm. CombiFlash Rf purification systems and RediSep Gold columns were obtained from Teledyne Isco, Inc., Lincoln, NE. Zebatm Spin Desalting Column (Cat. No. 89890, ThermoFisher Scientific), Amicon Ultra 0.5 mL 50 kDa MWCO filter (Cat. No. UFC505094, Sigma-Aldrich), Cytiva PD Miditrap G-25 (Cat. No. 28918008, Sigma-Aldrich), Stabilization buffer (Cat. No. CM02022, Cell Mosaic® Inc., Woburn, MA), N-acetylcysteine (Cat. No. A7250, Sigma-Aldrich). Chemicals were obtained from Sigma-Aldrich, Inc., St. Louis, MO; ThermoFisher Scientific, Waltham, MA; Cell Mosaic® Inc., Woburn, MA; MedChemExpress LLC, Monmouth Junction, NJ., and others.]

### Ellman's Thiol Test for the Determination of the Number of Free Thiols in an ADC Reduction

Prepare 40 mL solution of a 2 mM Ellman's reagent, dissolve 31.7 mg of Ellman's reagent (5,5′-Dithiobis(2-nitrobenzoic acid, ThermoScientific Cat #22582) and 164 mg of sodium acetate in 40 mL of water. Keep stock Ellman's solution at 4 C.

Desalting reduced antibody aliquot: Take two of spin columns (Zeba spin desalting columns Ref #89882) and break the bottom off and loosen the cap. Place them in spin centrifuge tubes. Spin in a microcentrifuge (Spectrafuge 24D) at 1.5×g for 1 minute to remove buffer. Discard the eluted buffer. Condition the column by adding 400 μL of buffer (pH 8.0 Borate buffer composed of 25 mM sodium tetraborate, 25 mM NaCl, 1 mM EDTA) conditioning for 5 minutes followed by centrifuging for 1 minute at 1.5×g. Discard the eluent in-between spins. Repeat conditioning and centrifugation.

Prepare Ellman's reagent: To make 1 mL of Ellman's reagent pipette 850 μL of water into a tube. Add 100 μL of 1M Tris-HCl pH 8.0 followed by 50 μL of Ellman's stock prepared above. Add 70 μL to the bottom of two new centrifuge tubes (Protein LoBind Tube, Eppendorf Cat #022431102) labelling one Sample and the other Control. Once the columns have been conditioned, move them to clean new centrifuge tubes labelled Sample and Control.

Sample and Control prep: The final concentration of thiols after dilution should be in the 10-50 μM range. Antibody reduction reactions are run at 10 mg/mL or 66.7 μM. The dilution is a 5× dilution which brings the concentration to 13.3 μM. Based on past data a reading of ~0.24 mAu will correspond to ~4 thiols per antibody when the control is ~0.03. If more equivalents of reducing agent are added the new concentration should be reflected in the next thiol test.

Prepare a control solution immediately prior to running the test. The control should have the same concentration of DTT or TCEP as the reaction with no antibody present. Pipette 24 μL of Borate Buffer (or appropriate buffer) into two tubes labelled Sample prep and Control prep. Add 6 μL of reduced antibody solution and 6 μL of control solution to each respective tube. Slowly load the 30 μL from each tube to the respective tube with the desalting column. Try to dispense the liquid to the middle of the desalting resin without touching the sides. Spin the tubes at 1.5 g for 2 minutes and analyze the flowthrough in a 100 μL cuvette on a spectrophotometer at 412 nm.

The concentration of antibody in the thiol test is calculated by the using the using C1×V1=C2×V2. Where C1 is the initial concentration (66.7 μM), V1 is the 6 μL taken from the sample, and V2 is the 30 μL it was diluted to. Solving for C2 give the concentration of the antibody in the thiol assay, which is 13.34 μM. The absorbance at 412 nm is used to determine to the concentration of free thiol in the sample. First, the absorbance of the control is subtracted from the sample. The control subtracted absorbance is then put into beers law A=ebc where A is the absorbance, e if the molar absorptivity of the antibody, b is the path length of the cuvette, and c is the Molar concentration. The molar absorptivity of the Ellman's reagent is 14,150 L/mol-cm. The path length of the cuvette is 1 cm. Calculate the molar concentration of free thiol in the sample.

Once the concentration is determined there is a dilution factor of 3.333 from the 30 μL going through the column to into 70 μL of Ellman's reagent. Divide the dilution-factor-adjusted thiol concentration the concentration of the antibody to calculate the number of free thiols per antibody.

### Determination of Drug-Antibody Ratio (DAR) by UV

To estimate the DAR, UV absorbance is measured in a quartz cuvette and the DAR is calculated from an absorbance ratio (R). For MMAE and MMAF ADCs, R=(Absorbance @ 248 nm)/(Absorbance @ 280 nm) and DAR=(21×R−9)/(1.615−0.1425×R). For deruxtecan and exatecan ADCs, R=(Absorbance @ 370 nm)/(Absorbance @ 280 nm) and DAR==(0.194−21×R)/(0.8097×R−2).

### Determination of Drug-Antibody Ratio (DAR) by HIC-HPLC

Another method that can be used to estimate the DAR of ADC's is through the use of HIC-HPLC analysis. Briefly, samples of ADC and unconjugated antibody are injected into an HPLC. The peaks observed at differing retention times correspond to different drug-antibody-ratios. The HPLC retention time and peak area are analyzed in a spreadsheet in which the peaks are initially arbitrarily set to DAR's of 1-8 for IgG1 and 1-10 for IgG2. The time between peaks is used in combination with the analyst attempting to fit these peaks to integers corresponding to a specific number of drugs bound to the antibody. The process in confounded by the fact that several different ADC's exist for each DAR. For example, there are 8 possible ACD's (maleimide binding sites) for a DAR of 1. In general, the different in retention time between different DAR's is larger than the differences in retention time within a group of ACD's having the same DAR. After the analyst sets the DAR for each peak, the peaks areas are summed, and the contribution of from each DAR peak area is calculated. The average DAR is then determined for the sample based on all peaks. Ouyan, J, Drug-to-antibody ratio (DAR) and drug load distribution by hydrophobic interaction chromatography and reversed phase high-performance liquid chromatography, Methods Mol. Biol. 2013; 1045:275-83.

### Preparation of Antibodies for Conjugation

A buffer exchange process was used to further purify antibody preparations prior to conjugation of the antibody to toxins. For example, 20A10 was concentrated by 50 kDa MWCO filter and switched to pH 6.0 MES buffer (50 mM) 3 times. The concentrate was then diluted with pH 6.0 MES buffer (50 mM) to its original concentration. The antibody solution was then filtered through a 0.2 μm PES syringe filter (Pall, PN4602). The filtrate was concentrated again using a 50 kDa MWCO filter and the buffer was switched to pH 8.0 borate buffer (25 mM Sodium Borate, 25 mM Sodium Chloride, 1 mM EDTA) 3 times.

### B. Small Scale (3 mg) Preparation of Antibody-Drug-Conjugates—General Protocol

A solution of unconjugated antibody was reduced with an excess of dithiothreitol (DTT) in a pH 8.0 borate buffer. After 2 hours of reducing at 37 °C, an aliquot was tested for the amount of free thiol present using an Ellman's thiol test. Depending on the antibody isotype, the number of free thiols varies. If analysis of free thiols using Ellman's reagent indicated an insufficient number of free thiols, then more DTT was added and incubated for an additional time period at 37 °C. The process of free thiol determination and adding more reducing agent was repeated until a sufficient number of free thiols was obtained. 20A10 required more reducing agent than C2 and required longer reducing times than C2. The reduced antibody was then placed in a pH 6.0 2-(N-morpholino) ethanesulfonic acid (MES) buffer and the free cysteines were alkylated with 11 molar equivalents of a maleimide-linked-toxic agent for about 1 hour. The crude ADC was then purified through a desalting column to remove low molecular weight contaminants such as excess toxic agent, DMSO and other small molecular weight contaminants. The drug-antibody-ratio (DAR) was calculated from the ratio of toxin UV absorbance @ 248 nm (or 370 nm in the case of deruxtecan) to antibody absorbance @ 280 nm. Analysis of the conjugates to determine the drug-antibody-ratio (DAR), i.e, the [Drug: mAb] molar ratio, was accomplished by hydrophobic interaction chromatography-high-performance liquid chromatography following a literature method (Effects of drug loading on the antitumor activity of a monoclonal antibody drug conjugate. Hamblett, K. J et al, Clin Cancer Res. 2004 Oct. 15; 10 (20): 7063-70.

This procedure was followed to generate small scale preparations of Compounds I-IX.

### C. Small-Scale (3 mg) Prep of C2-Deruxtecan

IgG1 C2 antibody (3.00 mg, 1.578 mL, 1.9 mg/mL, 20 nM) was thawed from −80 °C in a 37 °C water bath for 1 minute. The protein concentration was determined by nanodrop using an extinction coefficient of 14,000 L/g-cm for mouse IgG1 antibodies. The antibody solution was concentrated using centrifuge filters (Amicon Ultra 0.5 mL 50,000 mw cut off (Catalog #UFC505024) (14,000×g at 4 °C for 8 min) to obtain a volume of equal to or less than 100 μL. Then the buffer was exchanged to pH 8.0 borate buffer (25 mM sodium tetraborate, 25 mM NaCl, 1 mM EDTA) by concentrating to <100 μL and resuspending in approximately 300 μL fresh buffer. This process was repeated for a total of 3 times. The concentrated antibody (~100 uL) was transferred to a 0.5 mL screw cap centrifuge tube and was brought up to 300 μL with pH 8.0 borate buffer (25 mM sodium tetraborate, 25 mM sodium chloride, 1 mM EDTA) to make a 10 mg/mL antibody solution.

A solution of dithiothreitol (DTT) (6.48 mM) was prepared by weighing DTT (5 mg, 32.4 mmol) and dissolving it in 5 mL of pH 8.0 borate buffer (25 mM sodium tetraborate, 25 mM sodium chloride, 1 mM EDTA). The DTT solution (6.48 mM, 30.9 μL, 200 nmol, 10 equivalents) was added to the antibody solution and the tube was heated in a 37 °C water bath for 2 hr. The warm reaction mixture was periodically vortexed every 15 minutes. After 2 hours an aliquot of the reaction mixture (6 μL) was taken out for Ellman's thiol analysis, which showed 7.68 free thiols per antibody out of a total possible 8 thiols for an IgG1.

The antibody reduction mixture was cooled in an ice bath to 4 °C and the solution was transferred to a centrifuge filter (Amicon Ultra 0.5 mL 50,000 mw cut off) and the buffer was switched 3 times to pH 6.0 MES buffer (50 mM). [14,000×g, at 4C, for 8 min). The concentrated reduced antibody was diluted to 640 μL with pH 6.0 MES buffer (50 mM).

A stock solution of deruxtecan (10 mg/mL, 9.7 mM) in DMSO was prepared by weighing 1.80 mg, 1.74 μMoles and dissolving it in DMSO (180 μL). A solution of deruxtecan (2 mM, 110.2 μL, 220 nmoles) in 50% DMSO/pH 6.0 MES buffer (50 mM) was prepared by diluting the deruxtecan solution (22.8 μL, 10 mg/mL in DMSO) with DMSO (32.3 μL) and then further diluted with 50 mM MES pH 6.0 (50 mM, 55.1 μL) to make a solution of 2 mM deruxtecan in 50% DMSO/50% pH 6.0 MES buffer (50 mM). Deruxtecan solution (110.1 μL, 50% DMSO/50% pH 6.0 MES buffer, 2 mM, 73.4 nmoles, 11 equiv) was added to the tube containing 640 μL of reduced antibody. The reaction was mixed slowly on a vertical rotator for 1 hour at room temperature and monitored by hydrophobic interaction chromatography (HIC) HPLC, which indicates whether or not there is unreacted antibody remaining. The retention time of a trace of unreacted antibody is compared to the reaction product to determine if unreacted antibody remains and if so, the reaction is allowed to continue. For small scale the time is typically 1-2 hours. Large scale preparations can require allowing the reaction to take place over 8-12 hours.

After ~1 hour the reaction was purified on a desalting column (Cytiva PD miditrap g-25 medium, Cat #28918008). The top of the column was uncapped and the packing buffer was discarded. The column bottom was uncapped and the column was conditioned with Gibco PBS pH 7.4 (Ref #10010-023) (3×5 mL) by gravity flowthrough. The antibody solution (750 μL) was loaded onto the column and allowed to enter the resin bed fully, followed by PBS (250 μL). The purified ADC was then eluted from the column with pH 7.4 PBS (1.25 mL). Nanodrop protein concentration determination showed 1.76 mg/mL of antibody in 1.25 mL of PBS, or 2.2 mg (73% yield) of conjugated antibody recovered from an initial 3.0 mg of unconjugated antibody. The purified ADC was analyzed by UV-VIS spectroscopy at 370 nm and 280 nm to give a drug-antibody-ratio (DAR) of 6.7.

### D. Synthesis of 3C2B1-Deruxtecan

3C2B1-deruxtecan was synthesized by reacting the TCEP-reduced antibody with commercially available deruxtecan. Deruxtecan, a topoisomerase I inhibitor, is an exatecan derivative combined with a cleavable peptide linker and a maleimide group. Deruxtecan is MC-GGFG-MAC-dxd1 (mercaptocaproic acid amide of glycine-glycine-phenylalanine-glycine-methoxymethylamino-dxd1). The maleimide group of deruxtecan reacts with the free cysteine thiols of the reduced antibody. The payload is dxd1.

To a solution of the 3C2B1 antibody (1.99 mg, 13.3 nmoles) in pH 7.4 buffer (0.292 mL, 50 mM EPPS with 5 mM DTPA) was added a solution of TCEP (17.0 μL, 6.0 equiv, 79.6 nmoles, 4.68 mM) in the same pH 7.4 buffer. The tube was heated to 37C in a water bath for 90 min with vortexing every 20 min. After 90 min, an aliquot was removed and analyzed for free thiol content using an Elmann's ADC assay to give 8.57 free thiols. Deruxtecan (10.0 equiv, 132.6 nmoles, 13.7 μL, 9.67 mM, MedChemExpress LLC, Monmouth Junction, NJ, Cat. No.: HY-13631E) in DMSO was then mixed with DMSO (17.2 μL). The diluted deruxtecan solution in DMSO (30.9 μL) was then added to the antibody (309 μL). The reaction mixture was placed on a vertical rotator for 60 min at room temperature. The crude product was first purified on a Zeba Spin Column (Cat. #89890, equilibrated with pH 7.4 PBS (Gibco)) with a 40 μL of pH 7.4 PBS stacker to remove the excess deruxtecan, TCEP, and to buffer exchange. The eluted ADC was further purified on a Cytiva PD miditrap G-35 (Cat #28918008), equilibrated with pH 7.4 PBS (Gibco), and eluted with PBS (1.25 mL). Stabilization buffer (312.5 μL, Cell Mosaic, Inc., Woburn, MA) was added to give a solution of the purified ADC in PBS (1.65 mg, 55% yield, 1.06 mg/mL). The drug-antibody-ratio (DAR) was then determined using 2 methods (cuvette and HIC/HPLC. Cuvette DAR=6.9. HIC DAR=6.9. Average DAR=6.9.

### E. Large Scale (30 mg) Preparation of Antibody-Drug-Conjugates.

In a similar manner to the small-scale ADC preparation, a solution of unconjugated antibody was reduced with an excess of dithiothreitol (DTT) in a pH 8.0 borate buffer. After 2 hours of reducing, an aliquot was tested for the amount of free thiol present using an Ellman's thiol test. The reduced antibody was then placed in a pH 6.0 MES buffer and the free cysteines were alkylated with 11 molar equivalents of a maleimide-linked-toxic agent overnight. The crude ADC was then purified through a desalting column.

Aliquots of IgG1 C2 antibody (15.7 mL of 1.92 mg/mL, 30.1 mg, 200 nmoles) were thawed from −80 °C in a 37 °C water bath for 1 minute. The volumes were consolidated and the concentration was determined by nanodrop using the mouse IgG1 antibody setting with the background subtraction turned off. 30 mg antibody in 16.17 mL at 1.9 mg/mL 200 nmol. The solution was concentrated using 3 centrifuge filters (Amicon Ultra 4 mL 30,000 mw cut off (Ref #UFC803024)) and the buffer was exchanged to pH 8.0 borate buffer (25 mM sodium tetraborate, 25 mM NaCl, 1 mM EDTA) 3 times spinning in an Eppendorf 5804R centrifuge with a swing bucket rotor at 4000 g for 15-25 minutes. The concentrated antibody was transferred to a 5 mL screw cap tube and was brought up to 3 mL with pH 8.0 borate buffer (25 mM sodium tetraborate, 25 mM sodium chloride, 1 mM EDTA) to make a 10 mg/mL antibody solution.

A solution of DTT (6.48 mM) was prepared by weighing DTT (5 mg) and dissolving it in pH 8.0 borate buffer (5 mL) (25 mM sodium tetraborate, 25 mM sodium chloride, 1 mM EDTA). The DTT solution (6.48 mM, 617 μL, 4000 nmoles, 20 equiv) was added to the antibody solutions and the tubes were heated in a 37 °C in a shaker incubator for 2 hr. After 2 hours, an aliquot of the reaction mixture (6 μL) was taken out for Ellman's thiol analysis. Analysis of these results showed 7.12 moles free thiols per mole of antibody.

In a similar manner 20A10 was reduced with a total of 20 equivalents of DTT over a period of 3 hours with several iterations of free thiol testing.

The tube of reduced antibody was then cooled in an ice bath to 4 C, the solution was transferred equally to 3 centrifuge filters (Amicon Ultra 4 mL 30,000 to mw cut off (Ref #UFC803024)) and the buffer was exchanged to pH 6.0 MES (50 mM) buffer three times spinning in an Eppendorf 5804R centrifuge at 4000 g for 15-25 minutes. The concentrates were transferred to a 15 mL falcon tube and the concentrators were rinsed with buffer two times to bring the final volume to 6400 μL with pH 6.0 MES (50 mM) buffer.

A stock solution of deruxtecan (10 mg/mL, 9.67 mM) in DMSO was prepared by weighing deruxtecan (3.4 mg, 3.29 μMols) and dissolving it in DMSO (340 μL). A 1100 μL solution of 2 mM deruxtecan in 50% DMSO/pH 6.0 MES (50 mM) was prepared by diluting the DMSO stock deruxtecan solution (227.5 μL of 9.67 mM, 2.22 μMoles) with DMSO (322.5 μL) and then further diluted with pH 6.0 MES (50 mM, 550 μL) to make 1100 μL of 2.068 mg/mL (2 mM) deruxtecan in 50% DMSO/50% pH 6.0 MES (50 mM). The deruxtecan solution (2 mM, 1000 μL, 2200 nmoles, 11 equiv) was added to the tube containing 6400 μL of reduced antibody. The reaction was mixed on a vertical rotator for 18 hours at room temperature and monitored by HIC HPLC after 1 hour and the next day.

After 18 hour the reaction was purified on a desalting column (Cytiva Sephadex G-25 medium Cat #17003301). Sephadex g-25 medium grade gel (10.13 g) was swelled in Gibco PBS pH 7.4 (Gibco catalog #10010-023, 50 mL) in a round bottom flask for 3 hours at room temp. A 25 g Teledyne Isco Redi-Sep Sample Load Cartridge (Isco catalog #693873240) was attached to a ring stand and PBS was injected through the bottom to prime the cartridge. After 3 hours of swelling the resin, the flask was vacuum degassed with argon/vacuum 3 times. The swollen resin was then poured into the cartridge and the bottom was uncapped allowing the PBS to elute. The round bottom flask was washed with PBS to transfer all of the resin to the cartridge. A waste container was placed below the column. The column was conditioned with Gibco PBS pH 7.4 (Ref #10010-023) 3×50 mL by gravity flowthrough. A filter was placed on top of the cartridge and was pushed down the column packing tool being mindful to only slightly compress the gel. The column height was approximately 74 mm. Antibody solution (7500 μL) was loaded onto the column and allowed to enter the resin bed fully. PBS (2500 μL) was added to the column. A new 50 mL falcon tube was placed below the column and the purified ADC was eluted with PBS pH 7.4 (14 mL). For column efficiency 1 mL fractions were passed through the column until a total of 50 mL had passed through the column. The collected solution was analyzed by nanodrop to show 1 mg/mL antibody concentration in 17.5 mL PBS, or 17.5 mg of antibody, 58% yield from unconjugated C2.

### F. ADC Synthesis Results

Exemplary ADC compounds comprising the C2 or 20A10 antibodies coupled to MMAE, MMAF, exatecan or deruxtecan were synthesized as summarized in Table 8.

TABLE 8
ADC synthesis summary

| | | Equiv | Free | Drug Antibody Ratio | |
|---|---|---|---|---|---|
| Compound | Batch | DTT | Thiol | Spectroscopy | HIC HPLC |
| I | | 10 | 6.3 | 4.0 | 4.1 |
| II | A | 20 | 3.7 | 3.4 | 3.0 |
| | B* | 5 | 4.1 | 5.6 | 3.8 |
| III | | 5 | 4.5 | | 3.7 |
| IV | | 15 | 3.9 | | 3.8 |
| V | A | 20 | 7.2 | 4.2 | 4.6 |
| | B | 10 | 7.5 | 8.3 | 8.6 |
| VI | A | 20 | 4.8 | 5.0 | 4.6 |
| | B* | 5 | 3.4 | 5.4 | 4.7 |
| | C* | 10 | 6.4 | 8.3 | 9.9 |
| VII | | 10 | 7.8 | 6.7 | 8.1 |
| VIII | | 5 | 4.1 | 5.7 | 4.8 |
| IX | | 5 | 4.1 | 5.3 | 4.6 |

*Antibody filtered prior to conjugation

### G. Synthesis of C2-MC-GGGG-Aminomethyl-DXd

### Synthetic Scheme: C2-MC-GGGG-Aminomethyl-DXd

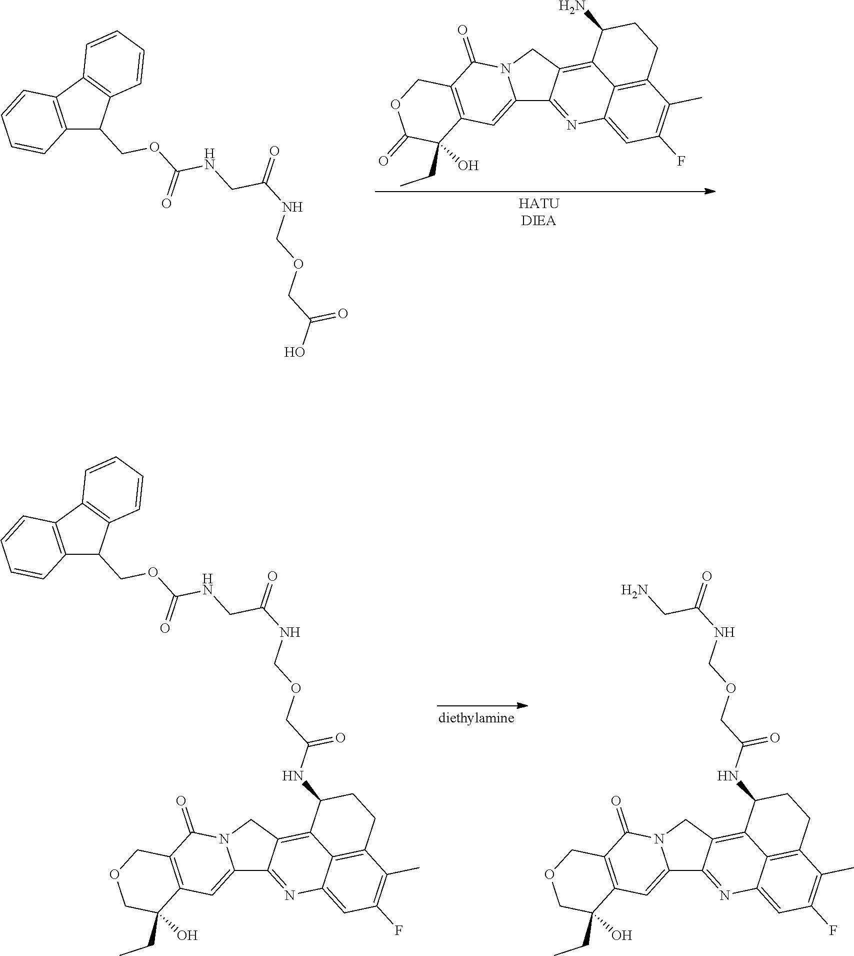 [SMILES: O=C(O)COCNC(=O)CNC(=O)OCC1c2ccccc2-c2ccccc21 ; CC[C@@]1(O)C(=O)OCc2c1cc1n(c2=O)Cc2c-1nc1cc(F)c(C)c3c1c2[C@@H](N)CC3 ; CC[C@@]1(O)COCc2c1cc1n(c2=O)Cc2c-1nc1cc(F)c(C)c3c1c2[C@@H](NC(=O)COCNC(=O)CNC(=O)OCC1c2ccccc2-c2ccccc21)CC3 ; CC[C@@]1(O)COCc2c1cc1n(c2=O)Cc2c-1nc1cc(F)c(C)c3c1c2[C@@H](NC(=O)COCNC(=O)CN)CC3]

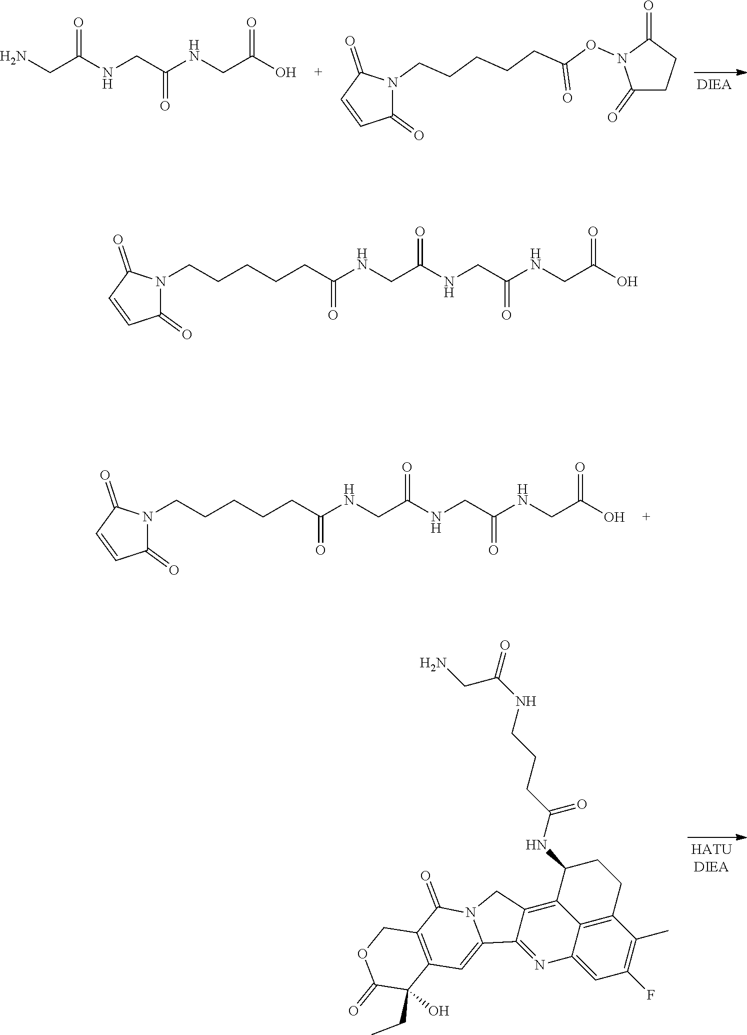

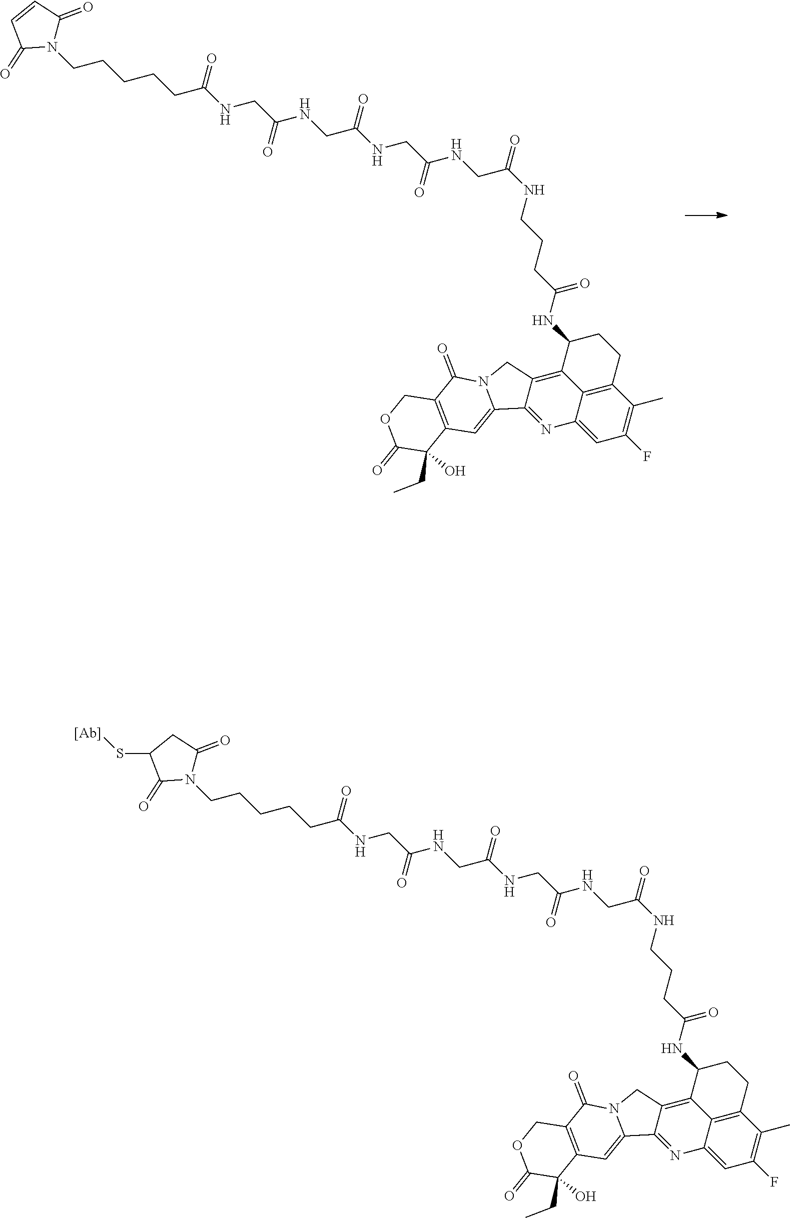

C2-MC-GGGG-aminomethyl-DXd was synthesized in a five-step reaction sequence. Fmoc-gly-2-(amiomethoxy) acetic acid was coupled with exatecan mesylate using hexafluorophosphate azabenzotriazole tetramethyl uronium (HATU). The Fmoc group was then removed using diethylamine (DEA). MC-GGG-OH was synthesized from commercially available GGG-OH and maleimidocaproic acid O-Su ester. MC-GGG-OH and Gly-aminomethyl-DXd were then coupled using HATU to give MC-GGGG-aminomethyl-DXd. The disulfide bonds in the antibody, C2, were then reduced with dithiothreitol (DTT) and alkylated using the MC-GGGG-aminomethyl-DXd to give the antibody drug conjugate.

### Synthetic Scheme: Gly-Aminomethyl-DXd

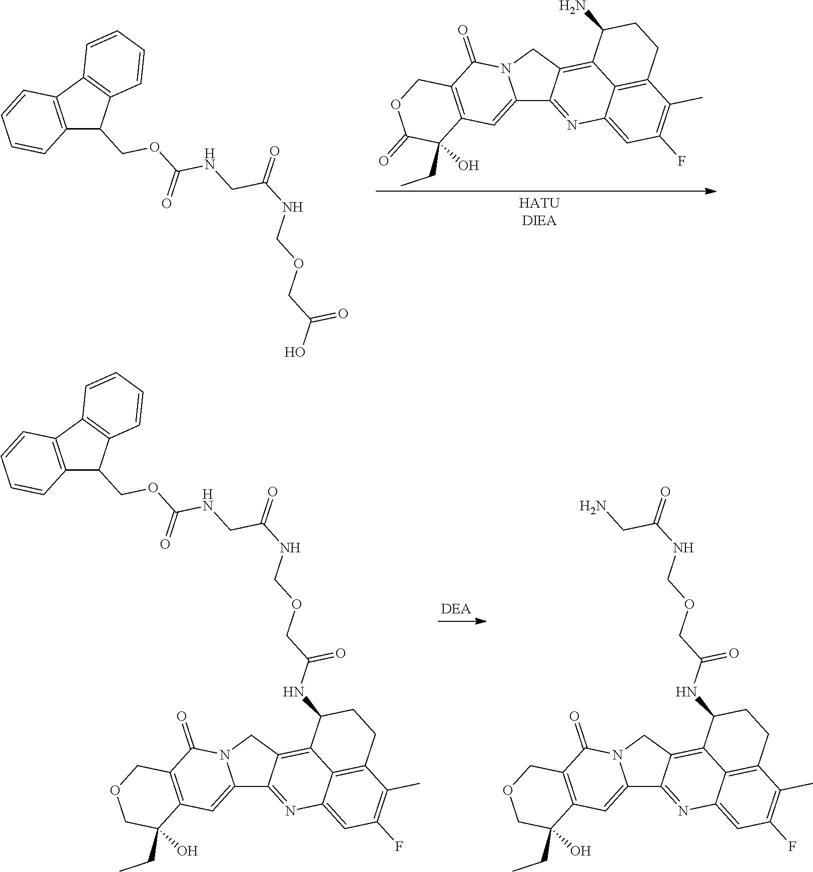 [SMILES: O=C(O)COCNC(=O)CNC(=O)OCC1c2ccccc2-c2ccccc21 ; CC[C@@]1(O)C(=O)OCc2c1cc1n(c2=O)Cc2c-1nc1cc(F)c(C)c3c1c2[C@@H](N)CC3 ; CC[C@@]1(O)COCc2c1cc1n(c2=O)Cc2c-1nc1cc(F)c(C)c3c1c2[C@@H](NC(=O)COCNC(=O)CNC(=O)OCC1c2ccccc2-c2ccccc21)CC3 ; CC[C@@]1(O)COCc2c1cc1n(c2=O)Cc2c-1nc1cc(F)c(C)c3c1c2[C@@H](NC(=O)COCNC(=O)CN)CC3]

### Formation of Fmoc-Gly-Aminomethyl-DXd

To an oven-dried glass vial containing a stir-bar, Fmoc-Gly-2-(aminomethoxy) acetic acid (88.4 mg) was dissolved in anhydrous dimethylformamide (DMF) (1.6 mL) under argon. The clear solution was then cooled in an ice-bath, followed by addition of HATU (91.7 mg) and N,N-diisopropylethylamine (DIEA) (75.8 μL). The reaction was then stirred for 30 min at OC. After this period, exatecan mesylate (100.0 mg) and additional DIEA (38.1 μL) were added and the reaction was then removed from the ice bath and allowed to stir at room temperature. After 18 h, the clear, dark reaction mixture was transferred dropwise to a stirring solution of 2% acetic acid (HOAc) (aq) (40 mL) in an Erlenmeyer flask, resulting in immediate precipitation of a tan solid. After the addition was complete, the solid was collected on a fritted glass funnel and washed with 2% HOAc (aq) (10 mL), followed by water (10 mL). The solid was then allowed to dry on the filter and then placed in a chamber under high vacuum for 2h. Following high vacuum, the solid was dissolved in dichloromethane (DCM) (200 mL), and then mixed with Celite (2.5 g), which was then rotovapped to dryness. The Celite plus compound was then loaded into an empty cartridge (12 g) and purified on a Combiflash using a 1% HOAc/DCM to 10% methanol (MeOH)/1% HOAc/DCM gradient. Fractions were analyzed by thin layer Chromatography (TLC) and High-performance liquid chromatography (HPLC). Fractions containing product were pooled together, rotovapped, then placed under high vacuum for an hour to give a resulting brown solid (137.3 mg). HPLC shows the product was contaminated with 2.3% of Fmoc-Gly-Mac-OH. The crude solid was suspended in ethyl acetate (EtOAc) (100 mL) and shook vigorously with saturated sodium bicarbonate (NaHCO3) (100 mL). The organic layer was then washed with brine (25 mL), dried over sodium sulfate (Na2SO4) and evaporated to a tan solid. The light brown solid collected was (102.2 mg) free of the contaminating carboxylic acid. 1H NMR (DMSO with 0.03% v/v TMS, 400 MHz): δ 7.86 (2H, d, J=7.42 Hz), 7.76 (1H, d, J=10.94 Hz), 7.68 (2H, d, J=7.45 Hz), 7.39 (2H, m), 7.30 (3H, m), 6.50 (1H, s), 5.40 (2H, dd, J=20.00, 15.00 Hz), 5.17 (2H, d, J=7.52 Hz), 4.63 (2H, d, J=7.38 Hz), 4.24 (3H, dt, J=6.00, 6.00 Hz), 3.62 (2H, d, J=6.81 Hz), 2.50 (10H, s), 2.37 (3H, s), 1.91 (4H, s), 1.83 (1H, i, J=6.50 Hz), 0.85 (3H, t, J=7.30 Hz).

Fmoc Cleavage of Fmoc-Gly-Aminomethyl-DXd with Diethylamine

Fmoc-Gly-aminomethyl-DXd (90.1 mg) was added to a vial, followed by DMF (100 uL). DEA (2.4 mL) was then added and the reaction allowed to stir at room temperature for 40 min. After 40 min, diethyl ether (12 mL) was added, resulting in precipitation of a solid, which was stirred for another 5 min. The solid was then collected on a fritted glass, washed twice with diethyl ether (10 mL), then placed under hivac briefly. After drying, the mass of tan solid was 67.8 mg. The solid was then suspended in DCM (100 mL) containing MeOH (2 mL) and loaded onto celite (1 g), which was dried under rotovap. The celite-loaded product was then purified on combiflash using a 4 g RediSep Gold silica cartridge, and running a linear gradient from neat DCM to 50% MeOH. Fractions containing the product were pooled and dried to give a tan solid (33.5 mg). 1H NMR (DMSO with 0.03% v/v TMS, 400 MHz): δ 9.18 (3H, t, J=7.00 Hz), 8.56 (3H, d, J=8.80 Hz), 8.01 (8H, s), 7.78 (3H, d, J=10.88 Hz), 7.32 (3H, s), 6.53 (3H, s), 5.58 (3H, ddd, J=8.86, 6.66, 5.38 Hz), 5.41 (6H, s), 5.17 (6H, dd, J=22.00, 20.00 Hz), 4.71 (7H, q, J=3.23 Hz), 4.05 (7H, s), 3.65 (6H, s), 3.17 (7H, qt, J=17.33, 6.28 Hz), 2.66 (OH, i, J=1.91 Hz), 2.38 (10H, d, J=1.83 Hz), 2.17 (6H, q, J=6.89 Hz), 1.86 (6H, p, J=6.67 Hz), 0.87 (10H, t, J=7.28 Hz).

### Synthetic Scheme: MC-GGG-OH

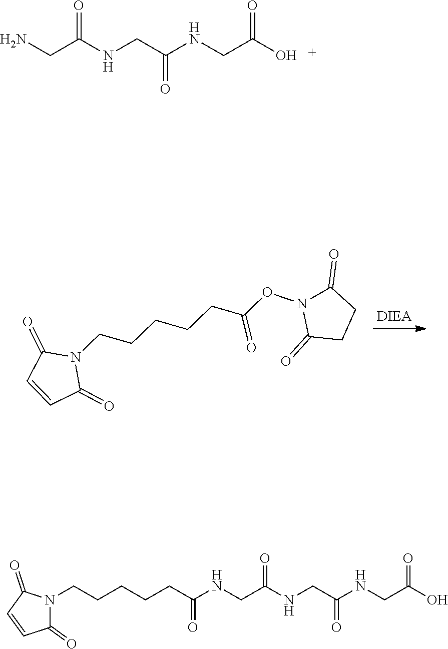

### Formation of MC-GGG-OH

GGG-OH peptide was added to a vial containing stir-bar (49.6 mg). Dimethyl sulfoxide (DMSO) (2 mL) was added to the reaction. DIEA (100 μL) was added to each reaction while stirring. N-succinimidyl 6-maleimidohexanoate (MC-OSu) (99.3 mg) was then added in one portion to the reaction. After 24 h, the reaction was transferred dropwise to a diethyl ether (50 mL) with stirring, resulting in formation of a fine precipitate that was allowed to settle. The supernatant was then decanted, fresh ether (50 mL) added, and decanted again. More ether (20 mL) was added and decanted a third time, resulting in a dry, white residue at the bottom of the flask. The solid was then resuspended in acetone (25 mL) via sonication and collected in a fritted-glass funnel. After drying, the mass of the white solid (69.3 mg) was weighed and analyzed. NMR shows the structure is consistent with the product. 1H NMR (DMSO with 0.03% v/v TMS, 400 MHz): δ 8.36 (3H, t, J=5.71 Hz), 8.30 (1H, s), 7.26 (2H, s), 3.98 (9H, m), 3.63 (4H, t, J=7.10 Hz), 2.37 (3H, t, J=7.47 Hz), 2.76 (10H, quint, J=1.75 Hz), 1.73 (6H, m), 1.44 (5H, m), 0.26 (OH, s).

### Synthetic Scheme: MC-GGGG-Aminomethyl-DXd

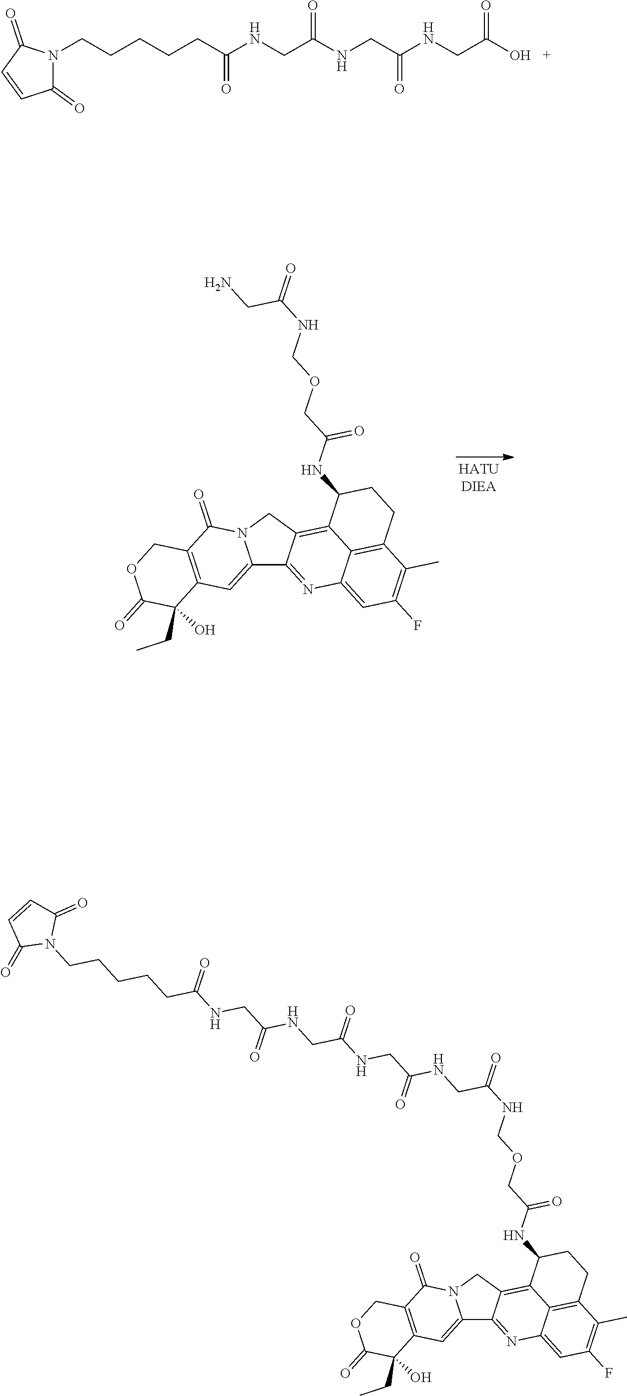

### Formation of MC-GGGG-Aminomethyl-DXd

MC-GGG-OH (33.3 mg) was added to a vial containing gly-aminomethyl-DXd (34.0 mg) along with a stir-bar. Anhydrous DMF (1 mL) was added to the vial under argon, and the vial was placed into an ice-bath. DIEA (27.8 μL) and HATU (38.0 mg) were added. The reaction was then removed from the ice-bath and allowed to stir at room temp. After 34 min, the reaction was removed from stirring and poured into a sep. funnel containing EtOAc (450 mL) and water (100 mL). The layers were separated and the organic layer was further washed with water (2×100 mL). The organic layer was then washed with sat. NaHCO₃ (3×100 mL), followed by brine (50 mL). The EtOAc layer was dried over anh. Na2SO4 and concentrated to a white solid. Analysis of the aqueous layer from the extraction observed the presence of product. Although some product was present in the EtOAc layer, it had limited solubility. The water layer was rotary evaporated to a white solid, which was extracted with anhydrous ethanol (~5 mL), then extracted again with methanol (~5 mL). Each extract was isolated in a tared vial and rotovapped. The ethanolic layer had a mass of 7 mg and methanolic layer had a mass of 68.3 mg. The methanolic, ethanolic, and EtOAc extracts were combined, dissolved in 10% MeOH/DCM (100 mL) and loaded onto celite (1.54 g). This mixture was then rotovapped then high-vacuumed prior to loading onto the combiflash. The product was eluted using a gradient from DCM to 20% MeOH/DCM. Fractions containing product were pooled and evaporated. The product was dried overnight under high vacuum. This material (15 mg) was stored at −20C until needed. 1H NMR (DMSO with 0.03% v/v TMS, 400 MHz): δ 8.68 (1H, t, J=6.71 Hz), 8.54 (3H, d, J=4.04 Hz), 8.50 (1H, d, J=8.89 Hz), 8.37 (3H, d, J=8.39 Hz), 8.15 (3H, q, J=6.98 Hz), 8.09 (1H, t, J=5.72 Hz), 8.05 (1H, t, J=5.72 Hz), 7.76 (1H, d, J=10.68 Hz), 7.36 (3H, dd, J=8.39, 4.27 Hz), 7.29 (1H, s), 6.98 (2H, s), 6.51 (1H, br s), 5.58 (1H, q, J=6.52 Hz), 5.41 (3H, s), 5.18 (3H, s), 4.62 (3H, d, J=6.49 Hz), 4.00 (3H, s), 3.72 (12H, m), 3.59 (2H, i, J=6.84 Hz), 3.35 (7H, t, J=7.10 Hz), 3.12 (3H, q, J=8.34 Hz), 2.49 (11H, s), 2.37 (4H, s), 2.18 (3H, m), 2.08 (3H, t, J=7.56 Hz), 1.85 (3H, o, J=7.34 Hz), 1.45 (6H, h, J=7.01 Hz), 1.25 (4H, d, J=6.47 Hz), 1.17 (4H, i, J=8.21 Hz), 0.86 (4H, t, J=7.25 Hz).

### Synthetic Scheme: C2-MC-GGGG-Aminomethyl-DXd

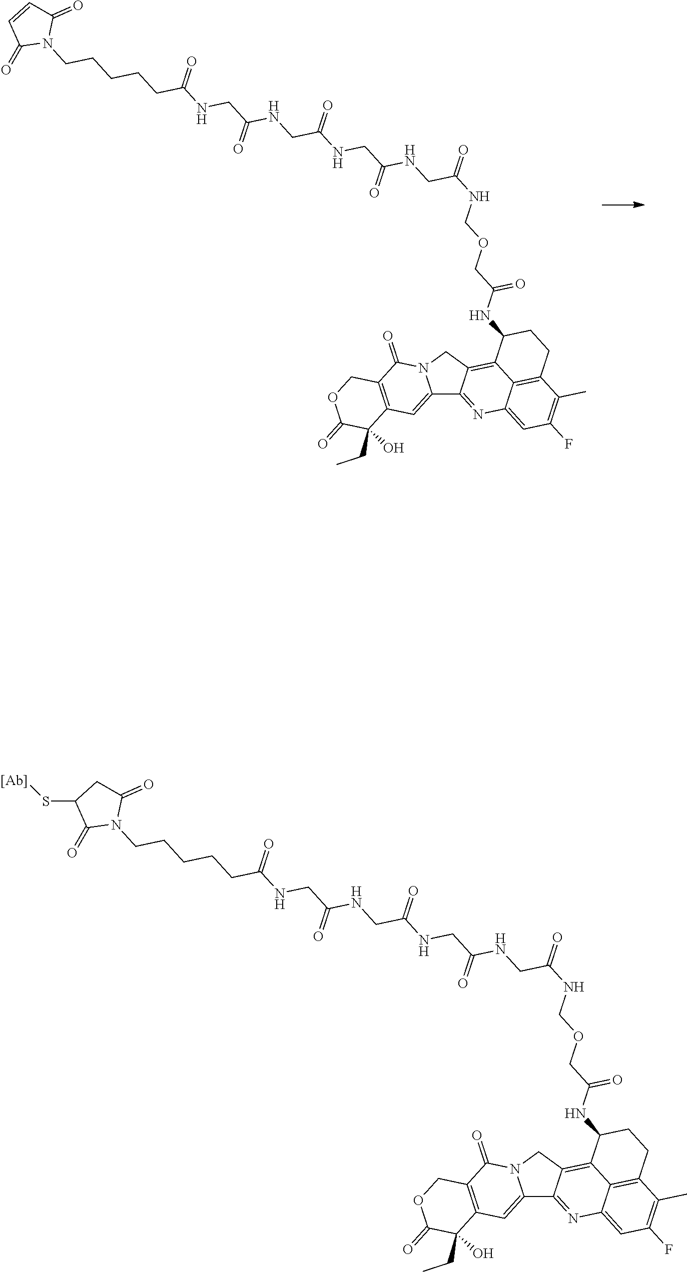

Reduction of C2 Antibody and Alkylation with MC-GGGG-Aminomethyl-DXd

To a solution of C2 antibody (7.22 mg/mL, 2.17 mg, 14.44 nmols, 300 μL) in pH 8.0 borate buffer (25 mM Sodium borate, 25 mM sodium chloride, 1 mM EDTA buffer), DTT (6.48 mM, 93.89 nmols, 14.5 μL, 6.5 equiv.) in pH 8.0 borate buffer was added for reduction. This was placed on a water bath at 37 C for 2 h with vortexing every 20 min. After 2 h, the solution was placed on ice and an aliquot was taken to determine free thiol content using an Ellman's test. This gave a free thiol content of 7.7 free thiols per antibody. A Desalting column was used to purify and buffer exchange the solution to pH 7.4 4-(2-Hydroxyethyl)-1-piperazinepropanesulfonic acid (EPPS) buffer (50 mM EPPS and 5 mM DTPA) with 40 μL of buffer used as a chaser to fully elute product. The solution was then further purified on a 50 kDa MWCO filter and pH 7.4 EPPS buffer. The solution was returned to the original volume in pH 7.4 EPPS buffer. MC-GGGG-aminomethyl-DXd (9.67 mM, 144.4 nmols, 62.1 μL) dissolved in DMSO was diluted to get an organic phase of 10% of the total volume, and added to the reduced antibody. The reaction was vertically rotated for 60 min at room temperature. Following this, A Sephadex G-25 column was used to purify the solution, with 1.25 mL of pH 7.4 PBS used to elute the ADC. Stabilization buffer (312.5 μL) was added to the solution and the concentration measured by nanodrop (1.035 mg/mL, 1.62 mg, 10.8 nmol, 1.5625 mL, yield: 54%). The drug-antibody-ratio (DAR) was calculated with cuvette and HIC/HPLC (Cuvette DAR: 6.4, HIC DAR: 6.5, AVG: 6.4)

### H. Synthesis of C2-MC-GGVG-Aminomethyl-DXd

### Synthetic Scheme: C2-MC-GGVG-Aminomethyl-DXd

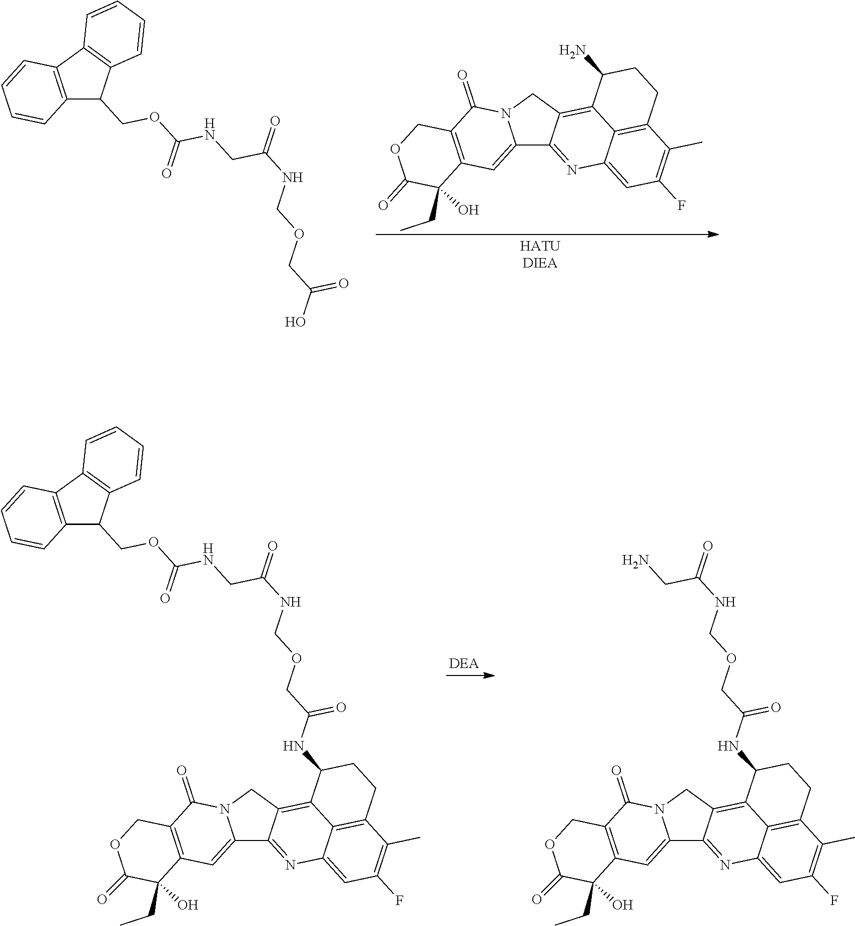 [SMILES: O=C(O)COCNC(=O)CNC(=O)OCC1c2ccccc2-c2ccccc21 ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)COCNC(=O)CNC(=O)OCC1c2ccccc2-c2ccccc21)CC3 ; CC[C@@]1(O)C(=O)OCc2c1cc1n(c2=O)Cc2c-1nc1cc(F)c(C)c3c1c2[C@@H](NC(=O)COCNC(=O)CN)CC3]

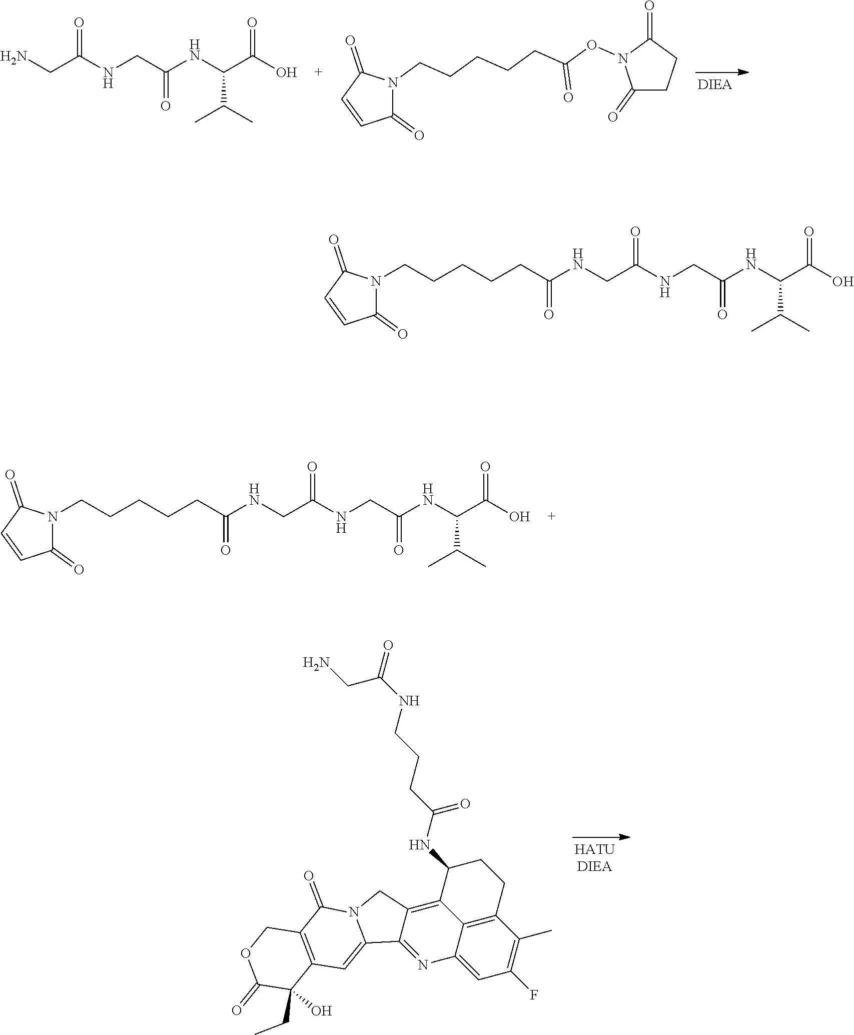

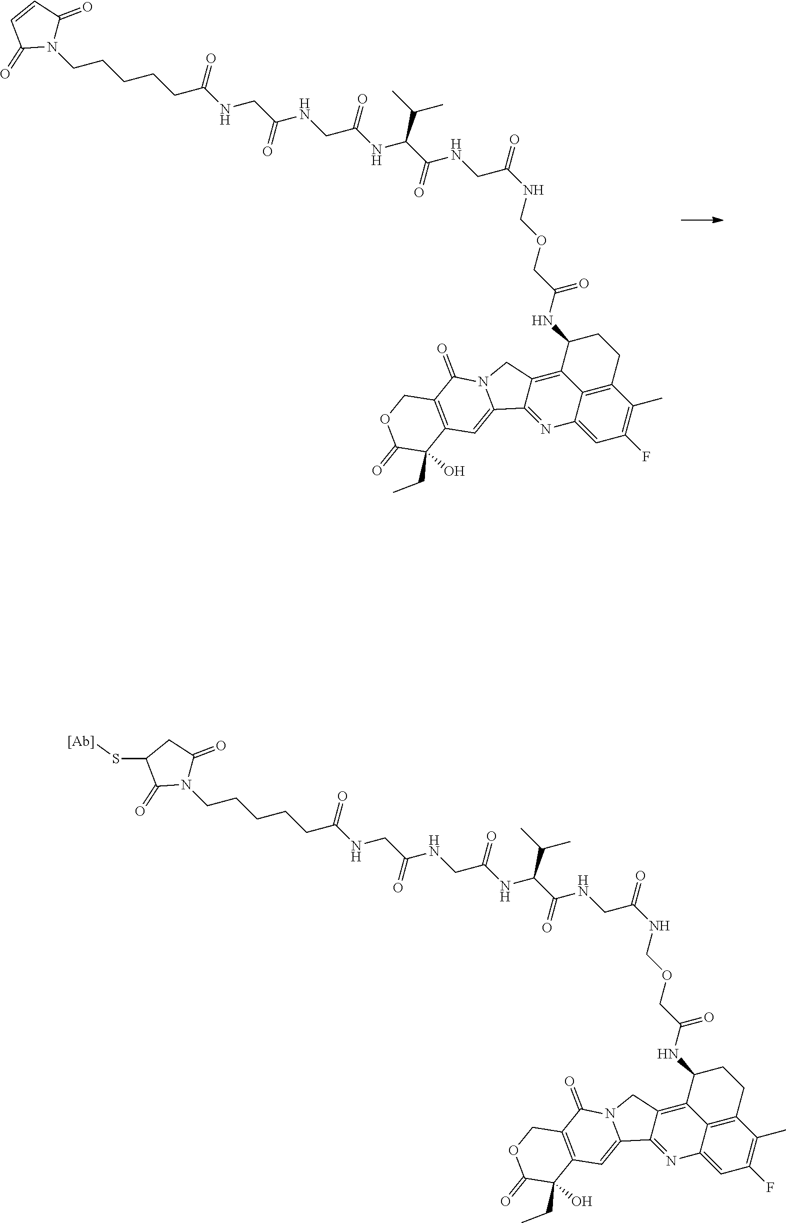

Fmoc-Gly-2-(aminomethoxy) acetic acid was coupled with exatecan using HATU. The Fmoc group was then removed using diethylamine. This product was coupled with MC-GGV-OH using HATU to provide MC-GGVG-aminomethyl-DXd which was subsequently reacted with reduced antibody to give C2-MC-GGVG-aminomethyl-DXd.

### Synthetic Scheme: MC-GGV

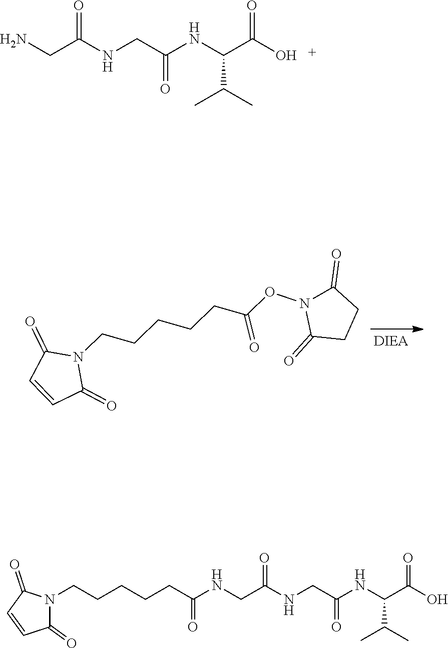

### Formation of MC-GGV

In a glass vial was placed glycine-glycine-valine (GGV, 61.5 mg), DMSO (2 mL) was added under argon followed by DIEA (100 μL) to reaction. The mixture was stirred for 5 min to dissolve. MC-OSu (103.8 mg) was added in one portion to the reaction and it was stirred at room temperature. After 17 h, the reaction was transferred dropwise to a stirring solution of diethyl ether (50 mL), resulting in formation of a translucent residue at the bottom of the flask. The supernatant was then decanted, 50 mL of fresh diethyl ether was added, and decanted again. More diethyl ether (50 mL) was added and decanted a third time, leaving a viscous, cloudy residue at the bottom of the flask. Acetone/ether (1:1, 50 mL) was added, resulting in a cloudy suspension and then was placed in an ice-bath. The suspension was transferred to a flask and rotovaped to afford a white solid. The solid was resuspended in ether/acetone (10:1) and sonicating, after which it was collected on fritted glass. Remaining solid in the flask was collected by adding acetone (10 mL) and sonicating, followed by filtration. After drying, the mass of the white solid weighed 58.0 mg, 1H NMR (DMSO with 0.03% v/v TMS, 400 MHz): δ 8.04 (2H, dt, J=20.56, 6.04 Hz), 7.86 (1H, d, J=8.56 Hz), 6.99 (2H, s), 4.11 (1H, dd, J=8.56, 5.62 Hz), 3.75 (2H, d, J=5.73 Hz), 3.66 (2H, d, J=5.62 Hz), 3.36 (4H, t, J=7.12 Hz), 2.10 (2H, t, J=7.77 Hz), 2.03 (1H, m), 1.47 (4H, h, J=7.33 Hz), 1.18 (2H, m), 0.85 (6H, dd, J=6.85, 1.34 Hz).

### Synthetic Scheme: MC-GGVG-Aminomethyl-DXd

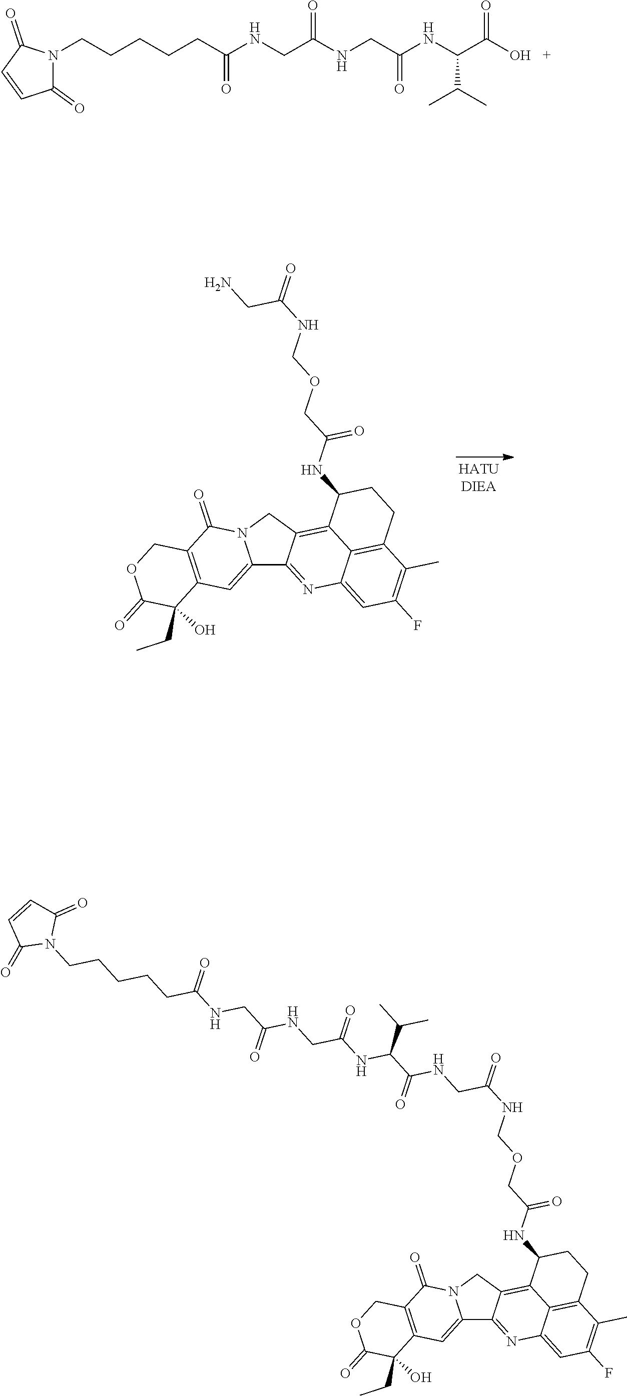

Coupling of MC-GGV-OH with Gly-Aminomethyl-DXd

MC-GGV (34.0 mg) was added to a vial containing Gly-aminomethyl-DXd (32.4 mg) along with a stir-bar. DMF (970 μL) was added to the vial under argon, and the vial was placed into an ice-bath. While in the ice-bath, DIEA (26.5 μL) and HATU (36.4 mg) were added. The reaction was then removed from the ice-bath and allowed to stir at room temp. After 43 min, the reaction was added dropwise to stirring methyl-t-butyl ether (MTBE, 100 mL). The mixture was stirred for 5 min at room temp, then allowed to settle. The solid was collected on fritted glass (66.4 mg after drying). This material was dissolved in DCM (100 mL) with a few milliters of methanol. Celite (1.03 g) was added and the mixture was rotovapped and then high-vacuumed. The solid cartridge was combiflashed on a 4 g silica gel column, elution with 0-->20% MeOH in DCM. Fractions containing the product were rotovapped, then high vacuumed for 1 h wrapped in foil, then placed in a −20C freezer, the mass of the light pink solid weighed 22.8 mg, 1H NMR (DMSO with 0.03% v/v TMS, 400 MHz): δ 8.64 (1H, t, J=6.48 Hz), 8.48 (1H, d, J=8.80 Hz), 8.22 (1H, t, J=6.03 Hz), 8.03 (2H, dt, J=20.51, 6.25 Hz), 7.84 (1H, d, J=8.19 Hz), 7.76 (1H, d, J=10.88 Hz), 7.30 (1H, s), 6.98 (2H, s), 6.50 (1H, s), 5.58 (1H, q, J=6.23 Hz), 5.41 (2H, s), 5.18 (2H, s), 4.61 (2H, d, J=7.07 Hz), 4.08 (1H, t, J=7.78 Hz), 4.00 (2H, s), 3.75 (2H, d, J=5.62 Hz), 3.69 (2H, d, J=5.76 Hz), 3.66 (2H, d, J=5.70 Hz), 3.35 (5H, t, J=7.24 Hz), 3.16 (3H, m), 2.38 (3H, d, J=1.83 Hz), 2.17 (2H, p, J=5.76 Hz), 2.07 (2H, t, J=7.55 Hz), 1.94 (1H, q, J=6.76 Hz), 1.85 (2H, i, J=7.53 Hz), 1.44 (5H, i, J=7.37 Hz), 1.16 (3H, m), 0.86 (4H, t, J=7.42 Hz), 0.80 (6H, dd, J=7.09, 4.55 Hz).

### Synthetic Scheme: C2-MC-GGVG-Aminomethyl-DXd

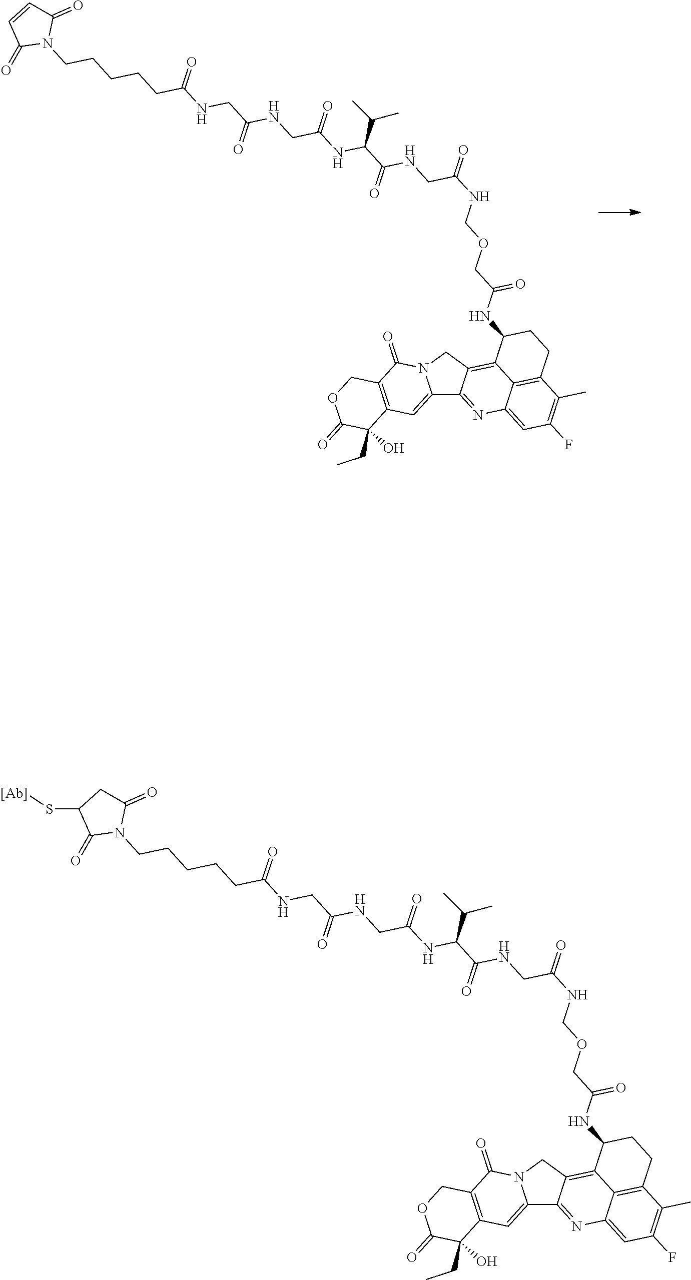

C2 Antibody Reduction and Alkylation with MC-GGVG-Aminomethyl-DXd.

A C2 antibody (7.326 mg/mL, 2.17 mg, 14.47 nmols, 300 uL) was reduced by adding dithiothreitol (DTT) (6.48 mM, 94.05 nmols, 14.5 μL, 6.5 equiv.). The solution was put on a hot water bath at 37 C for two h, with vortexing every 20 min. After 2 h, the solution was put on ice and an aliquot taken to assess free thiol content using an Ellman's test. This indicated a free thiol content of 6.0 free thiols per antibody. A Desalting column was used to buffer exchange the solution to pH 7.4 EPPS, with a 40 μL chaser of pH 7.4 EPPS used to fully elute product. The solution was further purified and concentrated using a 50 kDa MWCO filter and pH 7.4 EPPS buffer. MC-GGVG-aminomethyl-DXd was dissolved in DMSO (9.67 mM, 145 nmols, 10 equiv.) and further diluted in DMSO to achieve a final organic phase that is 20% of the total volume. The solution was then vertically rotated for 60 min at room temperature. N-acetylcysteine (100 mM, 289.4 nmoles, 2.9 μL, 20 equiv.) was dissolved in water and added to the reaction solution. This was vortexed and let stand for 15 min at room temperature. A sephadex G-25 column was then used to purify the solution, using an elution volume of 1.25 mL of pH 7.4 PBS. Stabilization buffer (312.5 μL) was added to the solution and concentration determined using nanodrop (1.236 mg/mL, 1.93 mg, 12.86 nmols, Yield: 64%). The drug-antibody ratio was calculated using cuvette and HIC/HPLC (cuvette DAR: 7.5, HIC DAR: 7.3, ACG: 7.4).

### I. Synthesis of C2-MC-GGVA-Aminomethyl-DXd

### Synthetic Scheme: C2-MC-GGVA-Aminomethyl-DXd

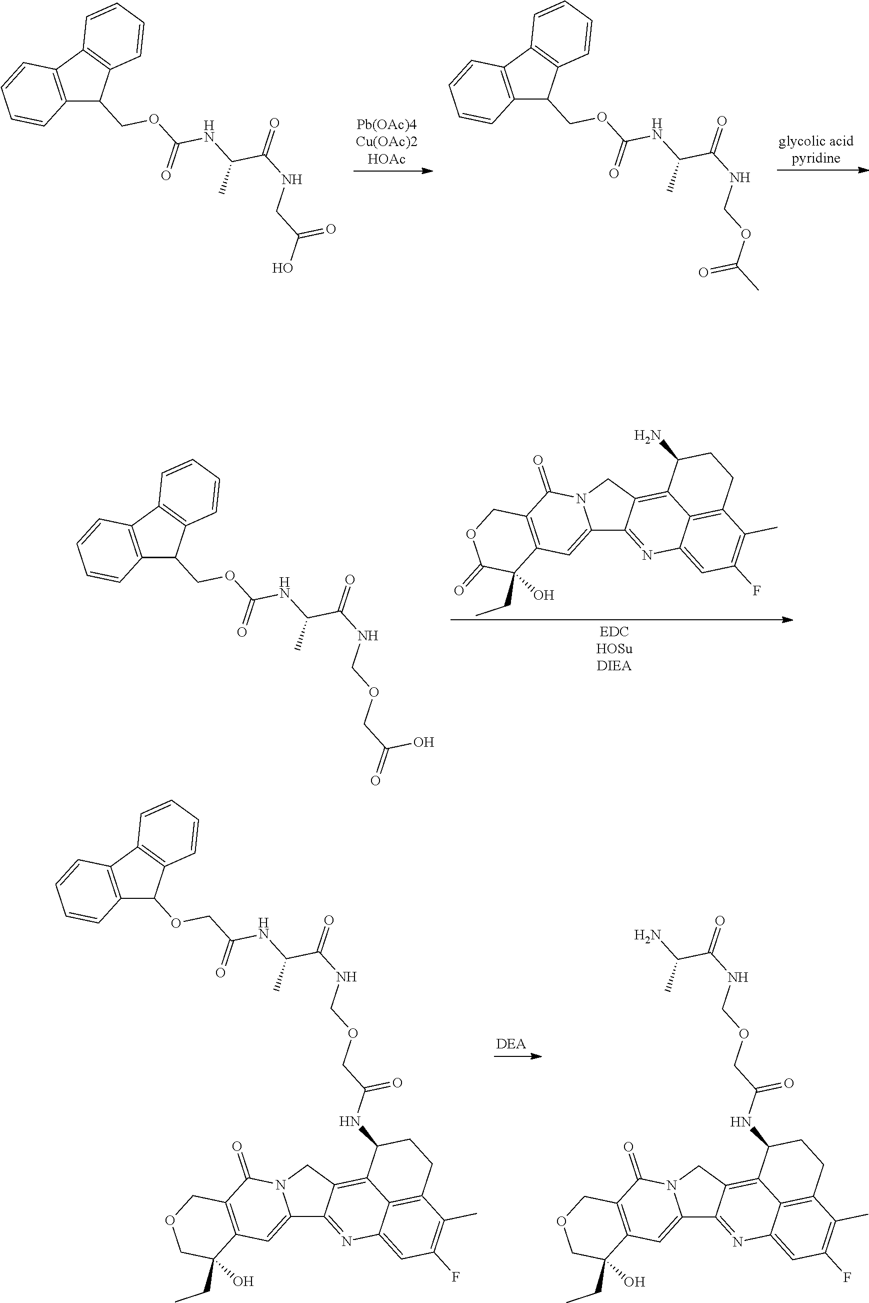 [SMILES: C[C@H](NC(=O)OCC1c2ccccc2-c2ccccc21)C(=O)NCC(=O)O ; CC(=O)OCNC(=O)[C@H](C)NC(=O)OCC1c2ccccc2-c2ccccc21 ; C[C@H](NC(=O)OCC1c2ccccc2-c2ccccc21)C(=O)NCOCC(=O)O ; CC[C@@]1(O)C(=O)OCc2c1cc1n(c2=O)Cc2c-1nc1cc(F)c(C)c3c1c2[C@@H](N)CC3 ; CC[C@@]1(O)COCc2c1cc1n(c2=O)Cc2c-1nc1cc(F)c(C)c3c1c2[C@@H](NC(=O)COCNC(=O)[C@H](C)NC(=O)COC1c2ccccc2-c2ccccc21)CC3 ; CC[C@@]1(O)COCc2c1cc1n(c2=O)Cc2c-1nc1cc(F)c(C)c3c1c2[C@@H](NC(=O)COCNC(=O)[C@H](C)N)CC3]

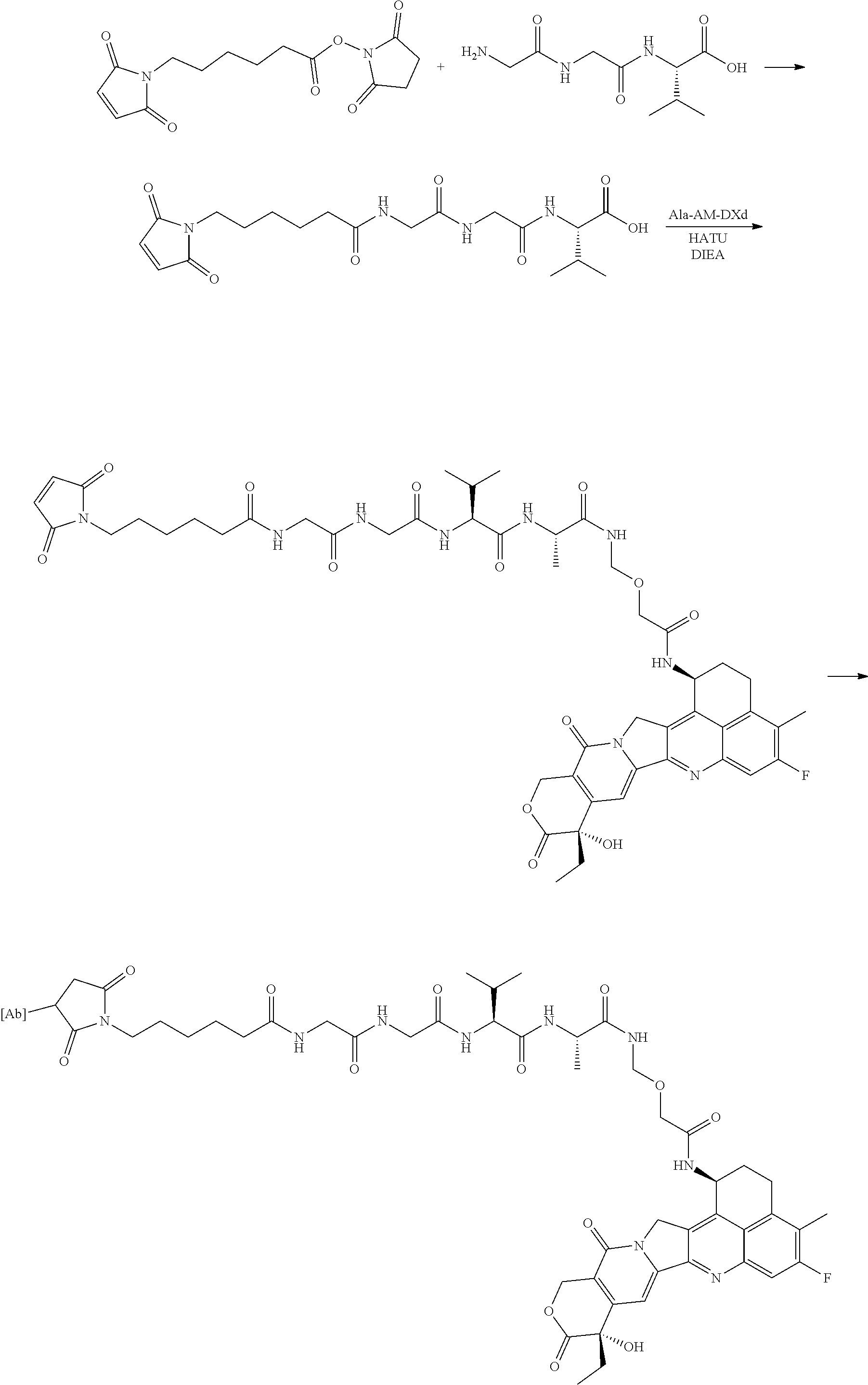

MC-GGVA-aminomethyl-DXd was synthesized in a 7-step reaction sequence. Fmoc-Ala-Gly-OH was oxidized to Fmoc-Ala-aminomethylglyocolic acid. The carboxylic acid was coupled with exatecan using EDC/HOSu. The Fmoc group was then deprotected using diethylamine. Maleimidocaproic acid N-hydroxysuccimide active ester was reacted with Gly-Gly-Val-OH to give MC-GGV-OH. The carboxylic acid was then amide-coupled with Ala-2-(aminomethoxy) acetic acid to provide MC-GGVA-aminomethyl-DXd. The reduced disulfide bonds in the antibody were then alkyated with the maleimide to give C2-MC-GGVA-aminomethyl-DXd.

### Synthetic Scheme: Fmoc-Ala-Aminomethylacetate

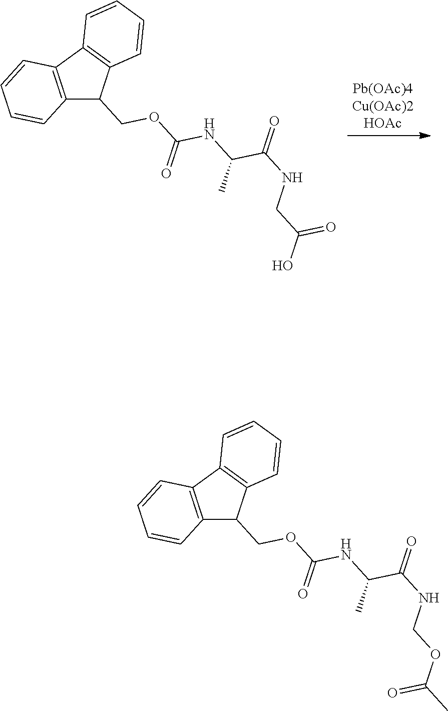 [SMILES: C[C@H](NC(=O)OCC1c2ccccc2-c2ccccc21)C(=O)NCC(=O)O ; CC(=O)OCNC(=O)[C@H](C)NC(=O)OCC1c2ccccc2-c2ccccc21]

### Fmoc-Ala-Gly-OH Oxidative Decarboxylation

In a vial, Fmoc-Ala-Gly-OH (737 mg) in DMF (10.0 mL) and acetic acid (260 μL) and was vacuum degassed under argon. Then Cu (OAc) 2 (134 mg) was added, and degassed. This was followed by addition of Pb (OAc) 4 (1.77 g), and degassing. The vial was placed on an aluminum heating block at 60 °C for 40 min. The reaction was then cooled to room temperature, diluted with EtOAc (150 mL), and washed with water (3×60 mL), with each being pooled together. The aqueous layer was then filtered through celite and back extracted with EtOAc (50 mL) twice, with the extractions being pooled. This was washed with brine (20 mL), dried over anhydrous Na2SO4, and evaporated to a colorless solid (790 mg). This was then dissolved in DCM (250 mL), celite (10 g) was added and the solvent was evaporated off. The celite was placed under a high vac for a few min and transferred to a solid load cartridge where it was combiflashed on a silica gel (40 g). The product was eluted with a gradient of DCM to 3% methanol in DCM and fractions with pure product were combined and evaporated. The dried product was weighed 662 mg, 1H NMR (DMSO with 0.03% v/v TMS, 400 MHz): δ 8.89 (3H, t, J=7.34 Hz), 7.88 (8H, d, J=7.51 Hz), 7.72 (7H, t, J=7.30 Hz), 7.59 (3H, d, J=7.57 Hz), 7.41 (8H, t, J=7.42 Hz), 7.32 (8H, t, J=7.48 Hz), 5.08 (6H, dd, J=6.63, 3.31 Hz), 4.24-4.29 (7H, 4.25 (s), 4.26 (s)), 4.17-4.23 (4H, 4.19 (s), 4.21 (d, J=6.73 Hz)), 4.05 (3H, t, J=7.30 Hz), 3.28-3.33 (12H, 3.29 (m), 3.32 (s)), 2.88 (OH, s), 2.72 (OH, s), 2.49 (10H, quint, J=1.91 Hz), 1.98 (9H, s), 1.89 (1H, s), 1.20 (11H, d, J=7.18 Hz).

### Synthetic Scheme: Fmoc-Ala-Aminomethylglycolate

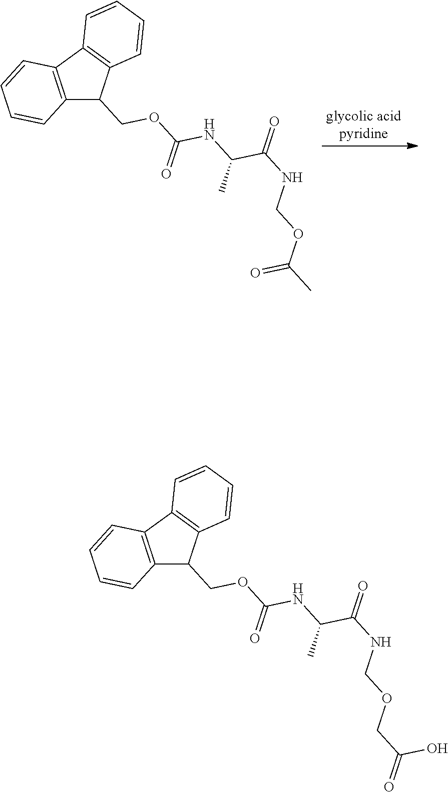 [SMILES: CC(=O)OCNC(=O)[C@H](C)NC(=O)OCC1c2ccccc2-c2ccccc21 ; C[C@H](NC(=O)OCC1c2ccccc2-c2ccccc21)C(=O)NCOCC(=O)O]

### Fmoc-Ala-Aminomethylglycolate

Under argon, Fmoc-Ala-aminomethyl acetate (379.1 mg), glycolic acid (753.9 mg), and pyridine p-toluene sulfonic acid (24.9 mg) were placed on a heating block and heated to 40C overnight with magnetic stirring. After 18 h, DCM (5 mL) was added. The reaction mixture containing white solid was transferred to a Erlenmeyer flask. EtOAc (800 mL) was added with stirring followed by water (80 mL). After stirring a few min, the solution clarified and the biphasic mixture was transferred to a separatory funnel where the layers were separated. The organic layer was washed with another two portion of H2O (2×80 mL), then brine (40 mL). The organic layer was paper-filtered into anhydrous Na2SO4, then filtered and evaporated to give the crude product as a white solid. After high-vacuum, the mass of white solid weighed 313.0 mg, 1H NMR (DMSO with 0.03% v/v TMS, 400 MHz): δ 8.70 (1H, t, J=6.67 Hz), 7.88 (2H, d, J=7.55 Hz), 7.72 (2H, t, J=8.93 Hz), 7.57 (1H, d, J=7.55 Hz), 7.41 (2H, t, J=7.44 Hz), 7.32 (2H, t, J=7.71 Hz), 4.59 (2H, m), 4.26 (2H, d, J=7.05 Hz), 4.20 (1H, t, J=6.83 Hz), 4.00 (1H, t, J=7.30 Hz), 1.22 (4H, d, J=7.17 Hz).

### Synthetic Scheme: Fmoc-Gly-Aminomethyl-DXd

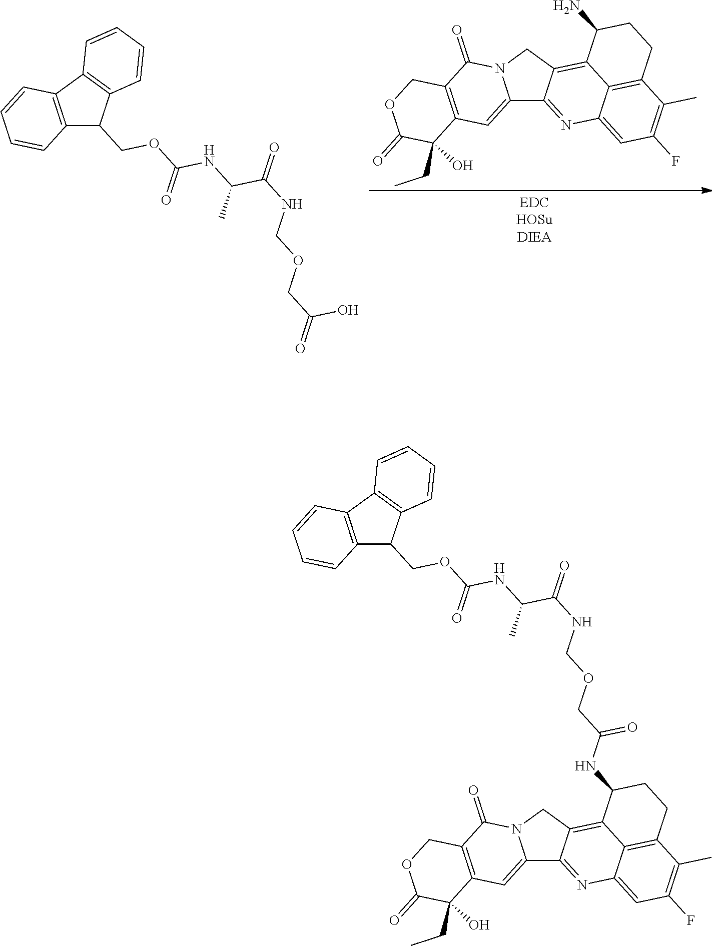 [SMILES: C[C@H](NC(=O)OCC1c2ccccc2-c2ccccc21)C(=O)NCOCC(=O)O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)OCC1c2ccccc2-c2ccccc21)CC3]

### Fmoc-Gly-Aminomethyl-DXd

Into a vial, Fmoc-ala-2-(aminomethoxy) acetic acid (74.2 mg) was added followed by anhydrous DMF (1.26 mL) under argon. DIEA (64.3 μL), HOSu (25.6 mg) and EDC-HCl (55.4 mg) were then added in that order. The reaction was stirred, and more DIEA (65 μL) was added after 12 min. After 30 min total, exatecan mesylate (101.1 mg) was added to the reaction mixture. After 2 h and 20 min, EDC was added and after 3 h and 30 min, the solution was transferred to a separatory funnel containing EtOAc (200 mL), and washed with citric acid (1M, 3×30 mL) followed by saturated NaHCO3 (3×30 mL), and then brine (30 mL) with the aqueous phase being removed. The organic layer was filtered onto sodium sulfate and transferred to a round bottom flask. The organic layer was then vacuumed off. The dried solid was dissolved in dichloromethane (DCM) and celite (1.5 g) was added and the solvent vacuumed off. The product (96.3 mg) was purified on combiflash with silica gel and eluted with 1% HOAc/DCM to 8% MeOH/1% HOAc/DCM gradient. 1H NMR (DMSO with 0.03% v/v TMS, 400 MHz): δ 8.77 (1H, s), 8.46 (1H, d, J=8.80 Hz), 8.46 (1H, d, J=8.77 Hz), 7.83 (2H, d, J=7.70 Hz), 7.83 (2H, d, J=7.49 Hz) 7.74 (1H, d, J=10.88 Hz), 7.74 (1H, d, J=10.90 Hz), 7.67 (2H, t, J=7.28 Hz), 7.67 (2H, t, J=7.15 Hz), 7.53 (1H, d, J=7.46 Hz), 7.53 (1H, d, J=7.37 Hz), 7.37 (2H, q, J=7.75 Hz), 7.28 (3H, t, J=10.10 Hz), 7.28 (5H, m), 6.50 (1H, br s), 5.54 (1H, q, J=6.05 Hz), 5.37-5.41 (2H, 5.39 (d, J=1.60 Hz), 5.40 (s)), 5.39 (2H, m), 5.16 (2H, q, J=13.57 Hz), 5.16 (2H, d, J=13.57 Hz), 4.61 (2H, i, J=6.48 Hz), 4.61 (2H, q, J=4.33 Hz), 4.20 (3H, m), 4.20 (2H, m), 3.99 (3H, s), 3.97-4.01 (2H, 3.98 (s), 3.99 (s)) 3.31 (7H, s), 3.31 (7H, br s), 3.16 (OH, s), 3.11 (3H, m), 2.49 (6H, quint, J=1.76 Hz), 2.37 (d, J=1.96 Hz)), 2.33-2.38 (3H, 2.35 (s), 2.35 (3H, br s), 2.15 (2H, s), 1.89 (2H, s), 2.15 (2H, m), 1.89 (3H, s), 1.83 (2H, i, J=7.11 Hz), 1.83 (2H, t, J=7.07 Hz), 1.18 (2H, d, J=7.17 Hz), 1.18 (3H, d, J=7.09 Hz), 0.84 (3H, m), 0.84 (3H, m).

### Synthetic Scheme: Ala-Aminomethyl-DXd

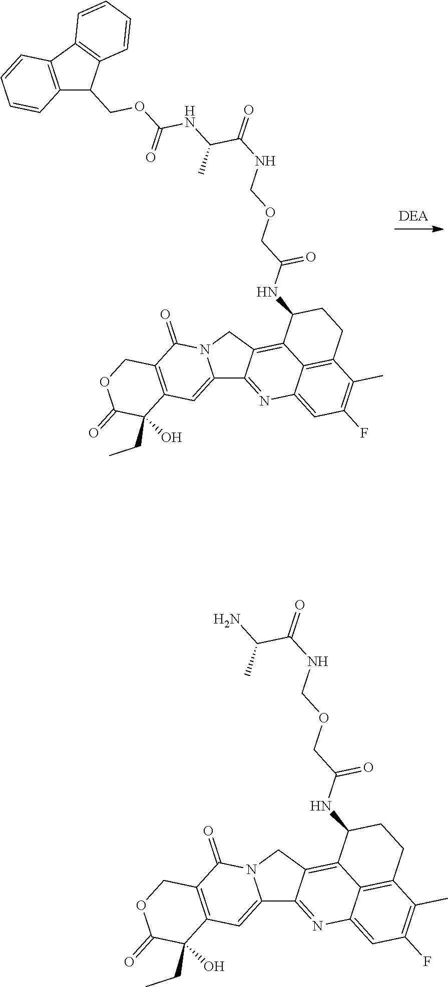 [SMILES: CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)OCC1c2ccccc2-c2ccccc21)CC3 ; CC[C@@]1(O)C(=O)OCc2c1cc1n(c2=O)Cc2c-1nc1cc(F)c(C)c3c1c2[C@@H](NC(=O)COCNC(=O)[C@H](C)N)CC3]

Fmoc Cleavage with Diethylamine

Anhydrous DMF (105 μL) was added to a vial containing Fmoc-Ala-aminomethyl-DXd (96.3 mg), along with a stir-bar. Diethylamine (2.5 mL) was added. After 41 min, diethyl ether (12 mL) was added, resulting in precipitation of solid. The suspension was stirred for 5 min and then the solid was then collected on fritted glass and washed with diethyl ether (2×10 mL). The solid was dried to give a 61.2 mg. This material was then dissolved in DCM (100 mL) and MeOH (5 mL) was added to a give a clear solution. Celite (1 g) was added and the solvent was evaporated. Combiflash was performed using a gradient from 5% MeOH/DCM to 45% MeOH DCM. After combiflash (4 g silica gel column), fractions containing product were pooled and evaporated to give a solid (45.3 mg). 1H NMR (DMSO with 0.03% v/v TMS, 400 MHz): δ 8.71 (1H, s), 8.50 (1H, d, J=8.77 Hz), 7.75 (1H, d, J=10.91 Hz), 7.28 (1H, s), 6.51 (1H, s), 5.58 (1H, ddd, J=8.70, 6.18, 4.73 Hz), 5.40 (2H, s), 5.17 (2H, s), 4.58 (2H, s), 3.99 (2H, s), 3.32 (6H, s), 3.20 (2H, m), 3.15 (2H, m), 2.49 (3H, s), 2.37 (3H, br s), 2.17 (2H, dddd, J=13.43, 6.41, 5.80, 3.51 Hz), 1.85 (3H, dd, J=11.52, 8.62 Hz), 1.03 (3H, d, J=6.87 Hz), 0.85 (3H, t, J=7.25 Hz).

### Synthetic Scheme: MC-GGV-OH

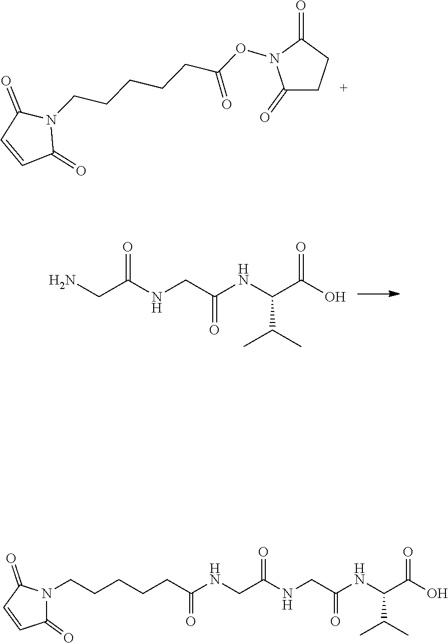

### Formation of MC-GGV-OH

GGV-OH (184.4 mg) was added to a vial. DMSO (793 μL) was added to the vial under argon, followed by DIEA (300 μL). The reaction was stirred for 5 min to dissolve. Then MC-OSu (298.3 mg) was added in one portion and the reaction was stirred at room temperature over the weekend. The solvent was rotovapped off at 60C, and then dried under high vacuum with a vacuum distillation adapter as a trap for 1 h. The thick oil was dissolved in acetone (1 mL) and transferred to a 150 mL Erlenmeyer flask. Diethyl ether (100 mL) was added, the flask was washed with acetone (1 mL), and methanol (1 mL). The washings were added to the ether layer, and cooled in the refrigerator for 3-4 h to fully ppt the crude product as an oil and a solid. The solid was collected on fritted glass. Acetone (10 mL) was added to suspend the oil, which turned into a white solid, collected on fritted glass. Dried under vacuum (188.5 mg). 1H NMR (DMSO with 0.03% v/v TMS, 400 MHz): δ 8.06 (1H, s), 8.01 (1H, s), 7.86 (1H, d, J=8.51 Hz), 6.99 (2H, s), 4.11 (1H, dd, J=8.51, 5.77 Hz), 3.75 (3H, d, J=5.72 Hz), 3.66 (3H, d, J=5.75 Hz), 3.36 (4H, t, J=7.11 Hz), 2.53 (OH, s), 2.49 (5H, quint, J=1.75 Hz), 2.09 (4H, m), 1.47 (6H, q, J=7.13 Hz), 1.19 (3H, m), 0.85 (8H, dd, J=6.79, 0.94 Hz).

### Synthetic Scheme: MC-GGVA-Aminomethyl-DXd

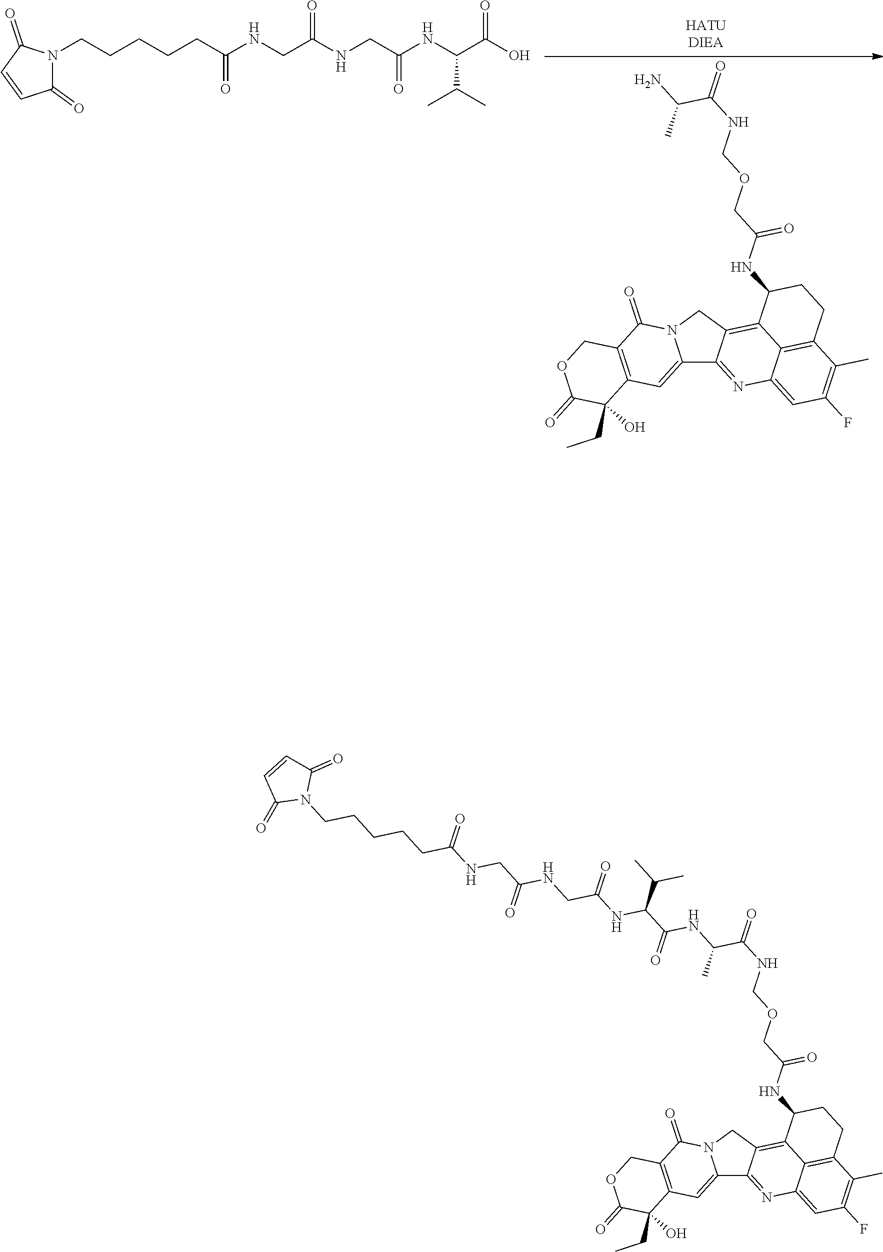 [SMILES: CC(C)[C@H](NC(=O)CNC(=O)CNC(=O)CCCCCN1C(=O)C=CC1=O)C(=O)O ; CC[C@@]1(O)C(=O)OCc2c1cc1n(c2=O)Cc2c-1nc1cc(F)c(C)c3c1c2[C@@H](NC(=O)COCNC(=O)[C@H](C)N)CC3 ; CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@@H](NC(=O)CNC(=O)CNC(=O)CCCCCN1C(=O)C=CC1=O)C(C)C)CC3]

### Synthesis of MC-GGVA-Aminomethyl-DXd

MC-GGV (6.3 mg) was added to a vial containing ala-aminomethyl-DXd (45.3 mg) along with a stir-bar. Anhydrous DMF (1.32 mL) was added under argon, and the vial was placed into an ice-bath. DIEA (36.2 μL) and HATU (50.8 mg) were added. The reaction was then removed from the ice-bath and allowed to stir at room temp. After 30 min, the reaction was added dropwise to stirring MTBE (100 mL). The suspension was stirred for 5 min at room temp, then allowed to settle. The solid was collected on fritted glass (105.2 mg, crude). This material was dissolved in DCM (100 mL) and MeOH (5 mL). Celite (1.5 g) was added and the mixture was rotovapped and then high-vacuumed. The material was combiflashed on 4 g silica, elution with 1%-->20% MeOH in DCM. Fraction 4 contained product and was rotovapped, then high vacuumed, then placed in a −20C freezer overnight. The dried solid was a light pink color (36.4 mg). 1H NMR (DMSO with 0.03% v/v TMS, 400 MHz): δ 8.64 (1H, t, J=6.48 Hz), 8.48 (1H, d, J=8.80 Hz), 8.22 (1H, t, J=6.03 Hz), 8.03 (2H, dt, J=20.51, 6.25 Hz), 7.84 (1H, d, J=8.19 Hz), 7.76 (1H, d, J=10.88 Hz), 7.30 (1H, s), 6.98 (2H, s), 6.50 (1H, s), 5.58 (1H, q, J=6.23 Hz), 5.41 (2H, s), 5.18 (2H, s), 4.61 (2H, d, J=7.07 Hz), 4.08 (1H, t, J=7.78 Hz), 4.00 (2H, s), 3.75 (2H, d, J=5.62 Hz), 3.69 (2H, d, J=5.76 Hz), 3.66 (2H, d, J=5.70 Hz), 3.35 (5H, t, J=7.24 Hz), 3.16 (3H, m), 2.38 (3H, d, J=1.83 Hz), 2.17 (2H, p, J=5.76 Hz), 2.07 (2H, t, J=7.55 Hz), 1.94 (1H, q, J=6.76 Hz), 1.85 (2H, i, J=7.53 Hz), 1.44 (5H, i, J=7.37 Hz), 1.16 (3H, m), 0.86 (4H, t, J=7.42 Hz), 0.80 (6H, dd, J=7.09, 4.55 Hz).

### Synthetic Scheme: C2-MC-GGVA-Methylamino-DXd

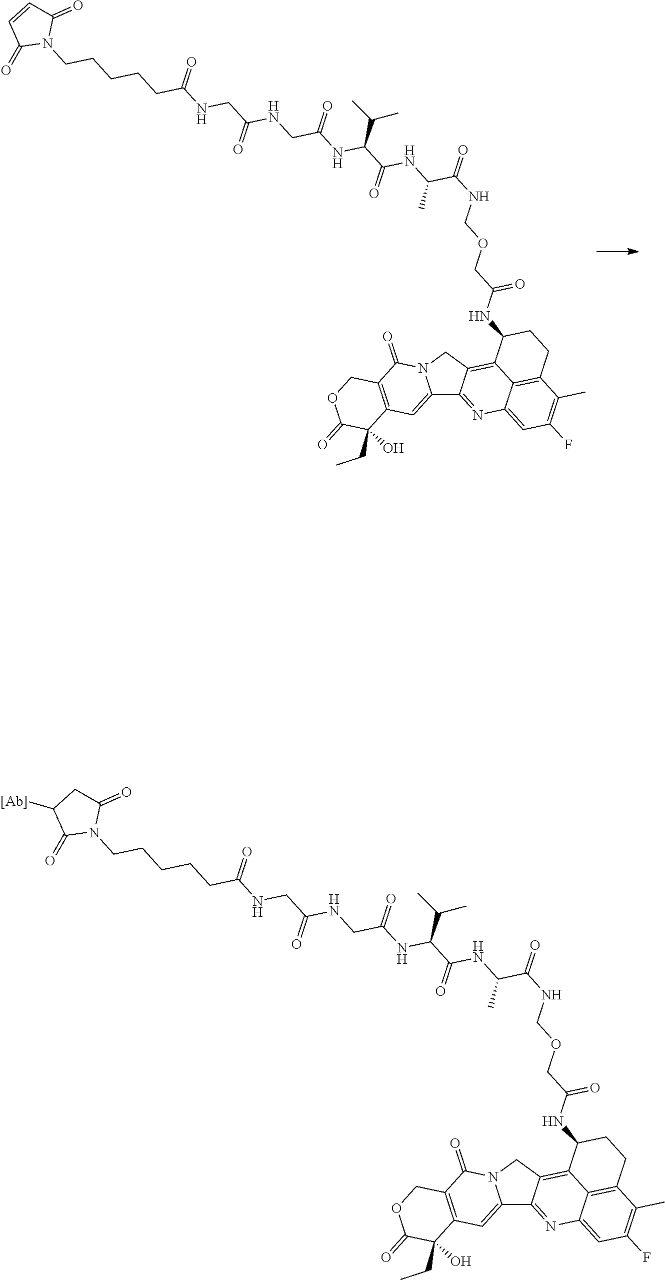

### Reduction and Alkylation of C2 Antibody

A C2 antibody (7.388 mg/mL, 2.22 mg, 14.78 nmols, 300 μL) in pH 8 borate buffer (25 mM Sodium borate, 25 mM sodium chloride, 1 mM EDTA buffer) was reduced with dithiothreitol (DTT) (6.48 mM, 96.07 nmols, 14.8 μL, 6.5 equiv.) also in borate buffer. The reaction was placed in a water bath at 37° for 2h. Following this, the solution was placed on ice and an aliquot taken to determine the free thiol content of the antibody using an Ellman's test. The free thiol content for this was 7.5 free thiols per antibody. A Desalting column was used to purify and buffer exchange the solution to a pH 7.4 EPPS buffer (50 mM EPPS and 5 mM DTPA). The solution was then purified further using a 50 kDa MWCO filter and pH 7.4 EPPS buffer. The solution was then brought back up to the original volume before purification in the same pH 7.4 EPPS buffer. MC-GGVA-aminomethyl-DXd (2.188 mg/mL, 148.0 μg, 67.6 μL, 10 equiv.) was dissolved in DMSO and diluted further with DMSO to get a total organic phase of 20% of the total volume. The drug was added to the antibody and put on a vertical rotator for 60 min at room temperature. After 1h, N-acetylcysteine (NAC) in water (100 mM, 295.6 nmols, 2.9 uL), was added to the antibody for end capping, vortexed, and let stand for 15 min at room temperature. A Sephadex G-25 column was used to purify the solution, with 1.25 mL of pH 7.4 PBS (Gibco, Cat. No. 10010049) being used to elute the product from the column. Stabilization buffer (312.5 μL) was added to the final solution, and concentration determined by nanodrop (1.218 mg/mL, 1.9 mg, 12.67 nmols, yield: 64%). The drug-antibody ratio was determined by cuvette and HIC/HPLC (Cuvette DAR: 7.7, HIC DAR: 7.7, AVG: 7.7).

### J. Synthesis of C2-MC-VA-Aminomethyl-DXd

### Synthetic Scheme: C2-MC-VA-Aminomethyl-DXd

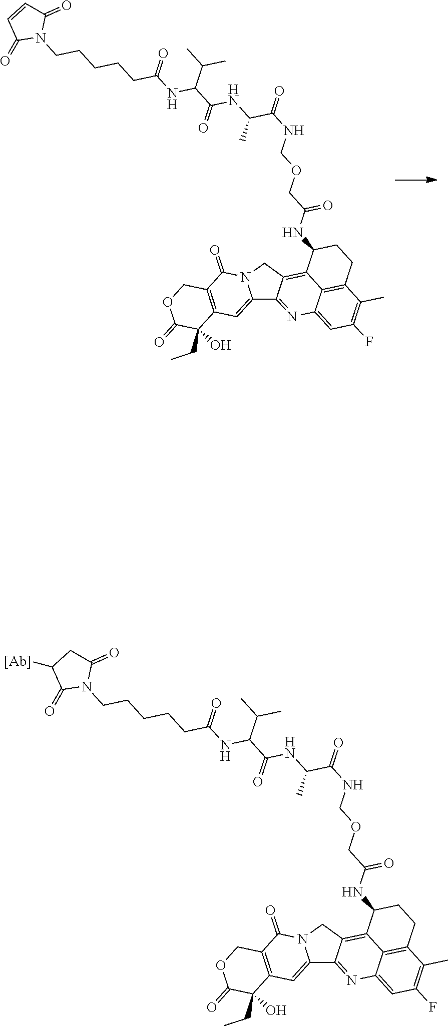

To a solution of C2 antibody (7.043 mg/mL, 2.11 mg, 14.09 nmols, 300 μL) in pH 8 Borate buffer, dithiothreitol (6.48 mM, 91.59 nmols, 14.1 μL) in the same buffer was added. The reaction mixture was placed on a hot water bath at 37° for 2 h with vortexing every 20 min. After 2 h, the solution was placed on ice and an aliquot was taken to assess free thiol content using an Ellman's test, resulting in 6.5 free thiols per antibody. A Desalting column was used to buffer exchange the solution to pH 7.4 EPPS buffer. A chaser of buffer (40 μL) was used to fully elute product from the column. A 50 kDa MWCO filter with pH 7.4 EPPS buffer was used to concentrate and purify the solution further. MC-VA-aminomethyl-DXd (2.188 mg/mL, 141 nmols, 57.1 μL) was dissolved in DMSO and further diluted using DMSO to achieve an organic volume that was 20% of the total. The drug was added to the antibody, vortexed, and put on a vertical rotator for 60 min at room temperature. After 60 min, N-acetylcysteine (100 mM, 281.8 nmols, 2.8 μL) was dissolved in water and added to the solution. This was vortexed and then let stand for 15 min at room temperature. A Sephadex G-25 column was used to purify the solution with pH 7.4 PBS (1.25 mL) being used to elute the product. Stabilization buffer (312.5 μL) was added to the solution and the concentration was measured by nanodrop (1.172 mg/mL, 1.83 mg, 12.2 nmols, Yield: 61%). The drug-antibody ratio was measured by cuvette and HIC/HPLC (Cuvette DAR 7.4, HIC DAR: 7.5, AVG DAR: 7.5).

### K. Synthesis of 3C2B1-MC-VA-PAB-Exatecan

### Synthetic Scheme: 3C2B1-MC-VA-PAB-Exatecan

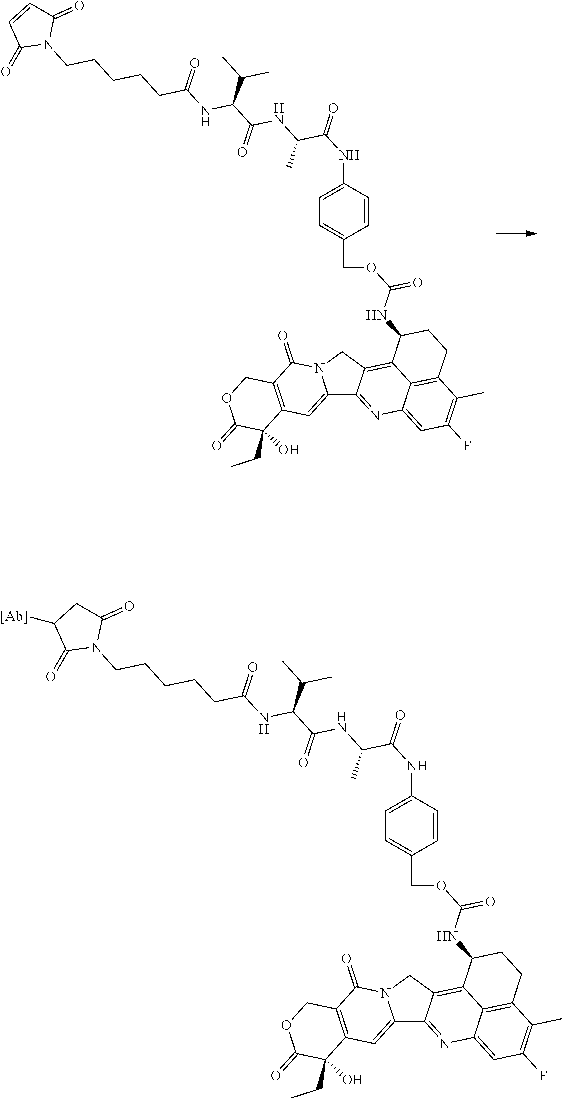

3C2B1-MC-VA-PAB-exatecan was synthesized from commercially available MC-VA-PAB-exatecan. 3C2B1 was reduced with TCEP, and then alkylated via Michael-Addition.

The antibody, 3C2B1, (6.0826 mg/mL, 2.05 mg, 13.7 nmols, 300 μL) in pH 7.4 buffer (50 mM EPPS with 5 mM DTPA) was reduced using tris(2-carboxyethyl)phosphine (TCEP, 4.68 mM, 48 nmols, 10.3 μL, 3.5 equiv.) dissolved in the same pH 7.4 buffer. The reaction was placed on a 37 °C water bath for 90 min, with vortexing every 20 min. After 90 min, the antibody was placed on ice and the free thiol content analyzed using an Ellman's test to get a free thiol content of 5.3. MC-VA-PAB-exatecan (9.67 mM, 137 nmols, 14.2 μL; MedchemExpress LLC, Monmouth NJ, Cat. No. HY-147270) was dissolved in DMSO and further diluted in DMSO to get an organic content of 10% of the total volume. The drug was added to the antibody solution and the reaction was put on a vertical rotator for 60 min. Following this, N-acetylcysteine in water (100 mM, 274 nmoles, 2.7 μL, 20 equiv.) was added to the antibody, vortexed, and allowed to stand at room temperature for 15 min. The solution was purified using a Cytiva PD Miditrap g-35 (Cat. No. 28918008). Following purification, a Desalting column was used to buffer exchange to pH 7.4 PBS (Gibco Cat. No. 10010023). A second Cytiva PD Miditrap g-35 was used to purify the ADC with an elution of 1.25 mL in pH 7.4 PBS. Stabilization buffer (312.5 μL) was added to the finished solution and concentration determined by nanodrop (1.322 mg/mL, 2.06 mg, 13.73 nmols, yield: 69%). The drug-antibody ratio was calculated by cuvette and HIC/HPLC (Cuvette DAR: 5.1, HIC DAR: 5.4, Avg. 5.2).

### L. Synthesis of C2-CL2A-SN38

### Synthetic Scheme: C2-CL2A-SN38

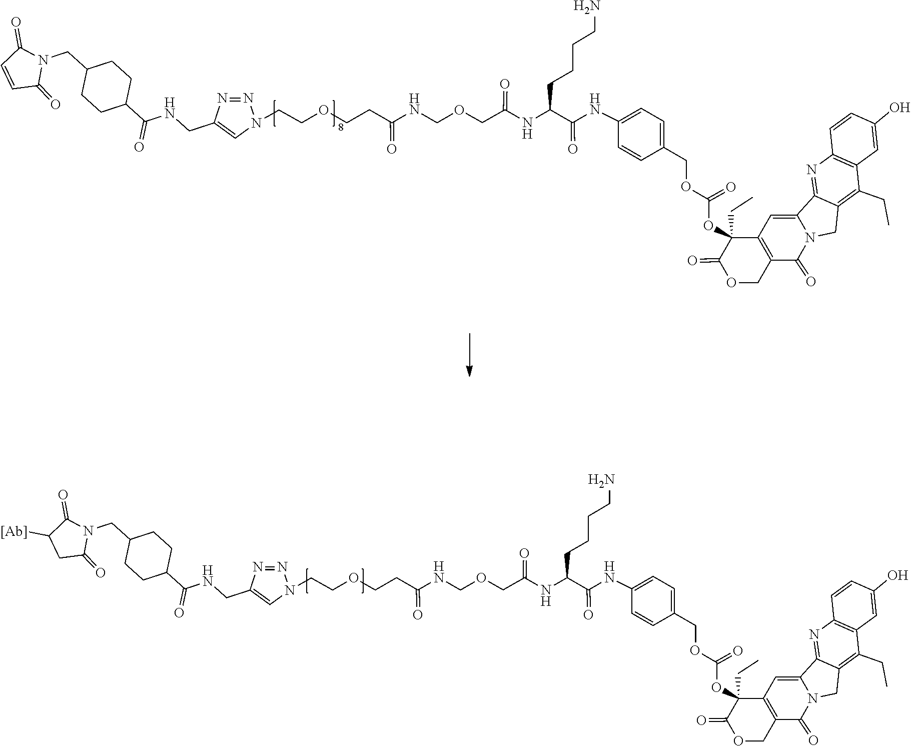

To a solution of C2 antibody (7.533 mg/mL, 2.26 mg, 15.07 nmols) in pH 8 borate buffer, dithiothreitol in the same buffer (DTT, 6.48 mM, 150.7 nmols, 10 eq., 23.3 μL) was added. The solution was heated for 2 h at 37 C, with vortexing every 20 min. After 90 min in the water bath, an aliquot was taken to determine the free thiol content using an Ellman's test, giving a free thiol of 7.13 per antibody. The reduced antibody was then purified with a Zeba Spin Desalting column (Cat #89890) with a 40 μL chaser of pH 6 MES buffer to elute the product. The solution was then buffer-exchanged to a pH 6 MES buffer on a 50 kDA MWCO filter. CL2A-SN38 (MedchemExpress LLC, Monmouth NJ-Cat. No. HY-128946) was dissolved in DMSO (9.67 mM, 150.7 nmols, 10 eq., 16.7 μL) and further diluted with DMSO such that the reaction was 10% v/v DMSO/buffer. The drug was then added to the antibody and placed on a vertical rotator for 60 min at room temperature. After 60 min, the antibody solution was purified on a Cytiva PD Miditrap g-35 (Cat #28918008) conditioned with pH 7.4 PBS buffer (Gibco; Ref: 10010-023). Stabilization buffer (312.5 μL) was added to the collected fraction to give the final solution of purified antibody in pH 7.4 PBS (1.306 mg/mL, 2.04 mg, 13.6 nmols, yield: 68%). The drug-antibody-ratio (DAR) was then determined using cuvette measurement and HIC, and concentration confirmed with nanodrop (Cuvette DAR: 8.9, HIC DAR: 7.4, Avg DAR: 8.2).

### M. Synthesis of C2-CL2E-SN38

### Synthetic Scheme: C2-CL2E-SN38

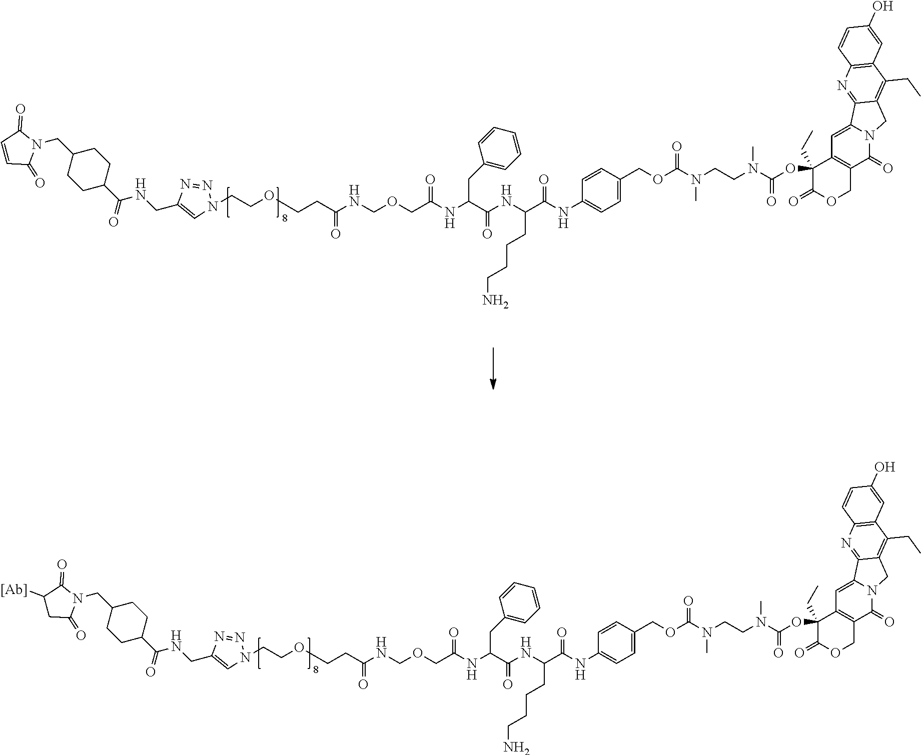

To a solution of C2 antibody (7.398 mg/mL, 2.22 mg, 14.8 nmols) in pH 7.4 buffer (50 mM EPPS with 5 mM DTPA), tris(2-carboxyethyl)phosphine (TCEP, 4.68 mM, 148 nmols, 31.6 μL 10 eq.) dissolved in the same buffer was added. The solution was placed in a water bath at 37 C for 90 min with vortexing every 20 min. After 90 min, the solution was put on ice and an aliquot was taken for an Ellman's test to get a free thiol of 8.05 per antibody. CL2E-SN38 (MedchemExpress LLC, Monmouth NJ-Cat. No. HY-139909A) was dissolved in DMSO (9.67 mM, 148 nmols, 10 eq., 15.3 μL), diluted with DMSO further to make the final DMSO 10% in the alkylation reaction, and then added to the antibody solution and vertically rotated for 60 min at room temperature. After 60 min, a Zeba desalting column (Ref #89890) was used to purify and buffer exchange the solution to pH 7.4 PBS (Gibco Cat #10010-023), with a 40 μL PBS chaser being used to fully elute the sample. Subsequently, the reaction mixture was placed a Cytiva PD Miditrap g-35 column (Cat #28918008) and eluted with pH 7.4 PBS (1.25 mL) to further purify the solution. Stabilization buffer (312.5 μL) was then added to give the final product (1.05 mg/mL, 1.69 mg, 10.9 nmols, yield: 55%). Concentration was measured by nanodrop and the drug-antibody ratio determined using cuvette and HIC/HPLC (Cuvette DAR: 6.2, HIC DAR: 6.3, AVG: 6.2).

### N. Synthesis of C2-MAC-Glucuronide-SN38

### Synthetic Scheme: C2-MAC-Glucuronide-SN38

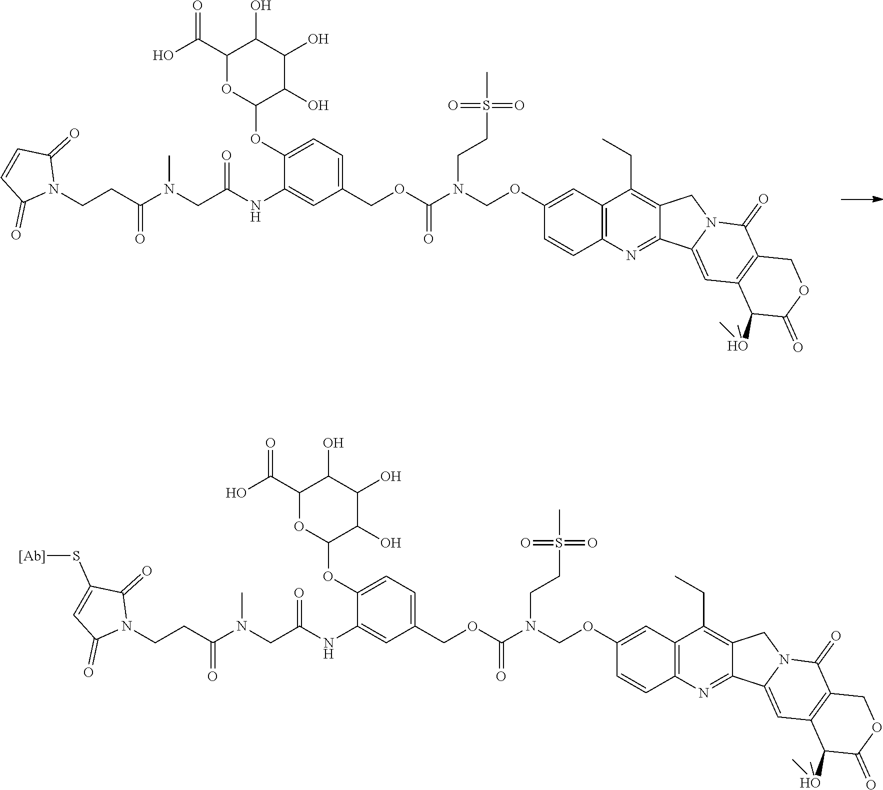

C2 antibody (2.04 mg, 13.60 nmols, 6.783 mg/mL) in pH 8 borate buffer was reduced by adding dithiothreitol (DTT) (10 eq., 6.48 mM, 136.0 nmols, 21.4 μL) in pH 8 borate buffer to the antibody solution. The solution was put in a water bath at 37 C for 2 h with vortexing every 20 min. After 2 h, the solution was put on ice and an aliquot was taken for a free thiol test using the Ellman's test to get a free thiol of 7.75 free thiols per antibody. The antibody solution was then desalted and buffer exchanged to a pH 6 MES (50 mM MES and 5 mM DTPA) buffer using a Zeba Spin Desalting Column (Cat #89890). The solution was then purified and concentrated further using a 50 kDa MWCO filter and pH 6 MES buffer. The solution was brough back up the original volume, and MAC-Glucuronide-SN38 (MedchemExpress LLC, Monmouth NJ-Cat. No. HY-128943) was dissolved in DMSO (9.67 mM, 136 nmols, 10 eq., 14.1 μL). The drug was diluted further with DMSO and added to the antibody solution. This was then vertically rotated for 60 min at room temperature. After 60 min, the column was purified using a Cytiva PD Miditrap g-35 (Cat #: 28918008) conditioned with pH 7.4 PBS (Gibco; Ref: 10010-023). Stabilization buffer (312.5 μL) was then added to the solution to give the final product (0.872 mg/mL, 1.31 mg, 1.5625 mL, Yield=44%). Concentration was measured by nanodrop and drug-antibody ratio determined using cuvette and HIC/HPLC (Cuvette DAR: 9.5, HIC DAR: 9.0, AVG: 8.8).

### O. Synthesis of C2-MC-VC-PAB-SN38

### Synthetic Scheme: C2-MC-VC-PAB-SN38

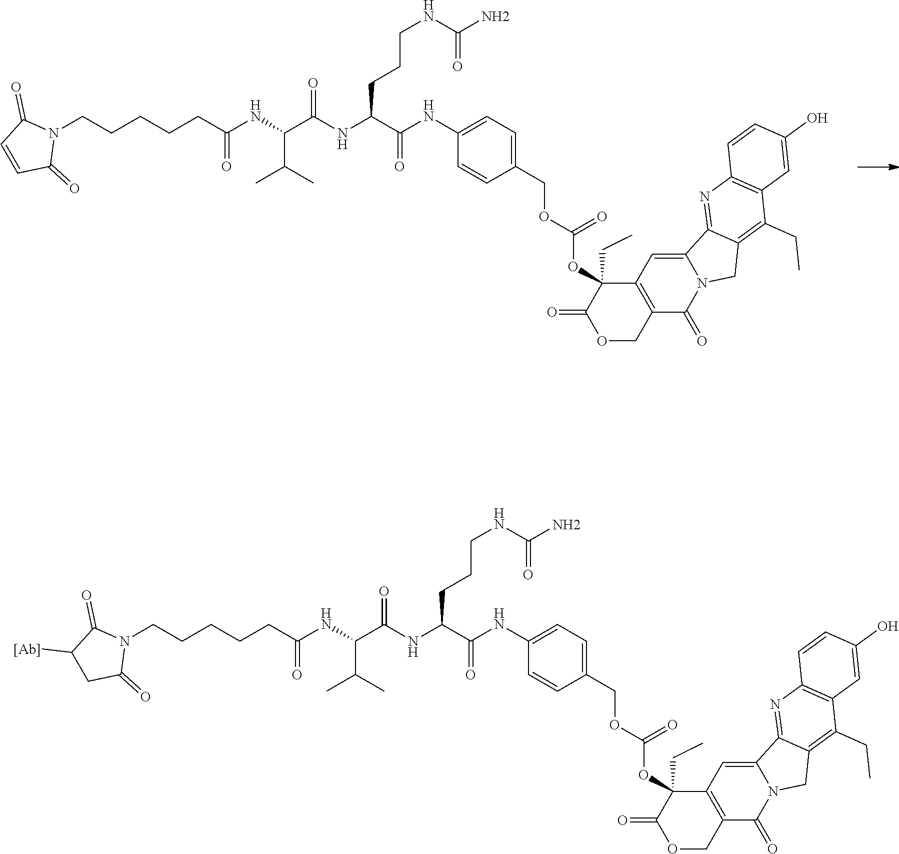

C2 antibody (2.24 mg, 14.94 nmols) in pH 7.4 buffer (50 mM EPPS with 5 mM DTPA) was reduced using tris(2-carboxyethyl)phosphine (TCEP, 7 eq, 22.3 μL, 104.58 nmols, 4.68 mM) in the same buffer solution. The antibody was put on a water bath at 37 C for 90 min. After 90 min, the antibody was placed on ice and an aliquot taken to determine the free thiol content using an Ellman's test. This gave a free thiol value of 6.23. MC-VC-PAB-SN38, (MedchemExpress LLC, Monmouth NJ-Cat. No. HY-131057) was dissolved in DMSO (10 eq., 149.4 nmols, 9.67 mM, 15.4 μL) and diluted further with DMSO (10%) before being added to the antibody solution. The reaction was placed on a vertical rotator for 60 min at room temperature. After 60 min, the solution was purified using a Cytiva PD Miditrap g-35 (Cat #28918008) conditioned with PBS pH 7.4 (Gibco Cat #10010-023). Stabilization buffer (312.5 μL) was added to the purified solution, and the concentration determined by nanodrop (1.573 mg/mL, 2.46 mg, 16.4 nmols, yield: 82%). The drug-to-antibody ratio was determined with cuvette and HIC/HPLC (cuvette DAR: 7.4, HIC DAR: 7.0, AVG: 7.2).

### P. Synthesis of C2-MC-VA-GAB-Exatecan

### Synthetic Scheme: C2-MC-VA-GAB-Exatecan

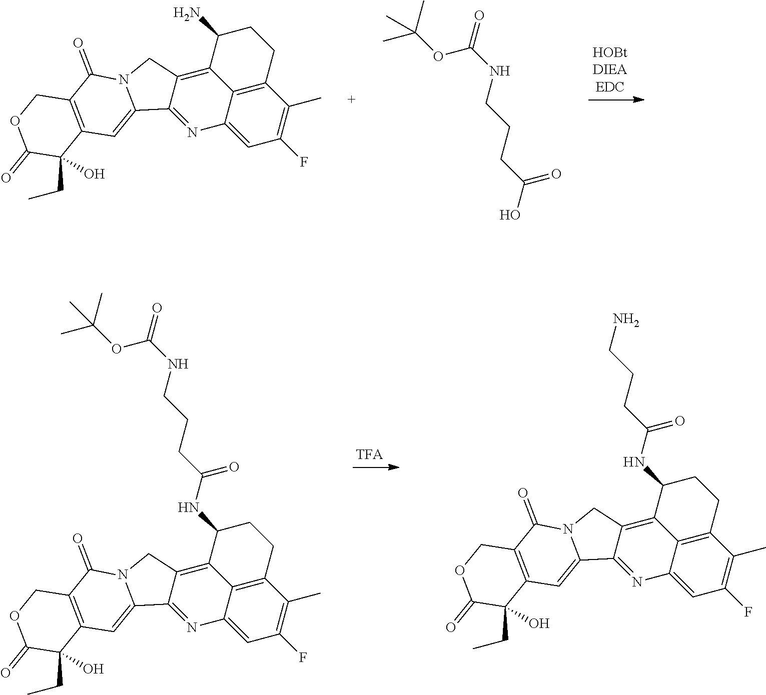 [SMILES: CC[C@@]1(O)C(=O)OCc2c1cc1n(c2=O)Cc2c-1nc1cc(F)c(C)c3c1c2[C@@H](N)CC3 ; CC(C)(C)OC(=O)NCCCC(=O)O ; CC[C@@]1(O)C(=O)OCc2c1cc1n(c2=O)Cc2c-1nc1cc(F)c(C)c3c1c2[C@@H](NC(=O)CCCNC(=O)OC(C)(C)C)CC3 ; CC[C@@]1(O)C(=O)OCc2c1cc1n(c2=O)Cc2c-1nc1cc(F)c(C)c3c1c2[C@@H](NC(=O)CCCN)CC3]

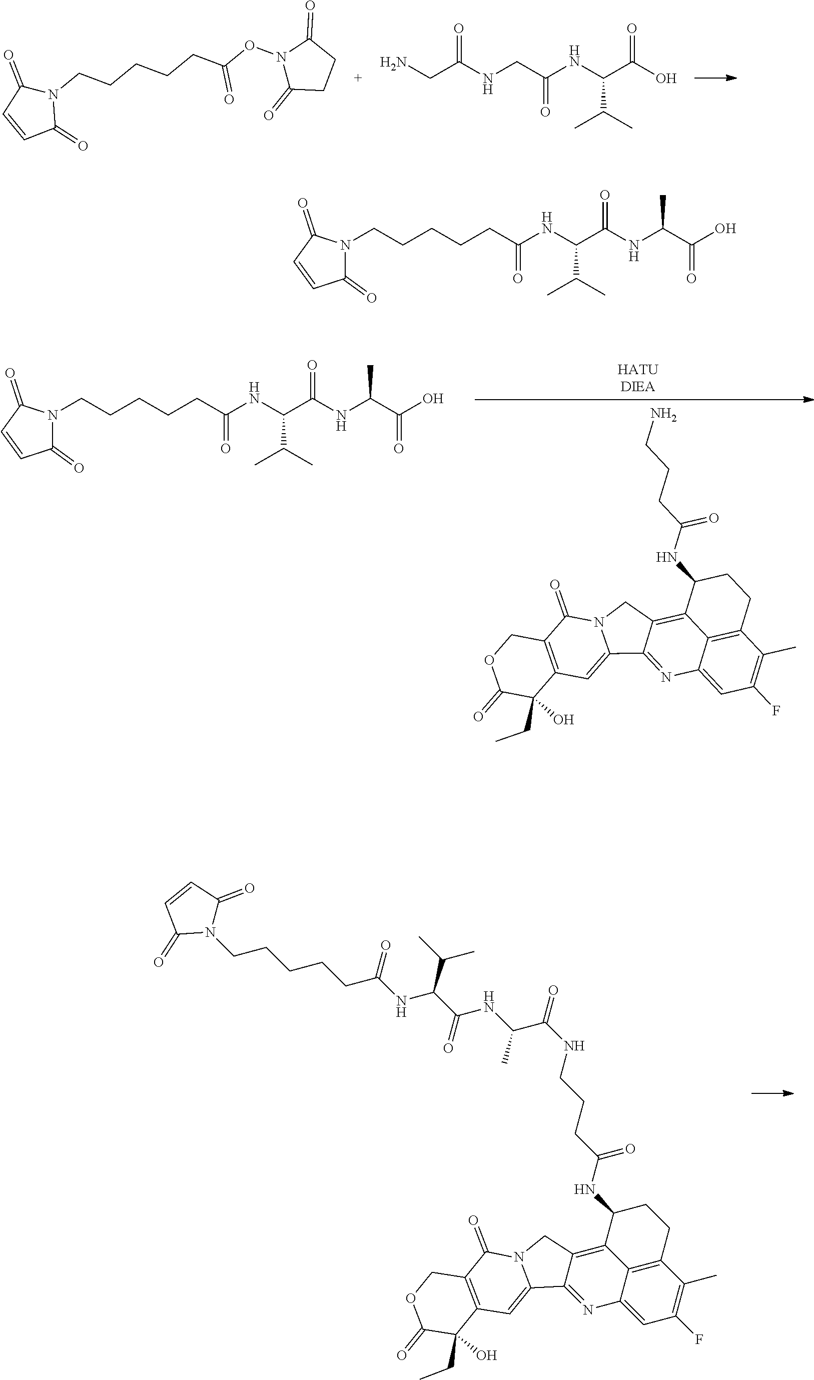

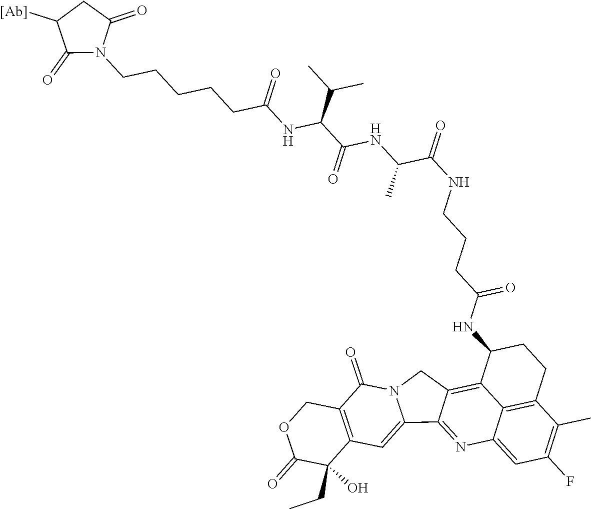

Exatecan was coupled with Boc-gamma-aminobutyric acid using EDC/HOBt. The Boc product was then deprotected using trifluoroacetic acid. Maleimidocaproic acid active ester was reacted with Gly-Gly-Val-OH to provide MC-GGV-OH. MCGGV-OH and gamma-aminobutyric acid amide of exatecan were then coupled using HATU to provide MC-VA-GAB-exatecan. This material was used to alkylate the cysteine-disulfide-reduced antibody to give C2-MV-VA-GAB-exatecan.

### Synthetic Scheme: Boc-GAB-Exatecan

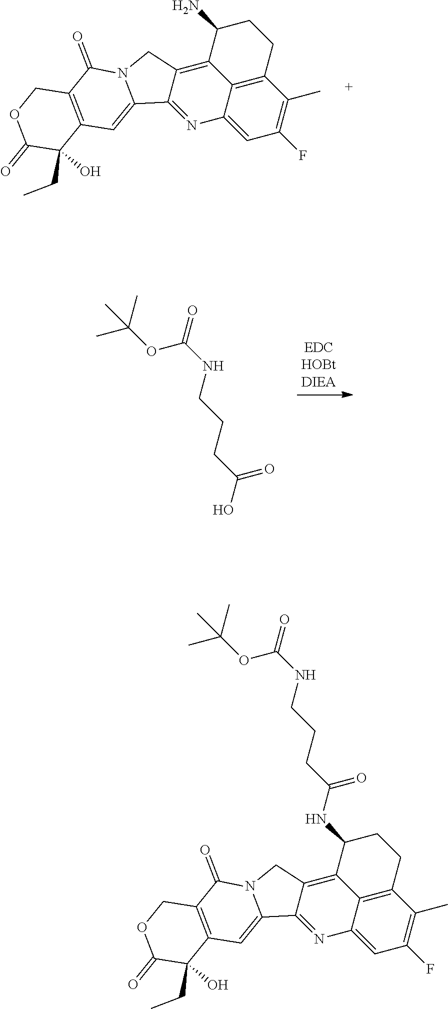

EDC Coupling of Boc-GAB with Exatecan Mesylate.

Exatecan mesylate (400.3 mg) was added to a 20-mL oven-dried vial, followed by 5.35 mL of anhydrous DMF under argon. 312 μL of DIEA was then added. 184.0 mg Boc-GABA was then added, followed by 14.3 mg HOBt and 223.8 mg EDC-HCl. The reaction was monitored by TLC and HPLC. After 2 h 33 min, poured into 400 mL EtOAc, washed with 1M Citric acid (3×40 mL), sat. NaHCO3 (3×40 mL), brine (40 mL). Dried over anh. Na2SO4. Evaporated to give 550 mg crude tan solid. Combiflashed on 40 g silica gel, loaded on 6 g celite; 0 to 60% (B: 20% MeOH/DCM)=0 to 12% MeOH/DCM. After analysis, fractions 2, 3, and 4 were combined and dried down in a tared 20-mL vial. After hivac overnight, the mass of yellow solid weighed 368.6 mg.

### Synthetic Scheme: GAB-Exatecan

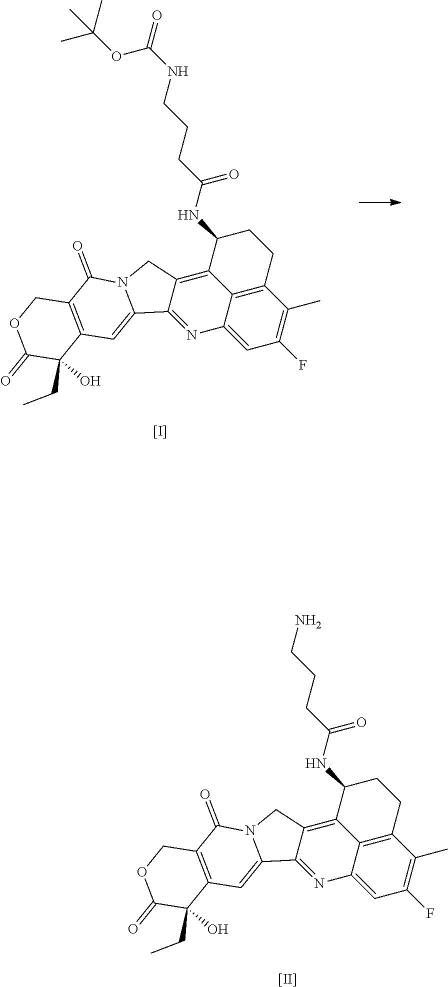

### TFA Cleavage of Boc-GAB-Exatecan

In a 30-mL scintillation vial 358.9 mg of Boc-GAB-exatecan was added, followed by 8.4 mL DCM along with stir bar. After all starting material had dissolved, 8.4 mL TFA was added and needle pierced through cap to let isobutylene out. HPLC shows reaction is completely done at 30 min. TLC still shows trace starting material in long wavelength. The reaction was stirred at room temp for 1 h, then transferred to a 500 mL flask with DCM and evaporated, and then dry on high vacuum to give 740 mg (202% yield) of a bright yellow oil. This material was dissolved in 5% MeOH/DCM (100 mL) with sonication, and celite (11.1 g) was added. The sample was evaporated then chromatographed on a combiflash (12 g silica gel), elution with 5% DCM to 20% MeOH/DCM. Fractions containing product were pooled and evaporated to yield a yellow solid weighing 348.7 mg, 1H NMR (400 MHz): δ 8.54 (1H, d, J=8.70 Hz), 7.77 (4H, dd, J=3.66, 1.53 Hz), 7.29 (1H, s), 6.53 (1H, s), 5.56 (1H, dtd, J=8.62, 5.04, 1.14 Hz), 5.41 (2H, s), 5.16 (2H, dq, J=3.43, 1.88 Hz), 3.16 (2H, dddd, J=7.10, 5.87, 4.73, 2.29 Hz), 2.83 (2H, dd, J=8.85, 6.56 Hz), 2.38 (3H, br s), 2.27 (2H, td, J=7.06, 1.53 Hz), 2.13 (2H, tdd, J=6.56, 4.88, 1.37 Hz), 1.85 (4H, m), 0.86 (3H, t, J=7.29 Hz).

### Synthetic Scheme: MC-VA-OH

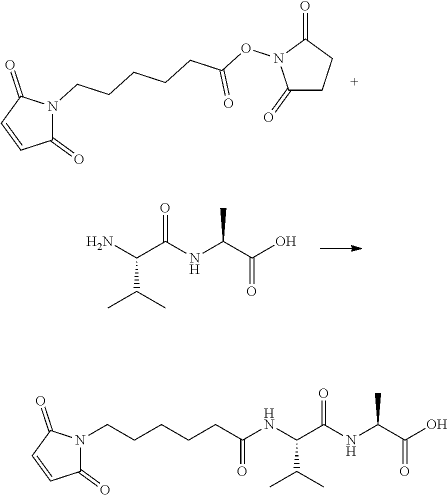

### Formation of MC-VA-OH

In a scintillation vial was placed MC-Osu (175.8 mg), Val-Ala-OH (103.7 mg), and DMF (10 mL). Stirred at room temp over the weekend. The cloudy reaction was crystal clear by Monday morning. The reaction was transferred to round bottom flask and rotoavapped with a 50C water bath and then placed under high vacuum for 10 min. Diethyl ether (Et2O, 60 mL) was added and the flask was placed in a sonication bath for 3-4 min at which point the product crystallized. The ether was decanted, fresh ether (60 mL) was added, sonicated for 3-4 min, and the process was repeated for a total of 3×60 mL of Et2O. The product was collected on fritted glass. HPLC analysis showed clean product: 114.5 mg, 1H NMR (400 MHz): δ 12.38 (1H, s), 8.18 (1H, d, J=6.94 Hz), 7.74 (1H, d, J=9.09 Hz), 6.99 (1H, s), 4.16 (2H, m), 3.35 (2H, t, J=7.00 Hz), 2.11 (2H, di, J=40.00, 8.00 Hz), 1.92 (1H, h, J=7.00 Hz), 1.46 (4H, m), 1.25 (3H, d, J=7.31 Hz), 1.16 (2H, dq, J=8.00, 8.00 Hz), 0.85 (3H, d, J=6.87 Hz), 0.80 (3H, d, J=6.56 Hz).

### Synthetic Scheme: MC-VA-GAB-Exatecan

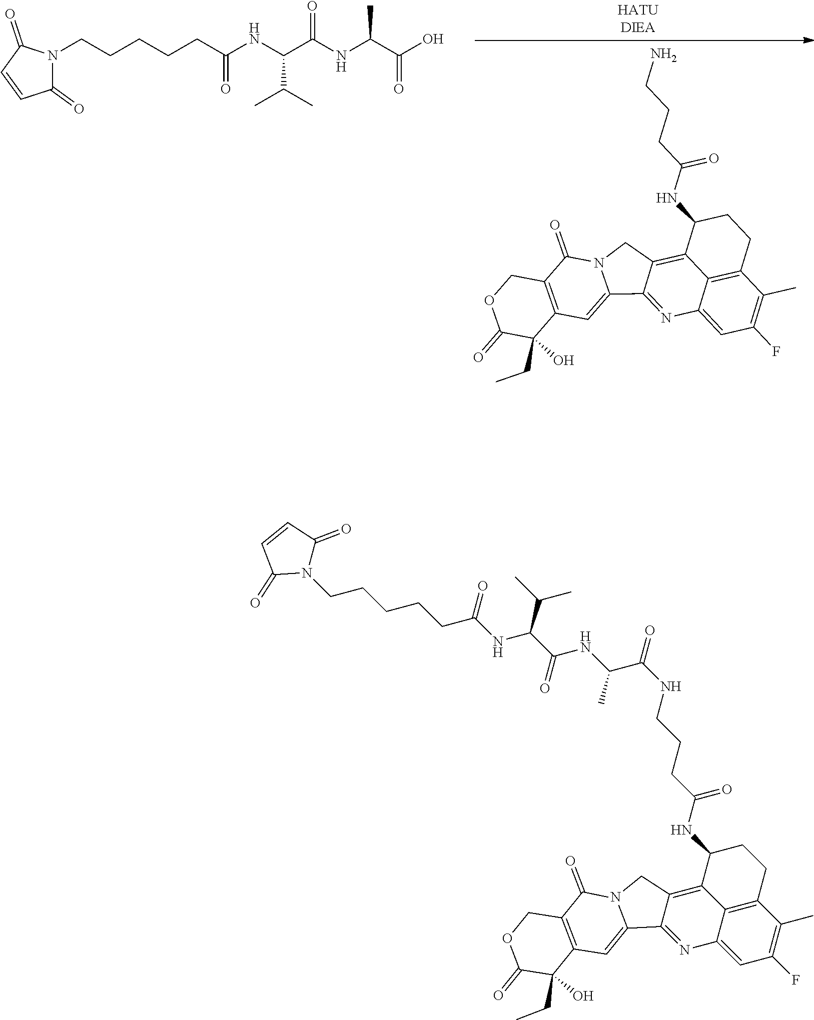 [SMILES: CC(C)[C@H](NC(=O)CCCCCN1C(=O)C=CC1=O)C(=O)N[C@@H](C)C(=O)O ; CC[C@@]1(O)C(=O)OCc2c1cc1n(c2=O)Cc2c-1nc1cc(F)c(C)c3c1c2[C@@H](NC(=O)CCCN)CC3 ; CC[C@@]1(O)C(=O)OCc2c1cc1n(c2=O)Cc2c-1nc1cc(F)c(C)c3c1c2[C@@H](NC(=O)CCCNC(=O)[C@H](C)NC(=O)[C@@H](NC(=O)CCCCCN1C(=O)C=CC1=O)C(C)C)CC3]

### HATU Coupling MC-VA-OH and GAB-Exatecan

MC-VA (50.7 mg) was added to a 4-mL vial containing GAB-exatecan (58.5 mg) along with a stir-bar. Anhydrous DMF (2.3 mL) was added to the vial under argon, and the vial was placed into an ice-bath. While in the ice-bath, DIEA (43.9 μL) and HATU (59.6 mg) were added. The reaction was then removed from the ice-bath and allowed to stir at room temp. The reaction was monitored by HPLC. After 18 min, the reaction was added dropwise to stirring MTBE (100 mL). The white suspension was stirred for 5 min at room temp, then allowed to settle. The solid was collected on 15-mL med. porosity fritted glass (88.7 mg after high vac 15 min). This material was dissolved in DCM (100 mL) and MeOH (5 mL) in a 200-mL RBF. Celite (1.5 g) was added and the mixture was rotovapped and then high-vacuumed. The solid cartridge was combiflashed on 4 g silica, elution with 1%-->20% MeOH in DCM. Fractions containing product were combined and rotovapped to give 49.9 mg. This product was stored at −20C freezer until needed. 1H NMR (DMSO with 0.03% v/v TMS, 400 MHz): δ 8.40 (1H, d, J=8.70 Hz), 7.86 (1H, d, J=7.17 Hz), 7.78 (3H, m), 7.29 (1H, s), 6.98 (2H, s), 6.51 (1H, s), 5.54 (1H, dtt, J=8.70, 4.92, 1.41 Hz), 5.41 (2H, qt, J=3.26, 1.72 Hz), 5.16 (2H, d, J=5.19 Hz), 4.15 (1H, t, J=7.10 Hz), 4.05 (1H, dd, J=8.55, 6.71 Hz), 3.34 (2H, s), 3.16 (3H, tdh, J=7.48, 5.19, 1.75 Hz), 3.05 (2H, br s), 2.38 (4H, br s), 2.15 (7H, m), 1.86 (4H, m), 1.68 (2H, tdd, J=7.78, 7.17, 6.56 Hz), 1.44 (5H, dddq, J=8.70, 6.94, 5.72, 2.26 Hz), 1.15 (6H, d, J=7.02 Hz), 0.85 (4H, t, J=7.32 Hz), 0.76 (7H, dd, J=10.45, 6.71 Hz).

### Synthetic Scheme: C2-MC-VA-GAB-Exatecan

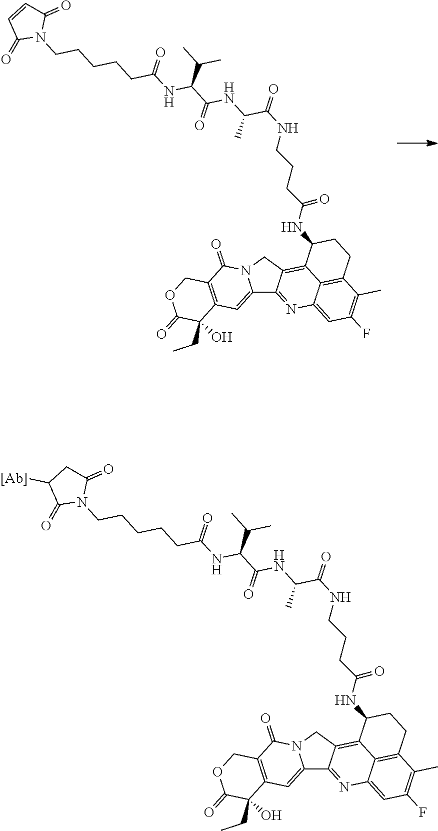

ADC alkylation via Michael-Addition with MC-VA-GAB-Exatecan

To a solution of C2 antibody (6.124 mg/mL, 1.83 mg, 12.55 nmols, 300 μL) in pH 8.0 borate buffer, dithiothreitol (DTT) (6.48 mM, 79.63 nmols, 12.29 nmols) was added. The solution was put on a hot water bath at 37° for 90 min with vortexing every 20 min. After 90 min, the solution was placed on ice and an aliquot was taken to determine the free thiol content of the antibody using an Ellman's test. This resulted in 6.82 free thiols per antibody. A Desalting column was used to buffer exchange the solution to a pH 7.4 EPPS buffer, with a chaser of buffer (40 μL) being used to fully elute product. A 50 kDa MWCO was used to purify the solution with a pH 7.4 EPPS buffer. Deruxtecan (9.67 mM, 125.5 nmols, 12.98 μL, 10 equiv.) was dissolved in DMSO and further diluted with DMSO to reach an organic phase volume that is 10% of the total. This was added to the antibody solution and put on a vertical rotator for 60 min at room temperature. Following the reaction N-acetylcysteine (NAC) (10 mM, 245 nmols, 2.45 μL) was dissolved in water and added to the reaction. The solution was vortexed and let stand for 15 min at room temperature. A Sephadex G-25 column was then used to purify the solution with an elution of pH 7.4 PBS (1.25 mL). Stabilization buffer (312.5 μL) was added to the solution and concentration measured using nanodrop (1.102 mg/mL, 1.722 mg, 11.8 nmols, Yield: 57%). The drug-antibody ratio was determined using cuvette and HIC/HPLC (Cuvette DAR: 7.0, HIC DAR: 7.3, AVG DAR: 7.2).

### Q. Synthesis of C2-MC-GGVA-GAB-Exatecan

### Synthetic Scheme: C2-MC-GGVA-GAB-Exatecan

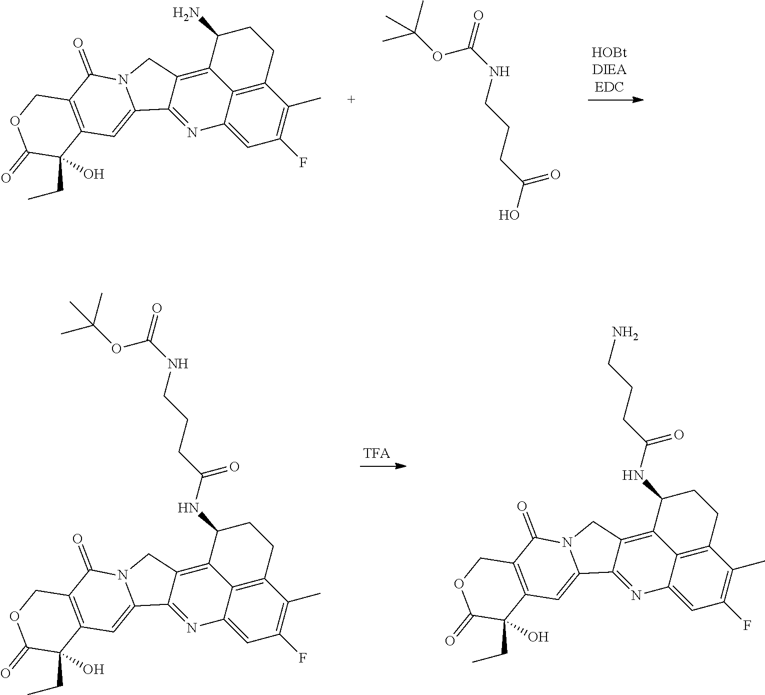 [SMILES: CC[C@@]1(O)C(=O)OCc2c1cc1n(c2=O)Cc2c-1nc1cc(F)c(C)c3c1c2[C@@H](N)CC3 ; CC(C)(C)OC(=O)NCCCC(=O)O ; CC[C@@]1(O)C(=O)OCc2c1cc1n(c2=O)Cc2c-1nc1cc(F)c(C)c3c1c2[C@@H](NC(=O)CCCNC(=O)OC(C)(C)C)CC3 ; CC[C@@]1(O)C(=O)OCc2c1cc1n(c2=O)Cc2c-1nc1cc(F)c(C)c3c1c2[C@@H](NC(=O)CCCN)CC3]

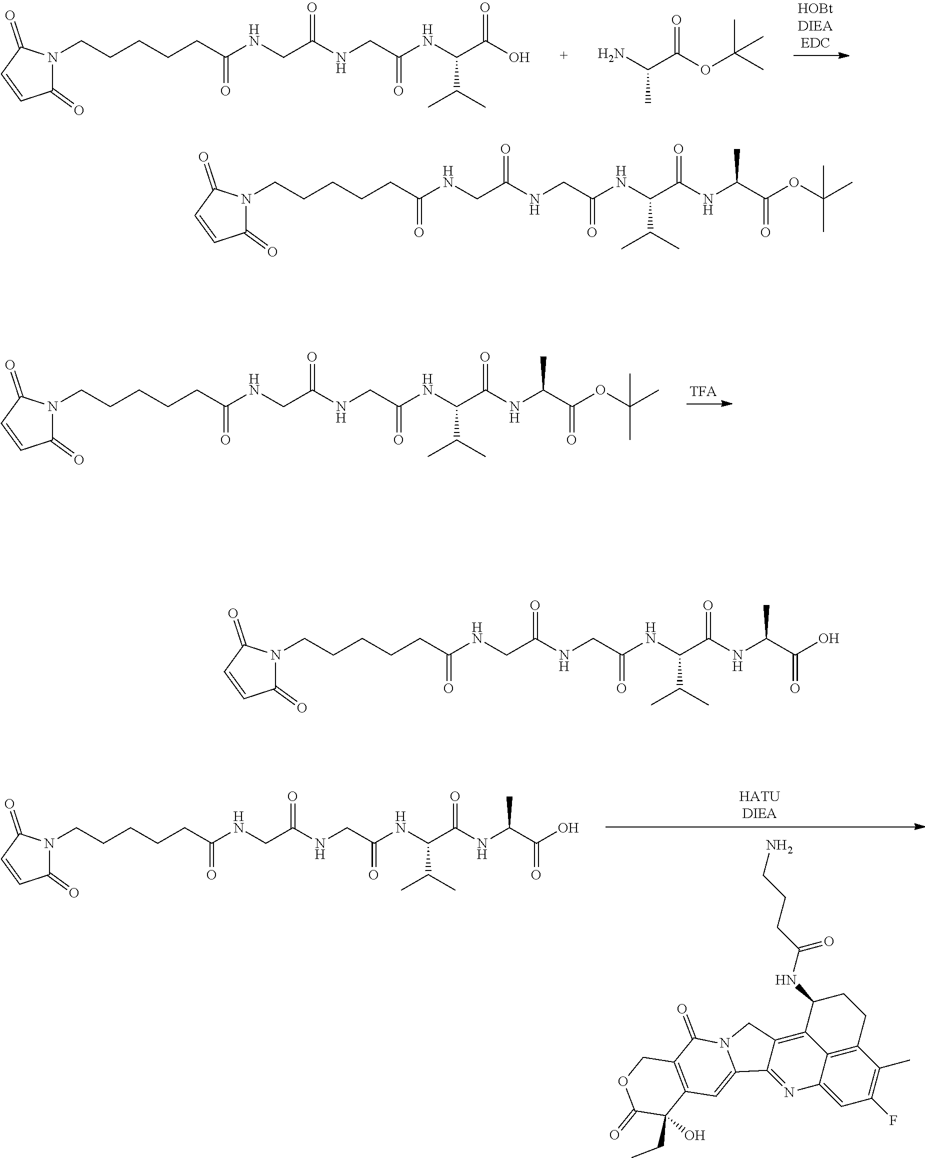

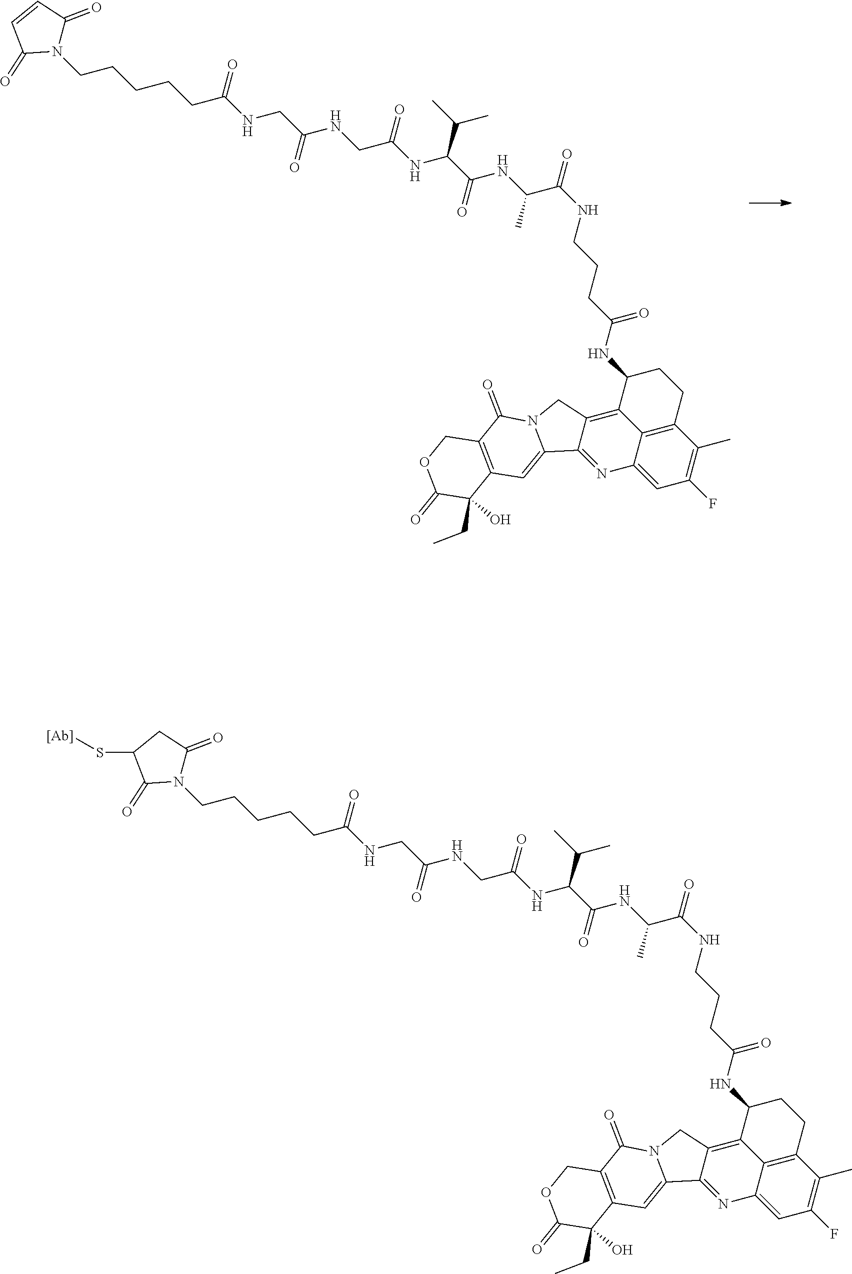

Exatecan was coupled with gamma-aminobutyric acid and the Fmoc was subsequently removed using diethylamine. In parallel, MC-GGV-OH was coupled with Ala-t-butyl ester and subsequently deprotected using trifluoroacetic acid. MV-GGVA-OH and GAB-exatecan were then amide-coupled using HATU to provide MC-GGVA-GAB-exatecan. The maleimide was then reacted with a reduced antibody to yield the ADC, C2-MV-GGVA-GAB-exatecan.

### Synthetic Scheme: MC-GGVA-O-t-Bu

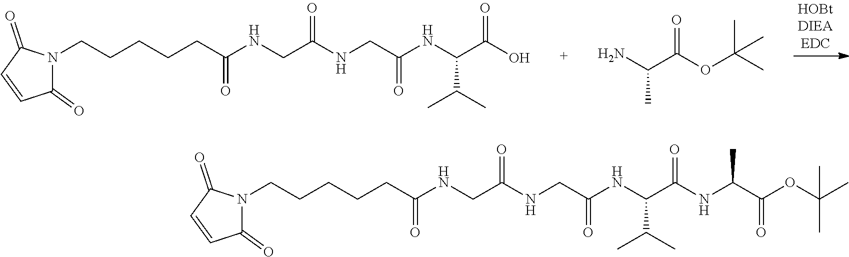

### Formation of MC-GGVA-O-t-Bu

Tert-butyl-alanine ester (41.0 mg, 45 μL) was added to a vial followed by anhydrous DMF (1.67 mL), under argon. DIEA (97.4 μL), MC-GGV-OH (100.3 mg), HOBt (4.5 mg), and EDC-HCL (60.0 mg) were the added to the vial in that order. After 3 h, the solution was transferred to a clean vial rotary evaporated to produce a thick oil. The residue was dissolved in EtOAc (200 mL), transferred to a separatory funnel, and washed with citric acid (1M, 3×10 mL), saturated NaHCO3 (3×10 mL), and brine (10 mL). The solution was dried over anhydrous Na2SO4, transferred to a clean vial and rotary evaporated to get a white solid (94.0 mg). 1H NMR (DMSO with 0.03% v/v TMS, 400 MHz): δ 8.29 (1H, d, J=6.71 Hz), 8.05 (1H, t, J=6.40 Hz), 8.00 (1H, t, J=5.68 Hz), 7.77 (1H, d, J=8.85 Hz), 6.99 (1H, s), 6.99 (1H, s), 4.20 (1H, dd, J=9.00, 6.64 Hz), 4.08 (1H, dd, J=7.32, 6.71 Hz), 3.74 (1H, d, J=5.57 Hz), 3.74 (1H, d, J=5.65 Hz), 3.66 (1H, d, J=5.74 Hz), 3.66 (1H, d, J=5.65 Hz), 3.36 (1H, dd, J=7.40, 6.71 Hz), 3.36 (1H, s), 3.31 (4H, s), 3.27-3.31 (1H, m), 2.49 (3H, quint, J=1.70 Hz), 2.09 (1H, dd, J=7.93, 7.02 Hz), 2.09 (1H, s), 1.93 (1H, dt, J=6.56, 2.48 Hz), 1.46 (3H, ddd, J=8.85, 8.24, 5.87 Hz), 1.46 (2H, d, J=7.40 Hz), 1.37 (6H, s), 1.36-1.37 (5H, 1.36 (s), 1.37 (s)), 1.23 (5H, 0.82 (2H, d, J=6.79 Hz), 1.22 (2H, d, J=7.28 Hz), m), 0.86 (2H, d, J=6.77 Hz), 0.86 (2H, d, J=6.87 Hz), 0.82 (2H, d, J=6.79 Hz).

### Synthetic Scheme: MC-GGVA-OH

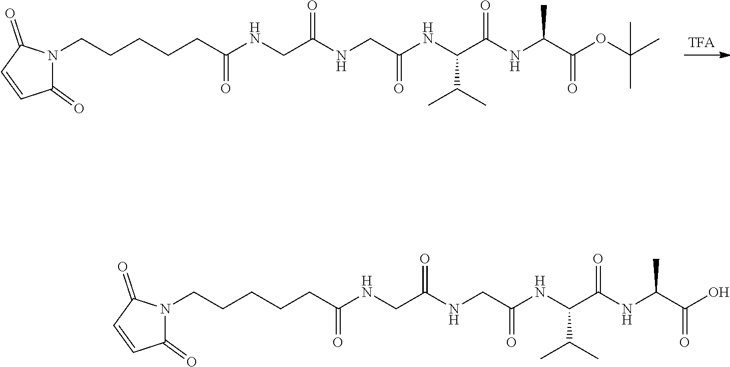 [SMILES: CC(C)[C@H](NC(=O)CNC(=O)CNC(=O)CCCCCN1C(=O)C=CC1=O)C(=O)N[C@@H](C)C(=O)OC(C)(C)C ; CC(C)[C@H](NC(=O)CNC(=O)CNC(=O)CCCCCN1C(=O)C=CC1=O)C(=O)N[C@@H](C)C(=O)O]

### Formation of MC-GGVA-OH

MC-GGVA-O-t-Bu was dissolved in dichloromethane (DCM) (2.46 mL) followed by trifluoroacetic acid (2.46 mL) and the pressure was relieved with a needle. After 2 h and 45 min, the reaction was transferred to a clean vial and rotary evaporated briefly. DCM (1 mL) was added to the vial followed by heptane (5 mL) and rotary evaporated again. This was repeated four times. The remaining solid yellow/white solid was then placed under high vacuum to dry the product (102 mg).

### Synthetic Scheme: MC-GGVA-GAB-Exatecan

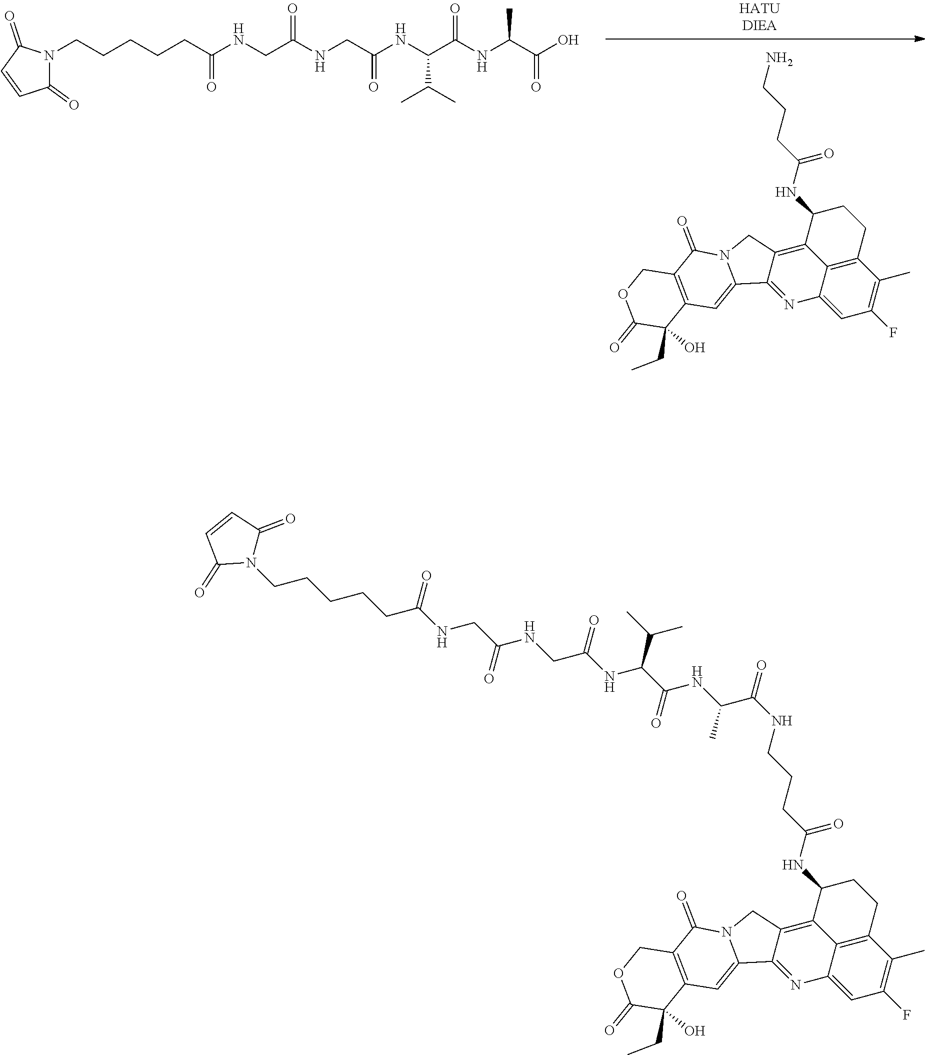 [SMILES: CC(C)[C@H](NC(=O)CNC(=O)CNC(=O)CCCCCN1C(=O)C=CC1=O)C(=O)N[C@@H](C)C(=O)O ; CC[C@@]1(O)C(=O)OCc2c1cc1n(c2=O)Cc2c-1nc1cc(F)c(C)c3c1c2[C@@H](NC(=O)CCCN)CC3 ; CC[C@@]1(O)C(=O)OCc2c1cc1n(c2=O)Cc2c-1nc1cc(F)c(C)c3c1c2[C@@H](NC(=O)CCCNC(=O)[C@H](C)NC(=O)[C@@H](NC(=O)CNC(=O)CNC(=O)CCCCCN1C(=O)C=CC1=O)C(C)C)CC3]

### Formation of MC-GGVA-GAB-Exatecan

GAB-exatecan (49.2 mg) was added to a vial followed by MC-GGVA (52.1 mg) and anhydrous dimethylformamide (DMF) (1.82 mL). The vial was then placed into an ice bath and while on ice, DIEA (34.8 μL) and HATU (49.4 mg) were added. The reaction was then removed from the ice bath and let stir at room temperature for 18 min. The reaction was then added dropwise to MTBE (100 mL), stirred for 5 min, and then allowed to settle. The solid (96.0 mg) was collected on medium porosity fritted glass. This was then dissolved in DCM (100 mL) and MeOH (5 mL). Celite (1.5 g) was added and then the solution was rotary evaporated. The solid cartridge was purified on a combiflash with a silica gel using a 1-20% MeOH in DCM gradient to elute the product. The fractions containing the pure product were rotary evaporated and then frozen at −20 °C. 1H NMR (DMSO with 0.03% v/v TMS, 400 MHz): δ 8.41 (1H, d, J=8.68 Hz), 8.04 (2H, dt, J=8.68, 5.62 Hz), 7.93 (1H, d, J=7.34 Hz), 7.79 (2H, t, J=4.00 Hz), 7.76 (1H, m), 7.72 (1H, t, J=6.00 Hz), 7.29 (1H, s), 6.98 (2H, s), 6.50 (1H, s), 5.55 (1H, m), 5.41 (2H, br s), 5.18 (2H, q, J=16.00 Hz), 4.15 (2H, m), 3.73 (2H, dd, J=5.62, 2.69 Hz), 3.66 (2H, d, J=5.75 Hz), 3.35 (4H, t, J=6.00 Hz), 3.16 (2H, m), 3.05 (2H, m), 2.38 (4H, br s), 2.11 (7H, m), 1.85 (4H, m), 1.68 (2H, dq, J=6.60, 6.60 Hz), 1.45 (5H, dddtd, J=7.70, 5.50, 4.40, 2.75, 1.96 Hz), 1.17 (6H, m), 0.86 (4H, ddd, J=7.95, 7.34, 1.59 Hz), 0.77 (7H, dd, J=10.64, 6.85 Hz).

### Synthetic Scheme: C2-MC-GGVA-GAB-Exatecan

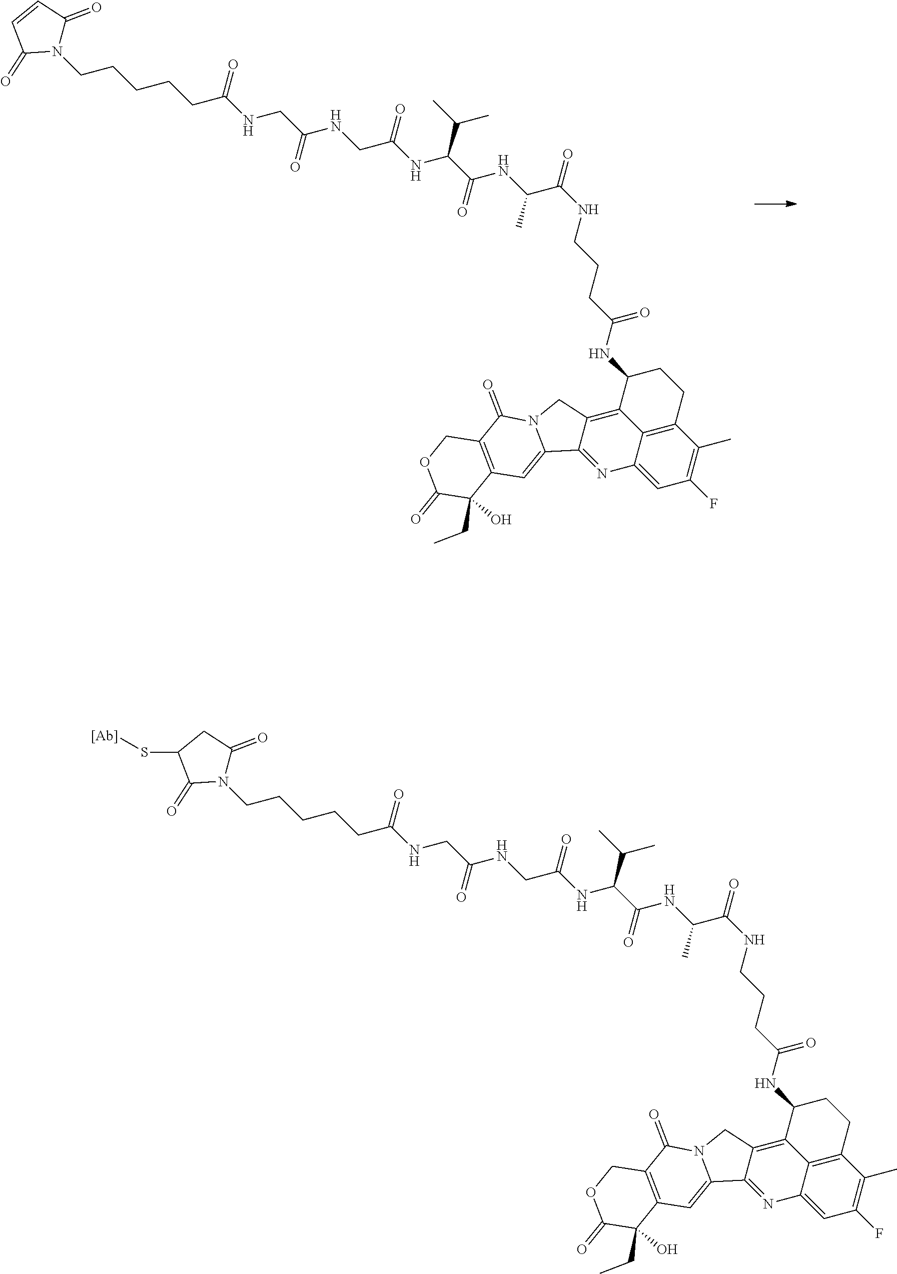

Reduction of C2-Antibody and Alkylation with MC-GGVA-GAB-Exatecan

C2 antibody (6.287 mg/mL, 1.89 mg, 12.57 nmols, 300 μL) in pH 8 borate buffer was reduced using dithiothreitol (6.48 mM, 81.71 nmols, 12.61 μL, 6.5 equiv.) in the same buffer. The solution was put on a hot water bath at 37 °C for 90 min, with vortexing every 20 min. After 90 min, the solution was placed on ice and an aliquot was taken to analyze free thiol content using an Ellman's test. The result of this was a free thiol content of 6.94 free thiols per antibody. A Desalting column was used to buffer exchange the solution to a pH 7.4 EPPS buffer with a chase of buffer (40 μL) to fully elute the product. The solution was further purified with a 50 kDa MWCO filter and pH 7.4 EPPS buffer. Deruxtecan (9.67 mM, 125.7 nmols, 13 μL, 10 equiv.) was dissolved in DMSO and further diluted with DMSO to get a total organic phase that is 10% of the total volume. This was added to the antibody and the solution was placed on a vertical rotator for 60 min at room temperature. N-acetylcysteine (100 mM, 251.4 nmols, 2.51 uL, 20 equiv.) was dissolved in water, added to the reaction and the solution was let stand for 15 min at room temperature. A Sephadex G-25 column was used to purify the solution using pH 7.4 PBS (1.25 mL) to elute from the column. Stabilization buffer (312.5 μL) was added to the solution and concentration determined by nanodrop (1.034 mg/mL, 1.62 mg, 10.8 nmols, Yield: 54%). The drug-to-antibody ratio was determined by cuvette and HIC/HPLC (Cuvette DAR: 6.3, HIC/HPLC DAR: 6.7. AVG DAR: 6.5)

### Example 6. In vitro cytotoxicity of anti-MUC1* ADCs

### Impedance Assay for Measuring Target Cell Killing by ADCs

An xCELLigence RTCA MP Instrument (Agilent) was used to assess real-time killing of target cancer cells). In the xCELLigence system, target cancer cells, which are adherent, are plated onto electrode array 96-well plates. Adherent cells insulate the electrode and increase the impedance. The number of adherent cancer cells is directly proportional to impedance. Antibodies and antibody-drug-conjugates are much smaller and do not significantly contribute to impedance. Therefore, increasing impedance reflects the growth of the cancer cells and decreasing impedance reflects the killing of the cancer cells.

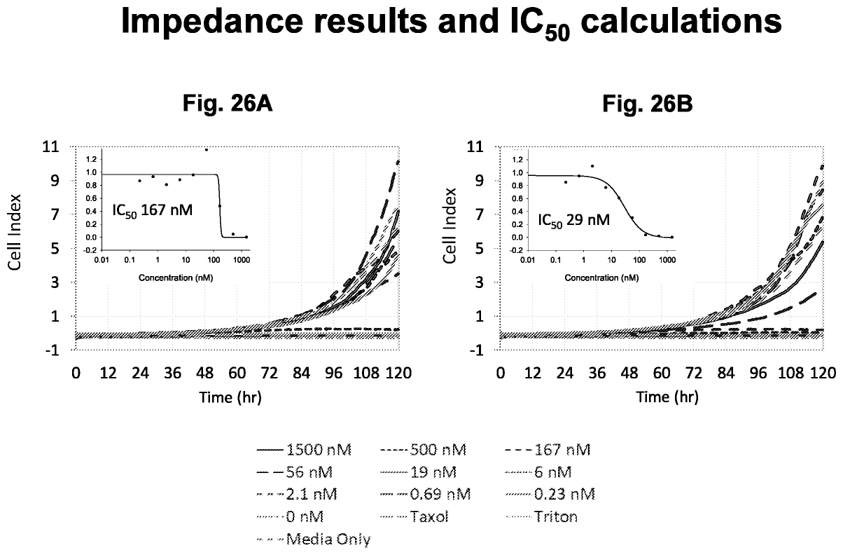

Target Cancer cells (Table 9) were seeded onto a multi-electrode well plate at a density of 5000 cells/well (100 μL of a 50,000 cells/mL stock) and incubated for 24 hr in a 37 °C/5% CO2 incubator. ADCs were prepared as a 2X solution in corresponding growth media and 100 μL of each concentration was added to target cells. As a positive control (100% cell killing) cells were treated with either 1% triton or 1 μM Taxol instead of ADC. After 40-120 hours incubation in a 37 °C/5% CO₂ incubator cell killing was assessed. In this assay, impedance is measured in real-time. When the adherent cancer cells are killed and come off the electrode surface, impedance (insulation) decreases. Exemplary impedance results and IC₅₀ calculations are presented in FIGS. 26A-26B.

ADCs I-IX had cytotoxic activity on MUC1* positive cell lines as shown in Table 10.

TABLE 9
Cancer Cells for testing anti-MUC* ADCs

| Cell line | Source |
|---|---|
| T47D | Breast Cancer, human ductal carcinoma |
| HPAFII | Pancreatic Cancer, human pancreatic adenocarcinoma |
| CFPAC-1 | Pancreatic Cancer, human pancreatic ductal adenocarcinoma |
| H1975 | Lung Cancer, human lung non-small cell carcinoma |
| H2110 | Lung Cancer, human lung non-small cell carcinoma |
| DU-145 | Prostate Cancer, human prostate carcinoma |

TABLE 10
ADC cytotoxic activity on MUC1* positive cancer cell
lines as determined by xCELLigence (IC50, nM)

| Com- | | T47D- | T47D- | HPAF- | HPAF-II | NCI- | DU- |
|---|---|---|---|---|---|---|---|
| pound | T47D | MUC1* | MUC1* 15% | II | MUC1* | H1975 | 145 |
| I | 156 | 1.8 | | | | 23 | |
| IIa | 124 | 18 | | 29 | 15 | 79 | |
| IIb | 21 | | | 2.8 | 4.0 | 11 | |
| Va | 121 | 1.4 | 28 | | | | |
| Vb | 89 | | 26 | 159 | 93 | | 203 |
| VIb | 61 | 17 | | 68 | 40 | 110 | |
| VIc | 42 | 19 | | 79 | 49 | 68 | |
| VII | 80 | 2.1 | | | | 181 | |
| VIII | 112 | 6.2 | 20 | 86 | 72 | 207 | 163 |
| IX | 30 | | | 13 | | 55 | 49 |

### PrestoBlue Live/Dead Cell Assay for Measuring Target Cell Killing by ADCs

Another method of measuring the killing activity of ADCs against target cancer cells is to use the PrestoBlue live/dead cell assay (ThermoFisher). Target cancer cells were seeded in a black-walled, clear-bottom 96-well tissue culture plate at a density of 5000 cells/well (100 μL of a 50,000 cells/mL stock) and incubated overnight in a 37 °C/5% CO₂ incubator. ADCs were prepared as a 2X solution in corresponding cancer cell growth media and 100 μL of each concentration was added to target cancer cells. As a positive control (100% killing) cancer cells were treated with 1 μM Taxol instead of ADC. After 72 hour-120 hour incubation in a 37 °C/5% CO₂ incubator, cell viability was measure by fluorescence using the PrestoBlue HS assay (ThermoFisher). Briefly, after the incubation period, 20 μL of PrestoBlue HS solution was added. Fluorescence (Ex 560 nm/Em 590 nm) was recorded after 30-90 min. incubation using a Tecan plate reader. Percentage viability was determined by PrestoBlue HS staining and normalized to 1 μM taxol treated cells.

The normalized fluorescence values were imported into the graph program, Sigma Plot, data plotted as ADC concentration versus normalized fluorescence signal and curve fitted using a four-parameter Hill equation: f=y0+a*x\hat{ }b/(c\hat{ }b+x\hat{ }b) to calculate IC50 values. Each ADC tested had cytotoxic activity on MUC1* positive cell lines as shown in Table 11. IC50 values for the ADCs were generally consistent between the xCELLigence and Presto Blue assays.

TABLE 11
ADC cytotoxic activity on MUC1* positive cancer cell
lines as determined by Presto Blue (IC50, nM)

| Com- | | T47D- | T47D- | | HPAFII- | | DU- |
|---|---|---|---|---|---|---|---|
| pound | T47D | MUC1* | MUC1* 15% | HPAFII | MUC1* | H1975 | 145 |
| I | 187 | 2.6 | | 112 | 63 | 89 | |
| IIa | 214 | 15 | | 95 | 34 | 70 | |
| IIb | 26 | | | 9.8 | | 2.4 | 17 |
| III | | 1.3 | | 100 | 90 | | |
| IV | | 4.6 | | 93 | 58 | | |
| Va | 175 | 3.0 | 68 | | | | |
| Vb | 212 | 3.4 | 54 | | 86 | | |
| VIa | 167 | 57 | | | | | |
| VIb | 77 | 11 | | 399 | 155 | 73 | |
| VIc | 67 | 13 | | 306 | 187 | 112 | |
| VII | 71 | 4.1 | | 161 | | 233 | |
| VIII | 153 | 8.0 | 25 | 180 | 62 | 19 | 160 |
| IX | 33 | | | 25 | | 26 | 38 |

### Example 7. ADC Activity Against Human Tumors in Mice

### ADC Activity Against T47D Breast Cancer Tumors

For breast cancer xenografts, female NOD/SCID mice (weight 18-22 g, 6-8 weeks old, Charles River Laboratories) previously implanted with a 90-day release estrogen pellet (Innovative Research Laboratories) were subcutaneously injected under isoflurane anesthesia (ISOTHESIA, 250 ml (HENRY SCHEIN, NDC: 11695-6776-2) with one million luciferase-positive T47D breast cancer cells expressing low levels of MUC1* (ATCC) or one million luciferase-positive T47D-MUC1* cells overproducing MUC1* using a BD 26G Insulin Syringes with Detachable Needle (Fisher cat: 329652) or BD 28G Lo-Dose U-100 Insulin Syringes (Fisher cat: 329461).

Tumor volume was measured from bioluminescence images acquired with a Xenogen IVIS-Spectrum system (Perkin Elmer). Mice were injected intraperitoneal into the scruff of the neck with 150 ul, 30 mg/mL, D-Luciferin (XenoLight™ D-Luciferin Potassium Salt; PerkinElmer P/N 122799), placed under isoflurane anesthesia, and photographed within 10 minutes of the Leciferin injection. Data was expressed graphically as Radiance (photons/sec) as a function of days post-tumor implantation.

Tumor mass was determined from tumors excised from mice receiving T47D cells on Day 95 and mice receiving T47D-MUC1* cells on Day 96.

Group selection was performed to evenly distribute xenografted mice to ensure mock-treated groups and ADC-treated groups had equivalent initial tumor sizes.

Humane criteria for euthanasia were followed when: the tumor burden reached or exceeded 20 mm in diameter (in line with the IACUC policy), tumors became ulcerated, tumor position interfered with normal ambulation, feeding/drink/or elimination; or the mice had a body condition score of 2 or weight loss >15% from pre-xenograft weight.

For the tubulin polymerization inhibitor ADCs, mice were injected intraperitoneal with C2-MMAE (Compound I) on Day 7 (5 mg/kg), Day 14 (5mk/kg) and Day 20 (10 mg/kg) post-xenograft; 20A10-MMAE (Compound II, batch A) on Day 7 (5 mg/kg), Day 14 (10mk/kg) and Day 20 (10 mg/kg) post-xenograft; or with phosphate-buffered saline (PBS) as a control.

For topoisomerase inhibitor ADCs, mice were injected intraperitoneal with 20 mg/kg C2-deruxtecan (Compound V, batch B), C2-exatecan (Compound VIII) or with phosphate-buffered saline (PBS) at Day 5-7 post-xenograft and then once per week thereafter for a total of seven ADC injections.

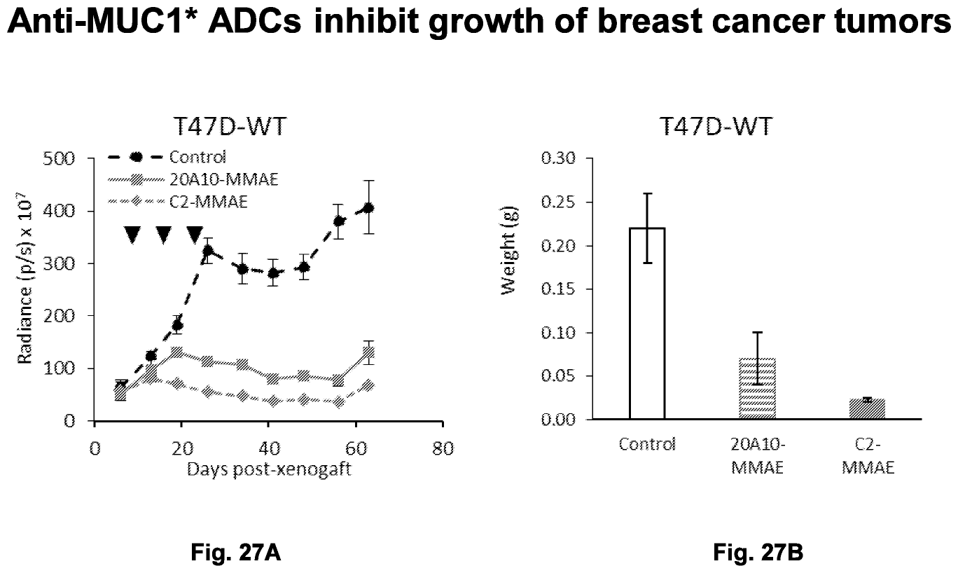

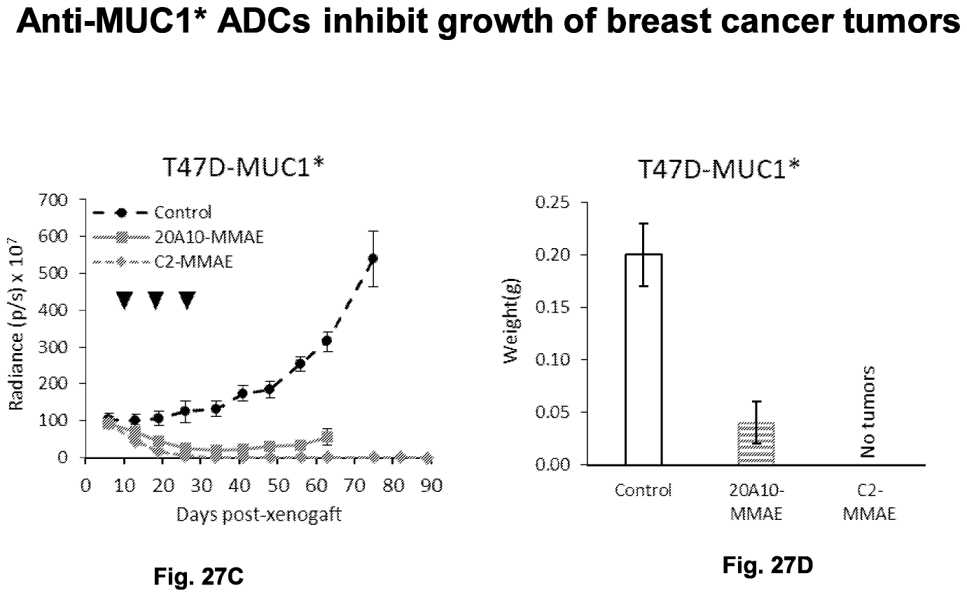

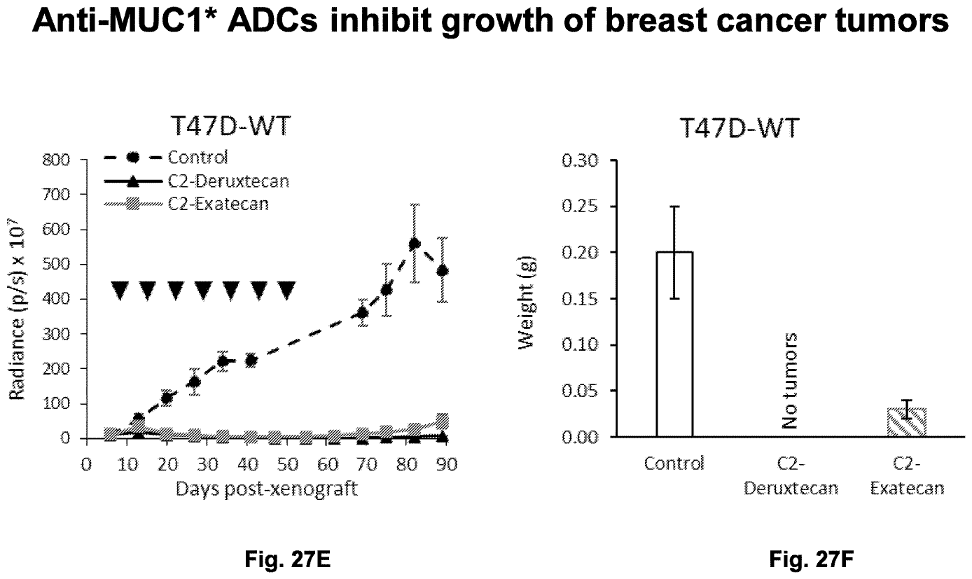

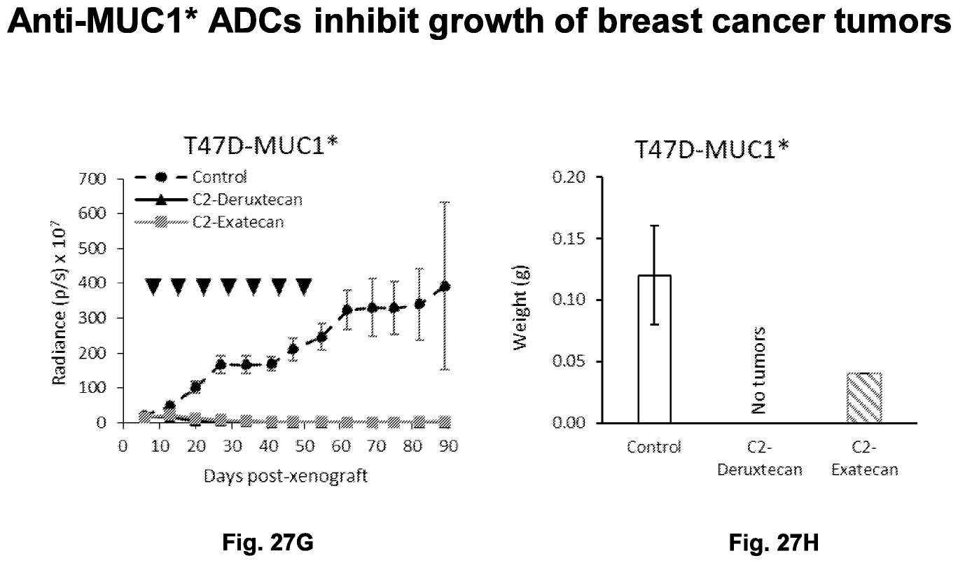

C2-MMAE, 20A10-MMAE, C2-deruxtecan, and C2-exatecan inhibit the growth of T47D xenograft tumors (FIG. 27A, FIG. 27B, FIG. 27E, FIG. 27F) and T47D-MUC1* xenograft tumors (FIG. 27C, FIG. 27D, FIG. 27G, and FIG. 27H) as determined by bioluminescent imaging and caliper measurements, indicating that anti-MUC1* ADCs may have efficacy in the treatment of MUC1* positive breast cancer.

### ADC Activity Against HPAF II Pancreatic Cancer Tumors

For pancreatic cancer xenografts, female NU/NU mice (weight 18-22 g, 6-8 weeks old, Charles River Laboratories) were subcutaneously injected under isoflurane anesthesia with one million HPAF II-MUC1* pancreatic cancer cells that were engineered to express luciferin and high levels MUC1*. Tumors were allowed to engraft for five (5) days before four (4) injections of C2-MMAE (Compound I) at a dose of 10 mg/kg on Day 5, Day 12, Day 19 and Day 26 or 7 weekly injections of C2-deruxtecan (Compound V, Batch B) or C2-exatecan (Compound VIII).

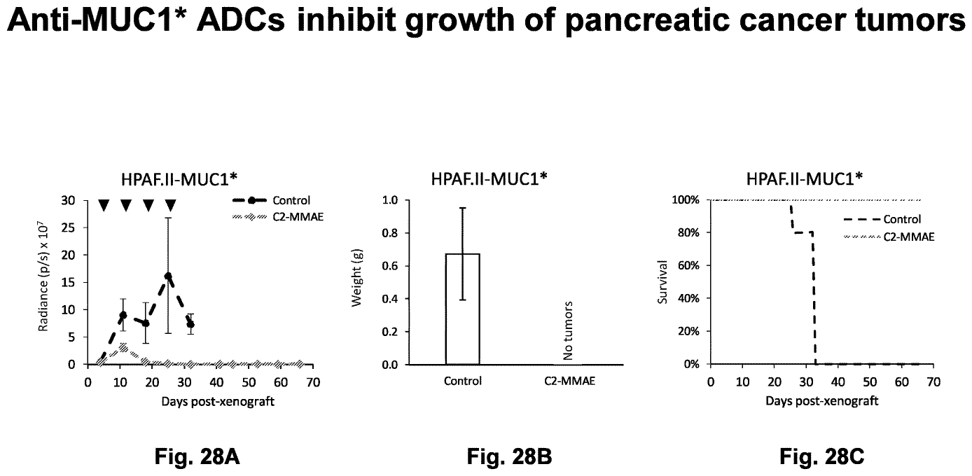

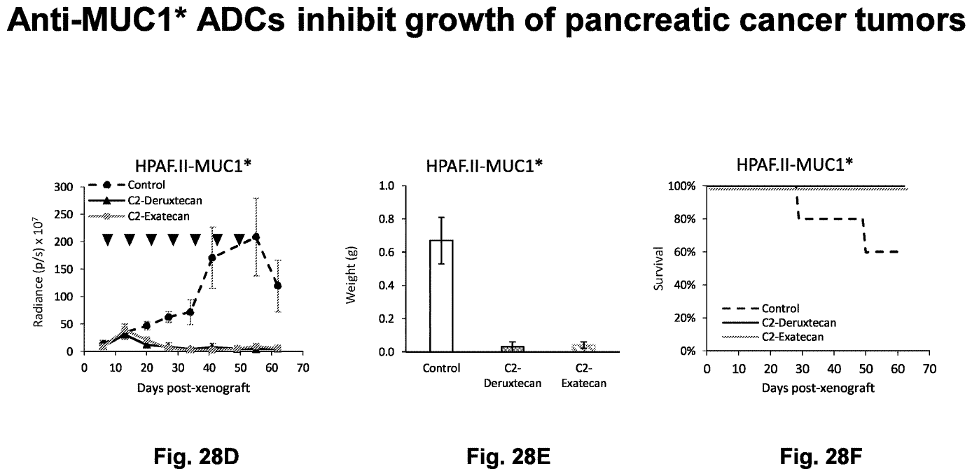

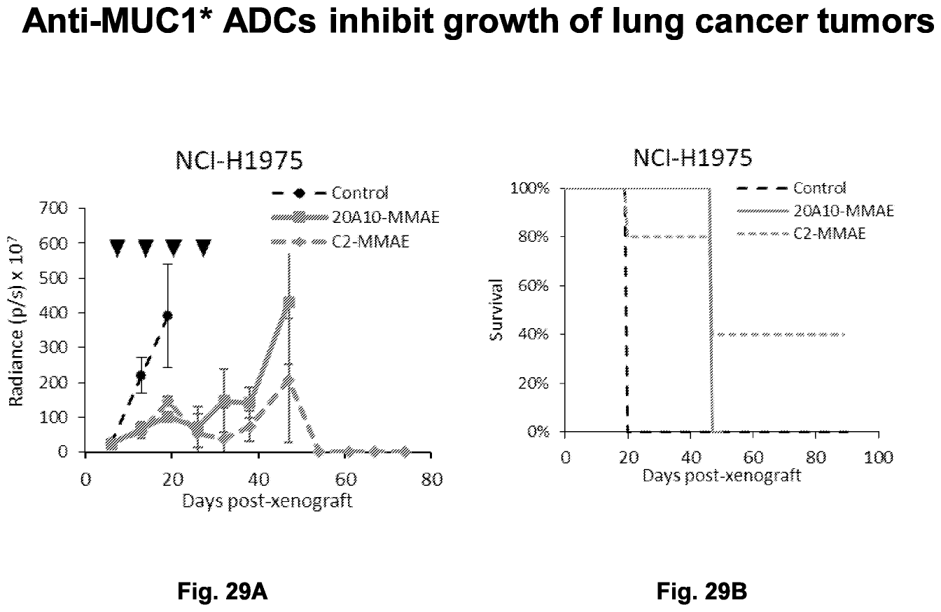

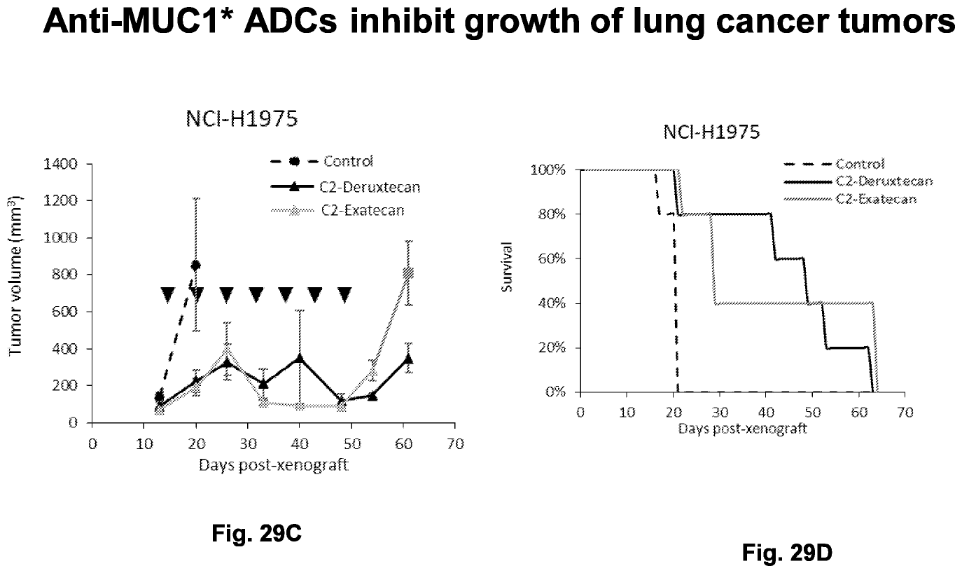

Anti-MUC1* ADCs with MMAE, deruxtecan, or exatecan toxins inhibited the growth of pancreatic cancer xenograft tumors as determined by bioluminescent imaging (FIG. 28A, FIG. 28D) and mass determination of tumors excised from HPAF II mice on Day 32 and from HPAF II-MUC1* mice on Day 62. (FIG. 28B, FIG. 28E). The lower average radiance value for the control mice on Day 30 is a consequence of sacrificing the control mouse with the largest tumor on day 25. The anti-MUC1* ADCs also increased survival in this pancreatic cancer model (FIG. 28C, FIG. 28F), indicating that anti-MUC1* ADCs may have efficacy in the treatment of MUC1* positive pancreatic cancer.

### ADC Activity Against H1975 Lung Cancer Tumors

For lung cancer xenografts, female NU/NU mice (weight 18-22 g, 6-8 weeks old, Charles River Laboratories) were subcutaneously injected under isoflurane anesthesia with one million luciferase-positive NCI-H1975 non-small cell lung cancer cells (ATCC). On Day 7 post tumor implantation, the animals were injected with 10 mg/kg C2-MMAE followed by three weekly C2-MMAE injections or with 20 mg/kg C2-deruxtecan or C2-exatecan followed by six weekly ADC injections. All untreated mice had to be sacrificed on Day 10 due to excess tumor burden.

Tumor growth in mice treated with the deruxtecan and exatecan ADCs was determined by measuring tumor size with a caliper. The two longest perpendicular axes in the x/y plane of each xenograft tumor were measured to the nearest 0.1 mm by two independent observers. The depth was assumed to be equivalent to the shortest of the perpendicular axes, defined as y. Measurements were made using a digital vernier caliper while mice were conscious and were calculated according to the following equation: Xenograft volume=xy²/2.

Anti-MUC1* ADCs with MMAE, deruxtecan, or exatecan toxins inhibited the growth of lung cancer xenograft tumors as determined by bioluminescent imaging (FIG. 29A) or direct caliper measurements (FIG. 29C). The anti-MUC1* ADCs also increased survival in this pancreatic cancer model (FIG. 29B, FIG. 29D), indicating that anti-MUC1* ADCs may have efficacy in the treatment of MUC1* positive lung cancer.

### Example 8. Cytotoxicity of Anti-MUC1* ADCs with Additional Linkers and Toxins

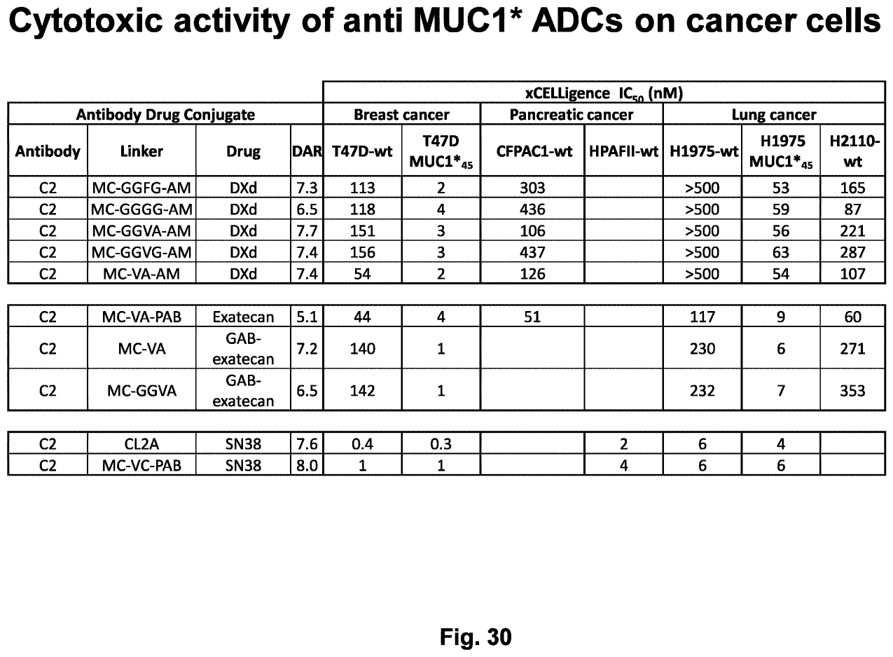

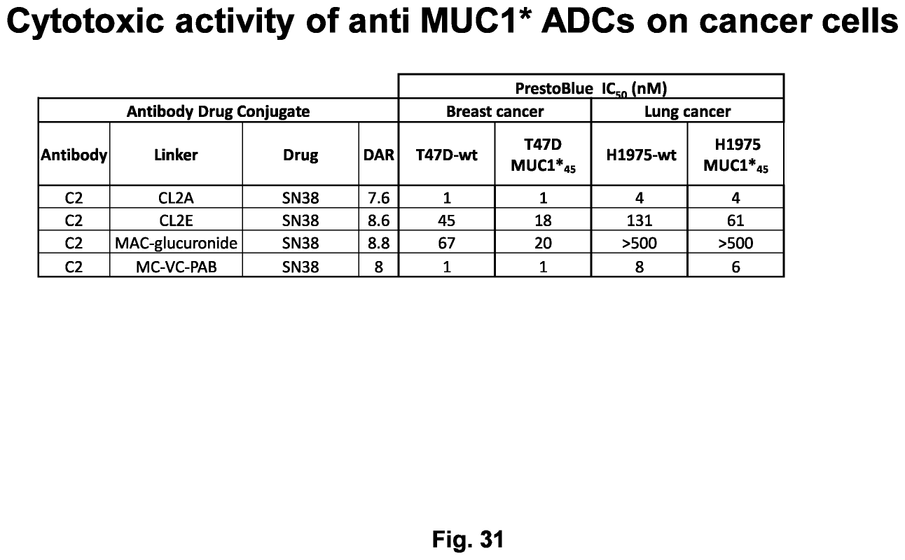

An anti-MUC1* antibody was conjugated to several toxins via a number of different linkers (compounds VIII-XXII). All of the resulting ADCs had cytotoxic activity against breast cancer, pancreatic cancer, and lung cancer cell lines (FIGS. 30-31).

Deruxtecan (MC-GGFG-AM-DXd) is cleaved by cathepsin enzymes located in the lysosome to release the DxD toxin. The cathepsins, particularly cathepsin B, cleave the peptide bond between the fourth glycine and the aminomethyl (AM) moiety. The resulting AM group on AM-DXd is unstable and self-immolates to ammonia and formaldyde, yielding the payload DXd. GGGG, GGVA, GGVG, and VA variants of deruxtecan were synthesized and conjugated to the C2 antibody to determine if the peptide linker sequence or length affects potency or cell line selectivity, as different cell lines express varying amounts and isoforms of cathepsin enzymes. The GGVA, GGVG, and VA linkers were expected to be more susceptible to cathepsin enzymes in some cells because those sequences are more common in natural cathepsin substrates. The results demonstrated that all four ADCs had cytotoxic activity against breast cancer, pancreatic cancer, and lung cancer cells. The IC₅₀ for C2-MC-VA-AM-DXd was three-fold lower in breast cancer and pancreatic cancer cell, while the three novel toxins with tetrapeptide linkers had similar activity to deruxtecan.

The C2-MC-VA-AM-DXd results demonstrated that a dipeptide linker could be substituted for the tetrapeptide linker of deruxtecan. To extend this observation to other dipeptides, toxins and self-immolating groups, we synthesized C2-MC-VA-PAB-exatecan and found that this ADC also had enhanced activity compared to deruxtecan on breast cancer, pancreatic cancer, and lung cancer cells.

C2-MC-VA-GAB-exatecan and C2-MC-GGVA-GAB-exatecan were synthesized to investigate the importance of the self-immolating group on the toxin. GAB exatecan does not self-immolate, but these ADCs still had cytotoxic activity against breast cancer, pancreatic cancer, and lung cancer cells, albeit with a higher IC₅₀ against cells that were not engineered to express MUC1*₄₅. This reduced activity was unexpected because and ADC with a GGFG GAB-exatecan linker toxin was reported to have picomolar activity. Nakada, Bioorg. Med. Chem. Lett. 2016.

The next group of MUC1* ADCs had an SN38 toxin. SN38, a topoisomerase inhibitor, is incorporated in sacituzumab govitecan, an FDA-approved Trop-2 ADC. The four SN38 ADCs had different linkers with different toxin release profiles: (1) the CL2A linker of sacituzumab govitecan and the MC-VC-PAB linker have hydrolysable carbonate groups; (2) the CL2E linker has a more stable carbamate group; and (3) the MAC-glucuronide linker is cleaved by glucuronidase enzymes expressed by some cancer cells, especially invasive cancer cells. All four SN38 ADCs had cytotoxic activity against breast cancer cells. The CL2A, CL2E and MC-VC-PAB ADCs also had cytotoxic activity against lung cancer cells. The ADCs with carbonate linkers were less dependent upon high MUC1* expression for their cytolytic activity.

Overall, these results demonstrate that MUC1* ADCs with seventeen diverse linker toxin combinations have cytotoxic activity on MUC1* positive cancer cells.

### Bystander Killing of Low Antigen Expressing Cancer Cells

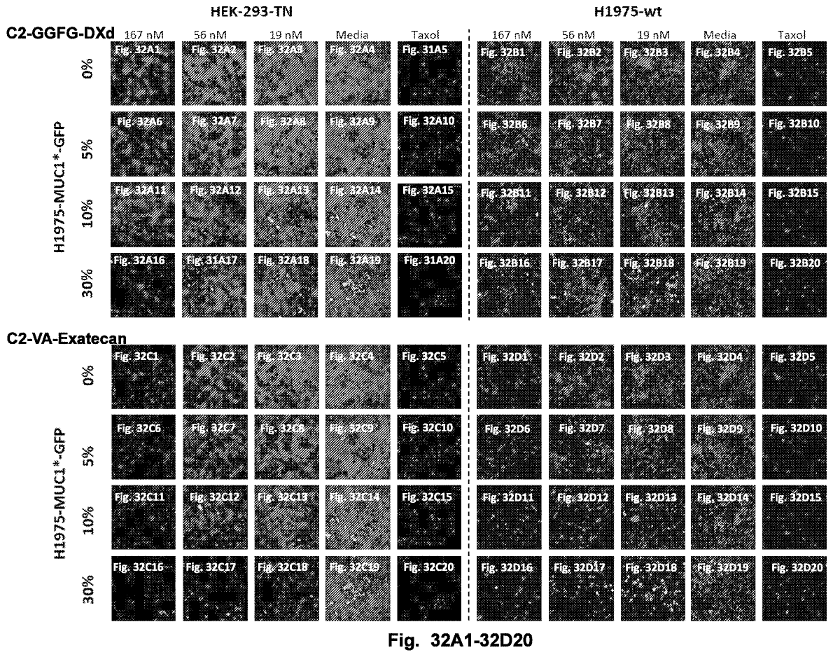

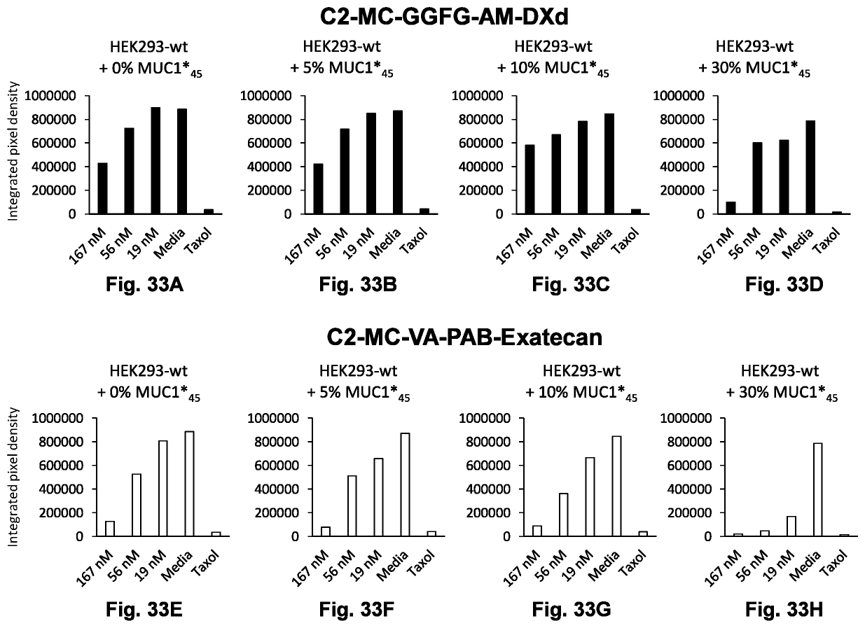

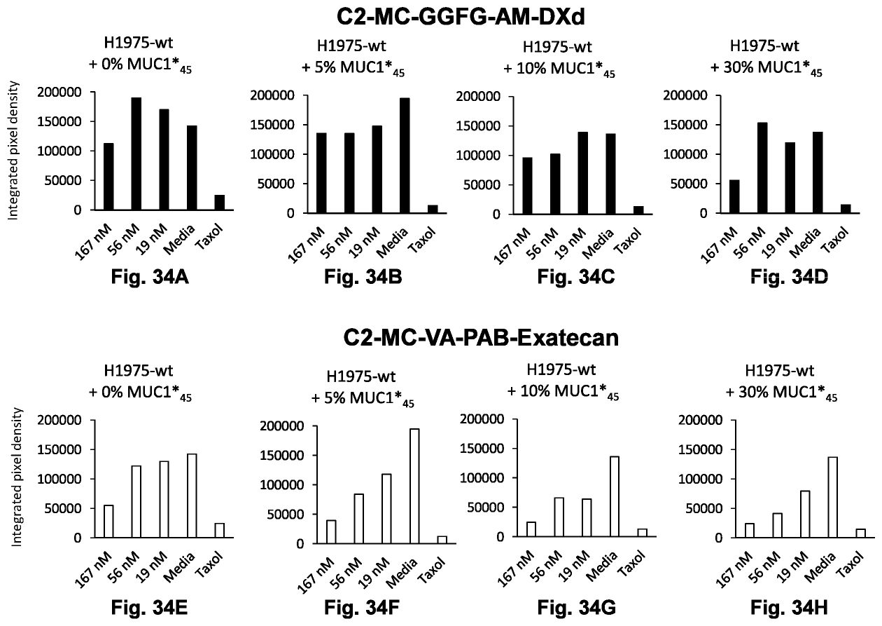

In another experiment, we investigated the killing of off-target cells (MUC1* negative HEK-293-TN cells) as well as low antigen expressing target cells (H1975 lung cancer) due to a “bystander” effect (FIGS. 32A1-32D20, FIGS. 33A-33H and FIGS. 34A-34H). To assess the killing of non-target cells adjacent to cells with high MUC1* expression, we doped non-target cells expressing an mCherry marker with varying percentages of target cells that express GFP. 56 nM C2-MC-VA-PAB-exatecan killed nearly all the neighboring non-target cells when the cell population was doped with 30% of the high antigen expressing target cells (FIG. 32C17). In comparison, a smaller percentage of the non-target cells were killed under the same conditions, but where the MUC1* ADC was C2-MC-GGFG-AM-DXd (FIG. 32A17). Furthermore, the killing of neighboring non-target cells is proportional to the concentration of H1975-MUC1*₄₅ target cells, which internalize the ADC and then release the toxin into the local non-target cell population (FIGS. 32A-32H). A limited amount of non-specific cytotoxicity was observed when pure populations of non-target HEK-293-TN cells were treated with C2-MC-GGFG-AM-DXd or C2-MC-VA-PAB-exatecan. At 167 nM and 56 nM MUC1*-ADC, C2-MC-VA-PAB-exatecan killed more HEK-293-TN non-target cells than C2-MC-GGFG-AM-DXd (compare FIG. 32A1 to FIG. 32C1).

These results are consistent with the idea that exatecan is a more potent toxin or has a higher bystander killing effect than Deruxtecan. The primary amine nitrogen of the free exatecan toxin is positively charged at times and neutral at times, which facilitates exatecan exiting the cell after cleavage from linker and also facilitates entry into neighboring cells. In contrast, DXd is neutral so doesn't as easily traverse the cell membrane to exit the target cell.

Although the killing of non-target cells could cause unwanted side effects, the killing of neighboring tumor cells via release of the cleaved toxin is desirable as it affords more complete killing of the tumor and, more importantly, it enables the killing of low antigen expressing cells that are often not sufficiently bound by antibody to elicit internalization of the ADC. We investigated the killing of low MUC1* expressing, mCherry H1975 lung cancer cells via bystander killing effects of C2-MC-VA-PAB-exatecan or C2-MC-GGFG-AM-DXd. At 56 nM, about 75% of the low MUC1* expressing cancer cells were killed by C2-MC-VA-PAB-exatecan when the cell population contained 30% high MUC1* expressing H1975-MUC1*-GFP cells (FIG. 32D17), compared to only about 25% when treated with C2-MC-GGFG-AM-DXd (FIG. 32B17). The killing of neighboring low MUC1* expressing target cells is also proportional to the concentration of high MUC1*₄₅ expressing target cells, although there is a steeper concentration dependence when the MUC1*-ADC is a MUC1* antibody conjugated to MC-VA-PAB-exatecan compared to a MUC1* antibody conjugated to MC-GGFG-AM-DXd (FIG. 34).

Table 12 shows the H scores of various untreated tumors, excised from mice and analyzed by IHC. The H score of T47D-wt breast cancers is 10, compared to 285, out of a possible 300 for T47D-MUC1*₄₅. H1975-wt lung cancer tumors have an H score of 40 and are also difficult to treat. However, if as few as 5%, 10% or 30% of the cells in the tumor express high levels of MUC1*, then the whole tumor could be killed by MUC1* ADCs with bystander killing. Tumors with H scores lower than 100 are difficult to effectively treat using antibody-targeted therapies. Therefore, treatment of tumors comprising a significant number of low MUC1* expressing cells, such as tumors having a low H score, including but not limited to those shown in Table 12, could be more effectively treated by a MUC1* antibody-MC-VA-PAB-exatecan than by a MUC1* antibody-MC-GGFG-AM-DXd.

TABLE 12
MUC1* expression in untreated tumors stained with C2

| Ex vivo tumors | Tissue | H-score |
|---|---|---|
| T47D-wt | Breast | 10 |
| T47D-MUC1*₄₅ 7.5% | | 30 |
| T47D-MUC1*₄₅ 15% | | 40 |
| T47D-MUC1*₄₅ 30% | | 80 |
| T47D-MUC1*₄₅ 100% | | 285 |
| H1975-wt | Lung | 40 |
| HPAFII-wt | Pancreatic | 220 |
| HPAFII-MUC1*₄₅ 100% | | 295 |

### Example 9. Differential Effects of C2 Versus 3C2B1 ADCs on MUC1* Cancer Cell Lines and Tumors of Different Subtypes

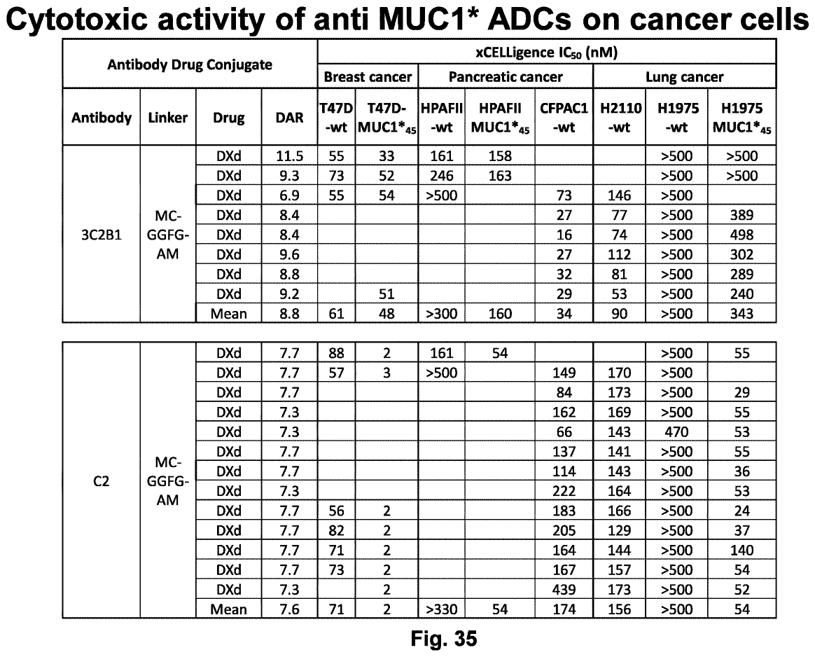

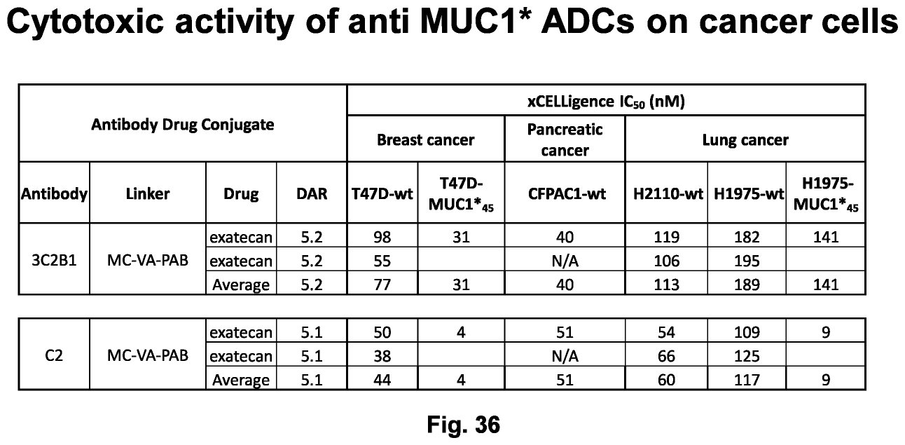

To directly compare the C2 and 3C2B1anti-MUC1* antibodies as ADC targeting domains, a panel of MUC1* positive cancer cell lines representing different cancer types were treated with C2-deruxtecan versus 3C2B1-deruxtcan (FIG. 35) or C2-MC-VA-PAB-exatecan versus 3C2B1-MC-VA-PAB-exatecan (FIG. 36). In some cases, the cancer cells were engineered to express high levels of MUC1*₄₅.

### C2-Deruxtecan Versus 3C2B1-Deruxtecan

Multiple batches were synthesized with DARs ranging from 6.9-11.5 for C2-deruxtecan and 7.3-7.7 for 3C2B1-deruxtecan.

### Breast Cancer T47D Cells

The average IC50 for 3C2B1-MC-GGFG-AM-DXd killing of breast cancer T47D-wt cells was 61 nM, where the average DAR for these experiments was 9.2. The average IC50 for C2-MC-GGFG-AM-DXd killing of breast cancer T47D-wt cells was 71 nM, where the average DAR for these experiments was 7.7. The lower IC50 for 3C2B1-MC-GGFG-AM-DXd might be due to the higher DAR for these ADCs. When the T47D breast cancer cells were engineered to express more MUC1*₄₅, then the IC50 of 3C2B1-MC-GGFG-AM-DXd was 48 nM. Surprisingly, the IC50 for C2-MC-GGFG-AM-DXd dropped to 2 nM when the cells were engineered to express MUC1*₄₅. This result is consistent with C2 being better able to recognize MUC1*₄₅ than 3C2B1.

### Pancreatic Cancer HPAFII Cells

Both C2-MC-GGFG-AM-DXd and 3C2B1-MC-GGFG-AM-DXd showed high IC50's for the killing of HPAFII-wt pancreatic cancer cells. However, the IC50 was decreased as the DAR increased.

When the HPAFII cells were engineered to express MUC1*₄₅, the average IC50 for 3C2B1-MC-GGFG-AM-DXd decreased from greater than 300 nM to 160 nM. However, the average IC50 for C2-MC-GGFG-AM-DXd dropped from greater than 330 nM to 54 nM for killing HPAFII cells engineered to express MUC1* 45.

### Pancreatic Cancer CFPAC1 Cells

In this CFPAC1-wt pancreatic cancer cell line, the IC50 for 3C2B1-MC-GGFG-AM-DXd was 34 nM on average which is significantly lower than >303 nM for HPAFII cells. Conversely, the IC50 for C2-MC-GGFG-AM-DXd was 5-fold higher than for the 3C2B1-ADC at 174 nM. This result is consistent with the results of IHC for C2 versus 3C2B1 for patient pancreatic cancer specimens, where 3C2B1 recognized pancreatic cancer tissues much better than C2.

### Lung Cancer H1975 Cells

A lung cancer cell line, H1975-wt or H1975-MUC1*₄₅, wherein the H1975 cells were transduced to express MUC1*₄₅, were tested. The average IC50 for both 3C2B1-MC-GGFG-AM-DXd and C2-MC-GGFG-AM-DXd killing of H1975-wt cells was greater than 500 nM. However, when H1975 cells were engineered to express MUC1*₄₅, then the C2-ADC IC50 dropped to 54 nM, while the 3C2B1-ADC improved slightly to 343 nM. These results are consistent with C2 recognizing MUC1*₄₅ better than 3C2B1 and implying that 3C2B1 recognizes a related but different epitope.

### Lung Cancer H2110 Cells

Another lung cancer cell line, H2110-wt, was tested for killing by 3C2B1-MC-GGFG-AM-DXd or C2-MC-GGFG-AM-DXd. Here, the average IC50 was 90 nM for 3C2B1-MC-GGFG-AM-DXd versus 156 nM for C2-MC-GGFG-AM-DXd. These results are consistent with the results of IHC for C2 versus 3C2B1 for patient lung cancer specimens, where 3C2B1 recognized a wide variety of lung cancer subtypes better than C2.

### C2-MC-VA-PAB-Exatecan Versus 3C2B1-MC-VA-PAB-Exatecan

The DARs for these conjugates were similar: 5.1 for C2-MC-VA-PAB-exatecan and 5.2 for 3C2B1-MC-VA-PAB-exatecan.

### Breast Cancer T47D Cells

The IC50 of 3C2B1-MC-VA-PAB-exatecan killing breast cancer T47D-wt cells was 77 nM and the IC50 of C2-MC-VA-PAB-exatecan was 44 nM. Recall that on the same cell line, when the toxin was DXd and the linker was MC-GGFG, the IC50s were 61 nM and 71 nM, respectively. When T47D cells were engineered to express more MUC1*₄₅, the IC50 for 3C2B1-MC-VA-PAB-exatecan was 31 nM and IC50 for C2-MC-VA-PAB-exatecan was 4 nM. On the same cell line, T47D-MUC1*₄₅, when the toxin was DXd and the linker was MC-GGFG, the IC50s were 48 nM and 2 nM, respectively. The drop in IC50 for 3C2B1 after more MUC1*₄₅ is added could indicate that higher antigen density decreases off-rate enabling greater ADC internalization.

### Pancreatic Cancer CFPAC1 Cells

The IC50 of 3C2B1-MC-VA-PAB-exatecan killing pancreatic cancer CFPAC1 cells was 40 nM and the IC50 of C2-MC-VA-PAB-exatecan was 51 nM. Recall that on the same cell line, when the toxin was DXd and the linker was MC-GGFG, the IC50s were 34 nM and 174 nM, respectively.

### Lung Cancer H2110 Cells

The IC50 of 3C2B1-MC-VA-PAB-exatecan killing lung cancer H2110-wt cells was 113 nM and the IC50 of C2-MC-VA-PAB-exatecan was 60 nM. Recall that on the same cell line, when the toxin was DXd and the linker was MC-GGFG, the IC50s were 90 nM and 156 nM, respectively.

### Lung Cancer H1975 Cells

In another lung cancer cell line, H1975, the IC50 of 3C2B1-MC-VA-PAB-exatecan was 189 nM and that of C2-MC-VA-PAB-exatecan was 117 nM. Recall that on the same cell line, the IC50 of both 3C2B1-GGFG-DXd and C2-MC-GGFG-AM-DXd was greater than 500 nM. These results are consistent with the idea that both C2 and 3C2B1 are more effective against lung cancers when conjugated to exatecan via MC-VA-PAB linker. When H1975 cells were engineered to express MUC1*₄₅, then the IC50 of 3C2B1-MC-VA-PAB-exatecan was 141 nM and that of C2-MC-VA-PAB-exatecan was 9 nM. This result is again consistent with antibody C2 recognizing a MUC1*₄₅ better than 3C2B1, while depending on the cancer subtype, wild-type cancer cells often express a MUC1* that is recognized better by 3C2B1 than C2.

Treating Human Cancers with C2 and 3C2B1 ADCs

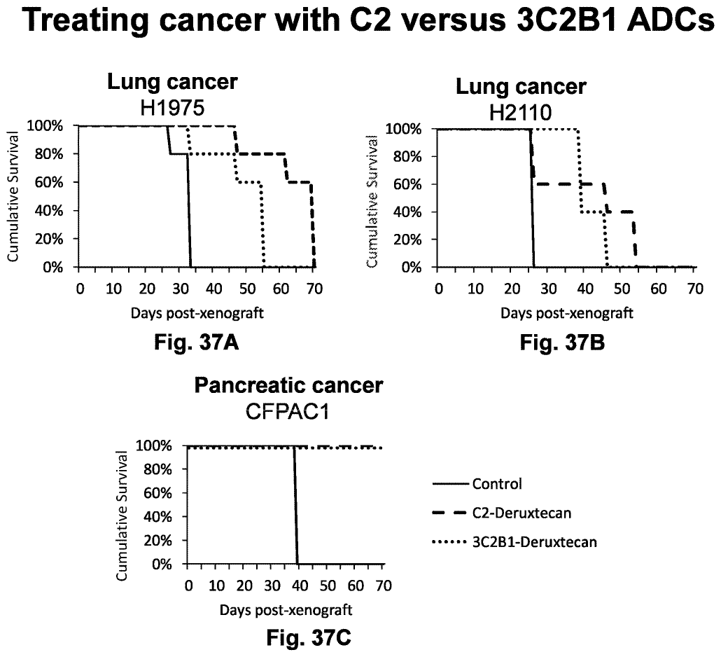

The efficacy of C2-deruxtecan and 3C2B1-deruxtecan were compared in mouse models of human cancers. Kaplan-Meier survival curves comparing efficacy on H1975 lung cancer (FIG. 37A), H2110 lung cancer (FIG. 37B), and CFPAC pancreatic cancer (FIG. 37C) demonstrate that both ADCs have in vivo efficacy and suggest that 3C2B1 ADCs may be preferred for treating lung cancers.

1.-33. (canceled)

34. An antibody drug complex (ADC) comprising an antibody or antigen-binding fragment thereof conjugated via a linker to a means for killing a cell,
wherein the antibody or antigen-binding fragment thereof binds to a peptide consisting of SEQ ID NO: 157 and to a peptide consisting of SEQ ID NO: 159,
wherein the antibody or antigen-binding fragment thereof comprises a binding region comprising six complementarity determining regions, heavy chain (HC) CDR1, HC CDR2, HC CDR3, light chain (LC) CDR1, LC CDR2, and LC CDR3, and wherein:
HC CDR1 comprises SEQ ID NO: 11;
HC CDR2 comprises SEQ ID NO: 12;
HC CDR3 comprises SEQ ID NO: 13;
LC CDR1 comprises SEQ ID NO: 14;
LC CDR2 comprises SEQ ID NO: 15; and
LC CDR3 comprises SEQ ID NO: 16; or
HC CDR1 comprises SEQ ID NO: 49;
HC CDR2 comprises SEQ ID NO: 50;
HC CDR3 comprises SEQ ID NO: 51;
LC CDR1 comprises SEQ ID NO: 52;
LC CDR2 comprises SEQ ID NO: 53; and
LC CDR3 comprises SEQ ID NO: 54.

35. The ADC of claim 34, wherein the antibody or antigen-binding fragment thereof comprises:
a HC variable domain having at least 80% sequence identity to SEQ ID NO: 9, and
a LC variable domain having at least 80% sequence identity to SEQ ID NO: 10.

36. The ADC of claim 34, wherein the antibody or antigen-binding fragment thereof comprises:
a HC variable domain having at least 80% sequence identity to SEQ ID NO: 47, and
a LC variable domain having at least 80% sequence identity to SEQ ID NO: 48.

37. The ADC of claim 34, wherein the means for killing a cell comprises a toxin and the linker and toxin covalently bound to a thiol sulphur(S) of the antibody or antigen-binding fragment thereof have the following structure:

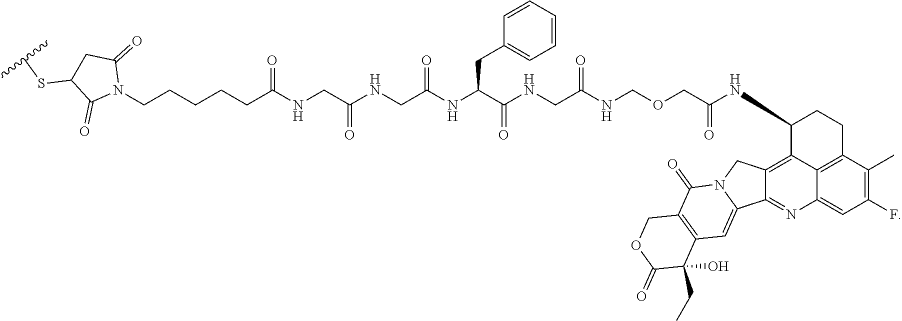 [SMILES: *SC1CC(=O)N(CCCCCC(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)C1=O]

38. The ADC of claim 37, wherein the drug to the antibody or antigen-binding fragment thereof ratio (DAR) of the ADC is an integer selected from 6-12.

39. The ADC of claim 34, wherein the means for killing a cell comprises a toxin and the toxin is an auristatin.

40. The ADC of claim 39, wherein the auristatin is monomethyl auristatin E (MMAE) or monomethyl auristatin F (MMAF).

41. The ADC of claim 34, wherein the means for killing a cell comprises a toxin and the toxin comprises a camptothecin derivative.

42. The ADC of claim 41, wherein the camptothecin derivative comprises any one of exatecan, GAB-exatecan, DXd, or SN38.

43. The ADC of claim 34, wherein the linker comprises a cleavable linker.

44. The ADC of claim 43, wherein the linker is cleavable by a cathepsin or a glucuronidase.

45. The ADC of claim 34, wherein the linker is selected from the group consisting of:
succinimidocaproyl-valine-citroline para-aminobenzyloxycarbonyl (SC-VC-PAB);
succinimidocaproyl-valine-alanine para-aminobenzyloxycarbonyl (SC-VA-PAB);
succinimidocaproyl-glycine-glycine-phenylalanine-glycine-aminomethyl (SC-GGFG-AM);
succinimidocaproyl-gly cine-glycine-glycine-glycine-aminomethyl MC-GGGG-AM);
succinimidocaproyl-glycine-glycine-valine-alanine-aminomethyl (SC-GGVA-AM);
succinimidocaproyl-glycine-glycine-valine glycine-aminomethyl (SC-GGVG-AM);
succinimidocaproyl-valine-alanine-aminomethyl (SC-VA-AM);
succinimidocaproyl-valine-alanine (SC-VA);
succinimidocaproyl-glycine-glycine-valine-alanine (SC-GGVA); and
succinimidocaproyl-(SC-MAC-glucuronide).

46. The ADC of claim 34, wherein the means for killing a cell comprises a toxin and the linker and toxin together covalently bound to a thiol sulphur(S) of the antibody or antigen-binding fragment thereof have a structure selected from the group consisting of:

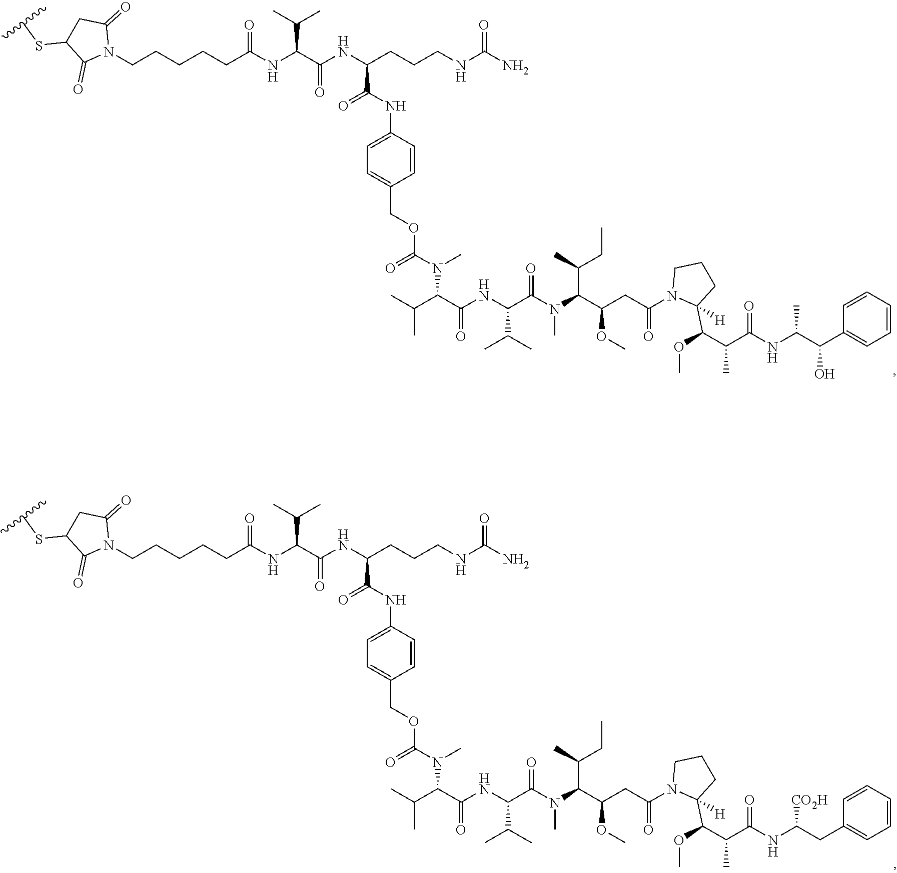

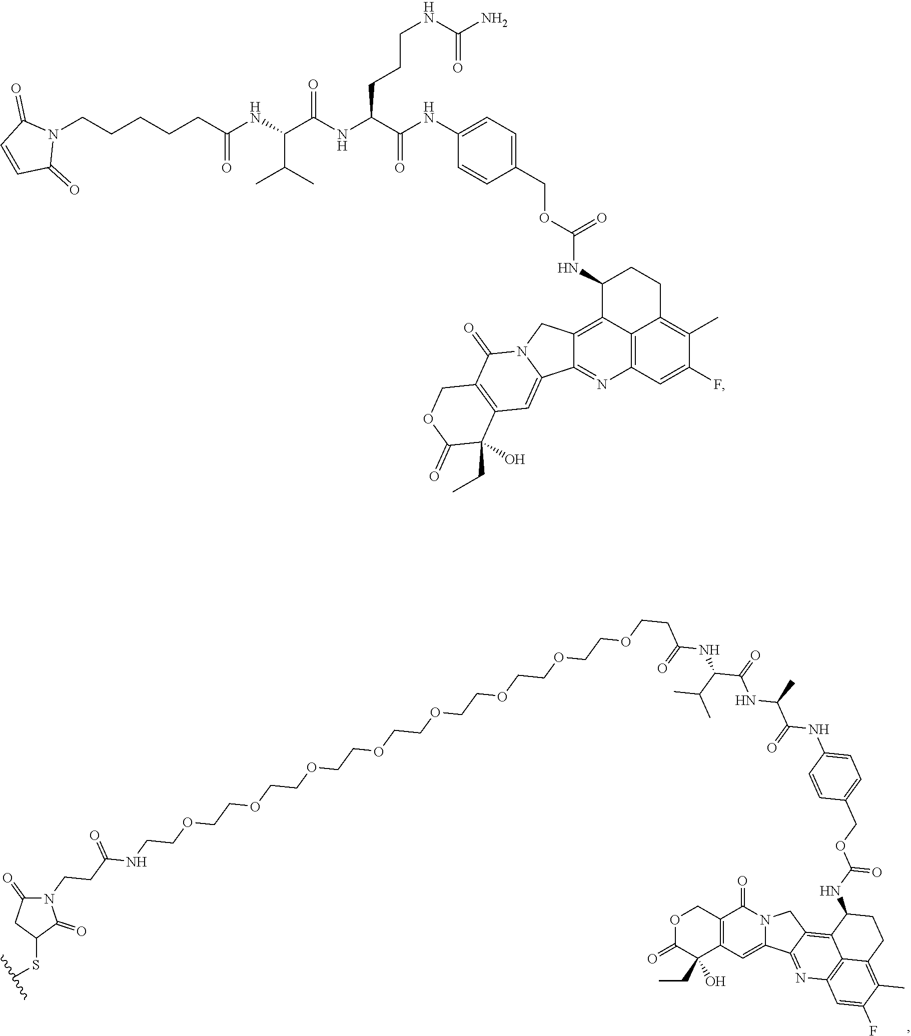

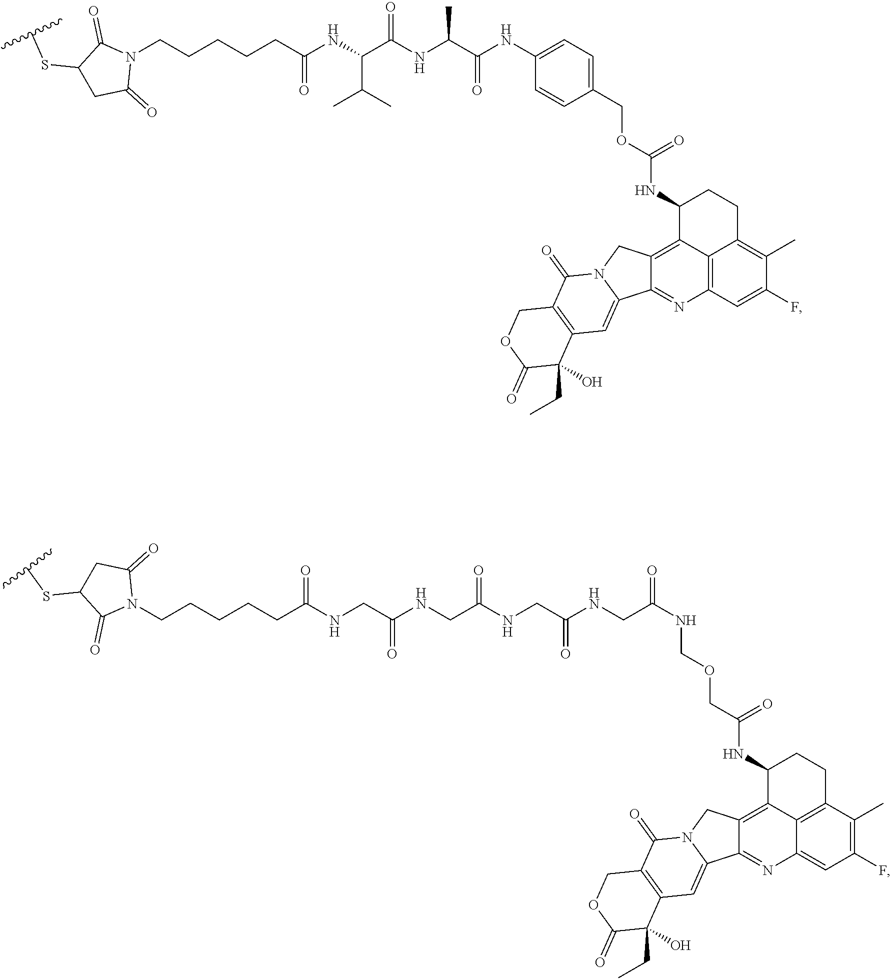

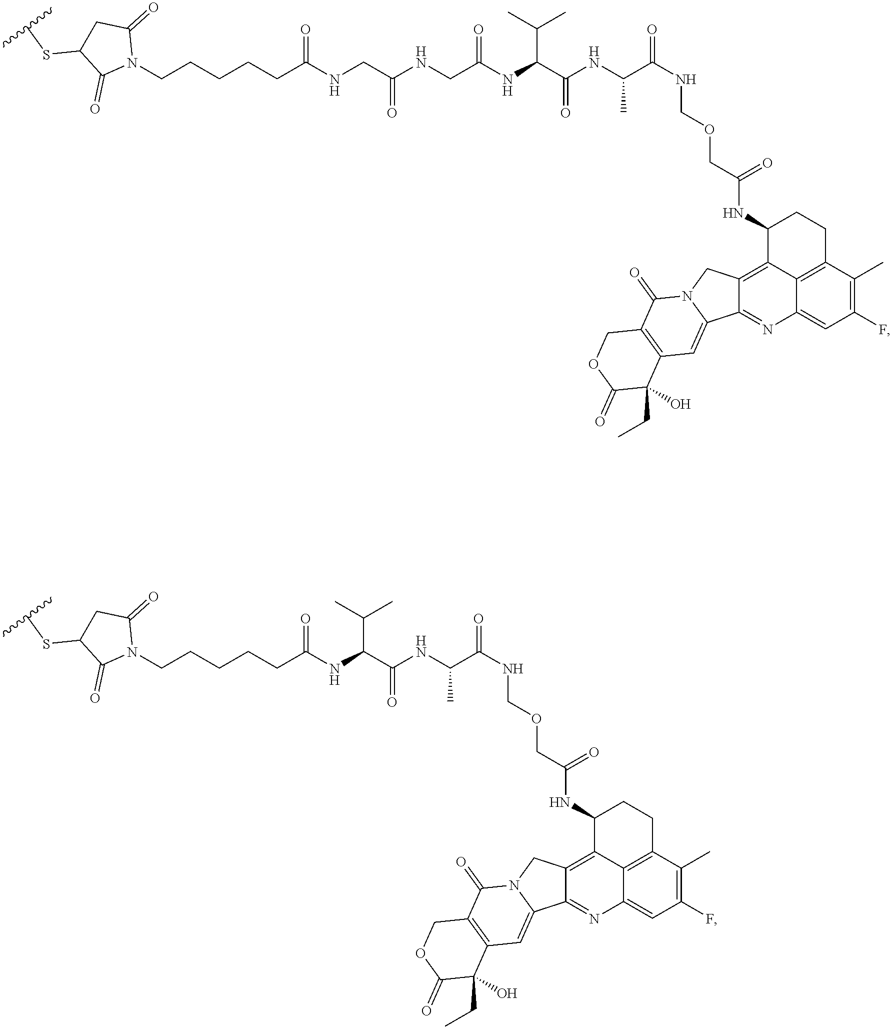

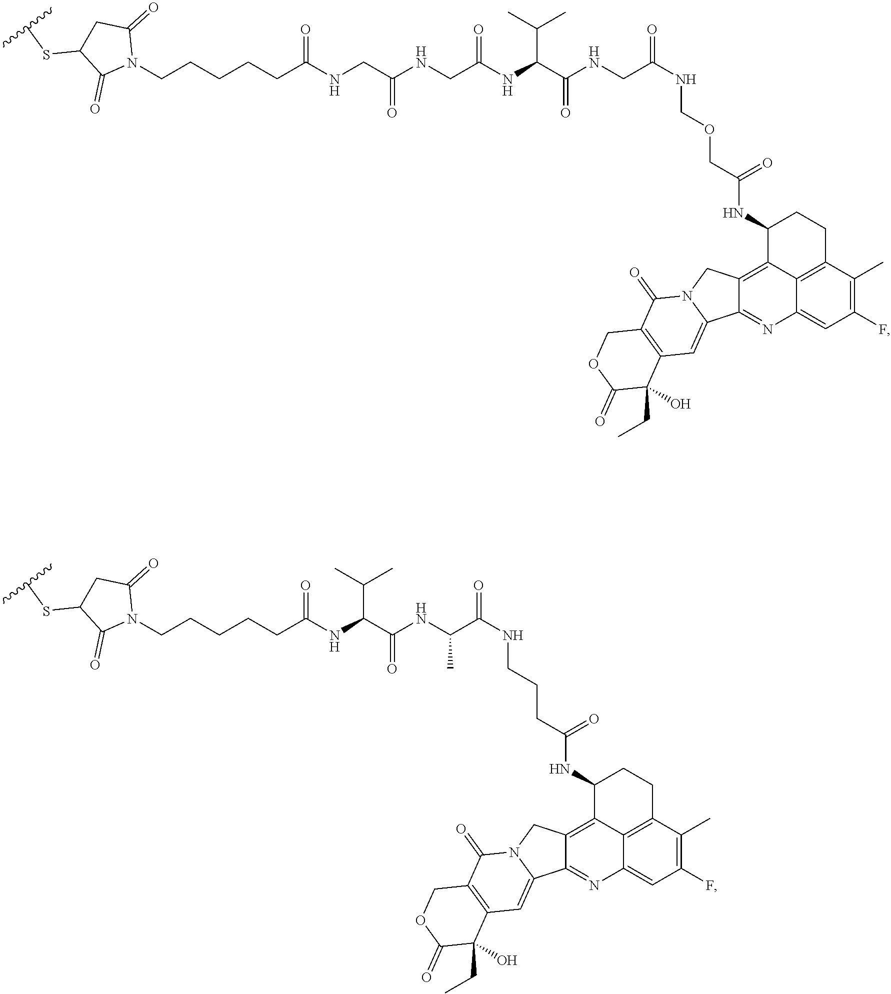

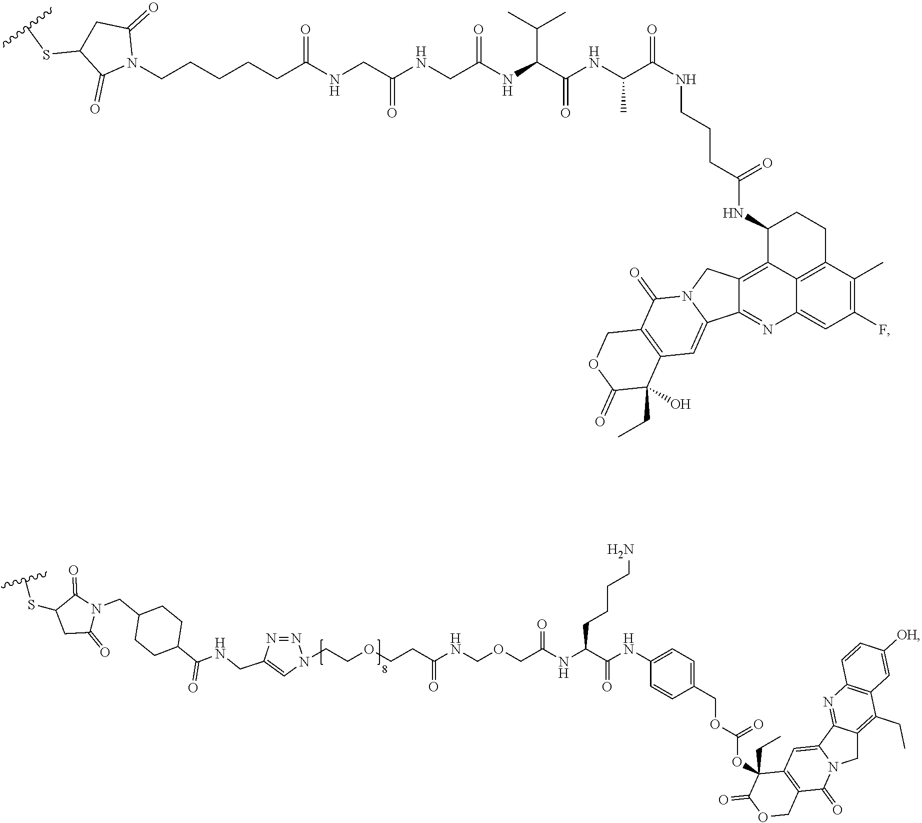

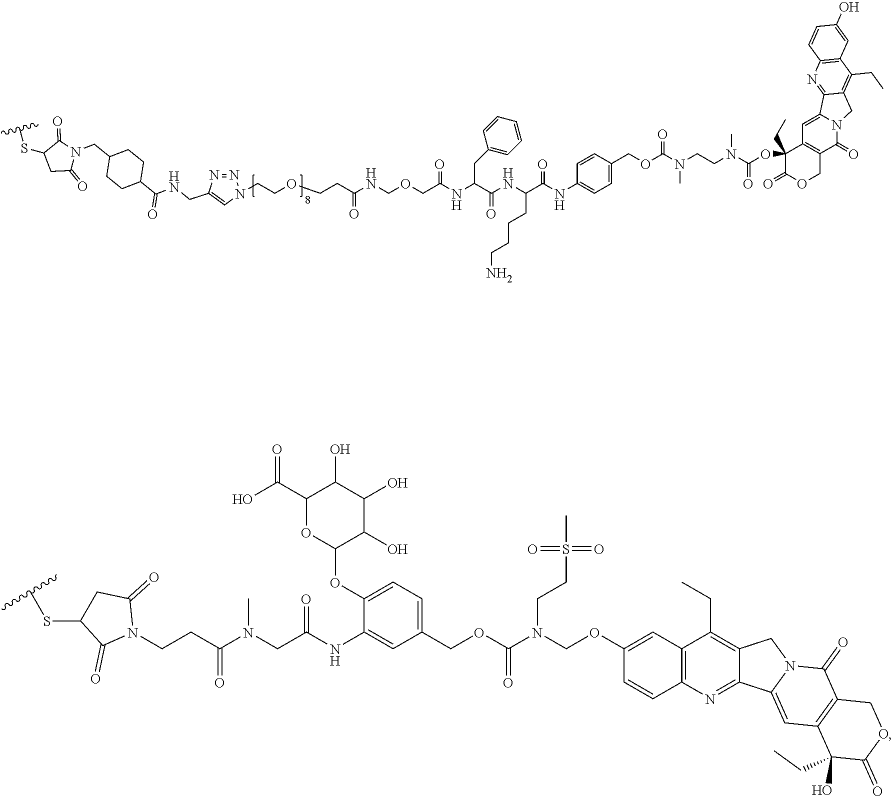

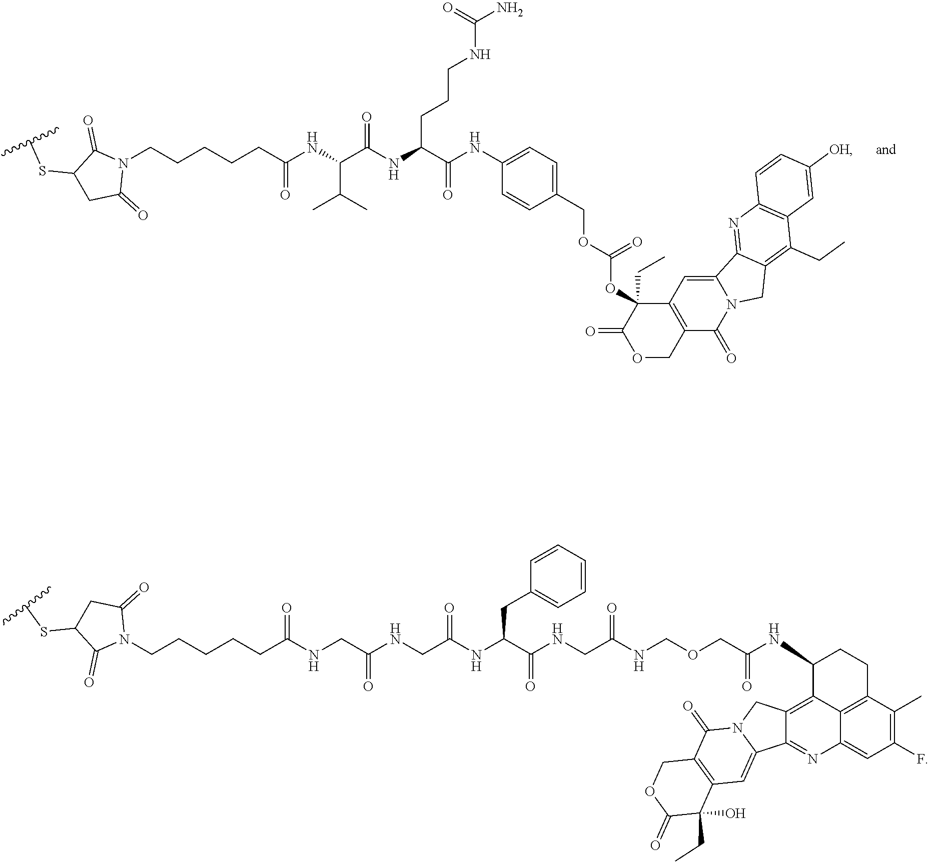

47. A method of treating a MUC1* positive cancer in a subject comprising administering the ADC of claim 34 to the subject.

48. The method of claim 47, wherein the antibody or antigen-binding fragment thereof of the ADC comprises:
HC CDR1 comprising SEQ ID NO: 11;
HC CDR2 comprising SEQ ID NO: 12;
HC CDR3 comprising SEQ ID NO: 13;
LC CDR1 comprising SEQ ID NO: 14;
LC CDR2 comprising SEQ ID NO: 15; and
LC CDR3 comprising SEQ ID NO: 16; and
a tissue section from the MUC1* positive cancer has an H-score of at least 150 when stained with an antibody comprising:
HC CDR1 comprising SEQ ID NO: 11;
HC CDR2 comprising SEQ ID NO: 12;
HC CDR3 comprising SEQ ID NO: 13;
LC CDR1 comprising SEQ ID NO: 14;
LC CDR2 comprising SEQ ID NO: 15; and
LC CDR3 comprising SEQ ID NO: 16.

49. The method of claim 47, wherein the antibody or antigen-binding fragment thereof of the ADC comprises:
HC CDR1 comprising SEQ ID NO: 49;
HC CDR2 comprising SEQ ID NO: 50;
HC CDR3 comprising SEQ ID NO: 51;
LC CDR1 comprising SEQ ID NO: 52;
LC CDR2 comprising SEQ ID NO: 53; and
LC CDR3 comprising SEQ ID NO: 54; and
a tissue section from the MUC1* positive cancer has an H-score of at least 150 when stained with an antibody comprising:
HC CDR1 comprising SEQ ID NO: 49;
HC CDR2 comprising SEQ ID NO: 50;
HC CDR3 comprising SEQ ID NO: 51;
LC CDR1 comprising SEQ ID NO: 52;
LC CDR2 comprising SEQ ID NO: 53; and
LC CDR3 comprising SEQ ID NO: 54.

50. The method of claim 47, wherein the antibody or antigen-binding fragment thereof of the ADC comprises:
HC CDR1 comprising SEQ ID NO: 11;
HC CDR2 comprising SEQ ID NO: 12;
HC CDR3 comprising SEQ ID NO: 13;
LC CDR1 comprising SEQ ID NO: 14;
LC CDR2 comprising SEQ ID NO: 15; and
LC CDR3 comprising SEQ ID NO: 16; and
a tissue section from the MUC1* positive cancer has an H-score of less than 150 when stained with an antibody comprising:
HC CDR1 comprising SEQ ID NO: 49;
HC CDR2 comprising SEQ ID NO: 50;
HC CDR3 comprising SEQ ID NO: 51;
LC CDR1 comprising SEQ ID NO: 52;
LC CDR2 comprising SEQ ID NO: 53; and
LC CDR3 comprising SEQ ID NO: 54.

51. The method of claim 47, wherein the antibody or antigen-binding fragment thereof of the ADC comprises:
HC CDR1 comprising SEQ ID NO: 49;
HC CDR2 comprising SEQ ID NO: 50;
HC CDR3 comprising SEQ ID NO: 51;
LC CDR1 comprising SEQ ID NO: 52;
LC CDR2 comprising SEQ ID NO: 53; and
LC CDR3 comprising SEQ ID NO: 54; and
a tissue section from the MUC1* positive cancer has an H-score of less than 150 when stained with an antibody comprising:
HC CDR1 comprising SEQ ID NO: 11;
HC CDR2 comprising SEQ ID NO: 12;
HC CDR3 comprising SEQ ID NO: 13;
LC CDR1 comprising SEQ ID NO: 14;
LC CDR2 comprising SEQ ID NO: 15; and
LC CDR3 comprising SEQ ID NO: 16.
